SEQUENCE LISTING (574 sequences)

SEQ ID NO: 1 - DNA, 18 bp, Escherichia coli
taatacgactcactatag

SEQ ID NO: 2 - DNA, 53 bp, Escherichia coli
taaccaggctttacactttatgcttccggctcgtataatgtgtggaaggatcc

SEQ ID NO: 3 - DNA, 47 bp, Escherichia coli
taaccaggctttacactttatgcttccggctcgtataatgtgtggaa

SEQ ID NO: 4 - DNA, 53 bp, Escherichia coli
taaaattcaaaaatttatttgctttcaggaaaatttttctgtataatagattc

SEQ ID NO: 5 - DNA, 32 bp, Escherichia coli
taattgatactttatgcttttttctgtataat

SEQ ID NO: 6 - DNA, 100 bp, Escherichia coli
aagctttcagtcgcgtaatgcttaggcacaggattgatttgtcgcaatgattgacacgattccgcttgacactgcgtaagttttgtgttataatggatcc

SEQ ID NO: 7 - DNA, 100 bp, Escherichia coli
aagcttaaggagagacaacttaaagagacttaaaagattaatttaaaatttatcaaaaagagtattgacttaaagtctaacctataggatacttggatcc

SEQ ID NO: 8 - DNA, 77 bp, Escherichia coli
aagctttgtgtggaattgtgagcggataacaattccacacattgacactttatgcttccggctcgtataatggatcc

SEQ ID NO: 9 - DNA, 75 bp, Escherichia coli
aagcttggaaaattttttttaaaaaagtcatgtgtggaattgtgagcggataacaattccacatataatggatcc

SEQ ID NO: 10 - DNA, 1285 bp, Escherichia coli
gacttcatatacccaagctttaaaaaaaaaatccttagctttcgctaaggatctccgtcaagccgtcaattgtctgattcgttaccaattatgacaacttgacggctacatcattcactttttcttcacaaccggcacgaaactcgctcgggctggccccggtgcattttttaaatactcgcgagaaatagagttgatcgtcaaaaccaacattgcgaccgacggtggcgataggcatccgggtagtgctcaaaagcagcttcgcctgactaatgcgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaacccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcagacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcgttaaacgagtatcccggcagcaggggatcattttgcgcttcagccatacttttcatactcccaccattcagagaagaaaccaattgtccatattgcatcagacattgccgtcactgcgtcttttactggctcttctcgctaacccaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtagatctatctgcgat

SEQ ID NO: 11 - DNA, 59 bp, Escherichia coli
taaaattcaaaaatttatttgctttcaggaaaatttttctgtataatagattcggatcc

SEQ ID NO: 12 - DNA, 38 bp, Escherichia coli
taattgatactttatgcttttttctgtataatggatcc

SEQ ID NO: 13 - DNA, 79 bp, Escherichia coli
gacttcatatacccaagcttggaaaattttttttaaaaaagtcttgacactttatgcttccggctcgtataatggatcc

SEQ ID NO: 14 - DNA, 23 bp, Escherichia coli
ggaaaattttttttaaaaaagtc

SEQ ID NO: 15 - DNA, 47 bp, Escherichia coli
tagcataaccccttggggcctctaaacgggtcttgaggggttttttg

SEQ ID NO: 16 - DNA, 49 bp, Escherichia coli
ttgtcacgtgagcggataacaatttcacacaggaaacagaattcttaat

SEQ ID NO: 17 - DNA, 43 bp, Escherichia coli
ttgtcacaaaccccgccaccggcggggtttttttctgcttaat

SEQ ID NO: 18 - DNA, 65 bp, Escherichia coli
ttgtcacaattctatggtgtatgcatttatttgcatacattcaatcaattggatcctgcattaat

SEQ ID NO: 19 - DNA, 42 bp, Escherichia coli
gtgagcggataacaatttcacacaggaaacagaattcttaat

SEQ ID NO: 20 - DNA, 36 bp, Escherichia coli
aaaccccgccaccggcggggtttttttctgcttaat

SEQ ID NO: 21 - DNA, 58 bp, Escherichia coli
aattctatggtgtatgcatttatttgcatacattcaatcaattggatcctgcattaat

SEQ ID NO: 22 - skipped

SEQ ID NO: 23 - DNA, 86 bp, Artificial sequence
  Synthetic amber suppressor gene sequence selection marker
aattcggggctatagctcagctgggagagcgcttgcatctaatgcaagaggtcagcggttcgatcccgcttagctccaccactgca

SEQ ID NO: 24 - DNA, 83 bp, Artificial sequence
  Synthetic amber suppressor sequence selection marker
aattcgcccggatagctcagtcggtagagcaggggattctaaatccccgtgtccttggttcgattccgagtccgggcactgca

SEQ ID NO: 25 - DNA, 876 bp, Artificial sequence
  Synthetic Rho- lgt with double amber mutation (lgt am-am allele of lgt gene) sequence selection marker
atgaccagtagctatctgcattagccggagtaggatccggtcattttctcaataggacccgtggcgcttcactggtacggcctgatgtatctggtgggtttcatttttgcaatgtggctggcaacacgacgggcgaatcgtccgggcagcggctggaccaaaaatgaagttgaaaacttactctatgcgggcttcctcggcgtcttcctcgggggacgtattggttatgttctgttctacaatttcccgcagtttatggccgatccgctgtatctgttccgtgtctgggacggcggcatgtctttccacggcggcctgattggcgttatcgtggtgatgattatcttcgcccgccgtactaaacgttccttcttccaggtctctgattttatcgcaccactcattccgtttggtcttggtgccgggcgtctgggcaactttattaacggtgaattgtggggccgcgttgacccgaacttcccgtttgccatgctgttccctggctcccgtacagaagatattttgctgctgcaaaccaacccgcagtggcaatccattttcgacacttacggtgtgctgccgcgccacccatcacagctttacgagctgctgctggaaggtgtggtgctgtttattatcctcaacctgtatattcgtaaaccacgcccaatgggagctgtctcaggtttgttcctgattggttacggcgcgtttcgcatcattgttgagtttttccgccagcccgacgcgcagtttaccggtgcctgggtgcagtacatcagcatggggcaaattctttccatcccgatgattgtcgcgggtgtgatcatgatggtctgggcatatcgtcgcagcccacagcaacacgtttcctga

SEQ ID NO: 26 - DNA, 1260 bp, Artificial sequence
  Synthetic murA with double amber mutation (murA am-am allele of murA gene) sequence selection marker
atggataaatttcgtgttcaggggccaacgaagctccagggcgaagtcacaatttccggcgctaaaaattagtagctgcctatcctttttgccgcactactggcggaagaaccggtagagatccagaacgtcccgaaactgaaagacgtcgatacatcaatgaagctgctaagccagctgggtgcgaaagtagaacgtaatggttctgtgcatattgatgcccgcgacgttaatgtattctgcgcaccttacgatctggttaaaaccatgcgtgcttctatctgggcgctggggccgctggtagcgcgctttggtcaggggcaagtttcactacctggcggttgtacgatcggtgcgcgtccggttgatctacacatttctggcctcgaacaattaggcgcgaccatcaaactggaagaaggttacgttaaagcttccgtcgatggtcgtttgaaaggtgcacatatcgtgatggataaagtcagcgttggcgcaacggtgaccatcatgtgtgctgcaaccctggcggaaggcaccacgattattgaaaacgcagcgcgtgaaccggaaatcgtcgataccgcgaacttcctgattacgctgggtgcgaaaattagcggtcagggcaccgatcgtatcgtcatcgaaggtgtggaacgtttaggcggcggtgtctatcgcgttctgccggatcgtatcgaaaccggtactttcctggtggcggcggcgatttctcgcggcaaaattatctgccgtaacgcgcagccagatactctcgacgccgtgctggcgaaactgcgtgacgctggagcggacatcgaagtcggcgaagactggattagcctggatatgcatggcaaacgtccgaaggctgttaacgtacgtaccgcgccgcatccggcattcccgaccgatatgcaggcccagttcacgctgttgaacctggtggcagaagggaccgggtttatcaccgaaacggtctttgaaaaccgctttatgcatgtgccagagctgagccgtatgggcgcgcacgccgaaatcgaaagcaataccgttatttgtcacggtgttgaaaaactttctggcgcacaggttatggcaaccgatctgcgtgcatcagcaagcctggtgctggctggctgtattgcggaagggacgacggtggttgatcgtatttatcacatcgatcgtggctacgaacgcattgaagacaaactgcgcgctttaggtgcaaatattgagcgtgtgaaaggcgaataa

SEQ ID NO: 27 - DNA, 1297 bp, Artificial sequence
  Synthetic dapA sequence selection marker
gccaggcgactgtcttcaatattacagccgcaactactgacatgacgggtgatggtgttcacaattccagggcgatcggcacccaacgcagtgatcaccagataatgttgcgatgacagtgtcaaactggttattcctttaaggggtgagttgttcttaaggaaagcataaaaaaaacatgcatacaacaatcagaacggttctgtctgcttgcttttaatgccataccaaacgtaccattgagacacttgtttgcacagaggatggcccatgttcacgggaagtattgtcgcgattgttactccgatggatgaaaaaggtaatgtctgtcgggctagcttgaaaaaactgattgattatcatgtcgccagcggtacttcggcgatcgtttctgttggcaccactggcgagtccgctaccttaaatcatgacgaacatgctgatgtggtgatgatgacgctggatctggctgatgggcgcattccggtaattgccgggaccggcgctaacgctactgcggaagccattagcctgacgcagcgcttcaatgacagtggtatcgtcggctgcctgacggtaaccccttactacaatcgtccgtcgcaagaaggtttgtatcagcatttcaaagccatcgctgagcatactgacctgccgcaaattctgtataatgtgccgtcccgtactggctgcgatctgctcccggaaacggtgggccgtctggcgaaagtaaaaaatattatcggaatcaaagaggcaacagggaacttaacgcgtgtaaaccagatcaaagagctggtttcagatgattttgttctgctgagcggcgatgatgcgagcgcgctggacttcatgcaattgggcggtcatggggttatttccgttacggctaacgtcgcagcgcgtgatatggcccagatgtgcaaactggcagcagaagggcattttgccgaggcacgcgttattaatcagcgtctgatgccattacacaacaaactatttgtcgaacccaatccaatcccggtgaaatgggcatgtaaggaactgggtcttgtggcgaccgatacgctgcgcctgccaatgacaccaatcaccgacagtggtcgtgagacggtcagagcggcgcttaagcatgccggtttgctgtaaagtttagggagatttgatggcttactctgttcaaaagtcgcgcctggcaaaggttgcgggtgtttcgcttgttttattactcgctgcctgtagttctgactcacgctataagcgtcaggtcagtggtgatgaagcctacctggaagcg

SEQ ID NO: 28 - DNA, 99 bp, Artificial sequence
  Synthetic lipoprotein promoter sequence
catggcgccgcttctttgagcgaacgatcaaaaataagtggcgccccatcaaaaaaatattctcaacataaaaaactttgtgtaatacttgtaacgctg

SEQ ID NO: 29 - DNA, 63 bp, Artificial sequence
  Synthetic lipoprotein promoter sequence
catggcgccccatcaaaaaaatattctcaacataaaaaactttgtgtaatacttgtaacgctg

SEQ ID NO: 30 - DNA, 32 bp, Artificial sequence
  Synthetic rrnC terminator sequence
gatccttagcgaaagctaaggattttttttac

SEQ ID NO: 31 - DNA, 32 bp, Artificial sequence
  Synthetic rrnC terminator sequence
gatccttagcgaaagctaaggatttttttttt

SEQ ID NO: 32 - DNA, 720 bp, Artificial sequence
  Synthetic OmpR regulator sequence
atgcaagagaactacaagattctggtggtcgatgacgacatgcgcctgcgtgcgctgctggaacgttatctcaccgaacaaggcttccaggttcgaagcgtcgctaatgcagaacagatggatcgcctgctgactcgtgaatctttccatcttatggtactggatttaatgttacctggtgaagatggcttgtcgatttgccgacgtcttcgtagtcagagcaacccgatgccgatcattatggtgacggcgaaaggggaagaagtggaccgtatcgtaggcctggagattggcgctgacgactacattccaaaaccgtttaacccgcgtgaactgctggcccgtatccgtgcggtgctgcgtcgtcaggcgaacgaactgccaggcgcaccgtcacaggaagaggcggtaattgctttcggtaagttcaaacttaacctcggtacgcgcgaaatgttccgcgaagacgagccgatgccgctcaccagcggtgagtttgcggtactgaaggcactggtcagccatccgcgtgagccgctctcccgcgataagctgatgaaccttgcccgtggtcgtgaatattccgcaatggaacgctccatcgacgtgcagatttcgcgtctgcgccgcatggtggaagaagatccagcgcatccgcgttacattcagaccgtctggggtctgggctacgtctttgtaccggacggctctaaagcatga

SEQ ID NO: 33 - DNA, 672 bp, Artificial sequence
  Synthetic PhoP regulator sequence
atgcgcgtactggttgttgaagacaatgcgttgttacgtcaccaccttaaagttcagattcaggatgctggtcatcaggtcgatgacgcagaagatgccaaagaagccgattattatctcaatgaacatataccggatattgcgattgtcgatctcggattgccagacgaggacggtctgtcactgattcgccgctggcgtagcaacgatgtttcactgccgattctggtattaaccgcccgtgaaagctggcaggacaaagtcgaagtattaagtgccggtgctgatgattatgtgactaaaccgtttcatattgaagaggtgatggcgcgaatgcaggcattaatgcggcgtaatagcggtctggcttcacaggtcatttcgctccccccgtttcaggttgatctctctcgccgtgaattatctattaatgacgaagtgatcaaactgaccgcgttcgaatacactattatggaaacgttgatacgcaataatggcaaagtggtcagcaaagattcgttaatgctccaactctatccggatgcggagctgcgggaaagccataccattgatgtactgatgggacgtctgcgcaaaaaaattcaggcacaatatccccaagaagtgattaccaccgttcgcggccagggctatctgttcgaattgcgctga

SEQ ID NO: 34 - DNA, 390 bp, Artificial sequence
  Synthetic ompF promoter sequence
gatcatcctgttacggaatattacattgcaacatttacgcgcaaaaactaatccgcattcttattgcggattagttttttcttagctaatagcacaattttcatactattttttggcattctggatgtctgaaagaagattttgtgccaggtcgataaagtttccatcagaaacaaaatttccgtttagttaatttaaatataaggaaatcatataaatagattaaaattgctgtaaatatcatcacgtctctatggaaatatgacggtgttcacaaagttccttaaattttacttttggttacatattttttctttttgaaaccaaatctttatctttgtagcactttcacggtagcgaaacgttagtttgaatggaaagatgcctgca

SEQ ID NO: 35 - DNA, 198 bp, Artificial sequence
  Synthetic ompC promoter sequence
tttaaaaaagttccgtaaaattcatattttgaaacatctatgtagataactgtaacatcttaaaagttttagtatcatattcgtgttggattattctgtatttttgcggagaatggacttgccgactggttaatgagggttaaccagtaagcagtggcataaaaaagcaataaaggcatataacagagggttaataac

SEQ ID NO: 36 - DNA, 200 bp, Artificial sequence
  Synthetic fadB promoter sequence
agtgattccattttttacccttctgtttttttgaccttaagtctccgcatcttagcacatcgttcatccagagcgtgatttctgccgagcgtgatcagatcggcatttctttaatcttttgtttgcatatttttaacacaaaatacacacttcgactcatctggtacgaccagatcaccttgcggattcaggagactgac

SEQ ID NO: 37 - DNA, 200 bp, Artificial sequence
  Synthetic phoPQ promoter sequence
gagctatcacgatggttgatgagctgaaataaacctcgtatcagtgccggatggcgatgctgtccggcctgcttattaagattatccgctttttattttttcactttacctcccctccccgctggtttatttaatgtttacccccataaccacataatcgcgttacactattttaataattaagacagggagaaataaaa

SEQ ID NO: 38 - DNA, 238 bp, Artificial sequence
  Synthetic mgtA promoter sequence
gcttcaacacgctcgcgggtgagctggctcacgccgctttcgttattcagcacccgggaaactgtagatttccccacgccgcttaagcgcgcgatatctttgatggtcagccgattttgcatcctgttgtcctgtaacgtgttgtttaattatttgagcctaacgttacccgtgcattcagcaatgggtaaagtctggtttatcgttggtttagttgtcagcaggtattatatcgcca

SEQ ID NO: 39 - DNA, 73 bp, Artificial sequence
  Synthetic Ptrp promoter sequence
gagctgttgacaattaatcatcgaactagttaactagtacgcaagttcacgtaaaaagggtatctagaattct

SEQ ID NO: 40 - DNA, 1353 bp, Artificial sequence
  Synthetic EnvZ sensor sequence
atgaggcgattgcgcttctcgccacgaagttcatttgcccgtacgttattgctcatcgtcaccttgctgttcgccagcctggtgacgacttatctggtggtgctgaacttcgcgattttgccgagcctccagcagtttaataaagtcctcgcgtacgaagtgcgtatgttgatgaccgacaaactgcaactggaggacggcacgcagttggttgtgcctcccgctttccgtcgggagatctaccgtgagctggggatctctctctactccaacgaggctgccgaagaggcaggtctgcgttgggcgcaacactatgaattcttaagccatcagatggcgcagcaactgggcggcccgacggaagtgcgcgttgaggtcaacaaaagttcgcctgtcgtctggctgaaaacctggctgtcgcccaatatctgggtacgcgtgccgctgaccgaaattcatcagggcgatttctctccgctgttccgctatacgctggcgattatgctattggcgataggcggggcgtggctgtttattcgtatccagaaccgaccgttggtcgatctcgaacacgcagccttgcaggttggtaaagggattattccgccgccgctgcgtgagtatggcgcttcggaggtgcgttccgttacccgtgcctttaaccatatggcggctggtgttaagcaactggcggatgaccgcacgctgctgatggcgggggtaagtcacgacttgcgcacgccgctgacgcgtattcgcctggcgactgagatgatgagcgagcaggatggctatctggcagaatcgatcaataaagatatcgaagagtgcaacgccatcattgagcagtttatcgactacctgcgcaccgggcaggagatgccgatggaaatggcggatcttaatgcagtactcggtgaggtgattgctgccgaaagtggctatgagcgggaaattgaaaccgcgctttaccccggcagcattgaagtgaaaatgcacccgctgtcgatcaaacgcgcggtggcgaatatggtggtcaacgccgcccgttatggcaatggctggatcaaagtcagcagcggaacggagccgaatcgcgcctggttccaggtggaagatgacggtccgggaattgcgccggaacaacgtaagcacctgttccagccgtttgtccgcggcgacagtgcgcgcaccattagcggcacgggattagggctggcaattgtgcagcgtatcgtggataaccataacgggatgctggagcttggcaccagcgagcggggcgggctttccattcgcgcctggctgccagtgccggtaacgcgggcgcagggcacgacaaaagaagggtaa

SEQ ID NO: 41 - DNA, 1461 bp, Artificial sequence
  Synthetic PhoQ sensor sequence
atgaaaaaattactgcgtctttttttcccgctctcgctgcgggtacgttttctgttggcaacggcagcggtagtactggtgctttcgcttgcctacggaatggtcgcgctgatcggttatagcgtcagtttcgataaaactacgtttcggctgttacgtggcgagagcaatctgttctatacccttgcgaagtgggaaaacaataagttgcatgtcgagttacccgaaaatatcgacaagcaaagccccaccatgacgctaatttatgatgagaacgggcagcttttatgggcgcaacgtgacgtgccctggctgatgaagatgatccagcctgactggctgaaatcgaatggttttcatgaaattgaagcggatgttaacgataccagcctcttgctgagtggagatcattcgatacagcaacagttgcaggaagtgcgggaagatgatgacgacgcggagatgacccactcggtggcagtaaacgtctacccggcaacatcgcggatgccaaaattaaccattgtggtggtggataccattccggtggagctaaaaagttcctatatggtctggagctggtttatctatgtgctctcagccaatctgctgttagtgatcccgctgctgtgggtcgccgcctggtggagtttacgccccatcgaagccctggcaaaagaagtccgcgaactggaagaacataaccgcgaattgctcaatccagccacaacgcgagaactgaccagtctggtacgaaacctgaaccgattgttaaaaagtgaacgcgaacgttacgacaaataccgtacgacgctcaccgacctgacccatagtctgaaaacgccactggcggtgctgcaaagtacgctgcgttctctgcgtagtgaaaagatgagcgtcagtgatgctgagccggtaatgctggagcaaatcagccgcatttcacagcaaattggctactacctgcatcgtgccagtatgcgcggcgggacattgctcagccgcgagctgcatccggtcgccccactgctggacaatctcacctcagcgctgaacaaagtgtatcaacgcaaaggggtcaatatctctctcgatatttcgccagagatcagctttgtcggtgagcagaacgattttgtcgaggtgatgggcaacgtgctggataatgcctgtaaatattgcctcgagtttgtcgaaatttctgcaaggcaaaccgacgagcatctctatattgtggtcgaggatgatggccccggtattccattaagcaagcgagaggtcattttcgaccgtggtcaacgggttgatactttacgccctgggcaaggtgtagggctggcggtagcccgcgaaatcaccgagcaatatgagggtaaaatcgtcgccggagagagcatgctgggcggtgcgcggatggaggtgatttttggtcgccagcattctgcgccgaaagatgaataa

SEQ ID NO: 42 - DNA, 490 bp, Artificial sequence
  Synthetic alpha-defensin-1 protein
ctatagaagacctgggacagaggactgctgtctgccctctctggtcaccctgcctagctagaggatctgtgaccccagccatgaggaccctcgccatccttgctgccattctcctggtggccctgcaggcccaggctgagccactccaggcaagagctgatgaggttgctgcagccccggagcagattgcagcggacatcccagaagtggttgtttcccttgcatgggacgaaagcttggctccaaagcatccaggctcaaggaaaaacatggcctgctattgcagaataccagcgtgcattgcaggagaacgtcgctatggaacctgcatctaccagggaagactctgggcattctgctgctgagcttgcagaaaaagaaaaatgagctcaaaatttgctttgagagctacagggaattgctattactcctgtaccttctgctcaatttcctttcctcatcccaaataaatgccttggtacaagaaaag

SEQ ID NO: 43 - DNA, 487 bp, Artificial sequence
  Synthetic alpha-defensin-3 protein
ccttgctatagaagacctgggacagaggactgctgtctgccctctctggtcaccctgcctagctagaggatctgtgaccccagccatgaggaccctcgccatccttgctgccattctcctggtggccctgcaggcccaggctgagccactccaggcaagagctgatgaggttgctgcagccccggagcagattgcagcggacatcccagaagtggttgtttcccttgcatgggacgaaagcttggctccaaagcatccaggctcaaggaaaaacatggactgctattgcagaataccagcgtgcattgcaggagaacgtcgctatggaacctgcatctaccagggaagactctgggcattctgctgctgagcttgcagaaaaagaaaaatgagctcaaaatttgctttgagagctacagggaattgctattactcctgtaccttctgctcaatttcctttcctcatctcaaataaatgccttgttac

SEQ ID NO: 44 - DNA, 542 bp, Artificial sequence
  Synthetic alpha-defensin-4 protein
gtctgccctctctgctcgccctgcctagcttgaggatctgtcaccccagccatgaggattatcgccctcctcgctgctattctcttggtagccctccaggtccgggcaggcccactccaggcaagaggtgatgaggctccaggccaggagcagcgtgggccagaagaccaggacatatctatttcctttgcatgggataaaagctctgctcttcaggtttcaggctcaacaaggggcatggtctgctcttgcagattagtattctgccggcgaacagaacttcgtgttgggaactgcctcattggtggtgtgagtttcacatactgctgcacgcgtgtcgattaacgttctgctgtccaagagaatgtcatgctgggaacgccatcatcggtggtgttagcttcacatgcttctgcagctgagcttgcagaatagagaaaaatgagctcataatttgctttgagagctacaggaaatggttgtttctcctatactttgtccttaacatctttcttgatcctaaatatatatctcgtaacaag

SEQ ID NO: 45 - DNA, 449 bp, Artificial sequence
  alpha-defensin-5 protein
atatccactcctgctctccctcctgcaggtgaccccagccatgaggaccatcgccatccttgctgccattctcctggtggccctgcaggcccaggctgagtcactccaggaaagagctgatgaggctacaacccagaagcagtctggggaagacaaccaggaccttgctatctcctttgcaggaaatggactctctgctcttagaacctcaggttctcaggcaagagccacctgctattgccgaaccggccgttgtgctacccgtgagtccctctccggggtgtgtgaaatcagtggccgcctctacagactctgctgtcgctgagcttcctagatagaaaccaaagcagtgcaagattcagttcaaggtcctgaaaaaagaaaaacattttactctgtgtaccttgtgtctttctaaatttctctctccaaaataaagttcaagcatt

SEQ ID NO: 46 - DNA, 475 bp, Artificial sequence
  alpha-defensin-6 protein
acacatctgctcctgctctctctcctccagcgaccctagccatgagaaccctcaccatcctcactgctgttctcctcgtggccctccaggccaaggctgagccactccaagctgaggatgatccactgcaggcaaaagcttatgaggctgatgcccaggagcagcgtggggcaaatgaccaggactttgccgtctcctttgcagaggatgcaagctcaagtcttagagctttgggctcaacaagggctttcacttgccattgcagaaggtcctgttattcaacagaatattcctatgggacctgcactgtcatgggtattaaccacagattctgctgcctctgagggatgagaacagagagaaatatattcataatttactttatgacctagaaggaaactgtcgtgtgtcccatacattgccatcaactttgtttcctcatctcaaataaagtcctttcagcaaaaaaaaaaaa

SEQ ID NO: 47 - DNA, 484 bp, Artificial sequence
  Synthetic beta-defensin-1 protein
tcccttcagttccgtcgacgaggttgtgcaatccaccagtcttataaatacagtgacgctccagcctctggaagcctctgtcagctcagcctccaaaggagccagcgtctccccagttcctgaaatcctgggtgttgcctgccagtcgccatgagaacttcctaccttctgctgtttactctctgcttacttttgtctgagatggcctcaggtggtaactttctcacaggccttggccacagatctgatcattacaattgcgtcagcagtggagggcaatgtctctattctgcctgcccgatctttaccaaaattcaaggcacctgttacagagggaaggccaagtgctgcaagtgagctgggagtgaccagaagaaatgacgcagaagtgaaatgaactttttataagcattcttttaataaaggaaaattgcttttgaagtatacctcctttgggccaaaaaaaaaaaaaaaaaaaaaaaaa

SEQ ID NO: 48 - DNA, 337 bp, Artificial sequence
  Synthetic beta-defensin-3 protein
tgagtctcagcgtggggtgaagcctagcagctatgaggatccattatcttctgtttgctttgctcttcctgtttttggtgcctgtcccaggtcatggaggaatcataaacacattacagaaatattattgcagagtcagaggcggccggtgtgctgtgctcagctgccttccaaaggaggaacagatcggcaagtgctcgacgcgtggccgaaaatgctgccgaagaaagaaataaaaaccctgaaacatgacgagagtgttgtaaagtgtggaaatgccttcttaaagtttataaaagtaaaatcaaattacatttttttttcaaaaaaaaaaaaa

SEQ ID NO: 49 - DNA, 336 bp, Artificial sequence
  Synthetic beta-defensin-4 protein
agactcagctcctggtgaagctcccagccatcagccatgagggtcttgtatctcctcttctcgttcctcttcatattcctgatgcctcttccaggtgtttttggtggtataggcgatcctgttacctgccttaagagtggagccatatgtcatccagtcttttgccctagaaggtataaacaaattggcacctgtggtctccctggaacaaaatgctgcaaaaagccatgaggaggccaagaagctgctgtggctgatgcggattcagaaagggctccctcatcagagacgtgcgacatgtaaaccaaattaaactatggtgtccaaagatacgca

SEQ ID NO: 50 - DNA, 691 bp, Artificial sequence
  Synthetic protegrin-1 protein
atggagacccagagagccagcctgtgcctggggcgctggtcactgtggcttctgctgctggcactcgtggtgccctcggccagcgcccaggccctcagctacagggaggccgtgcttcgtgctgtggatcgcctcaacgagcagtcctcggaagctaatctctaccgcctcctggagctggaccagccgcccaaggccgacgaggacccgggcaccccgaaacctgtgagcttcacggtgaaggagactgtgtgtcccaggccgacccggcagcccccggagctgtgtgacttcaaggagaacgggcgggtgaaacagtgtgtggggacagtcaccctggatcagatcaaggacccgctcgacatcacctgcaatgaggttcaaggtgtcaggggaggtcgcctgtgctattgtaggcgtaggttctgcgtctgtgtcggacgaggatgacggttgcgacggcaggctttccctcccccaattttcccggggccaggtttccgtcccccaatttttccgcctccacctttccggcccgcaccattcggtccaccaaggttccctggtagacggtgaaggatttgcaggcaactcacccagaaggcctttcggtacattaaaatcccagcaaggagacctaagcatctgctttgcccaggcccgcatctgtcaaataaattcttgtgaaacc

SEQ ID NO: 51 - DNA, 691 bp, Artificial sequence
  Synthetic protegrin-3 protein
atggagacccagagagccagcctgtgcctggggcgctggtcactgtggcttctgctgctggcactcgtggtgccctcggccagcgcccaggccctcagctacagggaggccgtgcttcgtgctgtggatcgcctcaacgagcagtcctcggaagctaatctctaccgcctcctggagctggaccagccgcccaaggccgacgaggacccgggcaccccgaaacctgtgagcttcacggtgaaggagactgtgtgtcccaggccgacccggcagcccccggagctgtgtgacttcaaggagaacgggcgggtgaaacagtgtgtggggacagtcaccctggatcagatcaaggacccgctcgacatcacctgcaatgaggttcaaggtgtcaggggaggtggcctgtgctattgtaggcgtaggttctgcgtctgtgtcggacgaggatgacggttgcgacggcaggctttccctcccccaattttcccggggccaggtttccgtcccccaatttttccgcctccacctttccggcccgcaccattcggtccaccaaggttccctggtagacggtgaaggatttgcaggcaactcacccagaaggcctttcggtacattaaaatcccagcaaggagacctaagcatctgctttgcccaggcccgcatctgtcaaataaattcttgtgaaacc

SEQ ID NO: 52 - DNA, 691 bp, Artificial sequence
  Synthetic protegrin-4 protein
atggagacccagagagccagcctgtgcctggggcgctggtcactgtggcttctgctgctggcactcgtggtgccctcggccagcgcccaggccctcagctacagggaggccgtgcttcgtgctgtggatcgcctcaacgagcagtcctcggaagctaatctctaccgcctcctggagctggaccagccgcccaaggccgacgaggacccgggcaccccgaaacctgtgagcttcacggtgaaggagactgtgtgtcccaggccgacccggcagcccccggagctgtgtgacttcaaggagaacgggcgggtgaaacagtgtgtggggacagtcaccctggatcagatcaaggacccgctcgacatcacctgcaatgaggttcaaggtgtcaggggaggtcgcctgtgctattgtaggggttggatctgcttctgtgtcggacgaggatgacggttgcgacggcaggctttccctcccccaattttcccggggccaggtttccgtcccccaatttttccgcctccacctttccggcccgcaccattcggtccaccaaggttccctggtagacggtgaaggatttgcaggcaactcacccagaaggcctttcggcacattaaaatcccagcaaggagacctaagcatctgctttgcccaggcccgcatctgtcaaataaattcttgtgaaacc

SEQ ID NO: 53 - DNA, 55 bp, Artificial sequence
  DNA insert
gatcctaggtatttgaatttgcatttcaagagaatgcaaattcaaataccttttg

SEQ ID NO: 54 - DNA, 55 bp, Artificial sequence
  DNA insert
gatccataaacttaaacgtaaagttctcttacgtttaagtttatggaaaacagct

SEQ ID NO: 55 - DNA, 21 bp, Artificial sequence
  Beta-catenin target gene sequence
agccaatggcttggaatgaga

SEQ ID NO: 56 - DNA, 21 bp, Artificial sequence
  Beta-catenin target gene sequence
atcagctggcctggtttgata

SEQ ID NO: 57 - DNA, 21 bp, Artificial sequence
  Beta-catenin target gene sequence
ctgtgaacttgctcaggacaa

SEQ ID NO: 58 - DNA, 21 bp, Artificial sequence
  Beta-catenin target gene sequence
agcaatcagctggcctggttt

SEQ ID NO: 59 - DNA, 21 bp, Artificial sequence
  Beta-catenin target gene sequence
cctctgtgaacttgctcagga

SEQ ID NO: 60 - DNA, 21 bp, Artificial sequence
  Beta-catenin target gene sequence
ttccgaatgtctgaggacaag

SEQ ID NO: 61 - DNA, 21 bp, Artificial sequence
  Beta-catenin target gene sequence
ccaatggcttggaatgagact

SEQ ID NO: 62 - DNA, 21 bp, Artificial sequence
  Beta-catenin target gene sequence
ggtgctgactatccagttgat

SEQ ID NO: 63 - DNA, 21 bp, Artificial sequence
  Beta-catenin target gene sequence
caatcagctggcctggtttga

SEQ ID NO: 64 - DNA, 21 bp, Artificial sequence
  Beta-catenin target gene sequence
caccctggtgctgactatcca

SEQ ID NO: 65 - DNA, 21 bp, Artificial sequence
  Beta-catenin target gene sequence
caccaccctggtgctgactat

SEQ ID NO: 66 - DNA, 21 bp, Artificial sequence
  Beta-catenin target gene sequence
tgctttattctcccattgaaa

SEQ ID NO: 67 - DNA, 21 bp, Artificial sequence
  Beta-catenin target gene sequence
ctggtgctgactatccagttg

SEQ ID NO: 68 - DNA, 21 bp, Artificial sequence
  Beta-catenin target gene sequence
tctgtgctcttcgtcatctga

SEQ ID NO: 69 - DNA, 21 bp, Artificial sequence
  Beta-catenin target gene sequence
tgccatctgtgctcttcgtca

SEQ ID NO: 70 - DNA, 21 bp, Artificial sequence
  Beta-catenin target gene sequence
tggtgctgactatccagttga

SEQ ID NO: 71 - DNA, 21 bp, Artificial sequence
  Beta-catenin target gene sequence
cctggtgctgactatccagtt

SEQ ID NO: 72 - DNA, 21 bp, Artificial sequence
  Beta-catenin target gene sequence
accctggtgctgactatccag

SEQ ID NO: 73 - DNA, 21 bp, Artificial sequence
  Beta-catenin target gene sequence
gagcctgccatctgtgctctt

SEQ ID NO: 74 - DNA, 21 bp, Artificial sequence
  Beta-catenin target gene sequence
ctggtttgatactgacctgta

SEQ ID NO: 75 - DNA, 21 bp, Artificial sequence
  Beta-catenin target gene sequence
tggtttgatactgacctgtaa

SEQ ID NO: 76 - DNA, 21 bp, Artificial sequence
  Beta-catenin target gene sequence
tcgaggagtaacaatacaaat

SEQ ID NO: 77 - DNA, 21 bp, Artificial sequence
  Beta-catenin target gene sequence
accatgcagaatacaaatgat

SEQ ID NO: 78 - DNA, 21 bp, Artificial sequence
  Beta-catenin target gene sequence
aggagtaacaatacaaatgga

SEQ ID NO: 79 - DNA, 21 bp, Artificial sequence
  Beta-catenin target gene sequence
gtcgaggagtaacaatacaaa

SEQ ID NO: 80 - DNA, 21 bp, Artificial sequence
  Beta-catenin target gene sequence
ttgttgtaacctgctgtgata

SEQ ID NO: 81 - DNA, 21 bp, Artificial sequence
  Beta-catenin target gene sequence
gagtaatggtgtagaacacta

SEQ ID NO: 82 - DNA, 21 bp, Artificial sequence
  Beta-catenin target gene sequence
agtaatggtgtagaacactaa

SEQ ID NO: 83 - DNA, 21 bp, Artificial sequence
  Beta-catenin target gene sequence
cacactaaccaagctgagttt

SEQ ID NO: 84 - DNA, 21 bp, Artificial sequence
  Beta-catenin target gene sequence
tttggtcgaggagtaacaata

SEQ ID NO: 85 - DNA, 21 bp, Artificial sequence
  Beta-catenin target gene sequence
taccattccattgtttgtgca

SEQ ID NO: 86 - DNA, 21 bp, Artificial sequence
  Beta-catenin target gene sequence
tagggtaaatcagtaagaggt

SEQ ID NO: 87 - DNA, 21 bp, Artificial sequence
  Beta-catenin target gene sequence
ctaaccaagctgagtttccta

SEQ ID NO: 88 - DNA, 21 bp, Artificial sequence
  Beta-catenin target gene sequence
tggtcgaggagtaacaataca

SEQ ID NO: 89 - DNA, 21 bp, Artificial sequence
  Beta-catenin target gene sequence
ctggcctggtttgatactgac

SEQ ID NO: 90 - DNA, 21 bp, Artificial sequence
  Beta-catenin target gene sequence
taacctcacttgcaataatta

SEQ ID NO: 91 - DNA, 21 bp, Artificial sequence
  Beta-catenin target gene sequence
atcccactggcctctgataaa

SEQ ID NO: 92 - DNA, 21 bp, Artificial sequence
  Beta-catenin target gene sequence
gaccacaagcagagtgctgaa

SEQ ID NO: 93 - DNA, 21 bp, Artificial sequence
  Beta-catenin target gene sequence
cacaagcagagtgctgaaggt

SEQ ID NO: 94 - DNA, 21 bp, Artificial sequence
  Beta-catenin target gene sequence
ctaacctcacttgcaataatt

SEQ ID NO: 95 - DNA, 19 bp, Artificial sequence
  Beta-catenin target gene sequence
agctgatattgatggacag

SEQ ID NO: 96 - DNA, 23 bp, Artificial sequence
  Synthetic HPV target gene sequence
cggtgccagaaaccgttgaatcc

SEQ ID NO: 97 - DNA, 23 bp, Artificial sequence
  Synthetic HPV target gene sequence
cactgcaagacatagaaataacc

SEQ ID NO: 98 - DNA, 23 bp, Artificial sequence
  Synthetic HPV target gene sequence
aggtgcctgcggtgccagaaacc

SEQ ID NO: 99 - DNA, 23 bp, Artificial sequence
  Synthetic HPV target gene sequence
gcggtgccagaaaccgttgaatc

SEQ ID NO: 100 - DNA, 23 bp, Artificial sequence
  Synthetic HPV target gene sequence
tcactgcaagacatagaaataac

SEQ ID NO: 101 - DNA, 23 bp, Artificial sequence
  Synthetic HPV target gene sequence
cccatgctgcatgccataaatgt

SEQ ID NO: 102 - DNA, 23 bp, Artificial sequence
  Synthetic HPV target gene sequence
atgctgcatgccataaatgtata

SEQ ID NO: 103 - DNA, 23 bp, Artificial sequence
  Synthetic HPV target gene sequence
gtggtgtatagagacagtatacc

SEQ ID NO: 104 - DNA, 23 bp, Artificial sequence
  Synthetic HPV target gene sequence
gcgcgctttgaggatccaacacg

SEQ ID NO: 105 - DNA, 23 bp, Artificial sequence
  Synthetic HPV target gene sequence
ctgcggtgccagaaaccgttgaa

SEQ ID NO: 106 - DNA, 23 bp, Artificial sequence
  Synthetic HPV target gene sequence
ccccatgctgcatgccataaatg

SEQ ID NO: 107 - DNA, 23 bp, Artificial sequence
  Synthetic HPV target gene sequence
accccatgctgcatgccataaat

SEQ ID NO: 108 - DNA, 23 bp, Artificial sequence
  Synthetic HPV target gene sequence
aacactgggttatacaatttatt

SEQ ID NO: 109 - DNA, 23 bp, Artificial sequence
  Synthetic HPV target gene sequence
acgacgcagagaaacacaagtat

SEQ ID NO: 110 - DNA, 23 bp, Artificial sequence
  Synthetic HPV target gene sequence
aaggtgcctgcggtgccagaaac

SEQ ID NO: 111 - DNA, 23 bp, Artificial sequence
  Synthetic HPV target gene sequence
ggtgcctgcggtgccagaaaccg

SEQ ID NO: 112 - DNA, 23 bp, Artificial sequence
  Synthetic HPV target gene sequence
catgctgcatgccataaatgtat

SEQ ID NO: 113 - DNA, 23 bp, Artificial sequence
  Synthetic HPV target gene sequence
gacgcagagaaacacaagtataa

SEQ ID NO: 114 - DNA, 23 bp, Artificial sequence
  Synthetic HPV target gene sequence
ttcactgcaagacatagaaataa

SEQ ID NO: 115 - DNA, 23 bp, Artificial sequence
  Synthetic HPV target gene sequence
ggtgccagaaaccgttgaatcca

SEQ ID NO: 116 - DNA, 23 bp, Artificial sequence
  Synthetic HPV target gene sequence
tggcgcgctttgaggatccaaca

SEQ ID NO: 117 - DNA, 23 bp, Artificial sequence
  Synthetic HPV target gene sequence
tgtggtgtatagagacagtatac

SEQ ID NO: 118 - DNA, 23 bp, Artificial sequence
  Synthetic HPV target gene sequence
gtgcctgcggtgccagaaaccgt

SEQ ID NO: 119 - DNA, 23 bp, Artificial sequence
  Synthetic HPV target gene sequence
ctgcatgccataaatgtatagat

SEQ ID NO: 120 - DNA, 23 bp, Artificial sequence
  Synthetic HPV target gene sequence
gactccaacgacgcagagaaaca

SEQ ID NO: 121 - DNA, 23 bp, Artificial sequence
  Synthetic HPV target gene sequence
ctgggcactatagaggccagtgc

SEQ ID NO: 122 - DNA, 23 bp, Artificial sequence
  Synthetic HPV target gene sequence
tgctgcatgccataaatgtatag

SEQ ID NO: 123 - DNA, 23 bp, Artificial sequence
  Synthetic HPV target gene sequence
gtgccagaaaccgttgaatccag

SEQ ID NO: 124 - DNA, 23 bp, Artificial sequence
  Synthetic HPV target gene sequence
ttacagaggtatttgaatttgca

SEQ ID NO: 125 - DNA, 23 bp, Artificial sequence
  Synthetic HPV target gene sequence
gaggccagtgccattcgtgctgc

SEQ ID NO: 126 - DNA, 23 bp, Artificial sequence
  Synthetic HPV target gene sequence
attccggttgaccttctatgtca

SEQ ID NO: 127 - DNA, 23 bp, Artificial sequence
  Synthetic HPV target gene sequence
gatggagttaatcatcaacattt

SEQ ID NO: 128 - DNA, 23 bp, Artificial sequence
  Synthetic HPV target gene sequence
aagccagaattgagctagtagta

SEQ ID NO: 129 - DNA, 23 bp, Artificial sequence
  Synthetic HPV target gene sequence
catggacctaaggcaacattgca

SEQ ID NO: 130 - DNA, 23 bp, Artificial sequence
  Synthetic HPV target gene sequence
aaccacaacgtcacacaatgttg

SEQ ID NO: 131 - DNA, 23 bp, Artificial sequence
  Synthetic HPV target gene sequence
atggacctaaggcaacattgcaa

SEQ ID NO: 132 - DNA, 23 bp, Artificial sequence
  Synthetic HPV target gene sequence
taagcgactcagaggaagaaaac

SEQ ID NO: 133 - DNA, 23 bp, Artificial sequence
  Synthetic HPV target gene sequence
gaagccagaattgagctagtagt

SEQ ID NO: 134 - DNA, 23 bp, Artificial sequence
  Synthetic HPV target gene sequence
gagccgaaccacaacgtcacaca

SEQ ID NO: 135 - DNA, 23 bp, Artificial sequence
  Synthetic HPV target gene sequence
acgtcacacaatgttgtgtatgt

SEQ ID NO: 136 - DNA, 23 bp, Artificial sequence
  Synthetic HPV target gene sequence
gaaccacaacgtcacacaatgtt

SEQ ID NO: 137 - DNA, 23 bp, Artificial sequence
  Synthetic HPV target gene sequence
aggcaacattgcaagacattgta

SEQ ID NO: 138 - DNA, 23 bp, Artificial sequence
  Synthetic HPV target gene sequence
aagacattgtattgcatttagag

SEQ ID NO: 139 - DNA, 23 bp, Artificial sequence
  Synthetic HPV target gene sequence
taaggcaacattgcaagacattg

SEQ ID NO: 140 - DNA, 23 bp, Artificial sequence
  Synthetic HPV target gene sequence
ccagcccgacgagccgaaccaca

SEQ ID NO: 141 - DNA, 23 bp, Artificial sequence
  Synthetic HPV target gene sequence
aagctcagcagacgaccttcgag

SEQ ID NO: 142 - DNA, 23 bp, Artificial sequence
  Synthetic HPV target gene sequence
gcccgacgagccgaaccacaacg

SEQ ID NO: 143 - DNA, 23 bp, Artificial sequence
  Synthetic HPV target gene sequence
ttccggttgaccttctatgtcac

SEQ ID NO: 144 - DNA, 23 bp, Artificial sequence
  Synthetic HPV target gene sequence
tgcatggacctaaggcaacattg

SEQ ID NO: 145 - DNA, 23 bp, Artificial sequence
  Synthetic HPV target gene sequence
ttccagcagctgtttctgaacac

SEQ ID NO: 146 - DNA, 23 bp, Artificial sequence
  Synthetic HPV target gene sequence
aacaccctgtcctttgtgtgtcc

SEQ ID NO: 147 - DNA, 23 bp, Artificial sequence
  Synthetic HPV target gene sequence
cttctatgtcacgagcaattaag

SEQ ID NO: 148 - DNA, 23 bp, Artificial sequence
  Synthetic HPV target gene sequence
acgagccgaaccacaacgtcaca

SEQ ID NO: 149 - DNA, 23 bp, Artificial sequence
  Synthetic HPV target gene sequence
ttgagctagtagtagaaagctca

SEQ ID NO: 150 - DNA, 23 bp, Artificial sequence
  Synthetic HPV target gene sequence
cagcagacgaccttcgagcattc

SEQ ID NO: 151 - DNA, 23 bp, Artificial sequence
  Synthetic HPV target gene sequence
agccagaattgagctagtagtag

SEQ ID NO: 152 - DNA, 23 bp, Artificial sequence
  Synthetic HPV target gene sequence
gtcacacaatgttgtgtatgtgt

SEQ ID NO: 153 - DNA, 23 bp, Artificial sequence
  Synthetic HPV target gene sequence
ccgacgagccgaaccacaacgtc

SEQ ID NO: 154 - DNA, 23 bp, Artificial sequence
  Synthetic HPV target gene sequence
aattccggttgaccttctatgtc

SEQ ID NO: 155 - DNA, 23 bp, Artificial sequence
  Synthetic HPV target gene sequence
attccagcagctgtttctgaaca

SEQ ID NO: 156 - DNA, 19 bp, Artificial sequence
  Synthetic HPV target gene sequence
taggtatttgaatttgcat

SEQ ID NO: 157 - DNA, 19 bp, Artificial sequence
  Synthetic HPV target gene sequence
gaggtatttgaatttgcat

SEQ ID NO: 158 - DNA, 20 bp, Artificial sequence
  MDR-1 target gene sequence
atgttgtctggacaagcact

SEQ ID NO: 159 - DNA, 19 bp, Artificial sequence
  k-Ras target gene sequence
gttggagctgttggcgtag

SEQ ID NO: 160 - DNA, 21 bp, Artificial sequence
  IL-6R target gene sequence
ctcctggaactcatctttcta

SEQ ID NO: 161 - DNA, 21 bp, Artificial sequence
  IL-6R target gene sequence
gctctcctgcttccggaagag

SEQ ID NO: 162 - DNA, 21 bp, Artificial sequence
  IL-6R target gene sequence
ctccacgactctggaaactat

SEQ ID NO: 163 - DNA, 21 bp, Artificial sequence
  IL-6R target gene sequence
cagaagttctcctgccagtta

SEQ ID NO: 164 - DNA, 21 bp, Artificial sequence
  IL-6R target gene sequence
ccggaagacaatgccactgtt

SEQ ID NO: 165 - DNA, 21 bp, Artificial sequence
  IL-6R target gene sequence
ctgaacggtcaaagacattca

SEQ ID NO: 166 - DNA, 21 bp, Artificial sequence
  IL-6R target gene sequence
cacaacatggatggtcaagga

SEQ ID NO: 167 - DNA, 21 bp, Artificial sequence
  IL-6R target gene sequence
atgcaggcacttactactaat

SEQ ID NO: 168 - DNA, 21 bp, Artificial sequence
  IL-6R target gene sequence
atcgggctgaacggtcaaaga

SEQ ID NO: 169 - DNA, 21 bp, Artificial sequence
  IL-6R target gene sequence
agctctcctgcttccggaaga

SEQ ID NO: 170 - DNA, 21 bp, Artificial sequence
  IL-6R target gene sequence
cagctctcctgcttccggaag

SEQ ID NO: 171 - DNA, 21 bp, Artificial sequence
  IL-6R target gene sequence
caggcacttactactaataaa

SEQ ID NO: 172 - DNA, 21 bp, Artificial sequence
  IL-6R target gene sequence
cacttgctggtggatgttccc

SEQ ID NO: 173 - DNA, 21 bp, Artificial sequence
  IL-6R target gene sequence
aacggtcaaagacattcacaa

SEQ ID NO: 174 - DNA, 21 bp, Artificial sequence
  IL-6R target gene sequence
tgcacaagctgcaccctcagg

SEQ ID NO: 175 - DNA, 21 bp, Artificial sequence
  IL-6R target gene sequence
atcctggagggtgacaaagta

SEQ ID NO: 176 - DNA, 21 bp, Artificial sequence
  IL-6R target gene sequence
tgggtctgacaataccgtaaa

SEQ ID NO: 177 - DNA, 21 bp, Artificial sequence
  IL-6R target gene sequence
aacgaagcgtttcacagctta

SEQ ID NO: 178 - DNA, 21 bp, Artificial sequence
  IL-6R target gene sequence
ccgctgtttcctataacagaa

SEQ ID NO: 179 - DNA, 21 bp, Artificial sequence
  IL-6R target gene sequence
acgaagcgtttcacagcttaa

SEQ ID NO: 180 - DNA, 21 bp, Artificial sequence
  IL-6R target gene sequence
ctgctgtgaaagggaaattta

SEQ ID NO: 181 - DNA, 21 bp, Artificial sequence
  IL-6R target gene sequence
aaccttgtggtatcagccata

SEQ ID NO: 182 - DNA, 21 bp, Artificial sequence
  IL-6R target gene sequence
cacagtgtggtgcttagatta

SEQ ID NO: 183 - DNA, 21 bp, Artificial sequence
  IL-6R target gene sequence
cagcttcgataccgacctgta

SEQ ID NO: 184 - DNA, 21 bp, Artificial sequence
  IL-6R target gene sequence
cagtgtggtgcttagattaaa

SEQ ID NO: 185 - DNA, 21 bp, Artificial sequence
  IL-6R target gene sequence
cccggcaggaatcctctggaa

SEQ ID NO: 186 - DNA, 21 bp, Artificial sequence
  IL-6R target gene sequence
cccgctgtttcctataacaga

SEQ ID NO: 187 - DNA, 21 bp, Artificial sequence
  IL-6R target gene sequence
aaccacgaggatcagtacgaa

SEQ ID NO: 188 - DNA, 21 bp, Artificial sequence
  IL-6R target gene sequence
acctgccgtcttactgaacta

SEQ ID NO: 189 - DNA, 21 bp, Artificial sequence
  IL-6R target gene sequence
accacgaggatcagtacgaaa

SEQ ID NO: 190 - DNA, 21 bp, Artificial sequence
  IL-6R target gene sequence
acagcttgtgatgactgaata

SEQ ID NO: 191 - DNA, 21 bp, Artificial sequence
  IL-6R target gene sequence
aggatcagtacgaaagttcta

SEQ ID NO: 192 - DNA, 21 bp, Artificial sequence
  IL-6R target gene sequence
aacccgctgtttcctataaca

SEQ ID NO: 193 - DNA, 21 bp, Artificial sequence
  IL-6R target gene sequence
cagtacgaaagttctacagaa

SEQ ID NO: 194 - DNA, 21 bp, Artificial sequence
  IL-6R target gene sequence
tacgcgagtgacaatttctca

SEQ ID NO: 195 - DNA, 21 bp, Artificial sequence
  IL-6R target gene sequence
acgaaagttctacagaagcaa

SEQ ID NO: 196 - DNA, 21 bp, Artificial sequence
  IL-6R target gene sequence
caggcacttactactaataaa

SEQ ID NO: 197 - DNA, 21 bp, Artificial sequence
  IL-6R target gene sequence
cacttgctggtggatgttccc

SEQ ID NO: 198 - DNA, 21 bp, Artificial sequence
  IL-6R target gene sequence
aacggtcaaagacattcacaa

SEQ ID NO: 199 - DNA, 21 bp, Artificial sequence
  IL-6R target gene sequence
tgcacaagctgcaccctcagg

SEQ ID NO: 200 - DNA, 21 bp, Artificial sequence
  IL-7 target gene sequence
taagagagtcataaaccttaa

SEQ ID NO: 201 - DNA, 21 bp, Artificial sequence
  IL-7 target gene sequence
aacaaggtccaagatacctaa

SEQ ID NO: 202 - DNA, 21 bp, Artificial sequence
  IL-7 target gene sequence
aagattgaacctgcagaccaa

SEQ ID NO: 203 - DNA, 21 bp, Artificial sequence
  IL-7 target gene sequence
aagagatttcaagagatttaa

SEQ ID NO: 204 - DNA, 21 bp, Artificial sequence
  IL-7 target gene sequence
aagcgcaaagtagaaactgaa

SEQ ID NO: 205 - DNA, 21 bp, Artificial sequence
  IL-7 target gene sequence
tagcatcatctgattgtgata

SEQ ID NO: 206 - DNA, 21 bp, Artificial sequence
  IL-7 target gene sequence
taagataataatatatgttta

SEQ ID NO: 207 - DNA, 21 bp, Artificial sequence
  IL-7 target gene sequence
atggtcagcatcgatcaatta

SEQ ID NO: 208 - DNA, 21 bp, Artificial sequence
  IL-7 target gene sequence
ttgcctgaataatgaatttaa

SEQ ID NO: 209 - DNA, 21 bp, Artificial sequence
  IL-7 target gene sequence
atctgtgatgctaataaggaa

SEQ ID NO: 210 - DNA, 21 bp, Artificial sequence
  IL-7 target gene sequence
aacaaactatttcttatatat

SEQ ID NO: 211 - DNA, 21 bp, Artificial sequence
  IL-7 target gene sequence
aacatttatcaatcagtataa

SEQ ID NO: 212 - DNA, 21 bp, Artificial sequence
  IL-7 target gene sequence
atcaatcagtataattctgta

SEQ ID NO: 213 - DNA, 21 bp, Artificial sequence
  IL-7 target gene sequence
aaggtatcagttgcaataata

SEQ ID NO: 214 - DNA, 21 bp, Artificial sequence
  IL-7 target gene sequence
cggatcctacggaagttatgg

SEQ ID NO: 215 - DNA, 21 bp, Artificial sequence
  IL-7 target gene sequence
gaccatgttccatgtttcttt

SEQ ID NO: 216 - DNA, 21 bp, Artificial sequence
  IL-7 target gene sequence
aacctaaatgacctttattaa

SEQ ID NO: 217 - DNA, 21 bp, Artificial sequence
  IL-7 target gene sequence
caggagactaggaccctataa

SEQ ID NO: 218 - DNA, 21 bp, Artificial sequence
  IL-7 target gene sequence
tagggtcttattcgtatctaa

SEQ ID NO: 219 - DNA, 21 bp, Artificial sequence
  IL-7 target gene sequence
atgagccaatatgcttaatta

SEQ ID NO: 220 - DNA, 21 bp, Artificial sequence
  IL-7 target gene sequence
gccaatatgcttaattagaaa

SEQ ID NO: 221 - DNA, 21 bp, Artificial sequence
  IL-7 target gene sequence
cagcatcgatgaattggacaa

SEQ ID NO: 222 - DNA, 21 bp, Artificial sequence
  IL-7 target gene sequence
ttgcctgaataatgaatttaa

SEQ ID NO: 223 - DNA, 21 bp, Artificial sequence
  IL-7 target gene sequence
ctgatagtaattgcccgaata

SEQ ID NO: 224 - DNA, 21 bp, Artificial sequence
  IL-7 target gene sequence
aagggtttgcttgtactgaat

SEQ ID NO: 225 - DNA, 21 bp, Artificial sequence
  IL-7 target gene sequence
aacatgtatgtgatgatacaa

SEQ ID NO: 226 - DNA, 21 bp, Artificial sequence
  IL-7 target gene sequence
ttgcaacatgtaataatttaa

SEQ ID NO: 227 - DNA, 21 bp, Artificial sequence
  IL-7 target gene sequence
aagagactactgagagaaata

SEQ ID NO: 228 - DNA, 21 bp, Artificial sequence
  IL-7 target gene sequence
aagaatctactggttcatata

SEQ ID NO: 229 - DNA, 21 bp, Artificial sequence
  IL-7 target gene sequence
tgccgtcagcatatacatata

SEQ ID NO: 230 - DNA, 21 bp, Artificial sequence
  IL-7 target gene sequence
agggctcacggtgatggataa

SEQ ID NO: 231 - DNA, 21 bp, Artificial sequence
  IL-7 target gene sequence
cgcctcccgcagaccatgttc

SEQ ID NO: 232 - DNA, 21 bp, Artificial sequence
  IL-7 target gene sequence
tccgtgctgctcgcaagttga

SEQ ID NO: 233 - DNA, 21 bp, Artificial sequence
  IL-7 target gene sequence
gcctcccgcagaccatgttcc

SEQ ID NO: 234 - DNA, 21 bp, Artificial sequence
  IL-7 target gene sequence
cctcccgcagaccatgttcca

SEQ ID NO: 235 - DNA, 21 bp, Artificial sequence
  IL-7 target gene sequence
ctcccgcagaccatgttccat

SEQ ID NO: 236 - DNA, 21 bp, Artificial sequence
  IL-7 target gene sequence
tcccgcagaccatgttccatg

SEQ ID NO: 237 - DNA, 21 bp, Artificial sequence
  IL-7 target gene sequence
cccgcagaccatgttccatgt

SEQ ID NO: 238 - DNA, 21 bp, Artificial sequence
  IL-7 target gene sequence
ccgcagaccatgttccatgtt

SEQ ID NO: 239 - DNA, 21 bp, Artificial sequence
  IL-7 target gene sequence
cgcagaccatgttccatgttt

SEQ ID NO: 240 - DNA, 21 bp, Artificial sequence
  IL-7 target gene sequence
gcagaccatgttccatgtttc

SEQ ID NO: 241 - DNA, 21 bp, Artificial sequence
  IL-7 target gene sequence
cagaccatgttccatgtttct

SEQ ID NO: 242 - DNA, 21 bp, Artificial sequence
  IL-7 target gene sequence
agaccatgttccatgtttctt

SEQ ID NO: 243 - DNA, 21 bp, Artificial sequence
  IL-13Ra-1 target gene sequence
aacctgatcctccacatatta

SEQ ID NO: 244 - DNA, 21 bp, Artificial sequence
  IL-13Ra-1 target gene sequence
cctgatcctccacatattaaa

SEQ ID NO: 245 - DNA, 21 bp, Artificial sequence
  IL-13Ra-1 target gene sequence
agaaatgtttggagaccagaa

SEQ ID NO: 246 - DNA, 21 bp, Artificial sequence
  IL-13Ra-1 target gene sequence
caaataatggtcaaggataat

SEQ ID NO: 247 - DNA, 21 bp, Artificial sequence
  IL-13Ra-1 target gene sequence
ttcctgatcctggcaagattt

SEQ ID NO: 248 - DNA, 21 bp, Artificial sequence
  IL-13Ra-1 target gene sequence
taaagaaatgtttggagacca

SEQ ID NO: 249 - DNA, 21 bp, Artificial sequence
  IL-13Ra-1 target gene sequence
atgtttggagaccagaatgat

SEQ ID NO: 250 - DNA, 21 bp, Artificial sequence
  IL-13Ra-1 target gene sequence
ctccaattcctgatcctggca

SEQ ID NO: 251 - DNA, 21 bp, Artificial sequence
  IL-13Ra-1 target gene sequence
caagaagactctaatgatgta

SEQ ID NO: 252 - DNA, 21 bp, Artificial sequence
  IL-13Ra-1 target gene sequence
cacagtcagagtaagagtcaa

SEQ ID NO: 253 - DNA, 21 bp, Artificial sequence
  IL-13Ra-1 target gene sequence
acccagggtatcatagttcta

SEQ ID NO: 254 - DNA, 21 bp, Artificial sequence
  IL-13Ra-1 target gene sequence
ctgctttgaaatttccagaaa

SEQ ID NO: 255 - DNA, 21 bp, Artificial sequence
  IL-13Ra-1 target gene sequence
atcatagttctaagaatgaaa

SEQ ID NO: 256 - DNA, 21 bp, Artificial sequence
  IL-13Ra-1 target gene sequence
aaggcttaagatcattatatt

SEQ ID NO: 257 - DNA, 21 bp, Artificial sequence
  IL-13Ra-1 target gene sequence
aactacttataagaaagtaaa

SEQ ID NO: 258 - DNA, 21 bp, Artificial sequence
  IL-13Ra-1 target gene sequence
cacagaacatctagcaaacaa

SEQ ID NO: 259 - DNA, 21 bp, Artificial sequence
  IL-13Ra-1 target gene sequence
ctcgttcttgttcaatcctaa

SEQ ID NO: 260 - DNA, 21 bp, Artificial sequence
  IL-13Ra-1 target gene sequence
aacttgtaggttcacatatta

SEQ ID NO: 261 - DNA, 21 bp, Artificial sequence
  IL-13Ra-1 target gene sequence
aaccatttctgcaaatttaaa

SEQ ID NO: 262 - DNA, 21 bp, Artificial sequence
  IL-13Ra-1 target gene sequence
ctcagtgtagtgccaatgaaa

SEQ ID NO: 263 - DNA, 21 bp, Artificial sequence
  IL-13Ra-1 target gene sequence
caggccttagggactcataaa

SEQ ID NO: 264 - DNA, 21 bp, Artificial sequence
  IL-13Ra-1 target gene sequence
aagtatgacatctatgagaaa

SEQ ID NO: 265 - DNA, 21 bp, Artificial sequence
  IL-13Ra-1 target gene sequence
gtggaggtcaataatactcaa

SEQ ID NO: 266 - DNA, 21 bp, Artificial sequence
  IL-13Ra-1 target gene sequence
cagagtataggtaaggagcaa

SEQ ID NO: 267 - DNA, 21 bp, Artificial sequence
  IL-18 target gene sequence
ttgaatgaccaagttctcttc

SEQ ID NO: 268 - DNA, 21 bp, Artificial sequence
  IL-18 target gene sequence
ctctctgtgaaggatagtaaa

SEQ ID NO: 269 - DNA, 21 bp, Artificial sequence
  IL-18 target gene sequence
ccgcagtaatacggaatataa

SEQ ID NO: 270 - DNA, 21 bp, Artificial sequence
  IL-18 target gene sequence
caaggaaatgatgtttattga

SEQ ID NO: 271 - DNA, 21 bp, Artificial sequence
  IL-18 target gene sequence
cagactgataatatacatgta

SEQ ID NO: 272 - DNA, 21 bp, Artificial sequence
  IL-18 target gene sequence
ttggccgacttcactgtacaa

SEQ ID NO: 273 - DNA, 21 bp, Artificial sequence
  IL-18 target gene sequence
ccagaccagactgataatata

SEQ ID NO: 274 - DNA, 21 bp, Artificial sequence
  IL-18 target gene sequence
aagatggagtttgaatcttca

SEQ ID NO: 275 - DNA, 21 bp, Artificial sequence
  IL-18 target gene sequence
acgctttactttatacctgaa

SEQ ID NO: 276 - DNA, 21 bp, Artificial sequence
  IL-18 target gene sequence
tacaaccgcagtaatacggaa

SEQ ID NO: 277 - DNA, 21 bp, Artificial sequence
  IL-18 target gene sequence
ctgcatgatttatagagtaaa

SEQ ID NO: 278 - DNA, 21 bp, Artificial sequence
  IL-18 target gene sequence
cccgaggctgcatgatttata

SEQ ID NO: 279 - DNA, 21 bp, Artificial sequence
  IL-18 target gene sequence
cacgctttactttatacctga

SEQ ID NO: 280 - DNA, 21 bp, Artificial sequence
  IL-18 target gene sequence
cgcctgtatttccataacaga

SEQ ID NO: 281 - DNA, 21 bp, Artificial sequence
  IL-18 target gene sequence
cgcagtaatacggaatataaa

SEQ ID NO: 282 - DNA, 21 bp, Artificial sequence
  IL-18 target gene sequence
tacatgtacaaagacagtgaa

SEQ ID NO: 283 - DNA, 21 bp, Artificial sequence
  IL-18 target gene sequence
caggcctgacatcttctgcaa

SEQ ID NO: 284 - DNA, 21 bp, Artificial sequence
  IL-18 target gene sequence
ttcgaggatatgactgatatt

SEQ ID NO: 285 - DNA, 21 bp, Artificial sequence
  IL-18 target gene sequence
ctgtatttccataacagaata

SEQ ID NO: 286 - DNA, 21 bp, Artificial sequence
  IL-18 target gene sequence
gaggatatgactgatattgat

SEQ ID NO: 287 - DNA, 21 bp, Artificial sequence
  IL-18 target gene sequence
caagttctcttcgttgacaaa

SEQ ID NO: 288 - DNA, 21 bp, Artificial sequence
  IL-18 target gene sequence
cactaacttacatcaaagtta

SEQ ID NO: 289 - DNA, 21 bp, Artificial sequence
  IL-18 target gene sequence
accgcagtaatacggaatata

SEQ ID NO: 290 - DNA, 21 bp, Artificial sequence
  IL-18 target gene sequence
ctctcactaacttacatcaaa

SEQ ID NO: 291 - DNA, 21 bp, Artificial sequence
  CCL20 target gene sequence
atcatctttcacacaaagaaa

SEQ ID NO: 292 - DNA, 21 bp, Artificial sequence
  CCL20 target gene sequence
aacagacttgggtgaaatata

SEQ ID NO: 293 - DNA, 21 bp, Artificial sequence
  CCL20 target gene sequence
atggaattggacatagcccaa

SEQ ID NO: 294 - DNA, 21 bp, Artificial sequence
  CCL20 target gene sequence
gagggtttagtgcttatctaa

SEQ ID NO: 295 - DNA, 21 bp, Artificial sequence
  CCL20 target gene sequence
ctcactggacttgtccaatta

SEQ ID NO: 296 - DNA, 21 bp, Artificial sequence
  CCL20 target gene sequence
atcatagtttgctttgtttaa

SEQ ID NO: 297 - DNA, 21 bp, Artificial sequence
  CCL20 target gene sequence
ttgtttaagcatcacattaaa

SEQ ID NO: 298 - DNA, 21 bp, Artificial sequence
  CCL20 target gene sequence
aagcatcacattaaagttaaa

SEQ ID NO: 299 - DNA, 21 bp, Artificial sequence
  CCL20 target gene sequence
cccaaagaactgggtactcaa

SEQ ID NO: 300 - DNA, 21 bp, Artificial sequence
  CCL20 target gene sequence
cacattaaagttaaactgtat

SEQ ID NO: 301 - DNA, 21 bp, Artificial sequence
  CCL20 target gene sequence
cagatctgttctttgagctaa

SEQ ID NO: 302 - DNA, 21 bp, Artificial sequence
  CCL20 target gene sequence
ttggtttagtgcaaagtataa

SEQ ID NO: 303 - DNA, 21 bp, Artificial sequence
  CCL20 target gene sequence
cagaccgtattcttcatccta

SEQ ID NO: 304 - DNA, 21 bp, Artificial sequence
  CCL20 target gene sequence
aacattaataagacaaatatt

SEQ ID NO: 305 - DNA, 21 bp, Artificial sequence
  CCL20 target gene sequence
gaccgtattcttcatcctaaa

SEQ ID NO: 306 - DNA, 21 bp, Artificial sequence
  CCL20 target gene sequence
aagcttgtgacattaatgcta

SEQ ID NO: 307 - DNA, 21 bp, Artificial sequence
  CCL20 target gene sequence
caataagctattgtaaagata

SEQ ID NO: 308 - DNA, 21 bp, Artificial sequence
  CCL20 target gene sequence
atcatctttcacacgaagaaa

SEQ ID NO: 309 - DNA, 21 bp, Artificial sequence
  CCL20 target gene sequence
agctattgtaaagatatttaa

SEQ ID NO: 310 - DNA, 21 bp, Artificial sequence
  CCL20 target gene sequence
cagcctaagagtcaagaagat

SEQ ID NO: 311 - DNA, 21 bp, Artificial sequence
  CCL20 target gene sequence
cccagtggacttgtcaatgga

SEQ ID NO: 312 - DNA, 21 bp, Artificial sequence
  CCL20 target gene sequence
atgaagttgattcatattgca

SEQ ID NO: 313 - DNA, 21 bp, Artificial sequence
  CCL20 target gene sequence
aagttgattcatattgcatca

SEQ ID NO: 314 - DNA, 21 bp, Artificial sequence
  CCL20 target gene sequence
tcacattagagttaagttgta

SEQ ID NO: 315 - DNA, 21 bp, Artificial sequence
  CCL20 target gene sequence
cacattagagttaagttgtat

SEQ ID NO: 316 - DNA, 21 bp, Artificial sequence
  CCL20 target gene sequence
tatgttatttatagatctgaa

SEQ ID NO: 317 - DNA, 21 bp, Artificial sequence
  CCL20 target gene sequence
atgtttagctatttaatgtta

SEQ ID NO: 318 - DNA, 21 bp, Artificial sequence
  CCL20 target gene sequence
ttagtggaaggattaatatta

SEQ ID NO: 319 - DNA, 21 bp, Artificial sequence
  CCL20 target gene sequence
acccagcactgagtacatcaa

SEQ ID NO: 320 - DNA, 21 bp, Artificial sequence
  CCL20 target gene sequence
tatgtttaagggaatagttta

SEQ ID NO: 321 - DNA, 21 bp, Artificial sequence
  CCL20 target gene sequence
atgaagttgattcatattgca

SEQ ID NO: 322 - DNA, 21 bp, Artificial sequence
  CCL20 target gene sequence
tgaagttgattcatattgcat

SEQ ID NO: 323 - DNA, 21 bp, Artificial sequence
  CCL20 target gene sequence
gaagttgattcatattgcatc

SEQ ID NO: 324 - DNA, 21 bp, Artificial sequence
  CCL20 target gene sequence
aagttgattcatattgcatca

SEQ ID NO: 325 - DNA, 21 bp, Artificial sequence
  CCL20 target gene sequence
agttgattcatattgcatcat

SEQ ID NO: 326 - DNA, 21 bp, Artificial sequence
  CCL20 target gene sequence
gttgattcatattgcatcata

SEQ ID NO: 327 - DNA, 21 bp, Artificial sequence
  CCL20 target gene sequence
ttgattcatattgcatcatag

SEQ ID NO: 328 - DNA, 21 bp, Artificial sequence
  CCL20 target gene sequence
tgattcatattgcatcatagt

SEQ ID NO: 329 - DNA, 21 bp, Artificial sequence
  CCL20 target gene sequence
tcaatgctatcatctttcaca

SEQ ID NO: 330 - DNA, 21 bp, Artificial sequence
  CCL20 target gene sequence
caatgctatcatctttcacac

SEQ ID NO: 331 - DNA, 21 bp, Artificial sequence
  CCL20 target gene sequence
taatgaagttgattcatattg

SEQ ID NO: 332 - DNA, 21 bp, Artificial sequence
  CCL20 target gene sequence
aatgaagttgattcatattgc

SEQ ID NO: 333 - DNA, 21 bp, Artificial sequence
  CCL20 target gene sequence
agcatgaaatttgagattgga

SEQ ID NO: 334 - DNA, 21 bp, Artificial sequence
  CCL20 target gene sequence
tacagagcctctgaaagacca

SEQ ID NO: 335 - DNA, 21 bp, Artificial sequence
  CCL20 target gene sequence
cactacagagcctctgaaaga

SEQ ID NO: 336 - DNA, 21 bp, Artificial sequence
  CCL20 target gene sequence
ctgacagcatgaaatttgaga

SEQ ID NO: 337 - DNA, 21 bp, Artificial sequence
  CCL20 target gene sequence
atctctgtggtgggcatgaga

SEQ ID NO: 338 - DNA, 21 bp, Artificial sequence
  CCL20 target gene sequence
catgaaatttgagattggaga

SEQ ID NO: 339 - DNA, 21 bp, Artificial sequence
  CCL20 target gene sequence
tctggctgaggttggctctta

SEQ ID NO: 340 - DNA, 21 bp, Artificial sequence
  CCL20 target gene sequence
gtgggctacatcctaggcctt

SEQ ID NO: 341 - DNA, 21 bp, Artificial sequence
  CCL20 target gene sequence
cagcttcctgctaaaccacaa

SEQ ID NO: 342 - DNA, 21 bp, Artificial sequence
  CCL20 target gene sequence
caagagtgagttcaactcata

SEQ ID NO: 343 - DNA, 21 bp, Artificial sequence
  CCL20 target gene sequence
ctggttcctgacagcatgaaa

SEQ ID NO: 344 - DNA, 21 bp, Artificial sequence
  CCL20 target gene sequence
tggctgggactatatatataa

SEQ ID NO: 345 - DNA, 21 bp, Artificial sequence
  CCL20 target gene sequence
gagggcaattgctatatctta

SEQ ID NO: 346 - DNA, 21 bp, Artificial sequence
  CCL20 target gene sequence
cagcagccaaacgacaagcaa

SEQ ID NO: 347 - DNA, 21 bp, Artificial sequence
  CCL20 target gene sequence
caagggtttccttaaggacaa

SEQ ID NO: 348 - DNA, 21 bp, Artificial sequence
  CCL20 target gene sequence
cagatacttgtaaggaggaaa

SEQ ID NO: 349 - DNA, 21 bp, Artificial sequence
  CCL20 target gene sequence
aagaaatggattagtcagtaa

SEQ ID NO: 350 - DNA, 21 bp, Artificial sequence
  CCL20 target gene sequence
aaggaaagcacaagaagccaa

SEQ ID NO: 351 - DNA, 21 bp, Artificial sequence
  CCL20 target gene sequence
ctggctgaggttggctcttaa

SEQ ID NO: 352 - DNA, 21 bp, Artificial sequence
  CCL20 target gene sequence
aacctgggatctaaagaaaca

SEQ ID NO: 353 - DNA, 21 bp, Artificial sequence
  CCL20 target gene sequence
aagggcttgggtatcaaagaa

SEQ ID NO: 354 - DNA, 21 bp, Artificial sequence
  CCL20 target gene sequence
caggctccgaagatacttcta

SEQ ID NO: 355 - DNA, 21 bp, Artificial sequence
  CCL20 target gene sequence
cccaatatataaattgcctaa

SEQ ID NO: 356 - DNA, 21 bp, Artificial sequence
  CCL20 target gene sequence
ctgacccagcttcctgctaaa

SEQ ID NO: 357 - DNA, 21 bp, Artificial sequence
  Chitinase-3 target gene sequence
acccacatcatctacagcttt

SEQ ID NO: 358 - DNA, 21 bp, Artificial sequence
  Chitinase-3 target gene sequence
catcatctacagctttgccaa

SEQ ID NO: 359 - DNA, 21 bp, Artificial sequence
  Chitinase-3 target gene sequence
cagctggtcccagtaccggga

SEQ ID NO: 360 - DNA, 21 bp, Artificial sequence
  Chitinase-3 target gene sequence
caccaaggaggcagggaccct

SEQ ID NO: 361 - DNA, 21 bp, Artificial sequence
  Chitinase-3 target gene sequence
ccggttcaccaaggaggcagg

SEQ ID NO: 362 - DNA, 21 bp, Artificial sequence
  Chitinase-3 target gene sequence
agctggtcccagtaccgggaa

SEQ ID NO: 363 - DNA, 21 bp, Artificial sequence
  Chitinase-3 target gene sequence
caggccggttcaccaaggagg

SEQ ID NO: 364 - DNA, 21 bp, Artificial sequence
  Chitinase-3 target gene sequence
ggccggttcaccaaggaggca

SEQ ID NO: 365 - DNA, 21 bp, Artificial sequence
  Chitinase-3 target gene sequence
taggtttgacagatacagcaa

SEQ ID NO: 366 - DNA, 21 bp, Artificial sequence
  Chitinase-3 target gene sequence
aaccctgttaaggaatgcaaa

SEQ ID NO: 367 - DNA, 21 bp, Artificial sequence
  Chitinase-3 target gene sequence
atcaagtaggcaaatatctta

SEQ ID NO: 368 - DNA, 21 bp, Artificial sequence
  Chitinase-3 target gene sequence
cgcagctttgtcagcaggaaa

SEQ ID NO: 369 - DNA, 21 bp, Artificial sequence
  Chitinase-3 target gene sequence
ttggatcaagtaggcaaatat

SEQ ID NO: 370 - DNA, 21 bp, Artificial sequence
  Chitinase-3 target gene sequence
ttgagggaccatactaattat

SEQ ID NO: 371 - DNA, 21 bp, Artificial sequence
  Chitinase-3 target gene sequence
gaggacaaggagagtgtcaaa

SEQ ID NO: 372 - DNA, 21 bp, Artificial sequence
  Chitinase-3 target gene sequence
tgcgtacaagctggtctgcta

SEQ ID NO: 373 - DNA, 21 bp, Artificial sequence
  Chitinase-3 target gene sequence
caggagtttaatctcttgcaa

SEQ ID NO: 374 - DNA, 21 bp, Artificial sequence
  Chitinase-3 target gene sequence
atcaaggaactgaatgcggaa

SEQ ID NO: 375 - DNA, 21 bp, Artificial sequence
  Chitinase-3 target gene sequence
caccctgatcaaggaactgaa

SEQ ID NO: 376 - DNA, 21 bp, Artificial sequence
  Chitinase-3 target gene sequence
cacttggatcaagtaggcaaa

SEQ ID NO: 377 - DNA, 21 bp, Artificial sequence
  Chitinase-3 target gene sequence
caggattgagggaccatacta

SEQ ID NO: 378 - DNA, 21 bp, Artificial sequence
  Chitinase-3 target gene sequence
aactatgacaagctgaataaa

SEQ ID NO: 379 - DNA, 21 bp, Artificial sequence
  Chitinase-3 target gene sequence
atgcaaattctcagactctaa

SEQ ID NO: 380 - DNA, 21 bp, Artificial sequence
  Chitinase-3 target gene sequence
atccttcccttaggaacttaa

SEQ ID NO: 381-382 - skipped (2 entries)

SEQ ID NO: 383 - DNA, 65 bp, Artificial sequence
  H3 hairpin sequence
ggatccaggagtaacaatacaaatggattcaagagatccatttgtattgttactcctttgtcgac

SEQ ID NO: 384 - DNA, 21 bp, Artificial sequence
  Chemically synthesized primer
ctgatctgtgcacggaactga

SEQ ID NO: 385 - DNA, 25 bp, Artificial sequence
  Chemically synthesized primer
tgtctaagtttttctgctggattca

SEQ ID NO: 386 - DNA, 25 bp, Artificial sequence
  Chemically synthesized primer
ttggaacttacagaggtgcctgcgc

SEQ ID NO: 387 - DNA, 61 bp, Artificial sequence
  HPV shRNA sequence
ggatcctaggtatttgaatttgcatttcaagagaatgcaaattcaaataccttttgtcgac

SEQ ID NO: 388 - DNA, 61 bp, Artificial sequence
  HPV shRNA sequence
gtcgacaaaaggtatttgaatttgcattctcttgaaatgcaaattcaaatacctaggatcc

SEQ ID NO: 389 - DNA, 61 bp, Artificial sequence
  HPV shRNA sequence
ggatcctcagaaaaacttagacaccttcaagagaggtgtctaagtttttctgtttgtcgac

SEQ ID NO: 390 - DNA, 61 bp, Artificial sequence
  HPV shRNA sequence
gtcgacaaacagaaaaacttagacacctctcttgaaggtgtctaagtttttctgaggatcc

SEQ ID NO: 391 - skipped

SEQ ID NO: 392 - DNA, 54 bp, Artificial sequence
  HPVH1 construct
gatcctaggtatttgaatttgcatttcaagagaatgcaaattcaaatacctttt

SEQ ID NO: 393 - DNA, 55 bp, Artificial sequence; identical to SEQ ID NO: 54
  HPVH1 construct

SEQ ID NO: 394 - DNA, 21 bp, Artificial sequence
  CCL20  siRNA sequence
gcuugugacauuaaugcuatt

SEQ ID NO: 395 - DNA, 21 bp, Artificial sequence
  CCL20 siRNA sequence
uagcauuaaugucacaagctt

SEQ ID NO: 396 - DNA, 21 bp, Artificial sequence
  CCL20 siRNA sequence
auaagcuauuguaaagauatt

SEQ ID NO: 397 - DNA, 21 bp, Artificial sequence
  CCL20 siRNA sequence
uaucuuuacaauagcuuautg

SEQ ID NO: 398 - DNA, 21 bp, Artificial sequence
  CCL20 siRNA sequence
caucuuucacacgaagaaatt

SEQ ID NO: 399 - DNA, 21 bp, Artificial sequence
  CCL20 siRNA sequence
uuucuucgugugaaagaugat

SEQ ID NO: 400 - DNA, 21 bp, Artificial sequence
  CCL20 siRNA sequence
cuauuguaaagauauuuaatt

SEQ ID NO: 401 - DNA, 21 bp, Artificial sequence
  CCL20 siRNA sequence
uuaaauaucuuuacaauagct

SEQ ID NO: 402 - DNA, 21 bp, Artificial sequence
  CCL20 siRNA sequence
gccuaagagucaagaagautt

SEQ ID NO: 403 - DNA, 21 bp, Artificial sequence
  CCL20 siRNA sequence
aucuucuugacucuuaggctg

SEQ ID NO: 404 - DNA, 21 bp, Artificial sequence
  CCL20 siRNA sequence
caguggacuugucaauggatt

SEQ ID NO: 405 - DNA, 21 bp, Artificial sequence
  CCL20 siRNA sequence
uccauugacaaguccacuggg

SEQ ID NO: 406 - DNA, 21 bp, Artificial sequence
  CCL20 siRNA sequence
gaaguugauucauauugcatt

SEQ ID NO: 407 - DNA, 21 bp, Artificial sequence
  CCL20 siRNA sequence
ugcaauaugaaucaacuucat

SEQ ID NO: 408 - DNA, 21 bp, Artificial sequence
  CCL20 siRNA sequence
guugauucauauugcaucatt

SEQ ID NO: 409 - DNA, 21 bp, Artificial sequence
  CCL20 siRNA sequence
ugaugcaauaugaaucaactt

SEQ ID NO: 410 - DNA, 21 bp, Artificial sequence
  CCL20 siRNA sequence
acauuagaguuaaguuguatt

SEQ ID NO: 411 - DNA, 21 bp, Artificial sequence
  CCL20 siRNA sequence
uacaacuuaacucuaauguga

SEQ ID NO: 412 - DNA, 21 bp, Artificial sequence
  CCL20 siRNA sequence
cauuagaguuaaguuguautt

SEQ ID NO: 413 - DNA, 21 bp, Artificial sequence
  CCL20 siRNA sequence
auacaacuuaacucuaaugtg

SEQ ID NO: 414 - DNA, 21 bp, Artificial sequence
  CCL20 siRNA sequence
uguuauuuauagaucugaatt

SEQ ID NO: 415 - DNA, 21 bp, Artificial sequence
  CCL20 siRNA sequence
uucagaucuauaaauaacata

SEQ ID NO: 416 - DNA, 21 bp, Artificial sequence
  CCL20 siRNA sequence
guuuagcuauuuaauguuatt

SEQ ID NO: 417 - DNA, 21 bp, Artificial sequence
  CCL20 siRNA sequence
uaacauuaaauagcuaaacat

SEQ ID NO: 418 - DNA, 21 bp, Artificial sequence
  CCL20 siRNA sequence
aguggaaggauuaauauuatt

SEQ ID NO: 419 - DNA, 21 bp, Artificial sequence
  CCL20 siRNA sequence
uaauauuaauccuuccacuaa

SEQ ID NO: 420 - DNA, 21 bp, Artificial sequence
  CCL20 siRNA sequence
ccagcacugaguacaucaatt

SEQ ID NO: 421 - DNA, 21 bp, Artificial sequence
  CCL20 siRNA sequence
uugauguacucagugcugggt

SEQ ID NO: 422 - DNA, 21 bp, Artificial sequence
  CCL20 siRNA sequence
uguuuaagggaauaguuuatt

SEQ ID NO: 423 - DNA, 21 bp, Artificial sequence
  CCL20 siRNA sequence
uaaacuauucccuuaaacata

SEQ ID NO: 424 - DNA, 21 bp, Artificial sequence
  Claudin-2 siRNA sequence
gcugggacuauauauauaatt

SEQ ID NO: 425 - DNA, 21 bp, Artificial sequence
  Claudin-2 siRNA sequence
uuauauauauagucccagcca

SEQ ID NO: 426 - DNA, 21 bp, Artificial sequence
  Claudin-2 siRNA sequence
gggcaauugcuauaucuuatt

SEQ ID NO: 427 - DNA, 21 bp, Artificial sequence
  Claudin-2 siRNA sequence
uaagauauagcaauugccctc

SEQ ID NO: 428 - DNA, 21 bp, Artificial sequence
  Claudin-2 siRNA sequence
gcagccaaacgacaagcaatt

SEQ ID NO: 429 - DNA, 21 bp, Artificial sequence
  Claudin-2 siRNA sequence
uugcuugucguuuggcugctg

SEQ ID NO: 430 - DNA, 21 bp, Artificial sequence
  Claudin-2 siRNA sequence
aggguuuccuuaaggacaatt

SEQ ID NO: 431 - DNA, 21 bp, Artificial sequence
  Claudin-2 siRNA sequence
uuguccuuaaggaaacccutg

SEQ ID NO: 432 - DNA, 21 bp, Artificial sequence
  Claudin-2 siRNA sequence
gaaauggauuagucaguaatt

SEQ ID NO: 433 - DNA, 21 bp, Artificial sequence
  Claudin-2 siRNA sequence
uuacugacuaauccauuuctt

SEQ ID NO: 434 - DNA, 21 bp, Artificial sequence
  Claudin-2 siRNA sequence
ggcuccgaagauacuucuatt

SEQ ID NO: 435 - DNA, 21 bp, Artificial sequence
  Claudin-2 siRNA sequence
uagaaguaucuucggagcctg

SEQ ID NO: 436 - DNA, 21 bp, Artificial sequence
  IL6-RA siRNA sequence
ccuggagggugacaaaguatt

SEQ ID NO: 437 - DNA, 21 bp, Artificial sequence
  IL6-RA siRNA sequence
uacuuugucacccuccaggat

SEQ ID NO: 438 - DNA, 21 bp, Artificial sequence
  IL6-RA siRNA sequence
ggucugacaauaccguaaatt

SEQ ID NO: 439 - DNA, 21 bp, Artificial sequence
  IL6-RA siRNA sequence
uuuacgguauugucagaccca

SEQ ID NO: 440 - DNA, 21 bp, Artificial sequence
  IL6-RA siRNA sequence
gcuguuuccuauaacagaatt

SEQ ID NO: 441 - DNA, 21 bp, Artificial sequence
  IL6-RA siRNA sequence
uucuguuauaggaaacagcgg

SEQ ID NO: 442 - DNA, 21 bp, Artificial sequence
  IL6-RA siRNA sequence
gcugugaaagggaaauuuatt

SEQ ID NO: 443 - DNA, 21 bp, Artificial sequence
  IL6-RA siRNA sequence
uaaauuucccuuucacagcag

SEQ ID NO: 444 - DNA, 21 bp, Artificial sequence
  IL6-RA siRNA sequence
ccuugugguaucagccauatt

SEQ ID NO: 445 - DNA, 21 bp, Artificial sequence
  IL6-RA siRNA sequence
uauggcugauaccacaaggtt

SEQ ID NO: 446 - DNA, 21 bp, Artificial sequence
  IL6-RA siRNA sequence
gcuucgauaccgaccuguatt

SEQ ID NO: 447 - DNA, 21 bp, Artificial sequence
  IL6-RA siRNA sequence
uacaggucgguaucgaagctg

SEQ ID NO: 448 - DNA, 21 bp, Artificial sequence
  IL6-RA siRNA sequence
cggcaggaauccucuggaatt

SEQ ID NO: 449 - DNA, 21 bp, Artificial sequence
  IL6-RA siRNA sequence
uuccagaggauuccugccggg

SEQ ID NO: 450 - DNA, 21 bp, Artificial sequence
  IL6-RA siRNA sequence
ccacgaggaucaguacgaatt

SEQ ID NO: 451 - DNA, 21 bp, Artificial sequence
  IL6-RA siRNA sequence
uucguacugauccucguggtt

SEQ ID NO: 452 - DNA, 21 bp, Artificial sequence
  IL6-RA siRNA sequence
cacgaggaucaguacgaaatt

SEQ ID NO: 453 - DNA, 21 bp, Artificial sequence
  IL6-RA siRNA sequence
uuucguacugauccucguggt

SEQ ID NO: 454 - DNA, 21 bp, Artificial sequence
  IL6-RA siRNA sequence
gaucaguacgaaaguucuatt

SEQ ID NO: 455 - DNA, 21 bp, Artificial sequence
  IL6-RA siRNA sequence
uagaacuuucguacugaucct

SEQ ID NO: 456 - DNA, 21 bp, Artificial sequence
  IL6-RA siRNA sequence
guacgaaaguucuacagaatt

SEQ ID NO: 457 - DNA, 21 bp, Artificial sequence
  IL6-RA siRNA sequence
uucuguagaacuuucguactg

SEQ ID NO: 458 - DNA, 21 bp, Artificial sequence
  IL6-RA siRNA sequence
gaaaguucuacagaagcaatt

SEQ ID NO: 459 - DNA, 21 bp, Artificial sequence
  IL6-RA siRNA sequence
uugcuucuguagaacuuucgt

SEQ ID NO: 460 - DNA, 21 bp, Artificial sequence
  IL6-RA siRNA sequence
gggucugacaauaccguaatt

SEQ ID NO: 461 - DNA, 21 bp, Artificial sequence
  IL6-RA siRNA sequence
uuacgguauugucagacccag

SEQ ID NO: 462 - DNA, 21 bp, Artificial sequence
  IL13-RA1 siRNA sequence
agaagacucuaaugauguatt

SEQ ID NO: 463 - DNA, 21 bp, Artificial sequence
  IL13-RA1 siRNA sequence
uacaucauuagagucuucutg

SEQ ID NO: 464 - DNA, 21 bp, Artificial sequence
  IL13-RA1 siRNA sequence
cagucagaguaagagucaatt

SEQ ID NO: 465 - DNA, 21 bp, Artificial sequence
  IL13-RA1 siRNA sequence
uugacucuuacucugacugtg

SEQ ID NO: 466 - DNA, 21 bp, Artificial sequence
  IL13-RA1 siRNA sequence
cagaacaucuagcaaacaatt

SEQ ID NO: 467 - DNA, 21 bp, Artificial sequence
  IL13-RA1 siRNA sequence
uuguuugcuagauguucugtg

SEQ ID NO: 468 - DNA, 21 bp, Artificial sequence
  IL13-RA1 siRNA sequence
cuuguagguucacauauuatt

SEQ ID NO: 469 - DNA, 21 bp, Artificial sequence
  IL13-RA1 siRNA sequence
uaauaugugaaccuacaagtt

SEQ ID NO: 470 - DNA, 21 bp, Artificial sequence
  IL13-RA1 siRNA sequence
caguguagugccaaugaaatt

SEQ ID NO: 471 - DNA, 21 bp, Artificial sequence
  IL13-RA1 siRNA sequence
uuucauuggcacuacacugag

SEQ ID NO: 472 - DNA, 21 bp, Artificial sequence
  IL13-RA1 siRNA sequence
guaugacaucuaugagaaatt

SEQ ID NO: 473 - DNA, 21 bp, Artificial sequence
  IL13-RA1 siRNA sequence
uuucucauagaugucauactt

SEQ ID NO: 474 - DNA, 21 bp, Artificial sequence
  IL18 siRNA sequence
aggaaaugauguuuauugatt

SEQ ID NO: 475 - DNA, 21 bp, Artificial sequence
  IL18 siRNA sequence
ucaauaaacaucauuuccutg

SEQ ID NO: 476 - DNA, 21 bp, Artificial sequence
  IL18 siRNA sequence
ggccgacuucacuguacaatt

SEQ ID NO: 477 - DNA, 21 bp, Artificial sequence
  IL18 siRNA sequence
uuguacagugaagucggccaa

SEQ ID NO: 478 - DNA, 21 bp, Artificial sequence
  IL18 siRNA sequence
gauggaguuugaaucuucatt

SEQ ID NO: 479 - DNA, 21 bp, Artificial sequence
  IL18 siRNA sequence
ugaagauucaaacuccauctt

SEQ ID NO: 480 - DNA, 21 bp, Artificial sequence
  IL18 siRNA sequence
caaccgcaguaauacggaatt

SEQ ID NO: 481 - DNA, 21 bp, Artificial sequence
  IL18 siRNA sequence
uuccguauuacugcgguugta

SEQ ID NO: 482 - DNA, 21 bp, Artificial sequence
  IL18 siRNA sequence
cgaggcugcaugauuuauatt

SEQ ID NO: 483 - DNA, 21 bp, Artificial sequence
  IL18 siRNA sequence
uauaaaucaugcagccucggg

SEQ ID NO: 484 - DNA, 21 bp, Artificial sequence
  IL18 siRNA sequence
ccuguauuuccauaacagatt

SEQ ID NO: 485 - DNA, 21 bp, Artificial sequence
  IL18 siRNA sequence
ucuguuauggaaauacaggcg

SEQ ID NO: 486 - DNA, 21 bp, Artificial sequence
  IL18 siRNA sequence
cauguacaaagacagugaatt

SEQ ID NO: 487 - DNA, 21 bp, Artificial sequence
  IL18 siRNA sequence
uucacugucuuuguacaugta

SEQ ID NO: 488 - DNA, 21 bp, Artificial sequence
  IL18 siRNA sequence
cgaggauaugacugauauutt

SEQ ID NO: 489 - DNA, 21 bp, Artificial sequence
  IL18 siRNA sequence
aauaucagucauauccucgaa

SEQ ID NO: 490 - DNA, 21 bp, Artificial sequence
  IL18 siRNA sequence
ggauaugacugauauugautt

SEQ ID NO: 491 - DNA, 21 bp, Artificial sequence
  IL18 siRNA sequence
aucaauaucagucauaucctc

SEQ ID NO: 492 - DNA, 21 bp, Artificial sequence
  IL18 siRNA sequence
cuaacuuacaucaaaguuatt

SEQ ID NO: 493 - DNA, 21 bp, Artificial sequence
  IL18 siRNA sequence
uaacuuugauguaaguuagtg

SEQ ID NO: 494 - DNA, 21 bp, Artificial sequence
  IL18 siRNA sequence
cucacuaacuuacaucaaatt

SEQ ID NO: 495 - DNA, 21 bp, Artificial sequence
  IL18 siRNA sequence
uuugauguaaguuagugagag

SEQ ID NO: 496 - DNA, 21 bp, Artificial sequence
  IL-7 siRNA sequence
gauccuacggaaguuauggtt

SEQ ID NO: 497 - DNA, 21 bp, Artificial sequence
  IL-7 siRNA sequence
ccauaacuuccguaggauccg

SEQ ID NO: 498 - DNA, 21 bp, Artificial sequence
  IL-7 siRNA sequence
ccauguuccauguuucuuutt

SEQ ID NO: 499 - DNA, 21 bp, Artificial sequence
  IL-7 siRNA sequence
aaagaaacauggaacauggtc

SEQ ID NO: 500 - DNA, 21 bp, Artificial sequence
  IL-7 siRNA sequence
ccucccgcagaccauguuctt

SEQ ID NO: 501 - DNA, 21 bp, Artificial sequence
  IL-7 siRNA sequence
gaacauggucugcgggaggcg

SEQ ID NO: 502 - DNA, 21 bp, Artificial sequence
  IL-7 siRNA sequence
cucccgcagaccauguucctt

SEQ ID NO: 503 - DNA, 21 bp, Artificial sequence
  IL-7 siRNA sequence
ggaacauggucugcgggaggc

SEQ ID NO: 504 - DNA, 21 bp, Artificial sequence
  IL-7 siRNA sequence
ucccgcagaccauguuccatt

SEQ ID NO: 505 - DNA, 21 bp, Artificial sequence
  IL-7 siRNA sequence
uggaacauggucugcgggagg

SEQ ID NO: 506 - DNA, 21 bp, Artificial sequence
  IL-7 siRNA sequence
cccgcagaccauguuccautt

SEQ ID NO: 507 - DNA, 21 bp, Artificial sequence
  IL-7 siRNA sequence
auggaacauggucugcgggag

SEQ ID NO: 508 - DNA, 21 bp, Artificial sequence
  IL-7 siRNA sequence
ccgcagaccauguuccaugtt

SEQ ID NO: 509 - DNA, 21 bp, Artificial sequence
  IL-7 siRNA sequence
cauggaacauggucugcggga

SEQ ID NO: 510 - DNA, 21 bp, Artificial sequence
  IL-7 siRNA sequence
cgcagaccauguuccaugutt

SEQ ID NO: 511 - DNA, 21 bp, Artificial sequence
  IL-7 siRNA sequence
acauggaacauggucugcggg

SEQ ID NO: 512 - DNA, 21 bp, Artificial sequence
  IL-7 siRNA sequence
agaccauguuccauguuuctt

SEQ ID NO: 513 - DNA, 21 bp, Artificial sequence
  IL-7 siRNA sequence
gaaacauggaacauggucugc

SEQ ID NO: 514 - DNA, 21 bp, Artificial sequence
  IL-7 siRNA sequence
accauguuccauguuucuutt

SEQ ID NO: 515 - DNA, 21 bp, Artificial sequence
  IL-7 siRNA sequence
aagaaacauggaacaugguct

SEQ ID NO: 516 - DNA, 21 bp, Artificial sequence
  Chitinase3-like-1 siRNA sequence
ccacaucaucuacagcuuutt

SEQ ID NO: 517 - DNA, 21 bp, Artificial sequence
  Chitinase3-like-1 siRNA sequence
aaagcuguagaugaugugggt

SEQ ID NO: 518 - DNA, 21 bp, Artificial sequence
  Chitinase3-like-1 siRNA sequence
gguuugacagauacagcaatt

SEQ ID NO: 519 - DNA, 21 bp, Artificial sequence
  Chitinase3-like-1 siRNA sequence
uugcuguaucugucaaaccta

SEQ ID NO: 520 - DNA, 21 bp, Artificial sequence
  Chitinase3-like-1 siRNA sequence
ucaucuacagcuuugccaatt

SEQ ID NO: 521 - DNA, 21 bp, Artificial sequence
  Chitinase3-like-1 siRNA sequence
uuggcaaagcuguagaugatg

SEQ ID NO: 522 - DNA, 21 bp, Artificial sequence
  Chitinase3-like-1 siRNA sequence
cccuguuaaggaaugcaaatt

SEQ ID NO: 523 - DNA, 21 bp, Artificial sequence
  Chitinase3-like-1 siRNA sequence
uuugcauuccuuaacagggtt

SEQ ID NO: 524 - DNA, 21 bp, Artificial sequence
  Chitinase3-like-1 siRNA sequence
caaguaggcaaauaucuuatt

SEQ ID NO: 525 - DNA, 21 bp, Artificial sequence
  Chitinase3-like-1 siRNA sequence
uaagauauuugccuacuugat

SEQ ID NO: 526 - DNA, 21 bp, Artificial sequence
  Chitinase3-like-1 siRNA sequence
cagcuuugucagcaggaaatt

SEQ ID NO: 527 - DNA, 21 bp, Artificial sequence
  Chitinase3-like-1 siRNA sequence
uuuccugcugacaaagcugcg

SEQ ID NO: 528 - DNA, 21 bp, Artificial sequence
  Chitinase3-like-1 siRNA sequence
gguucaccaaggaggcaggtt

SEQ ID NO: 529 - DNA, 21 bp, Artificial sequence
  Chitinase3-like-1 siRNA sequence
ccugccuccuuggugaaccgg

SEQ ID NO: 530 - DNA, 21 bp, Artificial sequence
  Chitinase3-like-1 siRNA sequence
ggaucaaguaggcaaauautt

SEQ ID NO: 531 - DNA, 21 bp, Artificial sequence
  Chitinase3-like-1 siRNA sequence
auauuugccuacuugauccaa

SEQ ID NO: 532 - DNA, 21 bp, Artificial sequence
  Chitinase3-like-1 siRNA sequence
gagggaccauacuaauuautt

SEQ ID NO: 533 - DNA, 21 bp, Artificial sequence
  Chitinase3-like-1 siRNA sequence
auaauuaguauggucccucaa

SEQ ID NO: 534 - DNA, 21 bp, Artificial sequence
  Chitinase3-like-1 siRNA sequence
ggccgguucaccaaggaggtt

SEQ ID NO: 535 - DNA, 21 bp, Artificial sequence
  Chitinase3-like-1 siRNA sequence
ccuccuuggugaaccggcctg

SEQ ID NO: 536 - DNA, 21 bp, Artificial sequence
  Chitinase3-like-1 siRNA sequence
ggacaaggagagugucaaatt

SEQ ID NO: 537 - DNA, 21 bp, Artificial sequence
  Chitinase3-like-1 siRNA sequence
uuugacacucuccuugucctc

SEQ ID NO: 538 - DNA, 21 bp, Artificial sequence
  Chitinase3-like-1 siRNA sequence
ccgguucaccaaggaggcatt

SEQ ID NO: 539 - DNA, 21 bp, Artificial sequence
  Chitinase3-like-1 siRNA sequence
ugccuccuuggugaaccggcc

SEQ ID NO: 540 - DNA, 21 bp, Artificial sequence
  Chitinase3-like-1 siRNA sequence
cguacaagcuggucugcuatt

SEQ ID NO: 541 - DNA, 21 bp, Artificial sequence
  Chitinase3-like-1 siRNA sequence
uagcagaccagcuuguacgca

SEQ ID NO: 542 - DNA, 21 bp, Artificial sequence
  Chitinase3-like-1 siRNA sequence
ggaguuuaaucucuugcaatt

SEQ ID NO: 543 - DNA, 21 bp, Artificial sequence
  Chitinase3-like-1 siRNA sequence
uugcaagagauuaaacucctg

SEQ ID NO: 544 - DNA, 21 bp, Artificial sequence
  Chitinase3-like-1 siRNA sequence
caaggaacugaaugcggaatt

SEQ ID NO: 545 - DNA, 21 bp, Artificial sequence
  Chitinase3-like-1 siRNA sequence
uuccgcauucaguuccuugat

SEQ ID NO: 546 - DNA, 21 bp, Artificial sequence
  Chitinase3-like-1 siRNA sequence
cccugaucaaggaacugaatt

SEQ ID NO: 547 - DNA, 21 bp, Artificial sequence
  Chitinase3-like-1 siRNA sequence
uucaguuccuugaucagggtg

SEQ ID NO: 548 - DNA, 21 bp, Artificial sequence
  Chitinase3-like-1 siRNA sequence
cuuggaucaaguaggcaaatt

SEQ ID NO: 549 - DNA, 21 bp, Artificial sequence
  Chitinase3-like-1 siRNA sequence
uuugccuacuugauccaagtg

SEQ ID NO: 550 - DNA, 21 bp, Artificial sequence
  Chitinase3-like-1 siRNA sequence
ggauugagggaccauacuatt

SEQ ID NO: 551 - DNA, 21 bp, Artificial sequence
  Chitinase3-like-1 siRNA sequence
uaguauggucccucaaucctg

SEQ ID NO: 552 - DNA, 21 bp, Artificial sequence
  Chitinase3-like-1 siRNA sequence
gcaaauucucagacucuaatt

SEQ ID NO: 553 - DNA, 21 bp, Artificial sequence
  Chitinase3-like-1 siRNA sequence
uuagagucugagaauuugcat

SEQ ID NO: 554 - DNA, 21 bp, Artificial sequence
  Chitinase3-like-1 siRNA sequence
ccuucccuuaggaacuuaatt

SEQ ID NO: 555 - DNA, 21 bp, Artificial sequence
  Chitinase3-like-1 siRNA sequence
uuaaguuccuaagggaaggat

SEQ ID NO: 556 - DNA, 100 bp, Artificial sequence
  Synthetic oligonucleotide
gacttcatatacccaagcttggaaaattttttttaaaaaagtcttgacactttatgcttccggctcgtataatggatccaggagtaacaatacaaatgga

SEQ ID NO: 557 - DNA, 100 bp, Artificial sequence
  Synthetic oligonucleotide
ttcaagagatccatttgtattgttactcctttttttttttgtcgacgatccttagcgaaagctaaggattttttttttactcgagcggattactacatac

SEQ ID NO: 558 - DNA, 70 bp, Artificial sequence
  Synthetic oligonucleotide
gtatgtagtaatccgctcgagtaaaaaaaaaatccttagctttcgctaaggatcgtcgacaaaaaaaaaa

SEQ ID NO: 559 - DNA, 60 bp, Artificial sequence
  Synthetic oligonucleotide
aggagtaacaatacaaatggatctcttgaatccatttgtattgttactcctggatccatt

SEQ ID NO: 560 - DNA, 70 bp, Artificial sequence
  Synthetic oligonucleotide
atacgagccggaagcataaagtgtcaagacttttttaaaaaaaattttccaagcttgggtatatgaagtc

SEQ ID NO: 561 - DNA, 8884 bp, Artificial sequence
  Synthetic pKSII-inv-hly plasmid construct
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatcccccgggctgcagctgggccgtaagatcggcatttaatcgcgacaatccttttaaaaaaacagcgccgctcaattaacctgagcggcgttgttcttctggacgtttgctacttatggggcgagtctaggattgccggactcccattcgcgccccaaataatcagctcattaaactgttcttattgctatctgttatctggttatattgacagcgcacagagcgggaacgccaagtatgcaggccctggttgcagtgcgcctgtgtccatattcatggtttcaaaatccgtgctggtctttttgacccaatattcaccagattgccaatcagaactatacgcggtcaagctcccccactcgccccacaatgtcccgtcaggcgcacgcgttccgttggttgcacgtgaggattcaagaaccgcagacatatctgaaccttggcattgtctgctggcctcgagactggataccagcgatctgccgccatcgtatatccaccgatttgggtagaaccgataactcaccgaataacttgggaattttttacttttcgccgtcacagccacttcgctataggtttggtaggtaatcgtcacctgaccctgatcgttaaccgatacattgggtgtgaatgacgacgaccactcatactgagtattattagcaacatcgttatccatctgtaactggaatgtggcgtttttaaagatcgttttcgggaaccctttatccgtagcgaaattttgcccgttaaccagaataccggtcagcgtaggtaccgggaatagggatatttttttctgcaatgtactcagtatcagggtatcaacctgcggcgtgattgtgacatcaccgacactattcccaaccaccgtcgcggtatagctatctggctgctcggtaatggggctaatactcaccggcacaccgttttgagtaaaactcaagccctgcatcccactgataaaatggccattcttatcgacagggacaaaggataatgtggaactcatcgtgccatcagccaagatatccggtgtggagacggtgaaactggagcggccagcatctggaataggatctgccgtgaaattaaccgtcacactcggcacactgaacgcagccccatccactttcaccgttactgttgctacccccaacgtggtactggtcaatggtgcgctataagtgccgtcattgtgatccgtgataacgcccatattgcctaaggttgtgtcaaaagccacattcgcgccagcctgcgggtccccataggtatccttcaactccaacgtgatggttgaagccattagaccatcagcgatgatagatgtcggtaccgcagccagagtggatttatccgccgcgatagtacccttaacaaagtgggtatcaacactttgccgttgcccctccacttctgctgtgactaccgtcacgccatctgtcgtattggttaatgcaatgcgcgcgacgccatttgcatctgtcttttccgtgattttattcggtagcgcaccattattggtggttatcaccacctcctgcccggctaagggtttcccctcaaaatcagcaacggtgaactcaacggtgattgcagttttcccattagccggtgcgccatcaccaatgacggccgccgttaatgtcaactgaggctgctgaacggtgacgctcaatgtgaatgagttagatcggtttccttggtgatcaaccgcgagcgcactaagcgaataaaagttggctgtcaggtcgtccgttacccgactcacttgtgctgtgcgtttataaggcggtaaaaccaagttgaattgtgtggtactcagtggtgttaatgtgccgccagcggcaatcagttcggcatcactccagacaatttcccttacagcagatgccccttgtacttgtgcgttcacctgataaacctgacccggcaggccggagatagttgctggcgataatgtcagtttaaccacctgctgtttctgatactccaacacgatattattgttacgatcgacaaggttatagcggctctccgccagtagacgtgttcctgccaccgctgaagggctaagttgcgactgaaaactctcgcccaggcgatagttcatttggaggttccactgtgtttcatgcttactgcttttccccatacgctgatctaccccgacagtgagtagaggcacgggggtgtaattgatcccggcagtcacggcataagggttgcgttgcagattatctttaccaaataaagcaacacgctcaccggtgtattgctcatacatcaacttcccccccagttgtgggagtgcaggtaaataagcattcgcgcgcaaatcccccccagtggctgggcgctctttatagtcggagaaatcacgcgacgagtgccatccattgaggcgaaaatacccattggcagccaactgtaaataatcggtccaggcctcggcaccaagaccgatacggtggttgtggccggtcaaatcattatcataaaaagtattaagtccgtacagccaaccgttctccaatgtacgtatcccgacgccaaggttaagtgtgttgcggctgtctttattgcgaatacctaactgactaaaaaagaggaatgaagcagagtcataccaaggagccagccaatcaagagagctttcttttagcgaaaaatttttgtcaaaattcagattaacttgagccgtaccgaatcgatttaaccactgtttgatttcttgattaaccgcatcgcccaccattgagtgagcaacatcagatgccctgcctgatgcagctaacctggccccggtgcttatcatcttattcaccgcttcagtctcctgctccttattggcgcgatctattattgcagcatttctttctgtatccgatgcggaaaagggattgattgaactctccatttcattattaggatggagattttcaaatgcagatgaagagacagaataaggctggacctgttgcggtgcgttagcatcatatttttctgaagccccagccatgaacattccacatatcaaaaagatacaaataactattcgtgaaataatattaaatgaaattattttattaaaatacatagacattcccgcattccttatcaagagaaactcactgattggctggaaaaccatcataatttaaatgaaataaagcatacctgtcatacgtcaaactgcatgtgcgttggctgtgctcaacaacttgagttatttgaggtataactggccacaaacgagcatttgaaatcaccttgaccattaattaaagatgcaatagttgaaagtgaaacttgttttctaatttagtaaagacattaagaggatagcacttttttaaaaaaccagactgggcagattaaaaatattcaaaatatataataaaacagtctataccatacagcgatagaattgatttattgtaactaagcaggtgagaatatcaaaaaaaacaaaaatacaaaatgaactattatcatataaataatatcaattagaataagcccccttcatttgatgttgtcagttgtctgctgcggtttttatttctactttcagtctgaagtgttactccgcaatatccgcattaatcctgatggttgccttgatgactgcaggaattcgatccctcctttgattagtatattcctatcttaaagtgacttttatgttgaggcattaacatttgttaacgacgataaagggacagcaggactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatcgttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatcctgaaggtaacgaaattgttcaacataaaaactggagcgaaaacaataaaagcaagctagctcatttcacatcgtccatctatttgccaggtaacgcgagaaatattaatgtttacgctaaagaatgcactggtttagcttgggaatggtggagaacggtaattgatgaccggaacttaccacttgtgaaaaatagaaatatctccatctggggcaccacgctttatccgaaatatagtaataaagtagataatccaatcgaataattgtaaaagtaataaaaaattaagaataaaaccgcttaacacacacgaaaaaataagcttgttttgcactcttcgtaaattattttgtgaagaatgtagaaacaggcttattttttaatttttttagaagaattaacaaatgtaaaagaatatctgactgtttatccatataatataagcatatcccaaagtttaagccacctatagtttctactgcaaaacgtataatttagttcccacatatactaaaaaacgtgtccttaactctctctgtcagattagttgtaggtggcttaaacttagttttacgaattaaaaaggagcggtgaaatgaaaagtaaacttatttgtatcatcatggtaatagcttttcaggctcatttcactatgacggtaaaagcagattctgtcggggaagaaaaacttcaaaataatacacaagccaaaaagacccctgctgatttaaaagcttatcaagcttatcgataccgtcgacctcgagggggggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 562 - DNA, 8538 bp, Artificial sequence
  Synthetic pMBV40 plasmid construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgacggtatcgataagcttgataagcttttaaatcagcaggggtctttttggcttgtgtattattttgaagtttttcttccccgacagaatctgcttttaccgtcatagtgaaatgagcctgaaaagctattaccatgatgatacaaataagtttacttttcatttcaccgctcctttttaattcgtaaaactaagtttaagccacctacaactaatctgacagagagagttaaggacacgttttttagtatatgtgggaactaaattatacgttttgcagtagaaactataggtggcttaaactttgggatatgcttatattatatggataaacagtcagatattcttttacatttgttaattcttctaaaaaaattaaaaaataagcctgtttctacattcttcacaaaataatttacgaagagtgcaaaacaagcttattttttcgtgtgtgttaagcggttttattcttaattttttattacttttacaattattcgattggattatctactttattactatatttcggataaagcgtggtgccccagatggagatatttctatttttcacaagtggtaagttccggtcatcaattaccgttctccaccattcccaagctaaaccagtgcattctttagcgtaaacattaatatttctcgcgttacctggcaaatagatggacgatgtgaaatgagctagcttgcttttattgttttcgctccagtttttatgttgaacaatttcgttaccttcaggatcataatttacttcatcccaagaaatgttgaattgagcaacgtatcctccagagtgatcgatgttaatttttccatctgtataagcttttgaagttgtttcaatatattctgagttgtttttaataacagctaattcattgtcttttaggaagtttgttgtataagcaatgggaactcctggtgtttctcgattaaaagtagcgccttttttcaaaatatcgcgtaagtctccgaggttgccgtcgatgatttgaacttcatcttttgcggaacctccgtaaattacggctttgaaggaagaatttttgatgatatttgttagttctacatcacctgagacagattttccgcttacggcagcatcaaaagcagcttttactttagtactatgggaattagttgataatttcaaataaacttgacggccatacgccacacttgagatatatgcaggaggattttctgcattcactccaagcgcttgcaactgctctttagtaacagctttgccgaaaaatctggaaggtcttgtaggttcattaacattcacgttatagtaaatttgtttaaaactaatgacttcttcttgcattttcccttcactgattgcgccgaagtttacattcaagctattatttacagctttaaatgctgtaccaaatttcgcaattaattgtgattcactgtaagccatttcgtcatcataatcaatttttgcacttacatttggataagcttgagcatatttttcattccatctttccactaatgtatttactgcgttgttaacgtttgatttagtggcattttttacaacgattttattgtcttgattagtcatacctggcaaatcaatgctgagtgttaatgaatcacgttttacagggagaacatctggttgattttctactaattccgaattcgcttttacgagagcacctggataggttaggctcgaaattgcattcacaacttgaatgtctgcattattttgattgatggatttcttctttttctccacaacaatatattcatttccatctttgtaaccttttcttggcggcacatttgtcactgcatctccgtggtatactaatacattgtttttattgtaatccaatccttgtatatacttatcgatttcatccgcgtgtttcttttcgattggcgtcttaggacttgcaggcggagatgctggtggtgccatggatgaaattgaattttctttattgaatgcagatgcatcctttgcttcagtttgttgcgcaattggtagactaactaatataagtgtaataaaaactagcattatttttttcatgggtttcactctccttctacattttttaacctaataatgccaaataccgtttgccacccctctcttttgataattataatattggcgaaattcgcttctaaagatgaaacgcaatattatatgcttgctttatagctttattctagtcctgctgtccctttatcgtcgttaacaaatgttaatgcctcaacataaaagtcactttaagataggaatatactaatcaaaggagggatcgaattcctgcagtcatcaaggcaaccatcaggattaatgcggatattgcggagtaacacttcagactgaaagtagaaataaaaaccgcagcagacaactgacaacatcaaatgaagggggcttattctaattgatattatttatatgataatagttcattttgtatttttgttttttttgatattctcacctgcttagttacaataaatcaattctatcgctgtatggtatagactgttttattatatattttgaatatttttaatctgcccagtctggttttttaaaaaagtgctatcctcttaatgtctttactaaattagaaaacaagtttcactttcaactattgcatctttaattaatggtcaaggtgatttcaaatgctcgtttgtggccagttatacctcaaataactcaagttgttgagcacagccaacgcacatgcagtttgacgtatgacaggtatgctttatttcatttaaattatgatggttttccagccaatcagtgagtttctcttgataaggaatgcgggaatgtctatgtattttaataaaataatttcatttaatattatttcacgaatagttatttgtatctttttgatatgtggaatgttcatggctggggcttcagaaaaatatgatgctaacgcaccgcaacaggtccagccttattctgtctcttcatctgcatttgaaaatctccatcctaataatgaaatggagagttcaatcaatcccttttccgcatcggatacagaaagaaatgctgcaataatagatcgcgccaataaggagcaggagactgaagcggtgaataagatgataagcaccggggccaggttagctgcatcaggcagggcatctgatgttgctcactcaatggtgggcgatgcggttaatcaagaaatcaaacagtggttaaatcgattcggtacggctcaagttaatctgaattttgacaaaaatttttcgctaaaagaaagctctcttgattggctggctccttggtatgactctgcttcattcctcttttttagtcagttaggtattcgcaataaagacagccgcaacacacttaaccttggcgtcgggatacgtacattggagaacggttggctgtacggacttaatactttttatgataatgatttgaccggccacaaccaccgtatcggtcttggtgccgaggcctggaccgattatttacagttggctgccaatgggtattttcgcctcaatggatggcactcgtcgcgtgatttctccgactataaagagcgcccagccactgggggggatttgcgcgcgaatgcttatttacctgcactcccacaactgggggggaagttgatgtatgagcaatacaccggtgagcgtgttgctttatttggtaaagataatctgcaacgcaacccttatgccgtgactgccgggatcaattacacccccgtgcctctactcactgtcggggtagatcagcgtatggggaaaagcagtaagcatgaaacacagtggaacctccaaatgaactatcgcctgggcgagagttttcagtcgcaacttagcccttcagcggtggcaggaacacgtctactggcggagagccgctataaccttgtcgatcgtaacaataatatcgtgttggagtatcagaaacagcaggtggttaaactgacattatcgccagcaactatctccggcctgccgggtcaggtttatcaggtgaacgcacaagtacaaggggcatctgctgtaagggaaattgtctggagtgatgccgaactgattgccgctggcggcacattaacaccactgagtaccacacaattcaacttggttttaccgccttataaacgcacagcacaagtgagtcgggtaacggacgacctgacagccaacttttattcgcttagtgcgctcgcggttgatcaccaaggaaaccgatctaactcattcacattgagcgtcaccgttcagcagcctcagttgacattaacggcggccgtcattggtgatggcgcaccggctaatgggaaaactgcaatcaccgttgagttcaccgttgctgattttgaggggaaacccttagccgggcaggaggtggtgataaccaccaataatggtgcgctaccgaataaaatcacggaaaagacagatgcaaatggcgtcgcgcgcattgcattaaccaatacgacagatggcgtgacggtagtcacagcagaagtggaggggcaacggcaaagtgttgatacccactttgttaagggtactatcgcggcggataaatccactctggctgcggtaccgacatctatcatcgctgatggtctaatggcttcaaccatcacgttggagttgaaggatacctatggggacccgcaggctggcgcgaatgtggcttttgacacaaccttaggcaatatgggcgttatcacggatcacaatgacggcacttatagcgcaccattgaccagtaccacgttgggggtagcaacagtaacggtgaaagtggatggggctgcgttcagtgtgccgagtgtgacggttaatttcacggcagatcctattccagatgctggccgctccagtttcaccgtctccacaccggatatcttggctgatggcacgatgagttccacattatcctttgtccctgtcgataagaatggccattttatcagtgggatgcagggcttgagttttactcaaaacggtgtgccggtgagtattagccccattaccgagcagccagatagctataccgcgacggtggttgggaatagtgtcggtgatgtcacaatcacgccgcaggttgataccctgatactgagtacattgcagaaaaaaatatccctattcccggtacctacgctgaccggtattctggttaacgggcaaaatttcgctacggataaagggttcccgaaaacgatctttaaaaacgccacattccagttacagatggataacgatgttgctaataatactcagtatgagtggtcgtcgtcattcacacccaatgtatcggttaacgatcagggtcaggtgacgattacctaccaaacctatagcgaagtggctgtgacggcgaaaagtaaaaaattcccaagttattcggtgagttatcggttctacccaaatcggtggatatacgatggcggcagatcgctggtatccagtctcgaggccagcagacaatgccaaggttcagatatgtctgcggttcttgaatcctcacgtgcaaccaacggaacgcgtgcgcctgacgggacattgtggggcgagtgggggagcttgaccgcgtatagttctgattggcaatctggtgaatattgggtcaaaaagaccagcacggattttgaaaccatgaatatggacacaggcgcactgcaaccagggcctgcatacttggcgttcccgctctgtgcgctgtcaatataaccagataacagatagcaataagaacagtttaatgagctgattatttggggcgcgaatgggagtccggcaatcctagactcgccccataagtagcaaacgtccagaagaacaacgccgctcaggttaattgagcggcgctgtttttttaaaaggattgtcgcgattaaatgccgatcttacggcccagctgcagcccgggggatctatgcggtgtgaaataccgcacagatgcgtaaggagaaaataccgcatcaggcgccattcgccattcaggctgcgcaactgttgggaagggcgatcggtgcgggcctcttcgctattacgccaggacttcatatacccaagcttggaaaattttttttaaaaaagtcttgacactttatgcttccggctcgtataatggatccaggagtaacaatacaaatggattcaagagatccatttgtattgttactcctttttttttttgtcgacgatccttagcgaaagctaaggattttttttttactcgagcggattactacatacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 563 - DNA, 8427 bp, Artificial sequence
  Synthetic pMBV43 plasmid construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgacggtatcgataagcttgataagcttttaaatcagcaggggtctttttggcttgtgtattattttgaagtttttcttccccgacagaatctgcttttaccgtcatagtgaaatgagcctgaaaagctattaccatgatgatacaaataagtttacttttcatttcaccgctcctttttaattcgtaaaactaagtttaagccacctacaactaatctgacagagagagttaaggacacgttttttagtatatgtgggaactaaattatacgttttgcagtagaaactataggtggcttaaactttgggatatgcttatattatatggataaacagtcagatattcttttacatttgttaattcttctaaaaaaattaaaaaataagcctgtttctacattcttcacaaaataatttacgaagagtgcaaaacaagcttattttttcgtgtgtgttaagcggttttattcttaattttttattacttttacaattattcgattggattatctactttattactatatttcggataaagcgtggtgccccagatggagatatttctatttttcacaagtggtaagttccggtcatcaattaccgttctccaccattcccaagctaaaccagtgcattctttagcgtaaacattaatatttctcgcgttacctggcaaatagatggacgatgtgaaatgagctagcttgcttttattgttttcgctccagtttttatgttgaacaatttcgttaccttcaggatcataatttacttcatcccaagaaatgttgaattgagcaacgtatcctccagagtgatcgatgttaatttttccatctgtataagcttttgaagttgtttcaatatattctgagttgtttttaataacagctaattcattgtcttttaggaagtttgttgtataagcaatgggaactcctggtgtttctcgattaaaagtagcgccttttttcaaaatatcgcgtaagtctccgaggttgccgtcgatgatttgaacttcatcttttgcggaacctccgtaaattacggctttgaaggaagaatttttgatgatatttgttagttctacatcacctgagacagattttccgcttacggcagcatcaaaagcagcttttactttagtactatgggaattagttgataatttcaaataaacttgacggccatacgccacacttgagatatatgcaggaggattttctgcattcactccaagcgcttgcaactgctctttagtaacagctttgccgaaaaatctggaaggtcttgtaggttcattaacattcacgttatagtaaatttgtttaaaactaatgacttcttcttgcattttcccttcactgattgcgccgaagtttacattcaagctattatttacagctttaaatgctgtaccaaatttcgcaattaattgtgattcactgtaagccatttcgtcatcataatcaatttttgcacttacatttggataagcttgagcatatttttcattccatctttccactaatgtatttactgcgttgttaacgtttgatttagtggcattttttacaacgattttattgtcttgattagtcatacctggcaaatcaatgctgagtgttaatgaatcacgttttacagggagaacatctggttgattttctactaattccgaattcgcttttacgagagcacctggataggttaggctcgaaattgcattcacaacttgaatgtctgcattattttgattgatggatttcttctttttctccacaacaatatattcatttccatctttgtaaccttttcttggcggcacatttgtcactgcatctccgtggtatactaatacattgtttttattgtaatccaatccttgtatatacttatcgatttcatccgcgtgtttcttttcgattggcgtcttaggacttgcaggcggagatgctggtggtgccatggatgaaattgaattttctttattgaatgcagatgcatcctttgcttcagtttgttgcgcaattggtagactaactaatataagtgtaataaaaactagcattatttttttcatgggtttcactctccttctacattttttaacctaataatgccaaataccgtttgccacccctctcttttgataattataatattggcgaaattcgcttctaaagatgaaacgcaatattatatgcttgctttatagctttattctagtcctgctgtccctttatcgtcgttaacaaatgttaatgcctcaacataaaagtcactttaagataggaatatactaatcaaaggagggatcgaattcctgcagtcatcaaggcaaccatcaggattaatgcggatattgcggagtaacacttcagactgaaagtagaaataaaaaccgcagcagacaactgacaacatcaaatgaagggggcttattctaattgatattatttatatgataatagttcattttgtatttttgttttttttgatattctcacctgcttagttacaataaatcaattctatcgctgtatggtatagactgttttattatatattttgaatatttttaatctgcccagtctggttttttaaaaaagtgctatcctcttaatgtctttactaaattagaaaacaagtttcactttcaactattgcatctttaattaatggtcaaggtgatttcaaatgctcgtttgtggccagttatacctcaaataactcaagttgttgagcacagccaacgcacatgcagtttgacgtatgacaggtatgctttatttcatttaaattatgatggttttccagccaatcagtgagtttctcttgataaggaatgcgggaatgtctatgtattttaataaaataatttcatttaatattatttcacgaatagttatttgtatctttttgatatgtggaatgttcatggctggggcttcagaaaaatatgatgctaacgcaccgcaacaggtccagccttattctgtctcttcatctgcatttgaaaatctccatcctaataatgaaatggagagttcaatcaatcccttttccgcatcggatacagaaagaaatgctgcaataatagatcgcgccaataaggagcaggagactgaagcggtgaataagatgataagcaccggggccaggttagctgcatcaggcagggcatctgatgttgctcactcaatggtgggcgatgcggttaatcaagaaatcaaacagtggttaaatcgattcggtacggctcaagttaatctgaattttgacaaaaatttttcgctaaaagaaagctctcttgattggctggctccttggtatgactctgcttcattcctcttttttagtcagttaggtattcgcaataaagacagccgcaacacacttaaccttggcgtcgggatacgtacattggagaacggttggctgtacggacttaatactttttatgataatgatttgaccggccacaaccaccgtatcggtcttggtgccgaggcctggaccgattatttacagttggctgccaatgggtattttcgcctcaatggatggcactcgtcgcgtgatttctccgactataaagagcgcccagccactgggggggatttgcgcgcgaatgcttatttacctgcactcccacaactgggggggaagttgatgtatgagcaatacaccggtgagcgtgttgctttatttggtaaagataatctgcaacgcaacccttatgccgtgactgccgggatcaattacacccccgtgcctctactcactgtcggggtagatcagcgtatggggaaaagcagtaagcatgaaacacagtggaacctccaaatgaactatcgcctgggcgagagttttcagtcgcaacttagcccttcagcggtggcaggaacacgtctactggcggagagccgctataaccttgtcgatcgtaacaataatatcgtgttggagtatcagaaacagcaggtggttaaactgacattatcgccagcaactatctccggcctgccgggtcaggtttatcaggtgaacgcacaagtacaaggggcatctgctgtaagggaaattgtctggagtgatgccgaactgattgccgctggcggcacattaacaccactgagtaccacacaattcaacttggttttaccgccttataaacgcacagcacaagtgagtcgggtaacggacgacctgacagccaacttttattcgcttagtgcgctcgcggttgatcaccaaggaaaccgatctaactcattcacattgagcgtcaccgttcagcagcctcagttgacattaacggcggccgtcattggtgatggcgcaccggctaatgggaaaactgcaatcaccgttgagttcaccgttgctgattttgaggggaaacccttagccgggcaggaggtggtgataaccaccaataatggtgcgctaccgaataaaatcacggaaaagacagatgcaaatggcgtcgcgcgcattgcattaaccaatacgacagatggcgtgacggtagtcacagcagaagtggaggggcaacggcaaagtgttgatacccactttgttaagggtactatcgcggcggataaatccactctggctgcggtaccgacatctatcatcgctgatggtctaatggcttcaaccatcacgttggagttgaaggatacctatggggacccgcaggctggcgcgaatgtggcttttgacacaaccttaggcaatatgggcgttatcacggatcacaatgacggcacttatagcgcaccattgaccagtaccacgttgggggtagcaacagtaacggtgaaagtggatggggctgcgttcagtgtgccgagtgtgacggttaatttcacggcagatcctattccagatgctggccgctccagtttcaccgtctccacaccggatatcttggctgatggcacgatgagttccacattatcctttgtccctgtcgataagaatggccattttatcagtgggatgcagggcttgagttttactcaaaacggtgtgccggtgagtattagccccattaccgagcagccagatagctataccgcgacggtggttgggaatagtgtcggtgatgtcacaatcacgccgcaggttgataccctgatactgagtacattgcagaaaaaaatatccctattcccggtacctacgctgaccggtattctggttaacgggcaaaatttcgctacggataaagggttcccgaaaacgatctttaaaaacgccacattccagttacagatggataacgatgttgctaataatactcagtatgagtggtcgtcgtcattcacacccaatgtatcggttaacgatcagggtcaggtgacgattacctaccaaacctatagcgaagtggctgtgacggcgaaaagtaaaaaattcccaagttattcggtgagttatcggttctacccaaatcggtggatatacgatggcggcagatcgctggtatccagtctcgaggccagcagacaatgccaaggttcagatatgtctgcggttcttgaatcctcacgtgcaaccaacggaacgcgtgcgcctgacgggacattgtggggcgagtgggggagcttgaccgcgtatagttctgattggcaatctggtgaatattgggtcaaaaagaccagcacggattttgaaaccatgaatatggacacaggcgcactgcaaccagggcctgcatacttggcgttcccgctctgtgcgctgtcaatataaccagataacagatagcaataagaacagtttaatgagctgattatttggggcgcgaatgggagtccggcaatcctagactcgccccataagtagcaaacgtccagaagaacaacgccgctcaggttaattgagcggcgctgtttttttaaaaggattgtcgcgattaaatgccgatcttacggcccagctgcagcccgggggatctatgcggtgtgaaataccgcacagatgcgtaaggagaaaataccgcatcaggcgccattcgccattcaggctgcgcaactgttgggaagggcgatcggtgcgggcctcttcgctattacgccaggacttcatatacccaagcttggaaaattttttttaaaaaagtcttgacactttatgcttccggctcgtataatggatccaggagtaacaatacaaatggattcaagagatccatttgtattgttactcctttttttttttgtcgacgatccttagcgaaagctaaggattttttttttactcgagcggattactacatacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgatctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaatcatgacattaacctataaaaataggcgtatcacgaggccctttcgtc

SEQ ID NO: 564 - DNA, 8443 bp, Artificial sequence
  Synthetic pMBV43 plasmid construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgacggtatcgataagcttgataagcttttaaatcagcaggggtctttttggcttgtgtattattttgaagtttttcttccccgacagaatctgcttttaccgtcatagtgaaatgagcctgaaaagctattaccatgatgatacaaataagtttacttttcatttcaccgctcctttttaattcgtaaaactaagtttaagccacctacaactaatctgacagagagagttaaggacacgttttttagtatatgtgggaactaaattatacgttttgcagtagaaactataggtggcttaaactttgggatatgcttatattatatggataaacagtcagatattcttttacatttgttaattcttctaaaaaaattaaaaaataagcctgtttctacattcttcacaaaataatttacgaagagtgcaaaacaagcttattttttcgtgtgtgttaagcggttttattcttaattttttattacttttacaattattcgattggattatctactttattactatatttcggataaagcgtggtgccccagatggagatatttctatttttcacaagtggtaagttccggtcatcaattaccgttctccaccattcccaagctaaaccagtgcattctttagcgtaaacattaatatttctcgcgttacctggcaaatagatggacgatgtgaaatgagctagcttgcttttattgttttcgctccagtttttatgttgaacaatttcgttaccttcaggatcataatttacttcatcccaagaaatgttgaattgagcaacgtatcctccagagtgatcgatgttaatttttccatctgtataagcttttgaagttgtttcaatatattctgagttgtttttaataacagctaattcattgtcttttaggaagtttgttgtataagcaatgggaactcctggtgtttctcgattaaaagtagcgccttttttcaaaatatcgcgtaagtctccgaggttgccgtcgatgatttgaacttcatcttttgcggaacctccgtaaattacggctttgaaggaagaatttttgatgatatttgttagttctacatcacctgagacagattttccgcttacggcagcatcaaaagcagcttttactttagtactatgggaattagttgataatttcaaataaacttgacggccatacgccacacttgagatatatgcaggaggattttctgcattcactccaagcgcttgcaactgctctttagtaacagctttgccgaaaaatctggaaggtcttgtaggttcattaacattcacgttatagtaaatttgtttaaaactaatgacttcttcttgcattttcccttcactgattgcgccgaagtttacattcaagctattatttacagctttaaatgctgtaccaaatttcgcaattaattgtgattcactgtaagccatttcgtcatcataatcaatttttgcacttacatttggataagcttgagcatatttttcattccatctttccactaatgtatttactgcgttgttaacgtttgatttagtggcattttttacaacgattttattgtcttgattagtcatacctggcaaatcaatgctgagtgttaatgaatcacgttttacagggagaacatctggttgattttctactaattccgaattcgcttttacgagagcacctggataggttaggctcgaaattgcattcacaacttgaatgtctgcattattttgattgatggatttcttctttttctccacaacaatatattcatttccatctttgtaaccttttcttggcggcacatttgtcactgcatctccgtggtatactaatacattgtttttattgtaatccaatccttgtatatacttatcgatttcatccgcgtgtttcttttcgattggcgtcttaggacttgcaggcggagatgctggtggtgccatggatgaaattgaattttctttattgaatgcagatgcatcctttgcttcagtttgttgcgcaattggtagactaactaatataagtgtaataaaaactagcattatttttttcatgggtttcactctccttctacattttttaacctaataatgccaaataccgtttgccacccctctcttttgataattataatattggcgaaattcgcttctaaagatgaaacgcaatattatatgcttgctttatagctttattctagtcctgctgtccctttatcgtcgttaacaaatgttaatgcctcaacataaaagtcactttaagataggaatatactaatcaaaggagggatcgaattcctgcagtcatcaaggcaaccatcaggattaatgcggatattgcggagtaacacttcagactgaaagtagaaataaaaaccgcagcagacaactgacaacatcaaatgaagggggcttattctaattgatattatttatatgataatagttcattttgtattttgtttttttgatattctcacctgcttagttacaataaatcaattctatcgctgtatggtatagactgttttattatatattttgaatatttttaatctgcccagtctggttttttaaaaaagtgctatcctcttaatgtctttactaaattagaaaacaagtttcactttcaactattgcatctttaattaatggtcaaggtgatttcaaatgctcgtttgtggccagttatacctcaaataactcaagttgttgagcacagccaacgcacatgcagtttgacgtatgacaggtatgctttatttcatttaaattatgatggttttccagccaatcagtgagtttctcttgataaggaatgcgggaatgtctatgtattttaataaaataatttcatttaatattatttcacgaatagttatttgtatctttttgatatgtggaatgttcatggctggggcttcagaaaaatatgatgctaacgcaccgcaacaggtccagccttattctgtctcttcatctgcatttgaaaatctccatcctaataatgaaatggagagttcaatcaatcccttttccgcatcggatacagaaagaaatgctgcaataatagatcgcgccaataaggagcaggagactgaagcggtgaataagatgataagcaccggggccaggttagctgcatcaggcagggcatctgatgttgctcactcaatggtgggcgatgcggttaatcaagaaatcaaacagtggttaaatcgattcggtacggctcaagttaatctgaattttgacaaaaatttttcgctaaaagaaagctctcttgattggctggctccttggtatgactctgcttcattcctcttttttagtcagttaggtattcgcaataaagacagccgcaacacacttaaccttggcgtcgggatacgtacattggagaacggttggctgtacggacttaatactttttatgataatgatttgaccggccacaaccaccgtatcggtcttggtgccgaggcctggaccgattatttacagttggctgccaatgggtattttcgcctcaatggatggcactcgtcgcgtgatttctccgactataaagagcgcccagccactgggggggatttgcgcgcgaatgcttatttacctgcactcccacaactgggggggaagttgatgtatgagcaatacaccggtgagcgtgttgctttatttggtaaagataatctgcaacgcaacccttatgccgtgactgccgggatcaattacacccccgtgcctctactcactgtcggggtagatcagcgtatggggaaaagcagtaagcatgaaacacagtggaacctccaaatgaactatcgcctgggcgagagttttcagtcgcaacttagcccttcagcggtggcaggaacacgtctactggcggagagccgctataaccttgtcgatcgtaacaataatatcgtgttggagtatcagaaacagcaggtggttaaactgacattatcgccagcaactatctccggcctgccgggtcaggtttatcaggtgaacgcacaagtacaaggggcatctgctgtaagggaaattgtctggagtgatgccgaactgattgccgctggcggcacattaacaccactgagtaccacacaattcaacttggttttaccgccttataaacgcacagcacaagtgagtcgggtaacggacgacctgacagccaacttttattcgcttagtgcgctcgcggttgatcaccaaggaaaccgatctaactcattcacattgagcgtcaccgttcagcagcctcagttgacattaacggcggccgtcattggtgatggcgcaccggctaatgggaaaactgcaatcaccgttgagttcaccgttgctgattttgaggggaaacccttagccgggcaggaggtggtgataaccaccaataatggtgcgctaccgaataaaatcacggaaaagacagatgcaaatggcgtcgcgcgcattgcattaaccaatacgacagatggcgtgacggtagtcacagcagaagtggaggggcaacggcaaagtgttgatacccactttgttaagggtactatcgcggcggataaatccactctggctgcggtaccgacatctatcatcgctgatggtctaatggcttcaaccatcacgttggagttgaaggatacctatggggacccgcaggctggcgcgaatgtggcttttgacacaaccttaggcaatatgggcgttatcacggatcacaatgacggcacttatagcgcaccattgaccagtaccacgttgggggtagcaacagtaacggtgaaagtggatggggctgcgttcagtgtgccgagtgtgacggttaatttcacggcagatcctattccagatgctggccgctccagtttcaccgtctccacaccggatatcttggctgatggcacgatgagttccacattatcctttgtccctgtcgataagaatggccattttatcagtgggatgcagggcttgagttttactcaaaacggtgtgccggtgagtattagccccattaccgagcagccagatagctataccgcgacggtggttgggaatagtgtcggtgatgtcacaatcacgccgcaggttgataccctgatactgagtacattgcagaaaaaaatatccctattcccggtacctacgctgaccggtattctggttaacgggcaaaatttcgctacggataaagggttcccgaaaacgatctttaaaaacgccacattccagttacagatggataacgatgttgctaataatactcagtatgagtggtcgtcgtcattcacacccaatgtatcggttaacgatcagggtcaggtgacgattacctaccaaacctatagcgaagtggctgtgacggcgaaaagtaaaaaattcccaagttattcggtgagttatcggttctacccaaatcggtggatatacgatggcggcagatcgctggtatccagtctcgaggccagcagacaatgccaaggttcagatatgtctgcggttcttgaatcctcacgtgcaaccaacggaacgcgtgcgcctgacgggacattgtggggcgagtgggggagcttgaccgcgtatagttctgattggcaatctggtgaatattgggtcaaaaagaccagcacggattttgaaaccatgaatatggacacaggcgcactgcaaccagggcctgcatacttggcgttcccgctctgtgcgctgtcaatataaccagataacagatagcaataagaacagtttaatgagctgattatttggggcgcgaatgggagtccggcaatcctagactcgccccataagtagcaaacgtccagagaacaacgccgctcaggttaattgagcggcgttgtttttttaaaaggatttgtcgcgataagcgtgagctggcgttaaatgccgatcttacggcccagctgcagcccgggggatctatgcggtgtgaaataccgcacagatgcgtaaggagaaaataccgcatcaggcgccattcgccattcaggctgcgcaactgttgggaagggcgatcggtgcgggcctcttcgctattacgccagacttcattatacccaagcttggaaaattttttttaaaaaagtcttgacactttatgcttccggctcgtataatggatccaggagtaacaatacaaatggattcaagagatccatttgtattgttactccttttttttttttgtcgacgatccttagcgaaagctaaggattttttttttactcgagcggattactacatacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgatctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaatcatgacattaacctataaaaataggcgtatcacgaggccctttcgtct

SEQ ID NO: 565 - DNA, 8427 bp, Artificial sequence
  Synthetic pMBV44 plasmid construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgacggtatcgataagcttgataagcttttaaatcagcaggggtctttttggcttgtgtattattttgaagtttttcttccccgacagaatctgcttttaccgtcatagtgaaatgagcctgaaaagctattaccatgatgatacaaataagtttacttttcatttcaccgctcctttttaattcgtaaaactaagtttaagccacctacaactaatctgacagagagagttaaggacacgttttttagtatatgtgggaactaaattatacgttttgcagtagaaactataggtggcttaaactttgggatatgcttatattatatggataaacagtcagatattcttttacatttgttaattcttctaaaaaaattaaaaaataagcctgtttctacattcttcacaaaataatttacgaagagtgcaaaacaagcttattttttcgtgtgtgttaagcggttttattcttaattttttattacttttacaattattcgattggattatctactttattactatatttcggataaagcgtggtgccccagatggagatatttctatttttcacaagtggtaagttccggtcatcaattaccgttctccaccattcccaagctaaaccagtgcattctttagcgtaaacattaatatttctcgcgttacctggcaaatagatggacgatgtgaaatgagctagcttgcttttattgttttcgctccagtttttatgttgaacaatttcgttaccttcaggatcataatttacttcatcccaagaaatgttgaattgagcaacgtatcctccagagtgatcgatgttaatttttccatctgtataagcttttgaagttgtttcaatatattctgagttgtttttaataacagctaattcattgtcttttaggaagtttgttgtataagcaatgggaactcctggtgtttctcgattaaaagtagcgccttttttcaaaatatcgcgtaagtctccgaggttgccgtcgatgatttgaacttcatcttttgcggaacctccgtaaattacggctttgaaggaagaatttttgatgatatttgttagttctacatcacctgagacagattttccgcttacggcagcatcaaaagcagcttttactttagtactatgggaattagttgataatttcaaataaacttgacggccatacgccacacttgagatatatgcaggaggattttctgcattcactccaagcgcttgcaactgctctttagtaacagctttgccgaaaaatctggaaggtcttgtaggttcattaacattcacgttatagtaaatttgtttaaaactaatgacttcttcttgcattttcccttcactgattgcgccgaagtttacattcaagctattatttacagctttaaatgctgtaccaaatttcgcaattaattgtgattcactgtaagccatttcgtcatcataatcaatttttgcacttacatttggataagcttgagcatatttttcattccatctttccactaatgtatttactgcgttgttaacgtttgatttagtggcattttttacaacgattttattgtcttgattagtcatacctggcaaatcaatgctgagtgttaatgaatcacgttttacagggagaacatctggttgattttctactaattccgaattcgcttttacgagagcacctggataggttaggctcgaaattgcattcacaacttgaatgtctgcattattttgattgatggatttcttctttttctccacaacaatatattcatttccatctttgtaaccttttcttggcggcacatttgtcactgcatctccgtggtatactaatacattgtttttattgtaatccaatccttgtatatacttatcgatttcatccgcgtgtttcttttcgattggcgtcttaggacttgcaggcggagatgctggtggtgccatggatgaaattgaattttctttattgaatgcagatgcatcctttgcttcagtttgttgcgcaattggtagactaactaatataagtgtaataaaaactagcattatttttttcatgggtttcactctccttctacattttttaacctaataatgccaaataccgtttgccacccctctcttttgataattataatattggcgaaattcgcttctaaagatgaaacgcaatattatatgcttgctttatagctttattctagtcctgctgtccctttatcgtcgttaacaaatgttaatgcctcaacataaaagtcactttaagataggaatatactaatcaaaggagggatcgaattcctgcagtcatcaaggcaaccatcaggattaatgcggatattgcggagtaacacttcagactgaaagtagaaataaaaaccgcagcagacaactgacaacatcaaatgaagggggcttattctaattgatattatttatatgataatagttcattttgtatttttgttttttttgatattctcacctgcttagttacaataaatcaattctatcgctgtatggtatagactgttttattatatattttgaatatttttaatctgcccagtctggttttttaaaaaagtgctatcctcttaatgtctttactaaattagaaaacaagtttcactttcaactattgcatctttaattaatggtcaaggtgatttcaaatgctcgtttgtggccagttatacctcaaataactcaagttgttgagcacagccaacgcacatgcagtttgacgtatgacaggtatgctttatttcatttaaattatgatggttttccagccaatcagtgagtttctcttgataaggaatgcgggaatgtctatgtattttaataaaataatttcatttaatattatttcacgaatagttatttgtatctttttgatatgtggaatgttcatggctggggcttcagaaaaatatgatgctaacgcaccgcaacaggtccagccttattctgtctcttcatctgcatttgaaaatctccatcctaataatgaaatggagagttcaatcaatcccttttccgcatcggatacagaaagaaatgctgcaataatagatcgcgccaataaggagcaggagactgaagcggtgaataagatgataagcaccggggccaggttagctgcatcaggcagggcatctgatgttgctcactcaatggtgggcgatgcggttaatcaagaaatcaaacagtggttaaatcgattcggtacggctcaagttaatctgaattttgacaaaaatttttcgctaaaagaaagctctcttgattggctggctccttggtatgactctgcttcattcctcttttttagtcagttaggtattcgcaataaagacagccgcaacacacttaaccttggcgtcgggatacgtacattggagaacggttggctgtacggacttaatactttttatgataatgatttgaccggccacaaccaccgtatcggtcttggtgccgaggcctggaccgattatttacagttggctgccaatgggtattttcgcctcaatggatggcactcgtcgcgtgatttctccgactataaagagcgcccagccactgggggggatttgcgcgcgaatgcttatttacctgcactcccacaactgggggggaagttgatgtatgagcaatacaccggtgagcgtgttgctttatttggtaaagataatctgcaacgcaacccttatgccgtgactgccgggatcaattacacccccgtgcctctactcactgtcggggtagatcagcgtatggggaaaagcagtaagcatgaaacacagtggaacctccaaatgaactatcgcctgggcgagagttttcagtcgcaacttagcccttcagcggtggcaggaacacgtctactggcggagagccgctataaccttgtcgatcgtaacaataatatcgtgttggagtatcagaaacagcaggtggttaaactgacattatcgccagcaactatctccggcctgccgggtcaggtttatcaggtgaacgcacaagtacaaggggcatctgctgtaagggaaattgtctggagtgatgccgaactgattgccgctggcggcacattaacaccactgagtaccacacaattcaacttggttttaccgccttataaacgcacagcacaagtgagtcgggtaacggacgacctgacagccaacttttattcgcttagtgcgctcgcggttgatcaccaaggaaaccgatctaactcattcacattgagcgtcaccgttcagcagcctcagttgacattaacggcggccgtcattggtgatggcgcaccggctaatgggaaaactgcaatcaccgttgagttcaccgttgctgattttgaggggaaacccttagccgggcaggaggtggtgataaccaccaataatggtgcgctaccgaataaaatcacggaaaagacagatgcaaatggcgtcgcgcgcattgcattaaccaatacgacagatggcgtgacggtagtcacagcagaagtggaggggcaacggcaaagtgttgatacccactttgttaagggtactatcgcggcggataaatccactctggctgcggtaccgacatctatcatcgctgatggtctaatggcttcaaccatcacgttggagttgaaggatacctatggggacccgcaggctggcgcgaatgtggcttttgacacaaccttaggcaatatgggcgttatcacggatcacaatgacggcacttatagcgcaccattgaccagtaccacgttgggggtagcaacagtaacggtgaaagtggatggggctgcgttcagtgtgccgagtgtgacggttaatttcacggcagatcctattccagatgctggccgctccagtttcaccgtctccacaccggatatcttggctgatggcacgatgagttccacattatcctttgtccctgtcgataagaatggccattttatcagtgggatgcagggcttgagttttactcaaaacggtgtgccggtgagtattagccccattaccgagcagccagatagctataccgcgacggtggttgggaatagtgtcggtgatgtcacaatcacgccgcaggttgataccctgatactgagtacattgcagaaaaaaatatccctattcccggtacctacgctgaccggtattctggttaacgggcaaaatttcgctacggataaagggttcccgaaaacgatctttaaaaacgccacattccagttacagatggataacgatgttgctaataatactcagtatgagtggtcgtcgtcattcacacccaatgtatcggttaacgatcagggtcaggtgacgattacctaccaaacctatagcgaagtggctgtgacggcgaaaagtaaaaaattcccaagttattcggtgagttatcggttctacccaaatcggtggatatacgatggcggcagatcgctggtatccagtctcgaggccagcagacaatgccaaggttcagatatgtctgcggttcttgaatcctcacgtgcaaccaacggaacgcgtgcgcctgacgggacattgtggggcgagtgggggagcttgaccgcgtatagttctgattggcaatctggtgaatattgggtcaaaaagaccagcacggattttgaaaccatgaatatggacacaggcgcactgcaaccagggcctgcatacttggcgttcccgctctgtgcgctgtcaatataaccagataacagatagcaataagaacagtttaatgagctgattatttggggcgcgaatgggagtccggcaatcctagactcgccccataagtagcaaacgtccagaagaacaacgccgctcaggttaattgagcggcgctgtttttttaaaaggattgtcgcgattaaatgccgatcttacggcccagctgcagcccgggggatctatgcggtgtgaaataccgcacagatgcgtaaggagaaaataccgcatcaggcgccattcgccattcaggctgcgcaactgttgggaagggcgatcggtgcgggcctcttcgctattacgccaggacttcatatacccaagcttggaaaattttttttaaaaaagtcttgacactttatgcttccggctcgtataatggatccaggagtaacaatacaaatggattcaagagatccatttgtattgttactcctttttttttttgtcgacgatccttagcgaaagctaaggattttttttttactcgagcggattactacatacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgatttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagatcatgacattaacctataaaaataggcgtatcacgaggccctttcgtc

SEQ ID NO: 566 - DNA, 18936 bp, Artificial sequence
  Synthetic pNJSZc plasmid construct
ggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaaaaaccgcgccatggtgtgtaggctggagctgcttcgaagttcctatactttctagagaataggaacttcggaataggaacttcaagatcccccacgctgccgcaagcactcagggcgcaagggctgctaaaggaaacggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgagtgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccagcttcaaaagcgctctgaagttcctatactttctagagaataggaacttcggaataggaactaaggaggatattcatatggaccatggcgcggcatgcaagctcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacagtatcgataagcttgataagcttttaaatcagcaggggtctttttggcttgtgtattattttgaagtttttcttccccgacagaatctgcttttaccgtcatagtgaaatgagcctgaaaagctattaccatgatgatacaaataagtttacttttcatttcaccgctcctttttaattcgtaaaactaagtttaagccacctacaactaatctgacagagagagttaaggacacgttttttagtatatgtgggaactaaattatacgttttgcagtagaaactataggtggcttaaactttgggatatgcttatattatatggataaacagtcagatattcttttacatttgttaattcttctaaaaaaattaaaaaataagcctgtttctacattcttcacaaaataatttacgaagagtgcaaaacaagcttattttttcgtgtgtgttaagcggttttattcttaattttttattacttttacaattattcgattggattatctactttattactatatttcggataaagcgtggtgccccagatggagatatttctatttttcacaagtggtaagttccggtcatcaattaccgttctccaccattcccaagctaaaccagtgcattctttagcgtaaacattaatatttctcgcgttacctggcaaatagatggacgatgtgaaatgagctagcttgcttttattgttttcgctccagtttttatgttgaacaatttcgttaccttcaggatcataatttacttcatcccaagaaatgttgaattgagcaacgtatcctccagagtgatcgatgttaatttttccatctgtataagcttttgaagttgtttcaatatattctgagttgtttttaataacagctaattcattgtcttttaggaagtttgttgtataagcaatgggaactcctggtgtttctcgattaaaagtagcgccttttttcaaaatatcgcgtaagtctccgaggttgccgtcgatgatttgaacttcatcttttgcggaacctccgtaaattacggctttgaaggaagaatttttgatgatatttgttagttctacatcacctgagacagattttccgcttacggcagcatcaaaagcagcttttactttagtactatgggaattagttgataatttcaaataaacttgacggccatacgccacacttgagatatatgcaggaggattttctgcattcactccaagcgcttgcaactgctctttagtaacagctttgccgaaaaatctggaaggtcttgtaggttcattaacattcacgttatagtaaatttgtttaaaactaatgacttcttcttgcattttcccttcactgattgcgccgaagtttacattcaagctattatttacagctttaaatgctgtaccaaatttcgcaattaattgtgattcactgtaagccatttcgtcatcataatcaatttttgcacttacatttggataagcttgagcatatttttcattccatctttccactaatgtatttactgcgttgttaacgtttgatttagtggcattttttacaacgattttattgtcttgattagtcatacctggcaaatcaatgctgagtgttaatgaatcacgttttacagggagaacatctggttgattttctactaattccgaattcgcttttacgagagcacctggataggttaggctcgaaattgcattcacaacttgaatgtctgcattattttgattgatggatttcttctttttctccacaacaatatattcatttccatctttgtaaccttttcttggcggcacatttgtcactgcatctccgtggtatactaatacattgtttttattgtaatccaatccttgtatatacttatcgatttcatccgcgtgtttcttttcgattggcgtcttaggacttgcaggcggagatgctggtggtgccatggatgaaattgaattttctttattgaatgcagatgcatcctttgcttcagtttgttgcgcaattggtagactaactaatataagtgtaataaaaactagcattatttttttcatgggtttcactctccttctacattttttaacctaataatgccaaataccgtttgccacccctctcttttgataattataatattggcgaaattcgcttctaaagatgaaacgcaatattatatgcttgctttatagctttattctagtcctgctgtccctttatcgtcgttaacaaatgttaatgcctcaacataaaagtcactttaagataggaatatactaatcaaaggagggatcgaattcctgcagtcatcaaggcaaccatcaggattaatgcggatattgcggagtaacacttcagactgaaagtagaaataaaaaccgcagcagacaactgacaacatcaaatgaagggggcttattctaattgatattatttatatgataatagttcattttgtattttgtttttttgatattctcacctgcttagttacaataaatcaattctatcgctgtatggtatagactgttttattatatattttgaatatttttaatctgcccagtctggttttttaaaaaagtgctatcctcttaatgtctttactaaattagaaaacaagtttcactttcaactattgcatctttaattaatggtcaaggtgatttcaaatgctcgtttgtggccagttatacctcaaataactcaagttgttgagcacagccaacgcacatgcagtttgacgtatgacaggtatgctttatttcatttaaattatgatggttttccagccaatcagtgagtttctcttgataaggaatgcgggaatgtctatgtattttaataaaataatttcatttaatattatttcacgaatagttatttgtatctttttgatatgtggaatgttcatggctggggcttcagaaaaatatgatgctaacgcaccgcaacaggtccagccttattctgtctcttcatctgcatttgaaaatctccatcctaataatgaaatggagagttcaatcaatcccttttccgcatcggatacagaaagaaatgctgcaataatagatcgcgccaataaggagcaggagactgaagcggtgaataagatgataagcaccggggccaggttagctgcatcaggcagggcatctgatgttgctcactcaatggtgggcgatgcggttaatcaagaaatcaaacagtggttaaatcgattcggtacggctcaagttaatctgaattttgacaaaaatttttcgctaaaagaaagctctcttgattggctggctccttggtatgactctgcttcattcctcttttttagtcagttaggtattcgcaataaagacagccgcaacacacttaaccttggcgtcgggatacgtacattggagaacggttggctgtacggacttaatactttttatgataatgatttgaccggccacaaccaccgtatcggtcttggtgccgaggcctggaccgattatttacagttggctgccaatgggtattttcgcctcaatggatggcactcgtcgcgtgatttctccgactataaagagcgcccagccactgggggggatttgcgcgcgaatgcttatttacctgcactcccacaactgggggggaagttgatgtatgagcaatacaccggtgagcgtgttgctttatttggtaaagataatctgcaacgcaacccttatgccgtgactgccgggatcaattacacccccgtgcctctactcactgtcggggtagatcagcgtatggggaaaagcagtaagcatgaaacacagtggaacctccaaatgaactatcgcctgggcgagagttttcagtcgcaacttagcccttcagcggtggcaggaacacgtctactggcggagagccgctataaccttgtcgatcgtaacaataatatcgtgttggagtatcagaaacagcaggtggttaaactgacattatcgccagcaactatctccggcctgccgggtcaggtttatcaggtgaacgcacaagtacaaggggcatctgctgtaagggaaattgtctggagtgatgccgaactgattgccgctggcggcacattaacaccactgagtaccacacaattcaacttggttttaccgccttataaacgcacagcacaagtgagtcgggtaacggacgacctgacagccaacttttattcgcttagtgcgctcgcggttgatcaccaaggaaaccgatctaactcattcacattgagcgtcaccgttcagcagcctcagttgacattaacggcggccgtcattggtgatggcgcaccggctaatgggaaaactgcaatcaccgttgagttcaccgttgctgattttgaggggaaacccttagccgggcaggaggtggtgataaccaccaataatggtgcgctaccgaataaaatcacggaaaagacagatgcaaatggcgtcgcgcgcattgcattaaccaatacgacagatggcgtgacggtagtcacagcagaagtggaggggcaacggcaaagtgttgatacccactttgttaagggtactatcgcggcggataaatccactctggctgcggtaccgacatctatcatcgctgatggtctaatggcttcaaccatcacgttggagttgaaggatacctatggggacccgcaggctggcgcgaatgtggcttttgacacaaccttaggcaatatgggcgttatcacggatcacaatgacggcacttatagcgcaccattgaccagtaccacgttgggggtagcaacagtaacggtgaaagtggatggggctgcgttcagtgtgccgagtgtgacggttaatttcacggcagatcctattccagatgctggccgctccagtttcaccgtctccacaccggatatcttggctgatggcacgatgagttccacattatcctttgtccctgtcgataagaatggccattttatcagtgggatgcagggcttgagttttactcaaaacggtgtgccggtgagtattagccccattaccgagcagccagatagctataccgcgacggtggttgggaatagtgtcggtgatgtcacaatcacgccgcaggttgataccctgatactgagtacattgcagaaaaaaatatccctattcccggtacctacgctgaccggtattctggttaacgggcaaaatttcgctacggataaagggttcccgaaaacgatctttaaaaacgccacattccagttacagatggataacgatgttgctaataatactcagtatgagtggtcgtcgtcattcacacccaatgtatcggttaacgatcagggtcaggtgacgattacctaccaaacctatagcgaagtggctgtgacggcgaaaagtaaaaaattcccaagttattcggtgagttatcggttctacccaaatcggtggatatacgatggcggcagatcgctggtatccagtctcgaggccagcagacaatgccaaggttcagatatgtctgcggttcttgaatcctcacgtgcaaccaacggaacgcgtgcgcctgacgggacattgtggggcgagtgggggagcttgaccgcgtatagttctgattggcaatctggtgaatattgggtcaaaaagaccagcacggattttgaaaccatgaatatggacacaggcgcactgcaaccagggcctgcatacttggcgttcccgctctgtgcgctgtcaatataaccagataacagatagcaataagaacagtttaatgagctgattatttggggcgcgaatgggagtccggcaatcctagactcgccccataagtagcaaacgtccagagaacaacgccgctcaggttaattgagcggcgttgtttttttaaaaggatttgtcgcgataagcgtgagctggcgttaaatgccgatcttacggcccagctgcagcccggctagtaacggccgccagtgtgctggaattcgcccttaatcggcatcattcaccaagcttgccaggcgactgtcttcaatattacagccgcaactactgacatggcgggtgatggtgttcactattccagggcgatcggcacccaacgcagtgatcaccagataatgttgcgatgacagtgtcaaactggttattccttcaaggggtgagttgttcttaagcatgccggtttgctgtaaagtttagggagatttgatggcttactctgttcaaaagtcgcgcctggcaaaggttgcgggtgtttcgcttgttttattactcgctgcctgtagttctgactcacgctataagcgtcaggtcagtggtgatgaagcctacctggaagcgccatggcatgcaagggcgaattctgcagatatccatcacactggcggccctagaccaggctttacactttatgcttccggctcgtataatgtgtggaaggatccaggagtaacaatacaaatggattcaagagatccatttgtattgttactcctttgtcgactggacagttcaagagactgtccatcaatatcagctttgtcacaaaccccgccaccggcggggtttttttctgctctagggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaaaaaccgcgccatggtgtgtaggctggagctgcttcgaagttcctatactttctagagaataggaacttcggaataggaacttcaagatcccccacgctgccgcaagcactcagggcgcaagggctgctaaaggaaacggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgagtgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccagcttcaaaagcgctctgaagttcctatactttctagagaataggaacttcggaataggaactaaggaggatattcatatggaccatggcgcggcatgcaagctcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacagtatcgataagcttgataagcttttaaatcagcaggggtctttttggcttgtgtattattttgaagtttttcttccccgacagaatctgcttttaccgtcatagtgaaatgagcctgaaaagctattaccatgatgatacaaataagtttacttttcatttcaccgctcctttttaattcgtaaaactaagtttaagccacctacaactaatctgacagagagagttaaggacacgttttttagtatatgtgggaactaaattatacgttttgcagtagaaactataggtggcttaaactttgggatatgcttatattatatggataaacagtcagatattcttttacatttgttaattcttctaaaaaaattaaaaaataagcctgtttctacattcttcacaaaataatttacgaagagtgcaaaacaagcttattttttcgtgtgtgttaagcggttttattcttaattttttattacttttacaattattcgattggattatctactttattactatatttcggataaagcgtggtgccccagatggagatatttctatttttcacaagtggtaagttccggtcatcaattaccgttctccaccattcccaagctaaaccagtgcattctttagcgtaaacattaatatttctcgcgttacctggcaaatagatggacgatgtgaaatgagctagcttgcttttattgttttcgctccagtttttatgttgaacaatttcgttaccttcaggatcataatttacttcatcccaagaaatgttgaattgagcaacgtatcctccagagtgatcgatgttaatttttccatctgtataagcttttgaagttgtttcaatatattctgagttgtttttaataacagctaattcattgtcttttaggaagtttgttgtataagcaatgggaactcctggtgtttctcgattaaaagtagcgccttttttcaaaatatcgcgtaagtctccgaggttgccgtcgatgatttgaacttcatcttttgcggaacctccgtaaattacggctttgaaggaagaatttttgatgatatttgttagttctacatcacctgagacagattttccgcttacggcagcatcaaaagcagcttttactttagtactatgggaattagttgataatttcaaataaacttgacggccatacgccacacttgagatatatgcaggaggattttctgcattcactccaagcgcttgcaactgctctttagtaacagctttgccgaaaaatctggaaggtcttgtaggttcattaacattcacgttatagtaaatttgtttaaaactaatgacttcttcttgcattttcccttcactgattgcgccgaagtttacattcaagctattatttacagctttaaatgctgtaccaaatttcgcaattaattgtgattcactgtaagccatttcgtcatcataatcaatttttgcacttacatttggataagcttgagcatatttttcattccatctttccactaatgtatttactgcgttgttaacgtttgatttagtggcattttttacaacgattttattgtcttgattagtcatacctggcaaatcaatgctgagtgttaatgaatcacgttttacagggagaacatctggttgattttctactaattccgaattcgcttttacgagagcacctggataggttaggctcgaaattgcattcacaacttgaatgtctgcattattttgattgatggatttcttctttttctccacaacaatatattcatttccatctttgtaaccttttcttggcggcacatttgtcactgcatctccgtggtatactaatacattgtttttattgtaatccaatccttgtatatacttatcgatttcatccgcgtgtttcttttcgattggcgtcttaggacttgcaggcggagatgctggtggtgccatggatgaaattgaattttctttattgaatgcagatgcatcctttgcttcagtttgttgcgcaattggtagactaactaatataagtgtaataaaaactagcattatttttttcatgggtttcactctccttctacattttttaacctaataatgccaaataccgtttgccacccctctcttttgataattataatattggcgaaattcgcttctaaagatgaaacgcaatattatatgcttgctttatagctttattctagtcctgctgtccctttatcgtcgttaacaaatgttaatgcctcaacataaaagtcactttaagataggaatatactaatcaaaggagggatcgaattcctgcagtcatcaaggcaaccatcaggattaatgcggatattgcggagtaacacttcagactgaaagtagaaataaaaaccgcagcagacaactgacaacatcaaatgaagggggcttattctaattgatattatttatatgataatagttcattttgtattttgtttttttgatattctcacctgcttagttacaataaatcaattctatcgctgtatggtatagactgttttattatatattttgaatatttttaatctgcccagtctggttttttaaaaaagtgctatcctcttaatgtctttactaaattagaaaacaagtttcactttcaactattgcatctttaattaatggtcaaggtgatttcaaatgctcgtttgtggccagttatacctcaaataactcaagttgttgagcacagccaacgcacatgcagtttgacgtatgacaggtatgctttatttcatttaaattatgatggttttccagccaatcagtgagtttctcttgataaggaatgcgggaatgtctatgtattttaataaaataatttcatttaatattatttcacgaatagttatttgtatctttttgatatgtggaatgttcatggctggggcttcagaaaaatatgatgctaacgcaccgcaacaggtccagccttattctgtctcttcatctgcatttgaaaatctccatcctaataatgaaatggagagttcaatcaatcccttttccgcatcggatacagaaagaaatgctgcaataatagatcgcgccaataaggagcaggagactgaagcggtgaataagatgataagcaccggggccaggttagctgcatcaggcagggcatctgatgttgctcactcaatggtgggcgatgcggttaatcaagaaatcaaacagtggttaaatcgattcggtacggctcaagttaatctgaattttgacaaaaatttttcgctaaaagaaagctctcttgattggctggctccttggtatgactctgcttcattcctcttttttagtcagttaggtattcgcaataaagacagccgcaacacacttaaccttggcgtcgggatacgtacattggagaacggttggctgtacggacttaatactttttatgataatgatttgaccggccacaaccaccgtatcggtcttggtgccgaggcctggaccgattatttacagttggctgccaatgggtattttcgcctcaatggatggcactcgtcgcgtgatttctccgactataaagagcgcccagccactgggggggatttgcgcgcgaatgcttatttacctgcactcccacaactgggggggaagttgatgtatgagcaatacaccggtgagcgtgttgctttatttggtaaagataatctgcaacgcaacccttatgccgtgactgccgggatcaattacacccccgtgcctctactcactgtcggggtagatcagcgtatggggaaaagcagtaagcatgaaacacagtggaacctccaaatgaactatcgcctgggcgagagttttcagtcgcaacttagcccttcagcggtggcaggaacacgtctactggcggagagccgctataaccttgtcgatcgtaacaataatatcgtgttggagtatcagaaacagcaggtggttaaactgacattatcgccagcaactatctccggcctgccgggtcaggtttatcaggtgaacgcacaagtacaaggggcatctgctgtaagggaaattgtctggagtgatgccgaactgattgccgctggcggcacattaacaccactgagtaccacacaattcaacttggttttaccgccttataaacgcacagcacaagtgagtcgggtaacggacgacctgacagccaacttttattcgcttagtgcgctcgcggttgatcaccaaggaaaccgatctaactcattcacattgagcgtcaccgttcagcagcctcagttgacattaacggcggccgtcattggtgatggcgcaccggctaatgggaaaactgcaatcaccgttgagttcaccgttgctgattttgaggggaaacccttagccgggcaggaggtggtgataaccaccaataatggtgcgctaccgaataaaatcacggaaaagacagatgcaaatggcgtcgcgcgcattgcattaaccaatacgacagatggcgtgacggtagtcacagcagaagtggaggggcaacggcaaagtgttgatacccactttgttaagggtactatcgcggcggataaatccactctggctgcggtaccgacatctatcatcgctgatggtctaatggcttcaaccatcacgttggagttgaaggatacctatggggacccgcaggctggcgcgaatgtggcttttgacacaaccttaggcaatatgggcgttatcacggatcacaatgacggcacttatagcgcaccattgaccagtaccacgttgggggtagcaacagtaacggtgaaagtggatggggctgcgttcagtgtgccgagtgtgacggttaatttcacggcagatcctattccagatgctggccgctccagtttcaccgtctccacaccggatatcttggctgatggcacgatgagttccacattatcctttgtccctgtcgataagaatggccattttatcagtgggatgcagggcttgagttttactcaaaacggtgtgccggtgagtattagccccattaccgagcagccagatagctataccgcgacggtggttgggaatagtgtcggtgatgtcacaatcacgccgcaggttgataccctgatactgagtacattgcagaaaaaaatatccctattcccggtacctacgctgaccggtattctggttaacgggcaaaatttcgctacggataaagggttcccgaaaacgatctttaaaaacgccacattccagttacagatggataacgatgttgctaataatactcagtatgagtggtcgtcgtcattcacacccaatgtatcggttaacgatcagggtcaggtgacgattacctaccaaacctatagcgaagtggctgtgacggcgaaaagtaaaaaattcccaagttattcggtgagttatcggttctacccaaatcggtggatatacgatggcggcagatcgctggtatccagtctcgaggccagcagacaatgccaaggttcagatatgtctgcggttcttgaatcctcacgtgcaaccaacggaacgcgtgcgcctgacgggacattgtggggcgagtgggggagcttgaccgcgtatagttctgattggcaatctggtgaatattgggtcaaaaagaccagcacggattttgaaaccatgaatatggacacaggcgcactgcaaccagggcctgcatacttggcgttcccgctctgtgcgctgtcaatataaccagataacagatagcaataagaacagtttaatgagctgattatttggggcgcgaatgggagtccggcaatcctagactcgccccataagtagcaaacgtccagagaacaacgccgctcaggttaattgagcggcgttgtttttttaaaaggatttgtcgcgataagcgtgagctggcgttaaatgccgatcttacggcccagctgcagcccggctagtaacggccgccagtgtgctggaattcgcccttaatcggcatcattcaccaagcttgccaggcgactgtcttcaatattacagccgcaactactgacatggcgggtgatggtgttcactattccagggcgatcggcacccaacgcagtgatcaccagataatgttgcgatgacagtgtcaaactggttattccttcaaggggtgagttgttcttaagcatgccggtttgctgtaaagtttagggagatttgatggcttactctgttcaaaagtcgcgcctggcaaaggttgcgggtgtttcgcttgttttattactcgctgcctgtagttctgactcacgctataagcgtcaggtcagtggtgatgaagcctacctggaagcgccatggcatgcaagggcgaattctgcagatatccatcacactggcggccctagaccaggctttacactttatgcttccggctcgtataatgtgtggaaggatccaggagtaacaatacaaatggattcaagagatccatttgtattgttactcctttgtcgactggacagttcaagagactgtccatcaatatcagctttgtcacaaaccccgccaccggcggggtttttttctgctctag

SEQ ID NO: 567 - DNA, 54 bp, Artificial sequence
  Chemically synthesized primer
ctgaaagtagaaataaaaaccgcagcatattttgaatatttttaatctgcccag

SEQ ID NO: 568 - DNA, 54 bp, Artificial sequence
  Chemically synthesized primer
ctgggcagattaaaaatattcaaaatatgctgcggtttttatttctactttcag

SEQ ID NO: 569 - DNA, 153 bp, Artificial sequence
  regulatory binding region within the inv promoter region
gacaactgacaacatcaaatgaagggggcttattctaattgatattatttatatgataatagttcattttgtattttgtttttttgatattctcacctgcttagttacaataaatcaattctatcgctgtatggtatagactgttttattata

SEQ ID NO: 570 - DNA, 1040 bp, Artificial sequence
  Synthetic opa52 pJS34 plasmid construct
ggtaccttgtgagcggataacaattccaggctttacactttatgcttccggctcgtataatgtgtggaattgtgagcggataacaatttcacacaggaggactagtctatgaacccggcgccgaaaaaaccgtccctgctgttttcctccctgctgttttcctccgcggcgcaggcggcaggtgaagaccatgggcgcggcccgtatgtgcaggcggatctggcttacgcctacgagcacattacccgcgattatcccgatgcagccggtgcaaacaaaggcaaaataagcacggtaagcgattatttcagaaacatccgtacgcattccatccaccccagggtgtcggtcggctacgacttcggcggctggcgcatcgccgcggattatgcccgttacaggaaatggcacaacaataaatattccgtgaacataaaagagttggaaagaaagaataataaaacttctggcggcgaccagcttaacataaaataccaaaagacggaacatcaggaaaacggcacattccacgccgtttcttctctcggcttgtcaaccgtttacgatttcagagtcaacgataaattcaaaccctatatcggtgtgcgtgtcggctacggacacgtcagacacggtatcgattcgactaaaaaaacgaaaaatactcttaccgcctaccatggtgctggcacaaaacctacgtattatgatgatatagattcgggaaaaaaccaaaaaaacacttatcgccaaaaccgcagcagccgccgcttgggcttcggcgcgatggcgggcgtgggcatagacgtcgcgcccggcctgaccttggacgccggctaccgctaccactattggggacgcctggaaaacacccgcttcaaaacccacgaagcctcattgggcgtgcgctaccgcttctgacatatggactcctgttgatagatccagtaatgacctcagaactccatctggatttgttcagaacgctcggttgccgccgggcgttttttattggtgagaatgcggccgcttgtttaaac

SEQ ID NO: 571 - protein, 270 aa, Artificial sequence
  Synthetic opa52 pJS34 plasmid construct
MNPAPKKPSLLFSSLLFSSAAQAAGEDHGRGPYVQADLAYAYEHITRDYPDAAGANKGKISTVSDYFRNIRTHSIHPRVSVGYDFGGWRIAADYARYRKWHNNKYSVNIKELERKNNKTSGGDQLNIKYQKTEHQENGTFHAVSSLGLSTVYDFRVNDKFKPYIGVRVGYGHVRHGIDSTKKTKNTLTAYHGAGTKPTYYDDIDSGKNQKNTYRQNRSSRRLGFGAMAGVGIDVAPGLTLDAGYRYHYWGRLENTRFKTHEASLGVRYRF

SEQ ID NO: 572 - DNA, 87 bp, Artificial sequence
  Synthetic PlacUV5 promoter sequence
ccaggctttacactttatgcttccggctcgtataatgtgtggaattgtgagcggataacaatttcacacaggaaacagaattctatg

SEQ ID NO: 573 - DNA, 65 bp, Artificial sequence
  Synthetic PlacUV5 promoter sequence
aagcttggaaaattttttttaaaaaagtcttgacactttatgcttccggctcgtataatggatcc

SEQ ID NO: 574 - DNA, 30 bp, Escherichia coli
gatccttagcgaaagctaaggatttttttt

(19) United States
(12) Patent Application Publication
(10) Pub. No.: US 2010/0189691 A1
(43) Pub. Date: Jul. 29, 2010

(54) E. Coli Mediated Gene Silencing of Beta-Catenin

(75) Inventors: Johannes H. Fruehauf, Newton, MA (US); Moreswhar B. Vaze, Bedford, MA (US); Floyd S. Laroux, Brookline, MA (US); Jessica A. Sexton, Brookline, MA (US)

Correspondence Address: MINTZ, LEVIN, COHN, FERRIS, GLOVSKY AND POPEO, P.C, ONE FINANCIAL CENTER, BOSTON, MA, 02111, US

(73) Assignee: Cequent Pharmaceuticals, Inc., Cambridge, MA (US)

(21) Appl. No.: 12/618,401

(22) Filed: Nov. 13, 2009

Related U.S. Application Data
(60) Provisional application No. 61/114,610, filed on Nov. 14, 2008.

(51) Int. Cl. A61K 35/74; C12N 1/21; C12N 15/74; A61P 35/00

(52) U.S. Cl. 424/93.2; 435/252.33; 435/320.1

(57) ABSTRACT

Methods are described for the delivery of one or more small interfering RNAs (siRNAs) to a eukaryotic cell using a bacterium or BTP. Methods are also described for using this bacterium to regulate gene expression in eukaryotic cells using RNA interference, and methods for treating viral diseases and disorders. The bacterium or BTP includes one or more siRNAs or one or more DNA molecules encoding one or more siRNAs. Vectors are also described for use with the bacteria of the invention for causing RNA interference in eukaryotic cells.

### CROSS REFERENCE TO RELATED APPLICATIONS

This application claims priority to, and the benefit of, U.S. Patent Application No. 61/114,610, filed Nov. 14, 2008, the contents of which are herein incorporated by reference in their entirety.

### BACKGROUND

Gene silencing through RNAi (RNA-interference) by use of short interfering RNA (siRNA) has emerged as a powerful tool for molecular biology and holds the potential tos be used for therapeutic gene silencing. Short hairpin RNA (shRNA) transcribed from small DNA plasmids within the target cell has also been shown to mediate stable gene silencing and achieve gene knockdown at levels comparable to those obtained by transfection with chemically synthesized siRNA (T. R. Brummelkamp, R. Bernards, R. Agami, Science 296, 550 (2002), P. J. Paddison, A. A. Caudiy, G. J. Hannon, PNAS 99, 1443 (2002)).

Possible applications of RNAi for therapeutic purposes are extensive and include silencing and knockdown of disease genes such as oncogenes or viral genes. One major obstacle for the therapeutic use of RNAi is the delivery of siRNA to the target cell (Zamore P D, Aronin N. Nature Medicine 9, (3):266-8 (2003)). In fact, delivery has been described as the major hurdle now for RNAi (Phillip Sharp, cited by Nature news feature, Vol 425, 2003, 10-12).

Therefore, new methods are needed for the safe and predictable administration of interfering RNAs to mammals.

### SUMMARY OF THE INVENTION

The present invention provides at least one invasive bacterium, or at least one invasive bacterial therapeutic particle (BTP), comprising a prokaryotic vector, said vector comprising one or more DNA molecules encoding one or more siRNAs, a modified P_{lacUV5} promoter, at least one Inv locus and at least one HlyA gene, wherein said siRNAs interfere with an mRNA of a gene target of interest and wherein said invasive bacterium has reduced RNase III activity when compared to wild-type bacterium. Preferably, the invasive bacterium is an invasive E. coli bacterium. Preferably, the siRNAs interfere with the mRNA of β-catenin. The present invention also provides at least one prokaryotic vector comprising one or more DNA molecules encoding one or more siRNAs, a interfere with an mRNA of a gene target of interest. Preferably, the siRNAs interfere with the mRNA of β-catenin.

The present invention also provides methods of using the various invasive bacterium, BTP and vectors provided in the invention. For example, the present invention provides methods of delivering one or more siRNAs to mammalian cells. The methods include introducing at least one invasive bacterium, or at least one invasive bacterial therapeutic particle (BTP), comprising a prokaryotic vector, said vector comprising one or more DNA molecules encoding one or more siRNAs, a modified P_{lacUV5} promoter, at least one Inv locus and at least one HlyA gene, wherein said siRNAs interfere with an mRNA of a gene target of interest and wherein said invasive bacterium has reduced RNase III activity when compared to wild-type bacterium. Preferably, the invasive bacterium is an invasive E. coli bacterium.

The present invention also provides methods of regulating gene expression in mammalian cells. The method includes introducing at least one invasive bacterium, or at least one invasive bacterial therapeutic particle (BTP), comprising a prokaryotic vector, said vector comprising one or more DNA molecules encoding one or more siRNAs, a modified P_{lacUV5} promoter, at least one Inv locus and at least one HlyA gene, wherein said siRNAs interfere with an mRNA of a gene target of interest and wherein said invasive bacterium has reduced RNase III activity when compared to wild-type bacterium, where the expressed siRNAs interfere with at least one mRNA of a gene of interest thereby regulating gene expression. Preferably, the invasive bacterium is an invasive E. coli bacterium. Preferably, the siRNAs interfere with the mRNA of β-catenin.

The present invention also provides methods of treating or preventing a disease or disorder in a mammal. The methods include regulating the expression of at least one gene in a cell known to cause a disease or disorder (e.g., known to increase proliferation, growth or dysplasia) by introducing to the cells of the mammal at least one invasive bacterium, or at least one invasive bacterial therapeutic particle (BTP), comprising a prokaryotic vector, said vector comprising one or more DNA molecules encoding one or more siRNAs, a modified P_{lacUV5} promoter, at least one Inv locus and at least one HlyA gene, wherein said siRNAs interfere with an mRNA of a gene target of interest and wherein said invasive bacterium has reduced RNase III activity when compared to wild-type bacterium, where the expressed siRNAs interfere with the mRNA of the gene known to cause a disease or disorder. Preferably, the invasive bacterium is an invasive E. coli bacterium. Preferably, the siRNAs interfere with the mRNA of β-catenin.

The expressed siRNAs can direct the multienzyme complex RNA-induced silencing complex of the cell to interact with the mRNA of one or more genes of interest (e.g., β-catenin). Preferably, the expression of β-catenin is reduced as compared to wild-type β-catenin expression or as compared to the expression of β-catenin prior to the administration or treatment with an invasive bacterium or BTP containing one or more DNA molecules encoding for one or more siRNAs. The reduced expression of β-catenin can be reduced expression of β-catenin mRNA or reduced expression of β-catenin protein. Preferably, the expression of β-catenin is reduced at least 50% as compared to wild-type β-catenin expression (when compared to a normal, healthy cell) or as compared to the expression of β-catenin prior to the administration or treatment with an invasive bacterium or BTP containing one or more DNA molecules encoding for one or more siRNAs; more preferably the expression of β-catenin is reduced at least 75% as compared to wild-type β-catenin expression or as compared to the expression of β-catenin prior to the administration or treatment with an invasive bacterium or BTP containing one or more DNA molecules encoding for one or more siRNAs; most preferably the expression of β-catenin is reduced at least 90% as compared to wild-type β-catenin expression or as compared to the expression of β-catenin prior to the administration or treatment with an invasive bacterium or BTP containing one or more DNA molecules encoding for one or more siRNAs.

Preferably, the disease or disorder can be, but is not limited to, a disease or disorder associated with the over expression of β-catenin. That is, a disease or disorder characterized by an increased expression (DNA, RNA or protein) of bcat in a cell or in a mammal in need of such treatment when compared to a normal (non-diseased) or wild-type cell or mammal. Preferably the disease or disorder to be treated is selected from the group consisting of colon cancer, rectal cancer, colorectal cancer, Crohn's disease, ulcerative colitis, familial adenomatous polyposis (FAP), Gardner's syndrome, hepatocellular carcinoma (HCC), basal cell carcinoma, pilomatricoma, medulloblastoma, and ovarian cancer.

The present invention also provides a composition containing at least one invasive bacterium or BTP and a pharmacetucally acceptable carrier.

The invasive bacterium or BTPs of the present invention can be attenuated, non-pathogenic or non-virulent bacterium

The mammalian cells can be ex vivo, in vivo or in vitro. The mammalian cells can be, but are not limited to, human, bovine, ovine, porcine, feline, buffalo, canine, goat, equine, donkey, deer, avian, bird, chicken, and primate cells. Preferably, the mammalian cells are human cells. In some preferred embodiments, the mammalian cells can be, but are not limited to, colon epithelial cells, rectal epithelial cells, intestinal epithelial cells, hepatocytes, skin epithelial cells, hair cells, neural cells, and ovarian cells.

The mammalian cells can be infected with about 10³ to 10¹¹ viable invasive bacterium or BTPs (or any integer within said ranges). Preferably, the mammalian cells can be infected with about 10⁵ to 10⁹ viable invasive bacterium or BTPs (or any integer within said ranges). The mammalian cells can be infected at a multiplicity of infection ranging from about 0.1 to 10⁶ (or any integer within said ranges). Preferably, the mammalian cells can be infected at a multiplicity of infection ranging from about 10² to 10⁴ (or any integer within said ranges).

The mammal can be, but is not limited to, human, bovine, ovine, porcine, feline, buffalo, canine, goat, equine, donkey, deer, avian, bird, chicken, and primate. Preferably, the mammal is a human.

The invasive bacterium comprises a deletion of a gene encoding RNase III. Preferably, the invasive bacteriuim comprises a deletion of an mix gene encoding RNase III. Preferably, the RNase III activity of the invasive bacterium is reduced at least 90% when compared to wild-type bacterium; more preferably the RNase III activity of the invasive bacterium is reduced at least 95% when compared to wild-type bacterium; most preferably the RNase III activity of the invasive bacterium is reduced at least 99% when compared to wild-type bacterium. Preferably, the invasive bacterium is an invasive E. coli bacterium.

The one or more DNA molecules can be transcribed into one or more shRNAs within the invasive bacterium. Preferably, the one or more shRNAs comprise a 3′ overhang or a blunt end. Preferably, the one or more shRNAs do not comprise or include a 5′ overhang (have a blunt end). Preferably, the one or more shRNAs comprise a 3′ overhang of 2-5 base pairs; more preferably, the one or more shRNAs comprise a 3′ overhang of no more than 2 base pairs (one or two base pair overhang); most preferably, the one or more shRNAs do not comprise or include a 3′ overhang (have a blunt end). The one or more shRNAs are processed into one or more siRNAs. Preferably, the one or more shRNAs are processed into one or more siRNAs within the mammalian cell.

The prokaryotic vector comprising the one or more DNA molecules encoding the one or more siRNAs can include one or more promoter sequences, enhancer sequences, terminator sequences, invasion factor sequences or lysis regulation sequences. The promoter can be a prokaryotic promoter. Preferably, the prokaryotic promoter is a T7 promoter, a P_{gapA} promoter, a P_{araBAD} promoter, a P_{tac} promoter, a P_{lacUV5} promoter, or a recA promoter. Preferably, the promoter is a prokaryotic promoter. Preferably the prokaryotic promoter is a modified P_{lacUV5} promoter. The modified modified P_{lacUV5} promoter can comprise the sequence of SEQ ID NO:573. Preferably, the modified P_{lacUV5} promoter can comprise an UP element. The UP element can comprise nucleotides 7-26 of SEQ ID NO:573. Preferably, the prokaryotic vector further comprises at least one terminator sequence. Preferably the terminator sequence comprises a consecutive series of thymidine base pairs. More preferably, the terminator sequence can comprise at least 5 consecutive thymidine base pairs. The terminator sequence preferably comprises less than 20 consecutive thymidine base pairs. The prokaryotic vector can further comprise a second terminator sequence. Preferably, the second terminator sequence can be an rrnC terminator sequence. Preferably, the rrnC terminator sequence can comprise the sequence of SEQ ID NO:30-31 or SEQ ID NO:574. Preferably, these two terminator sequences are adjacent in the prokaryotic vector (they are consecutive sequences). More preferably, the two terminator sequences are separated. Preferably, the prokaryotic vector comprises the sequence for verified pMBV43. Preferably, the sequence for verified pMBV43 is SEQ ID NO:564.

Unless otherwise defined, all technical and scientific terms used herein have the same meaning as commonly understood by one of ordinary skill in the art to which this invention belongs. In the specification, the singular forms also include the plural unless the context clearly dictates otherwise. Although methods and materials similar or equivalent to those described herein can be used in the practice or testing of the present invention, suitable methods and materials are described below. All publications, patent applications, patents, and other references mentioned herein are incorporated by reference. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

FIG. 1 is a graph showing a comparison between CEQ200 and CEQ221 at three doses in COST cells.

FIG. 2 is a schematic showing the RNase III substrate hairpin RNA structure with functional annotation.

FIG. 3 is a schematic showing the bacterial Class I RNase III cutting action of the hairpin precursor.

FIG. 4 is a schematic showing the second step of maturation (first Dicer-cleavage step).

FIG. 5 is a schematic showing the second Dicer cleavage step and maturation into active siRNA.

FIG. 6, Panel A is a graph showing that CEQ 505 was able to silence mammalian β-catenin up to 90% in a dose-dependent manner in Cos-7 cells. FIG. 6, Panel B is a graph showing that CEQ 221pNJSZc lamin (the equivalent strain targeting the lamin gene) was able to silence mammalian lamin up to 65% in a dose-dependent manner in SW480 cells.

FIG. 7 is a schematic showing H3-shRNA with strand wobbles.

FIG. 8, Panel A is a graph showing the invasive ability of the opa-expressing E. Coli strain at MOI 1. FIG. 8, Panel B is a graph showing the invasive ability of the opa-expressing E. Coli strain at MOI 10.

FIG. 9, Panel A is a graph showing the silencing of β-catenin mRNA using CEQ508 in human SW480 cells. FIG. 9, Panel B is a photograph showing the silencing of β-catenin protein using CEQ508 in human SW480 cells.

FIG. 10 is a photograph of an immunoblot showing the silencing of β-catenin protein using CEQ508 in human SW480 cells.

FIG. 11 is a graph showing the silencing of β-catenin mRNA using CEQ509 BTPs in COS-7 cells.

### DETAILED DESCRIPTION OF THE INVENTION

The invention pertains to compositions and methods of delivering small interfering RNAs (siRNAs) to eukaryotic cells using non-pathogenic or therapeutic strains of bacteria or bacterial therapeutic particles (BTPs). The bacteria or BTPs deliver DNA encoding siRNA, or siRNA itself, to effect RNA interference (RNAi) by invading into the eukaryotic host cells. Generally, to trigger RNA interference in a target cell, it is required to introduce siRNA into the cell. The siRNA is either introduced into the target cell directly or by transfection or can be transcribed within the target cell as hairpin-structured dsRNA (shRNA) from specific plasmids with RNA-polymerase III compatible promoters (e.g., U6, H1) (P. J. Paddison, A. A. Caudiy, G. J. Hannon, PNAS 99, 1443 (2002), T. R. Brummelkamp, R. Bernards, R. Agami, Science 296, 550 (2002)).

The interfering RNA of the invention regulates gene expression in eukaryotic cells. It silences or knocks down genes of interest inside target cells (e.g., decreases gene activity). The interfering RNA directs the cell-owned multienzyme-complex RISC(RNA-induced silencing complex) to the mRNA of the gene to be silenced. Interaction of RISC and mRNA results in degradation or sequestration of the mRNA. This leads to effective post-transcriptional silencing of the gene of interest. This method is referred to as Bacteria Mediated Gene Silencing (BMGS).

In the case of BMGS through delivery of siRNA expressing DNA plasmids, shRNA or siRNA are produced within the target cell after liberation of the eukaryotic transcription plasmid and trigger the highly specific process of mRNA degradation, which results in silencing of the targeted gene. Additionally, one or more cell-specific eukaryotic promoters may be used that limit the expression of siRNA or shRNA to specific target cells or tissues that are in particular metabolic states. In one embodiment of this method, the cell-specific promoter is albumin and the target cell or tissue is the liver. In another embodiment of this method, the cell-specific promoter is keratin and the specific target cell or tissue is the skin.

The non-virulent bacteria and BTPs of the invention have invasive properties (or are modified to have invasive properties) and may enter a mammalian host cell through various mechanisms. In contrast to uptake of bacteria or BTPs by professional phagocytes, which normally results in the destruction of the bacterium or BTP within a specialized lysosome, invasive bacteria or BTP strains have the ability to invade non-phagocytic host cells. Naturally occurring examples of such bacteria or BTPs are intracellular pathogens such as Yersinia, Rickettsia, Legionella, Brucella, Mycobacterium, Helicobacter, Coxiella, Chlamydia, Neisseria, Burkolderia, Bordetella, Borrelia, Listeria, Shigella, Salmonella, Staphylococcus, Streptococcus, Porphyromonas, Treponema, and Vibrio, but this property can also be transferred to other bacteria or BTPs such as E. coli, Lactobacillus, Lactococcus, or Bifidobacteriae, including probiotics through transfer of invasion-related genes (P. Courvalin, S. Goussard, C. Grillot-Courvalin, C.R. Acad. Sci. Paris 318, 1207 (1995)). In other embodiments of the invention, bacteria or BTPs used to deliver interfering RNAs to host cells include Shigella flexneri (D. R. Sizemore, A. A. Branstrom, J. C. Sadoff, Science 270, 299 (1995)), invasive E. coli (P. Courvalin, S. Goussard, C. Grillot-Courvalin, C.R. Acad. Sci. Paris 318, 1207 (1995), C. Grillot-Courvalin, S. Goussard, F. Huetz, D. M. Ojcius, P. Courvalin, Nat Biotechnol 16, 862 (1998)), Yersinia enterocolitica (A. Al-Mariri A, A. Tibor, P. Lestrate, P. Mertens, X. De Bolle, J. J. Letesson Infect Immun 70, 1915 (2002)) and Listeria monocytogenes (M. Hense, E. Domann, S. Krusch, P. Wachholz, K. E. Dittmar, M. Rohde, J. Wehland, T. Chakraborty, S. Weiss, Cell Microbiol 3, 599 (2001), S. Pilgrim, J. Stritzker, C. Schoen, A. Kolb-Maurer, G. Geginat, M. J. Loessner, I. Gentschev, W. Goebel, Gene Therapy 10, 2036 (2003)). Any invasive bacterium or BTP is useful for DNA transfer into eukaryotic cells (S. Weiss, T. Chakraborty, Curr Opinion Biotechnol 12, 467 (2001)).

BMGS is performed using the naturally invasive pathogen Salmonella typhimurium. In one aspect of this embodiment, the strains of Salmonella typhimurium include SL 7207 and VNP20009 (S. K. Hoiseth, B. A. D. Stocker, Nature 291, 238 (1981); Pawelek J M, Low K B, Bermudes D. Cancer Res. 57(20): 4537-44 (Oct. 15 1997)). In another embodiment of the invention, BMGS is performed using attenuated E. coli. In another aspect of this embodiment, the CEQ201strain is engineered to possess cell-invading properties through an invasion plasmid. In one aspect of the invention, this plasmid is a TRIP (Transkingdom RNA interference plasmid) plasmid or pNJSZ.

A double “trojan horse” technique is also used with an invasive and auxotrophic bacterium or BTP carrying a eukaryotic transcription plasmid. This plasmid is, in turn, transcribed by the target cell to form one or more hairpin RNA structures that triggers the intracellular process of RNAi. This method of the invention induces significant gene silencing of a variety of genes. In certain aspects of this embodiment, the genes include a transgene (GFP), a mutated oncogene (k-Ras) and a cancer related gene (β-catenin) in vitro.

Another aspect of BMGS according to this invention is termed Transkingdom RNAi (tkRNAi). In this aspect of the invention, siRNA is directly produced by the invasive bacteria, or accumulated in the BTPs after production in the bacteria, as opposed to the target cell. A transcription plasmid controlled by a prokaryotic promoter (e.g., T7) is inserted into the carrier bacteria through standard transformation protocols. siRNA is produced within the bacteria and is liberated within the mammalian target cell after bacterial lysis triggered either by auxotrophy or by timed addition of antibiotics.

Most bacteria contain a large number of RNA degrading enzymes, RNAses, which may degrade the siRNA causing a reduction in the activity of tkRNAi. In such cases where the RNAses of a specific bacterium will exhibit such degradation of siRNA, a targeted deletion of the gene encoding the RNAse of interest (e.g., theme gene encoding RNAse III) is performed to yield higher levels of siRNA per tkRNAi bacterium, resulting in more siRNA being delivered to the target cells, as well as more efficient gene silencing of the gene of interest within the target cell.

The RNAi methods of the invention, including BMGS and tkRNAi are used to create transient “knockdown” genetic animal models as opposed to genetically engineered knockout models to discover gene functions. The methods are also used as in vitro transfection tool for research and drug development

These methods use bacteria with desirable properties (invasiveness, attenuation, steerability) to perform BMGS and tkRNAi. Invasiveness as well as eukaryotic or prokaryotic transcription of one or several shRNA is conferred to a bacterium or BTP using plasmids (e.g., TRIP) and vectors as described in greater detail herein.

1. Bacterium and/or Bacterial Therapeutic Particles (BTPs)

The present invention provides at least one invasive bacterium, or at least one bacterial therapeutic particle (BTP), including one or more siRNAs or one or more DNA molecules encoding one or more siRNAs.

According to the invention, any microorganism that is capable of delivering a molecule, e.g., an RNA molecule or an RNA-encoding DNA molecule, into the cytoplasm of a target cell, such as by traversing the membrane and entering the cytoplasm of a cell, can be used to deliver RNA to such cells. In a preferred embodiment, the microorganism is a prokaryote. In an even more preferred embodiment, the prokaryote is a bacterium or BTP. Also within the scope of the invention are microorganisms other than bacteria that can be used for delivering RNA to a cell. For example, the microorganism can be a fungus, e.g., Cryptococcus neoformans, protozoan, e.g., Trypanosoma cruzi, Toxoplasma gondii, Leishmania donovani, and plasmodia.

In a preferred embodiment, the microorganism is a bacterium or BTP. A preferred invasive bacterium or BTP is capable of delivering at least one molecule, e.g., an RNA or RNA-encoding DNA molecule, to a target cells, such as by entering the cytoplasm of a eukaryotic cell. Preferably, the RNA is siRNA or shRNA and the RNA-encoding DNA molecule encodes for siRNA or shRNA.

BTPs are fragments of bacteria used for therapeutic or preventive purposes. BTPs may include particles known in the art as minicells. Minicells are small cells produced by cell division that is faulty near the pole. They are devoid of nucleoid and, therefore, unable to grow and form colonies (Alder et al., (1967) Proc. Nat. Acad. Sci. U.S.A. 57, 321-326; for reviews see Sullivan and Maddock, (2000) Curr. Biol. 10:R249-R252; Margolin, (2001) Curr. Biol. 11, R395-R398; Howard and Kruse, (2005) J. Cell Biol. 168, 533-536). Minicell formation results due to mutations causing a defect in selection of the site for the septum formation for cell division. Such mutations include null alleles of minC, minD (Davie et al, (1984) J. Bacteriol. 158, 1202-1203; de Boer et al., 1988) J. Bacteriol. 170, 2106-2112) and certain alleles of ftsZ (Bi and Lutkenhaus, (1992) J. Bacteriol. 174, 5414-5423). Overexpression fo FtsZ or MinC-MinD proteins has also been reported to cause the formation of minicells (Ward and Lutkenhaus, 1985; de Boer et al., 1988). Although minicells are devoid of nucleoid, they are capable of transcription and translation (Roozen et al., (1971) J. Bacteriol. 107, 21-33; Shepherd et al., (2001) J. Bacteriol. 183, 2527-34).

BTPs are distinct from bacteria in that they lack the bacterial genome and, therefore, provide a decreased risk of bacterial proliferation in patients. This is of particular value for immune-compromised patients. Furthermore, the inability of BTPs to proliferate allows for their use in sensitive tissues, e.g., the brain, and other areas of the body traditionally considered inaccessible to traditional siRNA. For example, the intraperitoneal delivery of bacteria can include the risk of adhesions and peritonitis, which is eliminated by utilizing BTPs. However, like the bacteria of this invention, BTPs contain the bacterial cell wall, some bacterial plasma contents and subcellular particles, one or more therapeutic components, e.g., one or more siRNAs, one or more invasion factors, one or more phagosome degradation factors, and one or more factors for targeting specific tissues. The BTPs are produced from bacteria that have produced and accumulated siRNAs inside the bacteria, and then segregate the bacterial fragment (BTP) during cell division. In one embodiment of this invention, BTPs are obtained by fermenting the bacteria, during which the BTPs form abundantly, followed by isolation of the BTPs from live bacteria using differential size filtration, which will retain the bacteria but allow passage and collection of BTPs. In another embodiment of this invention, BTPs are separated from bacteria by centrifugation. In another embodiment of this invention, live bacterial cells are lysed through activation of a death signal. Once isolated, the BTPs can be lyophilized and formulated for use.

As used herein, the term “invasive” when referring to a microorganism, e.g., a bacterium or BTP, refers to a microorganism that is capable of delivering at least one molecule, e.g., an RNA or RNA-encoding DNA molecule, to a target cell. An invasive microorganism can be a microorganism that is capable of traversing a cell membrane, thereby entering the cytoplasm of said cell, and delivering at least some of its content, e.g., RNA or RNA-encoding DNA, into the target cell. The process of delivery of the at least one molecule into the target cell preferably does not significantly modify the invasion apparatus.

Invasive microorganisms include microorganisms that are naturally capable of delivering at least one molecule to a target cell, such as by traversing the cell membrane, e.g., a eukaryotic cell membrane, and entering the cytoplasm, as well as microorganisms which are not naturally invasive and which have been modified, e.g., genetically modified, to be invasive. In another preferred embodiment, a microorganism that is not naturally invasive can be modified to become invasive by linking the bacterium or BTP to an “invasion factor”, also termed “entry factor” or “cytoplasm-targeting factor”. As used herein, an “invasion factor” is a factor, e.g., a protein or a group of proteins which, when expressed by a non-invasive bacterium or BTP, render the bacterium or BTP invasive. As used herein, an “invasion factor” is encoded by a “cytoplasm-targeting gene”.

In one embodiment of this invention, the microorganism is a naturally invasive bacterium or BTP selected from the group that includes, but is not limited to, Yersinia, Rickettsia, Legionella, Brucella, Mycobacterium, Helicobacter, Coxiella, Chlamydia, Neisseria, Burkolderia, Bordetella, Borrelia, Listeria, Shigella, Salmonella, Staphylococcus, Streptococcus, Porphyromonas, Treponema, Vibrio, E. coli, and Bifidobacteriae. Optionally, the naturally invasive bacterium or BTP is Yersinia expressing an invasion factor selected from the group including, but not limited to, invasin and YadA (Yersinia enterocolitica plasmid adhesion factor). Optionally, the naturally invasive bacterium or BTP is Rickettsia expressing the invasion factor RickA (actin polymerization protein). Optionally, the naturally invasive bacterium or BTP is Legionella expressing the invasion factor RaIF (guanine exchange factor). Optionally, the naturally invasive bacterium or BTP is Neisseria expressing an invasion factor selected from the group including, but not limited to, NadA (Neisseria adhesion/invasion factor), OpaA, OpaC and Opa52 (opacity-associated adhesions). Optionally, the naturally invasive bacterium or BTP is Listeria expressing an invasion factor selected from the group including, but not limited to, InlA (internalin factor), InlB (internalin factor), Hpt (hexose phosphate transporter), and ActA (actin polymerization protein). Optionally, the naturally invasive bacterium or BTP is Shigella expressing an invasion factor selected from the group including, but not limited to, the Shigella secreting factors IpaA (invasion plasmid antigen), IpaB, IpaC, IpgD, IpaB-IpaC complex, VirA, and IcsA. Optionally, the naturally invasive bacterium or BTP is Salmonella expressing an invasion factor selected from the group including, but not limited to, Salmonella secreting/exchange factors SipA, SipC, SpiC, SigD, SopB, SopE, SopE2, and SptP. Optionally, the naturally invasive bacterium or BTP is Staphylococcus expressing an invasion factor selected from the group including, but not limited to, the fibronectin binding proteins FnBPA and FnBPB. Optionally, the naturally invasive bacterium or BTP is Streptococcus expressing an invasion factor selected from the group including, but not limited to, the fibronectin binding proteins ACP, Fba, F2, Sfb1, Sfb2, SOF, and PFBP. Optionally, the naturally invasive bacterium or BTP is Porphyromonas gingivalis expressing the invasion factor FimB (integrin binding protein fibriae).

In another embodiment of this invention, the microorganism is a bacterium or BTP that is not naturally invasive but has been modified, e.g., genetically modified, to be invasive. Optionally, the bacterium or BTP that is not naturally invasive has been genetically modified to be invasive by expressing an invasion factor selected from the group including, but not limited to, invasin, YadA, RickA, RaIF, NadA, OpaA, OpaC, Opa52, InlA, InlB, Hpt, ActA, IpaA, IpaB, IpaC, IpgD, IpaB-IpaC complex, VirA, IcsA, SipA, SipC, SpiC, SigD, SopB, SopE, SopE2, SptP, FnBPA, FnBPB, ACP, Fba, F2, Sfb1, Sfb2, SOF, PFBP, and FimB.

In another embodiment of this invention, the microorganism is a bacterium or BTP that may be naturally invasive but has been modified, e.g., genetically modified, to express one or more additional invasion factors. Optionally, the invasion factor is selected from the group that includes, but is not limited to, invasin, YadA, RickA, RaIF, NadA, OpaA, OpaC, Opa52, InlA, InlB, Hpt, ActA, IpaA, IpaB, IpaC, IpgD, IpaB-IpaC complex, VirA, IcsA, SipA, SipC, SpiC, SigD, SopB, SopE, SopE2, SptP, FnBPA, FnBPB, ACP, Fba, F2, Sfb1, Sfb2, SOF, PFBP, and FimB.

Naturally invasive microorganisms, e.g., bacteria or BTPs, may have a certain tropism, i.e., preferred target cells. Alternatively, microorganisms, e.g., bacteria or BTPs can be modified, e.g., genetically, to mimic the tropism of a second microorganism. Optionally, the bacterium or BTP is Streptococcus and the preferred target cells are selected from the group including, but not limited to, pharyngeal epithelial cells, buccal epithelial cells of the tongue, and mucosal epithelial cells. Optionally, the bacterium or BTP is Porphyromonas and the preferred target cells are selected from the group including, but not limited to, oral epithelial cells. Optionally, the bacterium or BTP is Staphylococcus and the preferred target cells are mucosal epithelial cells. Optionally, the bacterium or BTP is Neisseria and the preferred target cells are selected from the group including, but not limited to, urethral epithelial cells and cervical epithelial cells. Optionally, the bacterium or BTP is E. coli and the preferred target cells are selected from the group, including but not limited to, intestinal epithelial cells, urethral epithelial cells, and the cells of the upper urinary tract. Optionally, the bacterium or BTP is Bordetella and the preferred target cells are respiratory epithelial cells. Optionally, the bacterium or BTP is Vibrio and the preferred target cells are intestinal epithelial cells. Optionally, the bacterium or BTP is Treponema and the preferred target cells are mucosal epithelial cells. Optionally, the bacterium or BTP is Mycoplasma and the preferred target cells are respiratory epithelial cells. Optionally, the bacterium or BTP is Helicobacter and the preferred target cells are the endothelial cells of the stomach. Optionally, the bacterium or BTP is Chlamydia and the preferred target cells are selected from the group including, but not limited to, conjunctival cells and urethral epithelial cells.

In another embodiment of this invention, the microorganism is a bacterium or BTP that has been modified, e.g., genetically modified, to have a certain tropism. Optionally, the preferred target cells are selected from the group including, but not limited to, pharyngeal epithelial cells, buccal epithelial cells of the tongue, mucosal epithelial cells, oral epithelial cells, epithelial cells of the urethra, cervical epithelial cells, intestinal epithelial cells, respiratory epithelial cells, cells of the upper urinary tract, epithelial cells of the stomach, and conjunctival cells. Optionally, the preferred target cells are dysplastic or cancerous epithelial cells. Optionally, the preferred target cells are activated or resting immune cells.

Delivery of at least one molecule into a target cell can be determined according to methods known in the art. For example, the presence of the molecule, by the decrease in expression of an RNA or protein silenced thereby, can be detected by hybridization or PCR methods, or by immunological methods that may include the use of an antibody.

Determining whether a microorganism is sufficiently invasive for use in the invention may include determining whether sufficient siRNA was delivered to host cells, relative to the number of microorganisms contacted with the host cells. If the amount of siRNA is low relative to the number of microorganisms used, it may be desirable to further modify the microorganism to increase its invasive potential.

Bacterial or BTP entry into cells can be measured by various methods. Intracellular bacteria or BTPs survive treatment by aminoglycoside antibiotics, whereas extracellular bacteria are rapidly killed. A quantitative estimate of bacterial or BTP uptake can be achieved by treating cell monolayers with the antibiotic gentamicin to inactivate extracellular bacteria or BTPs, then by removing said antibiotic before liberating the surviving intracellular organisms with gentle detergent and determining viable counts on standard bacteriological medium. Furthermore, bacterial or BTP entry into cells can be directly observed, e.g., by thin-section-transmission electron microscopy of cell layers or by immunofluorescent techniques (Falkow et al. (1992) Annual Rev. Cell Biol. 8:333). Thus, various techniques can be used to determine whether a specific bacterium or BTP is capable of invading a specific type of cell or to confirm bacterial invasion following modification of the bacteria or BTP, such modification of the tropism of the bacteria to mimic that of a second bacterium.

Bacteria or BTPs that can be used for delivering RNA according to the method of the invention are preferably non-pathogenic. However, pathogenic bacteria or BTP s can also be used, so long as their pathogenicity has been attenuated, to thereby render the bacteria non-harmful to a subject to which it is administered. As used herein, the term “attenuated bacterium or BTP” refers to a bacterium or BTP that has been modified to significantly reduce or eliminate its harmfulness to a subject. A pathogenic bacterium or BTP can be attenuated by various methods, set forth below.

Without wanting to be limited to a specific mechanism of action, the bacterium or BTP delivering the RNA into the eukaryotic cell can enter various compartments of the cell, depending on the type of bacterium or BTP. For example, the bacterium or BTP can be in a vesicle, e.g., a phagocytic vesicle. Once inside the cell, the bacterium or BTP can be destroyed or lysed and its contents delivered to the eukaryotic cell. A bacterium or BTP can also be engineered to express a phagosome degrading protein to allow leakage of RNA from the phagosome. In one embodiment of this invention, the bacterium or BTP expresses, either naturally or through modification, e.g., genetic modification, a protein that contributes to pore-formation, breakage or degradation of the phagosome. Optionally, the protein is a cholesterol-dependent cytolysin. Optionally, the protein is selected from the group consisting of listeriolysin, ivanolysin, streptolysin, sphingomyelinase, perfingolysin, botulinolysin, leukocidin, anthrax toxin, phospholipase, IpaB (invasion plasmid antigen), IpaH, IcsB (intercellular spread), DOT/Icm (defect in organelle trafficking/intracellular multiplication defective), DOTU (stabilization factor for the DOT/Icm complex), IcmF, and PmrA (multidrug resistance efflux Pump).

In some embodiments, the bacterium can stay alive for various times in the eukaryotic cell and may continue to produce RNA. The RNA or RNA-encoding DNA can then be released from the bacterium into the cell by, e.g., leakage. In certain embodiments of the invention, the bacterium can also replicate in the eukaryotic cell. In a preferred embodiment, bacterial replication does not kill the host cell. The invention is not limited to delivery of RNA or RNA-encoding DNA by a specific mechanism and is intended to encompass methods and compositions permitting delivery of RNA or RNA-encoding DNA by a bacterium independently of the mechanism of delivery.

In one embodiment, the bacterium or BTP for use in the present invention is non-pathogenic or non-virulent. In another aspect of this embodiment, the bacterium or BTP is therapeutic. In another aspect of this embodiment, the bacterium or BTP is an attenuated strain or derivative thereof selected from, but not limited to, Yersinia, Rickettsia, Legionella, Brucella, Mycobacterium, Helicobacter, Haemophilus, Coxiella, Chlamydia, Neisseria, Burkolderia, Bordetella, Borrelia, Listeria, Shigella, Salmonella, Staphylococcus, Streptococcus, Porphyromonas, Treponema, Vibrio, E. coli, and Bifidobacteriae. Optionally, the Yersinia strain is an attenuated strain of the Yersinia pseudotuberculosis species. Optionally, the Yersinia strain is an attenuated strain of the Yersinia enterocolitica species. Optionally, the Rickettsia strain is an attenuated strain of the Rickettsia coronii species. Optionally, the Legionella strain is an attenuated strain of the Legionella pneumophilia species. Optionally, the Mycobacterium strain is an attenuated strain of the Mycobacterium tuberculosis species. Optionally, the Mycobacterium strain is an attenuated strain of the Mycobacterium bovis BCG species. Optionally, the Helicobacter strain is an attenuated strain of the Helicobacter pylori species. Optionally, the Coxiella strain is an attenuated strain of Coxiella burnetti. Optionally, the Haemophilus strain is an attenuated strain of the Haemophilus influenza species. Optionally, the Chlamydia strain is an attenuated strain of the Chlamydia trachomatis species. Optionally, the Chlamydia strain is an attenuated strain of the Chlamydia pneumoniae species. Optionally, the Neisseria strain is an attenuated strain of the Neisseria gonorrheae species.

Optionally, the Neisseria strain is an attenuated strain of the Neisseria meningitides species. Optionally, the Burkolderia strain is an attenuated strain of the Burkolderia cepacia species. Optionally, the Bordetella strain is an attenuated strain of the Bordetella pertussis species. Optionally, the Borrelia strain is an attenuated strain of the Borrelia hermisii species. Optionally, the Listeria strain is an attenuated strain of the Listeria monocytogenes species. Optionally, the Listeria strain is an attenuated strain of the Listeria ivanovii species. Optionally, the Salmonella strain is an attenuated strain of the Salmonella enterica species. Optionally, the Salmonella strain is an attenuated strain of the Salmonella typhimurium species. Optionally, the Salmonella typhimurium strain is SL 7207 or VNP20009. Optionally, the Staphylococcus strain is an attenuated strain of the Staphylococcus aureus species. Optionally, the Streptococcus strain is an attenuated strain of the Streptococcus pyogenes species. Optionally, the Streptococcus strain is an attenuated strain of the Streptococcus mutans species. Optionally, the Streptococcus strain is an attenuated strain of the Streptococcus salivarius species. Optionally, the Streptococcus strain is an attenuated strain of the Streptococcus pneumonia species. Optionally, the Porphyromonas strain is an attenuated strain of the Porphyromonas gingivalis species. Optionally, the Pseudomonas strain is an attenuated strain of the Pseudomonas aeruginosa species. Optionally, the Treponema strain is an attenuated strain of the Treponema pallidum species. Optionally, the Vibrio strain is an attenuated strain of the Vibrio cholerae species. Optionally, the E. coli strain is MM294.

Set forth below are examples of bacteria that have been described in the literature as being naturally invasive (section 1.1), as well as bacteria which have been described in the literature as being naturally non-invasive bacteria (section 1.2), as well as bacteria which are naturally non-pathogenic or which are attenuated. Although some bacteria have been described as being non-invasive (section 1.2), these may still be sufficiently invasive for use according to the invention. Whether traditionally described as naturally invasive or non-invasive, any bacterial strain can be modified to modulate, in particular to increase, its invasive characteristics (e.g., as described in section 1.3).

### 1.1 Naturally Invasive Bacteria

The particular naturally invasive bacteria employed in the present invention are not critical thereto. Examples of such naturally occurring invasive bacteria include, but are not limited to, Shigella spp., Salmonella spp., Listeria spp., Rickettsia spp., and enteroinvasive Escherichia coli.

The particular Shigella strain employed is not critical to the present invention. Examples of Shigella strains that can be employed in the present invention include Shigella flexneri 2a (ATCC No. 29903), Shigella sonnei (ATCC No. 29930), and Shigella disenteriae (ATCC No. 13313). An attenuated Shigella strain, such as Shigella flexneri 2a 2457T aroA virG mutant CVD 1203 (Noriega et al. supra), Shigella flexneri M90T icsA mutant (Goldberg et al. Infect. Immun., 62:5664-5668 (1994)), Shigella flexneri Y SFL114 aroD mutant (Karnell et al. Vacc., 10:167-174 (1992)), and Shigella flexneri aroA aroD mutant (Verma et al. Vacc., 9:6-9 (1991)) are preferably employed in the present invention. Alternatively, new attenuated Shigella spp. strains can be constructed by introducing an attenuating mutation either singularly or in conjunction with one or more additional attenuating mutations.

At least one advantage to Shigella bacteria as delivery vectors is their tropism for lymphoid tissue in the colonic mucosal surface. In addition, the primary site of Shigella replication is believed to be within dendritic cells and macrophages, which are commonly found at the basal lateral surface of M cells in mucosal lymphoid tissues (reviewed by McGhee, J. R. et al. (1994) Reproduction, Fertility, & Development 6:369; Pascual, D. W. et al. (1994) Immunomethods 5:56). As such, Shigella vectors may provide a means to target RNA interference or deliver therapeutic molecules to these professional antigen-presenting cells. Another advantage of Shigella vectors is that attenuated Shigella strains deliver nucleic acid reporter genes in vitro and in vivo (Sizemore, D. R. et al. (1995) Science 270:299; Courvalin, P. et al. (1995) Comptes Rendus de 1 Academie des Sciences Serie III-Sciences de la Vie-Life Sciences 318:1207; Powell, R. J. et al. (1996) In: Molecular approaches to the control of infectious diseases. F. Brown, E. Norrby, D. Burton and J. Mekalanos, eds. Cold Spring Harbor Laboratory Press, New York. 183; Anderson, R. J. et al. (1997) Abstracts for the 97th General Meeting of the American Society for Microbiology:E.). On the practical side, the tightly restricted host specificity of Shigella stands to prevent the spread of Shigella vectors into the food chain via intermediate hosts. Furthermore, attenuated strains that are highly attenuated in rodents, primates and volunteers have been developed (Anderson et al. (1997) supra; Li, A. et al. (1992) Vaccine 10:395; Li, A. et al. (1993) Vaccine 11:180; Karnell, A. et al. (1995) Vaccine 13:88; Sansonetti, P. J. and J. Arondel (1989) Vaccine 7:443; Fontaine, A. et al. (1990) Research in Microbiology 141:907; Sansonetti, P. J. et al. (1991) Vaccine 9:416; Noriega, F. R. et al. (1994) Infection & Immunity 62:5168; Noriega, F. R. et al. (1996) Infection & Immunity 64:3055; Noriega, F. R. et al. (1996) Infection & Immunity 64:23; Noriega, F. R. et al. (1996) Infection & Immunity 64:3055; Kotloff, K. L. et al. (1996) Infection & Immunity 64:4542). This latter knowledge will allow the development of well-tolerated Shigella vectors for use in humans.

Attenuating mutations can be introduced into bacterial pathogens using non-specific mutagenesis either chemically, using agents such as N-methyl-N′-nitro-N-nitrosoguanidine, or using recombinant DNA techniques; classic genetic techniques, such as Tn10 mutagenesis, P22-mediated transduction, λ phage mediated crossover, and conjugational transfer; or site-directed mutagenesis using recombinant DNA techniques. Recombinant DNA techniques are preferable since strains constructed by recombinant DNA techniques are far more defined. Examples of such attenuating mutations include, but are not limited to:

(i) auxotrophic mutations, such as aro (Hoiseth et al. Nature, 291:238-239 (1981)), gua (McFarland et al. Microbiol. Path., 3:129-141 (1987)), nad (Park et al. J. Bact., 170:3725-3730 (1988), thy (Nnalue et al. Infect. Immun., 55:955-962 (1987)), and asd (Curtiss, supra) mutations;

(ii) mutations that inactivate global regulatory functions, such as cya (Curtiss et al. Infect. Immun., 55:3035-3043 (1987)), crp (Curtiss et al (1987), supra), phoP/phoQ (Groisman et al. Proc. Natl. Acad. Sci., USA, 86:7077-7081 (1989); and Miller et al. Proc. Natl. Acad. Sci., USA, 86:5054-5058 (1989)), phop^{c} (Miller et al. J. Bact., 172:2485-2490 (1990)) or ompR (Dorman et al. Infect. Immun., 57:2136-2140 (1989)) mutations;

(iii) mutations that modify the stress response, such as recA (Buchmeier et al. Mol. Micro., 7:933-936 (1993)), htrA (Johnson et al. Mol. Micro., 5:401-407 (1991)), htpR (Neidhardt et al. Biochem. Biophys. Res. Com., 100:894-900 (1981)), hsp (Neidhardt et al. Ann. Rev. Genet., 18:295-329 (1984)) and groEL (Buchmeier et al. Sci., 248:730-732 (1990)) mutations;

(iv) mutations in specific virulence factors, such as IsyA (Libby et al. Proc. Natl. Acad. Sci., USA, 91:489-493 (1994)), pag or prg (Miller et al (1990), supra; and Miller et al (1989), supra), iscA or virG (d'Hauteville et al. Mol. Micro., 6:833-841 (1992)), plcA (Mengaud et al. Mol. Microbiol., 5:367-72 (1991); Camilli et al. J. Exp. Med, 173:751-754 (1991)), and act (Brundage et al. Proc. Natl. Acad. Sci., USA, 90:11890-11894 (1993)) mutations;

(v) mutations that affect DNA topology, such as topA (Galan et al. Infect. Immun., 58:1879-1885 (1990));

(vi) mutations that disrupt or modify the cell cycle, such as min (de Boer et al. Cell, 56:641-649 (1989)).

(vii) introduction of a gene encoding a suicide system, such as sacB (Recorbet et al. App. Environ. Micro., 59:1361-1366 (1993); Quandt et al. Gene, 127:15-21 (1993)), nuc (Ahrenholtz et al. App. Environ. Micro., 60:3746-3751 (1994)), hok, gef, kil, or phlA (Molin et al. Ann. Rev. Microbiol., 47:139-166 (1993));

(viii) mutations that alter the biogenesis of lipopolysaccharide and/or lipid A, such as rFb (Raetz in Esherishia coli and Salmonella typhimurium, Neidhardt et al., Ed., ASM Press, Washington D.C. pp 1035-1063 (1996)), galE (Hone et al. J. Infect. Dis., 156:164-167 (1987)) and htrB (Raetz, supra), msbB (Reatz, supra)

(ix) introduction of a bacteriophage lysis system, such as lysogens encoded by P22 (Rennell et al. Virol, 143:280-289 (1985)), λ murein transglycosylase (Bienkowska-Szewczyk et al. Mol. Gen. Genet., 184:111-114 (1981)) or S-gene (Reader et al. Virol, 43:623-628 (1971)); and

The attenuating mutations can be either constitutively expressed or under the control of inducible promoters, such as the temperature sensitive heat shock family of promoters (Neidhardt et al. supra), or the anaerobically induced nirB promoter (Harborne et al. Mol. Micro., 6:2805-2813 (1992)) or repressible promoters, such as uapA (Gorfinkiel et al. J. Biol. Chem., 268:23376-23381 (1993)) or gcv (Stauffer et al. J. Bact., 176:6159-6164 (1994)).

The particular Listeria strain employed is not critical to the present invention. Examples of Listeria strains that can be employed in the present invention include Listeria monocytogenes (ATCC No. 15313). Attenuated Listeria strains, such as L. monocytogenes actA mutant (Brundage et al. supra) or L. monocytogenes plcA (Camilli et al. J. Exp. Med., 173:751-754 (1991)) are preferably used in the present invention. Alternatively, new attenuated Listeria strains can be constructed by introducing one or more attenuating mutations in groups (i) to (vii) as described for Shigella spp. above.

The particular Salmonella strain employed is not critical to the present invention. Examples of Salmonella strains that can be employed in the present invention include Salmonella typhi (ATCC No. 7251) and S. typhimurium (ATCC No. 13311). Attenuated Salmonella strains are preferably used in the present invention and include S. typhi-aroC-aroD (Hone et al. Vacc. 9:810 (1991) and S. typhimurium-aroA mutant (Mastroeni et al. Micro. Pathol. 13:477 (1992)). Alternatively, new attenuated Salmonella strains can be constructed by introducing one or more attenuating mutations as described for Shigella spp. above.

The particular Rickettsia strain employed is not critical to the present invention. Examples of Rickettsia strains which can be employed in the present invention include Rickettsia Rickettsiae (ATCC Nos. VR149 and VR891), Rickettsia prowaseckii (ATCC No. VR233), Rickettsia tsutsugamuchi (ATCC Nos. VR312, VR150 and VR609), Rickettsia mooseri (ATCC No. VR144), Rickettsia sibirica (ATCC No. VR151), and Rochalimaea quitana (ATCC No. VR358). Attenuated Rickettsia strains are preferably used in the present invention and can be constructed by introducing one or more attenuating mutations in groups (i) to (vii) as described for Shigella spp. above.

The particular enteroinvasive Escherichia strain employed is not critical to the present invention. Examples of enteroinvasive Escherichia strains which can be employed in the present invention include Escherichia coli strains 4608-58, 1184-68, 53638-C-17, 13-80, and 6-81 (Sansonetti et al. Ann. Microbiol. (Inst. Pasteur), 132A:351-355 (1982)). Attenuated enteroinvasive Escherichia strains are preferably used in the present invention and can be constructed by introducing one or more attenuating mutations in groups (i) to (vii) as described for Shigella spp. above.

Furthermore, since certain microorganisms other than bacteria can also interact with integrin molecules (which are receptors for certain invasion factors) for cellular uptake, such microorganisms can also be used for introducing RNA into target cells. For example, viruses, e.g., foot-and-mouth disease virus, echovirus, and adenovirus, and eukaryotic pathogens, e.g., Histoplasma capsulatum and Leishmania major interact with integrin molecules.

### 1.2 Less Invasive Bacteria

Examples of bacteria which can be used in the invention and which have been described in the literature as being non-invasive or at least less invasive than the bacteria listed in the previous section (1.1) include, but are not limited to, Yersinia spp., Escherichia spp., Klebsiella spp., Bordetella spp., Neisseria spp., Aeromonas spp., Franciesella spp., Corynebacterium spp., Citrobacter spp., Chlamydia spp., Hemophilus spp., Brucella spp., Mycobacterium spp., Legionella spp., Rhodococcus spp., Pseudomonas spp., Helicobacter spp., Vibrio spp., Bacillus spp., and Erysipelothrix spp. It may be necessary to modify these bacteria to increase their invasive potential.

The particular Yersinia strain employed is not critical to the present invention. Examples of Yersinia strains that can be employed in the present invention include Y enterocolitica (ATCC No. 9610) or Y. pestis (ATCC No. 19428). Attenuated Yersinia strains, such as Y. enterocolitica Ye03-R2 (al-Hendy et al. Infect. Immun, 60:870-875 (1992)) or Y. enterocolitica aroA (O'Gaora et al. Micro. Path., 9:105-116 (1990)) are preferably used in the present invention. Alternatively, new attenuated Yersinia strains can be constructed by introducing one or more attenuating mutations in groups (i) to (vii) as described for Shigella spp. above.

The particular Escherichia strain employed is not critical to the present invention. Examples of Escherichia strains that can be employed in the present invention include E. coli Nissle 1917, MM294, H10407 (Elinghorst et al. Infect. Immun, 60:2409-2417 (1992)), and E. coli EFC4, CFT325 and CPZ005 (Donnenberg et al. J. Infect. Dis., 169:831-838 (1994)). Attenuated Escherichia strains, such as the attenuated turkey pathogen E. coli 02 carAB mutant (Kwaga et al. Infect. Immun., 62:3766-3772 (1994)) or CEQ201 are preferably used in the present invention. Alternatively, new attenuated Escherichia strains can be constructed by introducing one or more attenuating mutations in groups (i) to (vii) as described for Shigella spp. above.

The particular Klebsiella strain employed is not critical to the present invention. Examples of Klebsiella strains that can be employed in the present invention include K. pneumoniae (ATCC No. 13884). Attenuated Klebsiella strains are preferably used in the present invention, and can be constructed by introducing one or more attenuating mutations in groups (i) to (vii) as described for Shigella spp. above.

The particular Bordetella strain employed is not critical to the present invention. Examples of Bordetella strains that can be employed in the present invention include B. bronchiseptica (ATCC No. 19395). Attenuated Bordetella strains are preferably used in the present invention, and can be constructed by introducing one or more attenuating mutations in groups (i) to (vii) as described for Shigella spp. above.

The particular Neisseria strain employed is not critical to the present invention. Examples of Neisseria strains that can be employed in the present invention include N. meningitidis (ATCC No. 13077) and N. gonorrhoeae (ATCC No. 19424). Attenuated Neisseria strains, such as N. gonorrhoeae MS11 aro mutant (Chamberlain et al. Micro. Path., 15:51-63 (1993)) are preferably used in the present invention. Alternatively, new attenuated Neisseria strains can be constructed by introducing one or more attenuating mutations in groups (i) to (vii) as described for Shigella spp. above.

The particular Aeromonas strain employed is not critical to the present invention. Examples of Aeromonas strains that can be employed in the present invention include A. eucrenophila (ATCC No. 23309). Alternatively, new attenuated Aeromonas strains can be constructed by introducing one or more attenuating mutations in groups (i) to (vii) as described for Shigella spp. above.

The particular Franciesella strain employed is not critical to the present invention. Examples of Franciesella strains that can be employed in the present invention include F. tularensis (ATCC No. 15482). Attenuated Franciesella strains are preferably used in the present invention, and can be constructed by introducing one or more attenuating mutations in groups (i) to (vii) as described for Shigella spp. above.

The particular Corynebacterium strain employed is not critical to the present invention. Examples of Corynebacterium strains that can be employed in the present invention include C. pseudotuberculosis (ATCC No. 19410). Attenuated Corynebacterium strains are preferably used in the present invention, and can be constructed by introducing one or more attenuating mutations in groups (i) to (vii) as described for Shigella spp. above.

The particular Citrobacter strain employed is not critical to the present invention. Examples of Citrobacter strains that can be employed in the present invention include C. freundii (ATCC No. 8090). Attenuated Citrobacter strains are preferably used in the present invention, and can be constructed by introducing one or more attenuating mutations in groups (i) to (vii) as described for Shigella spp. above.

The particular Chlamydia strain employed is not critical to the present invention. Examples of Chlamydia strains that can be employed in the present invention include C. pneumoniae (ATCC No. VR1310). Attenuated Chlamydia strains are preferably used in the present invention, and can be constructed by introducing one or more attenuating mutations in groups (i) to (vii) as described for Shigella spp. above.

The particular Hemophilus strain employed is not critical to the present invention. Examples of Hemophilus strains that can be employed in the present invention include H. sornnus (ATCC No. 43625). Attenuated Hemophilus strains are preferably used in the present invention, and can be constructed by introducing one or more attenuating mutations in groups (i) to (vii) as described for Shigella spp. above.

The particular Brucella strain employed is not critical to the present invention. Examples of Brucella strains that can be employed in the present invention include B. abortus (ATCC No. 23448). Attenuated Brucella strains are preferably used in the present invention, and can be constructed by introducing one or more attenuating mutations in groups (i) to (vii) as described for Shigella spp. above.

The particular Mycobacterium strain employed is not critical to the present invention. Examples of Mycobacterium strains that can be employed in the present invention include M. intracellulare (ATCC No. 13950) and M. tuberculosis (ATCC No. 27294). Attenuated Mycobacterium strains are preferably used in the present invention, and can be constructed by introducing one or more attenuating mutations in groups (i) to (vii) as described for Shigella spp. above.

The particular Legionella strain employed is not critical to the present invention. Examples of Legionella strains that can be employed in the present invention include L. pneumophila (ATCC No. 33156). Attenuated Legionella strains, such as a L. pneumophila mip mutant (Ott, FEMS Micro. Rev., 14:161-176 (1994)) are preferably used in the present invention. Alternatively, new attenuated Legionella strains can be constructed by introducing one or more attenuating mutations in groups (i) to (vii) as described for Shigella spp. above.

The particular Rhodococcus strain employed is not critical to the present invention. Examples of Rhodococcus strains that can be employed in the present invention include R. equi (ATCC No. 6939). Attenuated Rhodococcus strains are preferably used in the present invention, and can be constructed by introducing one or more attenuating mutations in groups (i) to (vii) as described for Shigella spp. above.

The particular Pseudomonas strain employed is not critical to the present invention. Examples of Pseudomonas strains that can be employed in the present invention include P. aeruginosa (ATCC No. 23267). Attenuated Pseudomonas strains are preferably used in the present invention, and can be constructed by introducing one or more attenuating mutations in groups (i) to (vii) as described for Shigella spp. above.

The particular Helicobacter strain employed is not critical to the present invention. Examples of Helicobacter strains that can be employed in the present invention include H. mustelae (ATCC No. 43772). Attenuated Helicobacter strains are preferably used in the present invention, and can be constructed by introducing one or more attenuating mutations in groups (i) to (vii) as described for Shigella spp. above.

The particular Salmonella strain employed is not critical to the present invention. Examples of Salmonella strains that can be employed in the present invention include Salmonella typhi (ATCC No. 7251) and S. typhimurium (ATCC No. 13311). Attenuated Salmonella strains are preferably used in the present invention and include S. typhi aroC aroD (Hone et al. Vacc., 9:810-816 (1991)) and S. typhimurium aroA mutant (Mastroeni et al. Micro. Pathol, 13:477-491 (1992))). Alternatively, new attenuated Salmonella strains can be constructed by introducing one or more attenuating mutations in groups (i) to (vii) as described for Shigella spp. above.

The particular Vibrio strain employed is not critical to the present invention. Examples of Vibrio strains that can be employed in the present invention include Vibrio cholerae (ATCC No. 14035) and Vibrio cincinnatiensis (ATCC No. 35912). Attenuated Vibrio strains are preferably used in the present invention and include V. cholerae RSI virulence mutant (Taylor et al. J. Infect. Dis., 170:1518-1523 (1994)) and V. cholerae ctxA, ace, zot, cep mutant (Waldor et al. J. Infect. Dis., 170:278-283 (1994)). Alternatively, new attenuated Vibrio strains can be constructed by introducing one or more attenuating mutations in groups (i) to (vii) as described for Shigella spp. above.

The particular Bacillus strain employed is not critical to the present invention. Examples of Bacillus strains that can be employed in the present invention include Bacillus subtilis (ATCC No. 6051). Attenuated Bacillus strains are preferably used in the present invention and include B. anthracis mutant pX01 (Welkos et al. Micro. Pathol, 14:381-388 (1993)) and attenuated BCG strains (Stover et al. Nat., 351:456-460 (1991)). Alternatively, new attenuated Bacillus strains can be constructed by introducing one or more attenuating mutations in groups (i) to (vii) as described for Shigella spp. above.

The particular Erysipelothrix strain employed is not critical to the present invention. Examples of Erysipelothrix strains that can be employed in the present invention include Erysipelothrix rhusiopathiae (ATCC No. 19414) and Erysipelothrix tonsillarum (ATCC No. 43339). Attenuated Erysipelothrix strains are preferably used in the present invention and include E. rhusiopathiae Kg-1a and Kg-2 (Watarai et al. J. Vet. Med. Sci., 55:595-600 (1993)) and E. rhusiopathiae ORVAC mutant (Markowska-Daniel et al. Int. J. Med. Microb. Virol. Parish. Infect. Dis., 277:547-553 (1992)). Alternatively, new attenuated Erysipelothrix strains can be constructed by introducing one or more attenuating mutations in groups (i) to (vii) as described for Shigella spp. above.

### 1.3. Methods for Increasing the Invasive Properties of a Bacterial Strain

Whether organisms have been traditionally described as invasive or non-invasive, these organisms can be engineered to increase their invasive properties, e.g., by mimicking the invasive properties of Shigella spp., Listeria spp., Rickettsia spp., or enteroinvasive E. coli spp. For example, one or more genes that enable the microorganism to access the cytoplasm of a cell, e.g., a cell in the natural host of said non-invasive bacteria, can be introduced into the microorganism.

Examples of such genes referred to herein as “cytoplasm-targeting genes” include genes encoding the proteins that enable invasion by Shigella or the analogous invasion genes of entero-invasive Escherichia, or listeriolysin O of Listeria, as such techniques are known to result in rendering a wide array of invasive bacteria capable of invading and entering the cytoplasm of animal cells (Formal et al. Infect. Immun, 46:465 (1984); Bielecke et al. Nature, 345:175-176 (1990); Small et al. In: Microbiology-1986, pages 121-124, Levine et al. Eds., American Society for Microbiology, Washington, D.C. (1986); Zychlinsky et al. Molec. Micro., 11:619-627 (1994); Gentschev et al. (1995) Infection & Immunity 63:4202; Isberg, R. R. and S. Falkow (1985) Nature 317:262; and Isberg, R. R. et al. (1987) Cell 50:769). Methods for transferring the above cytoplasm-targeting genes into a bacterial strain are well known in the art. Another preferred gene that can be introduced into bacteria to increase their invasive character encodes the invasin protein from Yersinia pseudotuberculosis, (Leong et al. EMBO J., 9:1979 (1990)). Invasin can also be introduced in combination with listeriolysin, thereby further increasing the invasive character of the bacteria relative to the introduction of either of these genes. The above genes have been described for illustrative purposes; however, it will be obvious to those skilled in the art that any gene or combination of genes, from one or more sources, that participates in the delivery of a molecule, in particular an RNA or RNA-encoding DNA molecule, from a microorganism into the cytoplasm of a cell, e.g., an animal cell, will suffice. Thus, such genes are not limited to bacterial genes, and include viral genes, such as influenza virus hemagglutinin HA-2 that promotes endosmolysis (Plank et al. J. Biol. Chem., 269:12918-12924 (1994)).

The above cytoplasm-targeting genes can be obtained by, e.g., PCR amplification from DNA isolated from an invasive bacterium carrying the desired cytoplasm-targeting gene. Primers for PCR can be designed from the nucleotide sequences available in the art, e.g., in the above-listed references and/or in GenBank, which is publicly available on the interne (www.ncbi.nlm.nih.gov/). The PCR primers can be designed to amplify a cytoplasm-targeting gene, a cytoplasm-targeting operon, a cluster of cytoplasm-targeting genes, or a regulon of cytoplasm-targeting genes. The PCR strategy employed will depend on the genetic organization of the cytoplasm-targeting gene or genes in the target invasive bacteria. The PCR primers are designed to contain a sequence that is homologous to DNA sequences at the beginning and end of the target DNA sequence. The cytoplasm-targeting genes can then be introduced into the target bacterial strain, e.g., by using Hfr transfer or plasmid mobilization (Miller, A Short Course in Bacterial Genetics, Cold Spring Harbor Laboratory Press, Cold Spring Harbor, N.Y. (1992); Bothwell et al. supra; and Ausubel et al. supra), bacteriophage-mediated transduction (de Boer, supra; Miller, supra; and Ausubel et al. supra), chemical transformation (Bothwell et al. supra; Ausubel et al. supra), electroporation (Bothwel et al. supra; Ausubel et al. supra; and Sambrook, Molecular Cloning: A Laboratory Manual, Cold Spring Harbor Laboratory Press, Cold Spring Harbor, N.Y.) and physical transformation techniques (Johnston et al. supra; and Bothwell, supra). The cytoplasm-targeting genes can be incorporated into lysogenic bacteriophage (de Boer et al. Cell, 56:641-649 (1989)), plasmids vectors (Curtiss et al. supra) or spliced into the chromosome (Hone et al. supra) of the target strain.

In addition to genetically engineering bacteria and BTPs to increase their invasive properties, as set forth above, bacteria and can also be modified by linking an invasion factor to the bacteria. Accordingly, in one embodiment, a bacterium is rendered more invasive by coating the bacterium, either covalently or non-covalently, with an invasion factor, e.g., the protein invasin, invasin derivatives, or a fragment thereof sufficient for invasiveness. In fact, it has been shown that non-invasive bacterial cells coated with purified invasin from Yersinia pseudotuberculosis or the carboxyl-terminal 192 amino acids of invasin are able to enter mammalian cells (Leong et al. (1990) EMBO J. 9:1979). Furthermore, latex beads coated with the carboxyl terminal region of invasin are efficiently internalized by mammalian cells, as are strains of Staphylococcus aureus coated with antibody-immobilized invasin (reviewed in Isberg and Tran van Nhieu (1994) Ann. Rev. Genet. 27:395). Alternatively, a bacterium can also be coated with an antibody, variant thereof, or fragment thereof, which binds specifically to a surface molecule recognized by a bacterial entry factor. For example, it has been shown that bacteria are internalized if they are coated with a monoclonal antibody directed against an integrin molecule, e.g., α5β1, known to be the surface molecule with which the bacterial invasin protein interacts (Isberg and Tran van Nhieu, supra). Such antibodies can be prepared according to methods known in the art. The antibodies can be tested for efficacy in mediating bacterial invasiveness by, e.g., coating bacteria with the antibody, contacting the bacteria with eukaryotic cells having a surface receptor recognized by the antibody, and monitoring the presence of intracellular bacteria, according to the methods described above. Methods for linking an invasion factor to the surface of a bacterium are known in the art and include cross-linking.

### 3. Plasmids and Vectors

The present invention also provides at least one vector or plasmid including at least one DNA molecule encoding one or more siRNAs and at least one promoter, wherein the expressed siRNAs interfere with at least one mRNA of a gene of interest. In one preferred embodiment, the present invention provides at least one prokaryotic vector including at least one DNA molecule encoding one or more siRNAs and at least one RNA-polymerase III compatible promoter or at least one prokaryotic promoter, wherein the expressed siRNAs interfere with at least one mRNA of a gene of interest.

The TRIP (transkingdom RNA interference plasmid) vectors and plasmids of the present invention include a multiple cloning site, a promoter sequence and a terminator sequence. The TRIP vectors and plasmids also include one or more sequences encoding for an invasion factor to permit the non-invasive bacterium or BTP to enter mammalian cells (e.g., the Inv locus that encodes invasion that permits the bacterium or BTP to enter β1-integrin-positive mammalian cells) (Young et al., J. Cell Biol. 116, 197-207 (1992)) and one or more sequences to permit the genetic material to escape from the entry vesicles (e.g., Hly A gene that encodes listeriolysin O) (Mathew et al., Gene Ther. 10, 1105-1115 (2003) and Grillot-Courvalin et al., Nat. Biotechnol. 16, 862-866 (1998)). TRIP is further described (including a vector/plasmid schematic) in PCT Publication No. WO 06/066048. In preferred embodiments, the TRIP vectors and plasmids will incorporate a hairpin RNA expression cassette encoding short hairpin RNA under the control of an appropriate promoter sequence and terminator sequence.

In the design of these constructs, an algorithm was utilized to take into account some known difficulties with the development of siRNA, namely: (1) Exclusion of disqualifying properties (SNPs, interferon motifs); (2) Exclusion of the sequence if there was homology in ref seq (19/21, >17 contiguous to any other genes) and (3) Exclusion of the sequence if there were significant miRNA seed type matches.

As described herein, the one or more DNA molecules encoding the one or more siRNAs are transcribed within the eukaryotic target cell or transcribed within the bacterium or BTP.

In embodiments where the DNA is transcribed within the eukaryotic cell, the one or more siRNAs are transcribed within the eukaryotic cells as shRNAs. The eukaryotic cell can be in vivo, in vitro or ex vivo. In one aspect of this embodiment, the one or more DNA molecules encoding the one or more siRNAs contain a eukaryotic promoter. Optionally, the eukaryotic promoter is a RNA-polymerase III promoter. Optionally, the RNA polymerase III promoter is a U6 promoter or an H1 promoter.

In embodiments where the DNA is transcribed within the bacterium or BTP, the one or more DNA molecules contain a prokaryotic promoter. Optionally, the prokaryotic promoter is an E. coli promoter. Preferably, the E. coli promoter can be a T7 promoter, lacUV5 promoter, modified lacUV5 promoter, RNA polymerase promoter, gapA promoter, pA1 promoter, lac regulated promoter, araC+ P_{araBAD} promoter, T5 promoter, P_{tac} promoter (Estrem et al, 1998, Proc. Natl. Acad. Sci. USA 95, 9761-9766; Meng et al., 2001, Nucleic Acids Res. 29, 4166-417; De Boer et al., 1983, Proc. Natl. Acad. Sci. USA 80, 21-25) or recA promoter.

Preferable, promoter sequences are recited in Table 1.

TABLE 1

| | | |
|---|---|---|
| Promoter | Sequence | SEQ ID NO: |

| | | |
|---|---|---|
| T7 promoter | TAATACGACTCACTATAG | 1 |
| lacUV5 promoter | TAACCAGGCTTTACACTTTATGCTTCCGGCTC | 2 |
| | GTATAATGTGTGGAAGGATCC | |
| RNA polymerase promoter | TAACCAGGCTTTACACTTTATGCTTCCGGCTC | 3 |
| | GTATAATGTGTGGAA | |
| RNA polymerase promoter | TAAAATTCAAAAATTTATTTGCTTTCAGGAAA | 4 |
| | ATTTTTCTGTATAATAGATTC | |
| RNA polymerase promoter | TAATTGATACTTTATGCTTTTTTCTGTATAAT | 5 |
| gapA promoter | AAGCTTTCAGTCGCGTAATGCTTAGGCACAGG | 6 |
| | ATTGATTTGTCGCAATGATTGACACGATTCCG | |
| | CTTGACACTGCGTAAGTTTTGTGTTATAATGG | |
| | ATCC | |
| pA1 promoter | AAGCTTAAGGAGAGACAACTTAAAGAGACTTA | 7 |
| | AAAGATTAATTTAAAATTTATCAAAAAGAGTA | |
| | TTGACTTAAAGTCTAACCTATAGGATACTTGG | |
| | ATCC | |
| lac regulated promoter | AAGCTTTGTGTGGAATTGTGAGCGGATAACAA | 8 |
| | TTCCACACATTGACACTTTATGCTTCCGGCTC | |
| | GTATAATGGATCC | |
| lac regulated promoter | AAGCTTGGAAAATTTTTTTTAAAAAAGTCATG | 9 |
| | TGTGGAATTGTGAGCGGATAACAATTCCACAT | |
| | ATAATGGATCC | |
| araC+ P_{araBAD} promoter | GACTTCATATACCCAAGCTTTAAAAAAAAAAT | 10 |
| | CCTTAGCTTTCGCTAAGGATCTCCGTCAAGCC | |
| | GTCAATTGTCTGATTCGTTACCAATTATGACA | |
| | ACTTGACGGCTACATCATTCACTTTTTCTTCA | |
| | CAACCGGCACGAAACTCGCTCGGGCTGGCCCC | |
| | GGTGCATTTTTTAAATACTCGCGAGAAATAGA | |
| | GTTGATCGTCAAAACCAACATTGCGACCGACG | |
| | GTGGCGATAGGCATCCGGGTAGTGCTCAAAAG | |
| | CAGCTTCGCCTGACTAATGCGTTGGTCCTCGC | |
| | GCCAGCTTAAGACGCTAATCCCTAACTGCTGG | |
| | CGGAAAAGATGTGACAGACGCGACGGCGACAA | |
| | GCAAACATGCTGTGCGACGCTGGCGATATCAA | |
| | AATTGCTGTCTGCCAGGTGATCGCTGATGTAC | |
| | TGACAAGCCTCGCGTACCCGATTATCCATCGG | |
| | TGGATGGAGCGACTCGTTAATCGCTTCCATGC | |
| | GCCGCAGTAACAATTGCTCAAGCAGATTTATC | |
| | GCCAGCAGCTCCGAATAGCGCCCTTCCCCTTG | |
| | CCCGGCGTTAATGATTTGCCCAAACAGGTCGC | |
| | TGAAATGCGGCTGGTGCGCTTCATCCGGGCGA | |
| | AAGAAACCCGTATTGGCAAATATTGACGGCCA | |
| | GTTAAGCCATTCATGCCAGTAGGCGCGCGGAC | |
| | GAAAGTAAACCCACTGGTGATACCATTCGCGA | |
| | GCCTCCGGATGACGACCGTAGTGATGAATCTC | |
| | TCCTGGCGGGAACAGCAAAATATCACCCGGTC | |
| | GGCAGACAAATTCTCGTCCCTGATTTTTCACC | |
| | ACCCCCTGACCGCGAATGGTGAGATTGAGAAT | |
| | ATAACCTTTCATTCCCAGCGGTCGGTCGATAA | |
| | AAAAATCGAGATAACCGTTGGCCTCAATCGGC | |
| | GTTAAACCCGCCACCAGATGGGCGTTAAACGA | |
| | GTATCCCGGCAGCAGGGGATCATTTTGCGCTT | |
| | CAGCCATACTTTTCATACTCCCACCATTCAGA | |
| | GAAGAAACCAATTGTCCATATTGCATCAGACA | |
| | TTGCCGTCACTGCGTCTTTTACTGGCTCTTCT | |
| | CGCTAACCCAACCGGTAACCCCGCTTATTAAA | |
| | AGCATTCTGTAACAAAGCGGGACCAAAGCCAT | |
| | GACAAAAACGCGTAACAAAAGTGTCTATAATC | |
| | ACGGCAGAAAAGTCCACATTGATTATTTGCAC | |
| | GGCGTCACACTTTGCTATGCCATAGCATTTTT | |
| | ATCCATAAGATTAGCGGATCCTACCTGACGCT | |
| | TTTTATCGCAACTCTCTACTGTAGATCTATCT | |
| | GCGAT | |
| T5 promoter | TAAATTCAAAAATTTATTTGCTTTCAGGAAA | 11 |
| | ATTTTTCTGTATAATAGATTCGGATCC | |
| recA promoter | TAATTGATACTTTATGCTTTTTTCTGTATAAT | 12 |
| | GGATCC | |
| P_{tac} promoter | GACTTCATATACCCAAGCTTGGAAAATTTTTT | 13 |
| | TTAAAAAAGTCTTGACACTTTATGCTTCCGGC | |
| | TCGTATAATGGATCC | |
| P_{atac} promoter | GGAAAATTTTTTTTAAAAAAGTC | 14 |

In embodiments where the DNA is transcribed within the bacterium or BTP, the E. coli promoter is associated with a terminator. Preferably, the E. coli terminator can be a T7 terminator, lacUV5 terminator, Rho-independent terminator, Rho-dependent terminator, or RNA polymerase terminator.

Preferable, terminator sequences are recited in Table 2.

TABLE 2

| | | |
|---|---|---|
| Terminator | Sequence | SEQ ID NO: |

| | | |
|---|---|---|
| T7 terminator | TAGCATAACCCCTTGGGGCCTCTAAACGGGTC | 15 |
| | TTGAGGGGTTTTTTG | |
| lacUV5 terminator | TTGTCACGTGAGCGGATAACAATTTCACACAG | 16 |
| | GAAACAGAATTCTTAAT | |
| Rho-independent terminator | TTGTCACAAACCCCGCCACCGGCGGGGTTTTT | 17 |
| | TTCTGCTTAAT | |
| Rho-dependent terminator | TTGTCACAATTCTATGGTGTATGCATTTATTT | 18 |
| | GCATACATTCAATCAATTGGATCCTGCATTAAT | |
| RNA polymerase terminator | GTGAGCGGATAACAATTTCACACAGGAAACAG | 19 |
| | AATTCTTAAT | |
| RNA polymerase terminator | AAACCCCGCCACCGGCGGGGTTTTTTTCTGCT | 20 |
| | TAAT | |
| RNA polymerase terminator | AATTCTATGGTGTATGCATTTATTTGCATACA | 21 |
| | TTCAATCAATTGGATCCTGCATTAAT | |

In additional embodiments, the vectors and plasmids of the present invention further include one or more enhancer sequences, selection markers, or lysis regulation system sequences.

In one aspect of the invention, the one or more DNA molecules contain a prokaryotic enhancer. Optionally, the prokaryotic enhancer is a T7 enhancer. Optionally, the T7 enhancer has the sequence GAGACAGG (SEQ ID NO: 22). In another aspect of this embodiment, the one or more DNA molecules contain a prokaryotic terminator.

In another aspect of the, the one or more DNA molecules are associated with one or more selection markers. In one aspect of this embodiment, the selection marker is an amber suppressor containing one or more mutations or a diamino pimelic acid (DAP) containing one or more mutations. Optionally, the dap gene is selected from, but not limited to, dapA and dapE.

Preferable, selection marker sequences are recited in Table 3.

TABLE 3

| | | |
|---|---|---|
| Selection Marker | Sequence | SEQ ID NO: |

| | | |
|---|---|---|
| amber suppressor gene | AATTCGGGGCTATAGCTCAGCTGGGAGAGCGCTT | 23 |
| sequence | GCATCTAATGCAAGAGGTCAGCGGTTCGATCCCG | |
| | CTTAGCTCCACCACTGCA | |
| amber suppressor sequence | AATTCGCCCGGATAGCTCAGTCGGTAGAGCAGGG | 24 |
| | GATTCTAAATCCCCGTGTCCTTGGTTCGATTCCG | |
| | AGTCCGGGCACTGCA | |
| Rho-lgt with double amber | ATGACCAGTAGCTATCTGCATTAGCCGGAGTAGG | 25 |
| mutation (lgt am-am allele | ATCCGGTCATTTTCTCAATAGGACCCGTGGCGCT | |
| of lgt gene) sequence | TCACTGGTACGGCCTGATGTATCTGGTGGGTTTC | |
| | ATTTTTGCAATGTGGCTGGCAACACGACGGGCGA | |
| | ATCGTCCGGGCAGCGGCTGGACCAAAAATGAAGT | |
| | TGAAAACTTACTCTATGCGGGCTTCCTCGGCGTC | |
| | TTCCTCGGGGGACGTATTGGTTATGTTCTGTTCT | |
| | ACAATTTCCCGCAGTTTATGGCCGATCCGCTGTA | |
| | TCTGTTCCGTGTCTGGGACGGCGGCATGTCTTTC | |
| | CACGGCGGCCTGATTGGCGTTATCGTGGTGATGA | |
| | TTATCTTCGCCCGCCGTACTAAACGTTCCTTCTT | |
| | CCAGGTCTCTGATTTTATCGCACCACTCATTCCG | |
| | TTTGGTCTTGGTGCCGGGCGTCTGGGCAACTTTA | |
| | TTAACGGTGAATTGTGGGGCCGCGTTGACCCGAA | |
| | CTTCCCGTTTGCCATGCTGTTCCCTGGCTCCCGT | |
| | ACAGAAGATATTTTGCTGCTGCAAACCAACCCGC | |
| | AGTGGCAATCCATTTTCGACACTTACGGTGTGCT | |
| | GCCGCGCCACCCATCACAGCTTTACGAGCTGCTG | |
| | CTGGAAGGTGTGGTGCTGTTTATTATCCTCAACC | |
| | TGTATATTCGTAAACCACGCCCAATGGGAGCTGT | |
| | CTCAGGTTTGTTCCTGATTGGTTACGGCGCGTTT | |
| | CGCATCATTGTTGAGTTTTTCCGCCAGCCCGACG | |
| | CGCAGTTTACCGGTGCCTGGGTGCAGTACATCAG | |
| | CATGGGGCAAATTCTTTCCATCCCGATGATTGTC | |
| | GCGGGTGTGATCATGATGGTCTGGGCATATCGTC | |
| | GCAGCCCACAGCAACACGTTTCCTGA | |
| murA with double amber | ATGGATAAATTTCGTGTTCAGGGGCCAACGAAGC | 26 |
| mutation (murA am-am | TCCAGGGCGAAGTCACAATTTCCGGCGCTAAAAA | |
| allele of murA gene) | TTAGTAGCTGCCTATCCTTTTTGCCGCACTACTG | |
| sequence | GCGGAAGAACCGGTAGAGATCCAGAACGTCCCGA | |
| | AACTGAAAGACGTCGATACATCAATGAAGCTGCT | |
| | AAGCCAGCTGGGTGCGAAAGTAGAACGTAATGGT | |
| | TCTGTGCATATTGATGCCCGCGACGTTAATGTAT | |
| | TCTGCGCACCTTACGATCTGGTTAAAACCATGCG | |
| | TGCTTCTATCTGGGCGCTGGGGCCGCTGGTAGCG | |
| | CGCTTTGGTCAGGGGCAAGTTTCACTACCTGGCG | |
| | GTTGTACGATCGGTGCGCGTCCGGTTGATCTACA | |
| | CATTTCTGGCCTCGAACAATTAGGCGCGACCATC | |
| | AAACTGGAAGAAGGTTACGTTAAAGCTTCCGTCG | |
| | ATGGTCGTTTGAAAGGTGCACATATCGTGATGGA | |
| | TAAAGTCAGCGTTGGCGCAACGGTGACCATCATG | |
| | TGTGCTGCAACCCTGGCGGAAGGCACCACGATTA | |
| | TTGAAAACGCAGCGCGTGAACCGGAAATCGTCGA | |
| | TACCGCGAACTTCCTGATTACGCTGGGTGCGAAA | |
| | ATTAGCGGTCAGGGCACCGATCGTATCGTCATCG | |
| | AAGGTGTGGAACGTTTAGGCGGCGGTGTCTATCG | |
| | CGTTCTGCCGGATCGTATCGAAACCGGTACTTTC | |
| | CTGGTGGCGGCGGCGATTTCTCGCGGCAAAATTA | |
| | TCTGCCGTAACGCGCAGCCAGATACTCTCGACGC | |
| | CGTGCTGGCGAAACTGCGTGACGCTGGAGCGGAC | |
| | ATCGAAGTCGGCGAAGACTGGATTAGCCTGGATA | |
| | TGCATGGCAAACGTCCGAAGGCTGTTAACGTACG | |
| | TACCGCGCCGCATCCGGCATTCCCGACCGATATG | |
| | CAGGCCCAGTTCACGCTGTTGAACCTGGTGGCAG | |
| | AAGGGACCGGGTTTATCACCGAAACGGTCTTTGA | |
| | AAACCGCTTTATGCATGTGCCAGAGCTGAGCCGT | |
| | ATGGGCGCGCACGCCGAAATCGAAAGCAATACCG | |
| | TTATTTGTCACGGTGTTGAAAAACTTTCTGGCGC | |
| | ACAGGTTATGGCAACCGATCTGCGTGCATCAGCA | |
| | AGCCTGGTGCTGGCTGGCTGTATTGCGGAAGGGA | |
| | CGACGGTGGTTGATCGTATTTATCACATCGATCG | |
| | TGGCTACGAACGCATTGAAGACAAACTGCGCGCT | |
| | TTAGGTGCAAATATTGAGCGTGTGAAAGGCGAAT | |
| | AA | |
| dapA sequence | GCCAGGCGACTGTCTTCAATATTACAGCCGCAAC | 27 |
| | TACTGACATGACGGGTGATGGTGTTCACAATTCC | |
| | AGGGCGATCGGCACCCAACGCAGTGATCACCAGA | |
| | TAATGTTGCGATGACAGTGTCAAACTGGTTATTC | |
| | CTTTAAGGGGTGAGTTGTTCTTAAGGAAAGCATA | |
| | AAAAAAACATGCATACAACAATCAGAACGGTTCT | |
| | GTCTGCTTGCTTTTAATGCCATACCAAACGTACC | |
| | ATTGAGACACTTGTTTGCACAGAGGATGGCCCAT | |
| | GTTCACGGGAAGTATTGTCGCGATTGTTACTCCG | |
| | ATGGATGAAAAAGGTAATGTCTGTCGGGCTAGCT | |
| | TGAAAAAACTGATTGATTATCATGTCGCCAGCGG | |
| | TACTTCGGCGATCGTTTCTGTTGGCACCACTGGC | |
| | GAGTCCGCTACCTTAAATCATGACGAACATGCTG | |
| | ATGTGGTGATGATGACGCTGGATCTGGCTGATGG | |
| | GCGCATTCCGGTAATTGCCGGGACCGGCGCTAAC | |
| | GCTACTGCGGAAGCCATTAGCCTGACGCAGCGCT | |
| | TCAATGACAGTGGTATCGTCGGCTGCCTGACGGT | |
| | AACCCCTTACTACAATCGTCCGTCGCAAGAAGGT | |
| | TTGTATCAGCATTTCAAAGCCATCGCTGAGCATA | |
| | CTGACCTGCCGCAAATTCTGTATAATGTGCCGTC | |
| | CCGTACTGGCTGCGATCTGCTCCCGGAAACGGTG | |
| | GGCCGTCTGGCGAAAGTAAAAAATATTATCGGAA | |
| | TCAAAGAGGCAACAGGGAACTTAACGCGTGTAAA | |
| | CCAGATCAAAGAGCTGGTTTCAGATGATTTTGTT | |
| | CTGCTGAGCGGCGATGATGCGAGCGCGCTGGACT | |
| | TCATGCAATTGGGCGGTCATGGGGTTATTTCCGT | |
| | TACGGCTAACGTCGCAGCGCGTGATATGGCCCAG | |
| | ATGTGCAAACTGGCAGCAGAAGGGCATTTTGCCG | |
| | AGGCACGCGTTATTAATCAGCGTCTGATGCCATT | |
| | ACACAACAAACTATTTGTCGAACCCAATCCAATC | |
| | CCGGTGAAATGGGCATGTAAGGAACTGGGTCTTG | |
| | TGGCGACCGATACGCTGCGCCTGCCAATGACACC | |
| | AATCACCGACAGTGGTCGTGAGACGGTCAGAGCG | |
| | GCGCTTAAGCATGCCGGTTTGCTGTAAAGTTTAG | |
| | GGAGATTTGATGGCTTACTCTGTTCAAAAGTCGC | |
| | GCCTGGCAAAGGTTGCGGGTGTTTCGCTTGTTTT | |
| | ATTACTCGCTGCCTGTAGTTCTGACTCACGCTAT | |
| | AAGCGTCAGGTCAGTGGTGATGAAGCCTACCTGG | |
| | AAGCG | |

Optionally, the amber suppressor is associated with a promoter or a terminator. Optionally, the promoter is a lipoprotein promoter. Preferable, promoter sequences are recited in Table 4.

TABLE 4

| Amber Suppressor | | |
|---|---|---|
| Promoter Sequence | Sequence | SEQ ID NO: |
| lipoprotein promoter | CATGGCGCCGCTTCTTTGAGCGAACGATCAAAAA | 28 |
| | TAAGTGGCGCCCCATCAAAAAAATATTCTCAACA | |
| | TAAAAAACTTTGTGTAATACTTGTAACGCTG | |
| lipoprotein promoter | CATGGCGCCCCATCAAAAAAATATTCTCAACATA | 29 |
| | AAAAACTTTGTGTAATACTTGTAACGCTG | |

Optionally, the terminator is an rrnC terminator. Preferable, terminator sequences are recited in Table 5.

TABLE 5

| Amber Suppressor | | |
|---|---|---|
| Terminator Sequence | Sequence | SEQ ID NO: |
| rrnC terminator | GATCCTTAGCGAAAGCTAAGGATTTTTTTTAC | 30 |
| rrnC terminator | GATCCTTAGCGAAAGCTAAGGATTTTTTTTTT | 31 |

Bacterial and BTP delivery is more attractive than viral delivery because they are more accessible to genetic manipulation, which allows the production of vector strains specifically tailored to certain applications. In one embodiment of the invention, the methods of the invention are used to create bacteria and BTPs that cause RNAi in a tissue specific manner.

Liberation of the siRNA encoding plasmid or the one or more siRNAs from the intracellular bacteria or BTPs occurs through active mechanisms. One mechanism involves the type III export system in S. typhimuriumm, a specialized multiprotein complex spanning the bacterial or BTP cell membrane whose functions include secretion of virulence factors to the outside of the cell to allow signaling towards the target cell, but which can also be used to deliver antigens into target cells (Rüssmann H. Int J Med Microbiol, 293:107-12 (2003)), or through bacterial lysis and liberation of bacterial or BTP contents into the cytoplasm. The lysis of intracellular bacteria or BTPs is triggered through various mechanisms, including addition of an intracellularly active antibiotic (tetracycline), naturally through bacterial metabolic attenuation (auxotrophy), or through a lysis regulation system or bacterial suicide system comprising a bacterial regulator, promoter and sensor that is sensitive to the environment, e.g., the pH, magnesium concentration, phosphate concentration, ferric ion concentration, osmolarity, anaerobic conditions, nutritional deficiency and general stress of the target cell or the host phagosome. When the bacteria or BTP lysis regulation system senses one or more of the above environmental conditions, bacterial or BTP lysis is triggered by one or more mechanisms including but not limited to antimicrobial proteins, bacteriophage lysins and autolysins expressed by the bacteria or BTP, either naturally or through modification, or through pore-forming proteins expressed by the bacteria or BTPs, either naturally or through modification, e.g., genetic modification, which break the phagosomes containing the bacteria or BTPs and liberate the siRNA-encoding plasmid or the one or more siRNAs.

The regulator of the lysis regulation system may be selected from the group that includes but is not limited to OmpR, ArcA, PhoP, PhoB, Fur, RstA, EvgA and RpoS. Preferable, lysis regulator sequences are recited in Table 6.

TABLE 6

| Lysis Regulation System | | |
|---|---|---|
| Regulator Sequence | Sequence | SEQ ID NO: |
| OmpR regulator | ATGCAAGAGAACTACAAGATTCTGGTGGTCGATG | 32 |
| | ACGACATGCGCCTGCGTGCGCTGCTGGAACGTTA | |
| | TCTCACCGAACAAGGCTTCCAGGTTCGAAGCGTC | |
| | GCTAATGCAGAACAGATGGATCGCCTGCTGACTC | |
| | GTGAATCTTTCCATCTTATGGTACTGGATTTAAT | |
| | GTTACCTGGTGAAGATGGCTTGTCGATTTGCCGA | |
| | CGTCTTCGTAGTCAGAGCAACCCGATGCCGATCA | |
| | TTATGGTGACGGCGAAAGGGGAAGAAGTGGACCG | |
| | TATCGTAGGCCTGGAGATTGGCGCTGACGACTAC | |
| | ATTCCAAAACCGTTTAACCCGCGTGAACTGCTGG | |
| | CCCGTATCCGTGCGGTGCTGCGTCGTCAGGCGAA | |
| | CGAACTGCCAGGCGCACCGTCACAGGAAGAGGCG | |
| | GTAATTGCTTTCGGTAAGTTCAAACTTAACCTCG | |
| | GTACGCGCGAAATGTTCCGCGAAGACGAGCCGAT | |
| | GCCGCTCACCAGCGGTGAGTTTGCGGTACTGAAG | |
| | GCACTGGTCAGCCATCCGCGTGAGCCGCTCTCCC | |
| | GCGATAAGCTGATGAACCTTGCCCGTGGTCGTGA | |
| | ATATTCCGCAATGGAACGCTCCATCGACGTGCAG | |
| | ATTTCGCGTCTGCGCCGCATGGTGGAAGAAGATC | |
| | CAGCGCATCCGCGTTACATTCAGACCGTCTGGGG | |
| | TCTGGGCTACGTCTTTGTACCGGACGGCTCTAAA | |
| | GCATGA | |
| PhoP regulator | ATGCGCGTACTGGTTGTTGAAGACAATGCGTTGT | 33 |
| | TACGTCACCACCTTAAAGTTCAGATTCAGGATGC | |
| | TGGTCATCAGGTCGATGACGCAGAAGATGCCAAA | |
| | GAAGCCGATTATTATCTCAATGAACATATACCGG | |
| | ATATTGCGATTGTCGATCTCGGATTGCCAGACGA | |
| | GGACGGTCTGTCACTGATTCGCCGCTGGCGTAGC | |
| | AACGATGTTTCACTGCCGATTCTGGTATTAACCG | |
| | CCCGTGAAAGCTGGCAGGACAAAGTCGAAGTATT | |
| | AAGTGCCGGTGCTGATGATTATGTGACTAAACCG | |
| | TTTCATATTGAAGAGGTGATGGCGCGAATGCAGG | |
| | CATTAATGCGGCGTAATAGCGGTCTGGCTTCACA | |
| | GGTCATTTCGCTCCCCCCGTTTCAGGTTGATCTC | |
| | TCTCGCCGTGAATTATCTATTAATGACGAAGTGA | |
| | TCAAACTGACCGCGTTCGAATACACTATTATGGA | |
| | AACGTTGATACGCAATAATGGCAAAGTGGTCAGC | |
| | AAAGATTCGTTAATGCTCCAACTCTATCCGGATG | |
| | CGGAGCTGCGGGAAAGCCATACCATTGATGTACT | |
| | GATGGGACGTCTGCGCAAAAAAATTCAGGCACAA | |
| | TATCCCCAAGAAGTGATTACCACCGTTCGCGGCC | |
| | AGGGCTATCTGTTCGAATTGCGCTGA | |

The promoter of the lysis regulation system may be selected from the group that includes but is not limited to ompF, ompC, fadB, phoPQ, mgtA, mgrB, psiB, phnD, Ptrp, sodA, sodB, sltA, sltB, asr, csgD, emrKY, yhiUV, acrAB, mdfA and to tolC. Preferable, lysis regulation system promoter sequences are recited in Table 7.

TABLE 7

| Lysis Regulation System | | |
|---|---|---|
| Promoter Sequence | Sequence | SEQ ID NO: |
| ompF promoter | GATCATCCTGTTACGGAATATTACATTGCAACAT | 34 |
| | TTACGCGCAAAAACTAATCCGCATTCTTATTGCG | |
| | GATTAGTTTTTTCTTAGCTAATAGCACAATTTTC | |
| | ATACTATTTTTTGGCATTCTGGATGTCTGAAAGA | |
| | AGATTTTGTGCCAGGTCGATAAAGTTTCCATCAG | |
| | AAACAAAATTTCCGTTTAGTTAATTTAAATATAA | |
| | GGAAATCATATAAATAGATTAAAATTGCTGTAAA | |
| | TATCATCACGTCTCTATGGAAATATGACGGTGTT | |
| | CACAAAGTTCCTTAAATTTTACTTTTGGTTACAT | |
| | ATTTTTTCTTTTTGAAACCAAATCTTTATCTTTG | |
| | TAGCACTTTCACGGTAGCGAAACGTTAGTTTGAA | |
| | TGGAAAGATGCCTGCA | |
| ompC promoter | TTTAAAAAAGTTCCGTAAAATTCATATTTTGAAA | 35 |
| | CATCTATGTAGATAACTGTAACATCTTAAAAGTT | |
| | TTAGTATCATATTCGTGTTGGATTATTCTGTATT | |
| | TTTGCGGAGAATGGACTTGCCGACTGGTTAATGA | |
| | GGGTTAACCAGTAAGCAGTGGCATAAAAAAGCAA | |
| | TAAAGGCATATAACAGAGGGTTAATAAC | |
| fadb promoter | AGTGATTCCATTTTTTACCCTTCTGTTTTTTTGA | 36 |
| | CCTTAAGTCTCCGCATCTTAGCACATCGTTCATC | |
| | CAGAGCGTGATTTCTGCCGAGCGTGATCAGATCG | |
| | GCATTTCTTTAATCTTTTGTTTGCATATTTTTAA | |
| | CACAAAATACACACTTCGACTCATCTGGTACGAC | |
| | CAGATCACCTTGCGGATTCAGGAGACTGAC | |
| phoPQ promoter | GAGCTATCACGATGGTTGATGAGCTGAAATAAAC | 37 |
| | CTCGTATCAGTGCCGGATGGCGATGCTGTCCGGC | |
| | CTGCTTATTAAGATTATCCGCTTTTTATTTTTTC | |
| | ACTTTACCTCCCCTCCCCGCTGGTTTATTTAATG | |
| | TTTACCCCCATAACCACATAATCGCGTTACACTA | |
| | TTTTAATAATTAAGACAGGGAGAAATAAAA | |
| mgtA promoter | GCTTCAACACGCTCGCGGGTGAGCTGGCTCACGC | 38 |
| | CGCTTTCGTTATTCAGCACCCGGGAAACTGTAGA | |
| | TTTCCCCACGCCGCTTAAGCGCGCGATATCTTTG | |
| | ATGGTCAGCCGATTTTGCATCCTGTTGTCCTGTA | |
| | ACGTGTTGTTTAATTATTTGAGCCTAACGTTACC | |
| | CGTGCATTCAGCAATGGGTAAAGTCTGGTTTATC | |
| | GTTGGTTTAGTTGTCAGCAGGTATTATATCGCCA | |
| Ptrp promoter | GAGCTGTTGACAATTAATCATCGAACTAGTTAAC | 39 |
| | TAGTACGCAAGTTCACGTAAAAAGGGTATCTAGA | |
| | ATTCT | |

The sensor of the lysis regulation system may be selected from the group that includes but is not limited to EnvZ, ArcB, PhoQ, PhoR, RstB and EvgS. Preferable, lysis regulation system sensor sequences are recited in Table 8.

TABLE 8

| Lysis Regulation System | | |
|---|---|---|
| Sensor Sequence | Sequence | SEQ ID NO: |
| EnvZ sensor | ATGAGGCGATTGCGCTTCTCGCCACGAAGTTCAT | 40 |
| | TTGCCCGTACGTTATTGCTCATCGTCACCTTGCT | |
| | GTTCGCCAGCCTGGTGACGACTTATCTGGTGGTG | |
| | CTGAACTTCGCGATTTTGCCGAGCCTCCAGCAGT | |
| | TTAATAAAGTCCTCGCGTACGAAGTGCGTATGTT | |
| | GATGACCGACAAACTGCAACTGGAGGACGGCACG | |
| | CAGTTGGTTGTGCCTCCCGCTTTCCGTCGGGAGA | |
| | TCTACCGTGAGCTGGGGATCTCTCTCTACTCCAA | |
| | CGAGGCTGCCGAAGAGGCAGGTCTGCGTTGGGCG | |
| | CAACACTATGAATTCTTAAGCCATCAGATGGCGC | |
| | AGCAACTGGGCGGCCCGACGGAAGTGCGCGTTGA | |
| | GGTCAACAAAAGTTCGCCTGTCGTCTGGCTGAAA | |
| | ACCTGGCTGTCGCCCAATATCTGGGTACGCGTGC | |
| | CGCTGACCGAAATTCATCAGGGCGATTTCTCTCC | |
| | GCTGTTCCGCTATACGCTGGCGATTATGCTATTG | |
| | GCGATAGGCGGGGCGTGGCTGTTTATTCGTATCC | |
| | AGAACCGACCGTTGGTCGATCTCGAACACGCAGC | |
| | CTTGCAGGTTGGTAAAGGGATTATTCCGCCGCCG | |
| | CTGCGTGAGTATGGCGCTTCGGAGGTGCGTTCCG | |
| | TTACCCGTGCCTTTAACCATATGGCGGCTGGTGT | |
| | TAAGCAACTGGCGGATGACCGCACGCTGCTGATG | |
| | GCGGGGGTAAGTCACGACTTGCGCACGCCGCTGA | |
| | CGCGTATTCGCCTGGCGACTGAGATGATGAGCGA | |
| | GCAGGATGGCTATCTGGCAGAATCGATCAATAAA | |
| | GATATCGAAGAGTGCAACGCCATCATTGAGCAGT | |
| | TTATCGACTACCTGCGCACCGGGCAGGAGATGCC | |
| | GATGGAAATGGCGGATCTTAATGCAGTACTCGGT | |
| | GAGGTGATTGCTGCCGAAAGTGGCTATGAGCGGG | |
| | AAATTGAAACCGCGCTTTACCCCGGCAGCATTGA | |
| | AGTGAAAATGCACCCGCTGTCGATCAAACGCGCG | |
| | GTGGCGAATATGGTGGTCAACGCCGCCCGTTATG | |
| | GCAATGGCTGGATCAAAGTCAGCAGCGGAACGGA | |
| | GCCGAATCGCGCCTGGTTCCAGGTGGAAGATGAC | |
| | GGTCCGGGAATTGCGCCGGAACAACGTAAGCACC | |
| | TGTTCCAGCCGTTTGTCCGCGGCGACAGTGCGCG | |
| | CACCATTAGCGGCACGGGATTAGGGCTGGCAATT | |
| | GTGCAGCGTATCGTGGATAACCATAACGGGATGC | |
| | TGGAGCTTGGCACCAGCGAGCGGGGCGGGCTTTC | |
| | CATTCGCGCCTGGCTGCCAGTGCCGGTAACGCGG | |
| | GCGCAGGGCACGACAAAAGAAGGGTAA | |
| PhoQ sensor | ATGAAAAAATTACTGCGTCTTTTTTTCCCGCTCT | 41 |
| | CGCTGCGGGTACGTTTTCTGTTGGCAACGGCAGC | |
| | GGTAGTACTGGTGCTTTCGCTTGCCTACGGAATG | |
| | GTCGCGCTGATCGGTTATAGCGTCAGTTTCGATA | |
| | AAACTACGTTTCGGCTGTTACGTGGCGAGAGCAA | |
| | TCTGTTCTATACCCTTGCGAAGTGGGAAAACAAT | |
| | AAGTTGCATGTCGAGTTACCCGAAAATATCGACA | |
| | AGCAAAGCCCCACCATGACGCTAATTTATGATGA | |
| | GAACGGGCAGCTTTTATGGGCGCAACGTGACGTG | |
| | CCCTGGCTGATGAAGATGATCCAGCCTGACTGGC | |
| | TGAAATCGAATGGTTTTCATGAAATTGAAGCGGA | |
| | TGTTAACGATACCAGCCTCTTGCTGAGTGGAGAT | |
| | CATTCGATACAGCAACAGTTGCAGGAAGTGCGGG | |
| | AAGATGATGACGACGCGGAGATGACCCACTCGGT | |
| | GGCAGTAAACGTCTACCCGGCAACATCGCGGATG | |
| | CCAAAATTAACCATTGTGGTGGTGGATACCATTC | |
| | CGGTGGAGCTAAAAAGTTCCTATATGGTCTGGAG | |
| | CTGGTTTATCTATGTGCTCTCAGCCAATCTGCTG | |
| | TTAGTGATCCCGCTGCTGTGGGTCGCCGCCTGGT | |
| | GGAGTTTACGCCCCATCGAAGCCCTGGCAAAAGA | |
| | AGTCCGCGAACTGGAAGAACATAACCGCGAATTG | |
| | CTCAATCCAGCCACAACGCGAGAACTGACCAGTC | |
| | TGGTACGAAACCTGAACCGATTGTTAAAAAGTGA | |
| | ACGCGAACGTTACGACAAATACCGTACGACGCTC | |
| | ACCGACCTGACCCATAGTCTGAAAACGCCACTGG | |
| | CGGTGCTGCAAAGTACGCTGCGTTCTCTGCGTAG | |
| | TGAAAAGATGAGCGTCAGTGATGCTGAGCCGGTA | |
| | ATGCTGGAGCAAATCAGCCGCATTTCACAGCAAA | |
| | TTGGCTACTACCTGCATCGTGCCAGTATGCGCGG | |
| | CGGGACATTGCTCAGCCGCGAGCTGCATCCGGTC | |
| | GCCCCACTGCTGGACAATCTCACCTCAGCGCTGA | |
| | ACAAAGTGTATCAACGCAAAGGGGTCAATATCTC | |
| | TCTCGATATTTCGCCAGAGATCAGCTTTGTCGGT | |
| | GAGCAGAACGATTTTGTCGAGGTGATGGGCAACG | |
| | TGCTGGATAATGCCTGTAAATATTGCCTCGAGTT | |
| | TGTCGAAATTTCTGCAAGGCAAACCGACGAGCAT | |
| | CTCTATATTGTGGTCGAGGATGATGGCCCCGGTA | |
| | TTCCATTAAGCAAGCGAGAGGTCATTTTCGACCG | |
| | TGGTCAACGGGTTGATACTTTACGCCCTGGGCAA | |
| | GGTGTAGGGCTGGCGGTAGCCCGCGAAATCACCG | |
| | AGCAATATGAGGGTAAAATCGTCGCCGGAGAGAG | |
| | CATGCTGGGCGGTGCGCGGATGGAGGTGATTTTT | |
| | GGTCGCCAGCATTCTGCGCCGAAAGATGAATAA | |

The lysis regulation system may comprise any combination of one or more of the above regulators, promoters and sensors.

In one example of this embodiment, the lysis regulation system comprises OmpR as the regulator, ompF as the promoter and EnvZ as the sensor and the stimulus is reduced osmolarity. In another example of this embodiment, the lysis regulation system comprises OmpR as the regulator, ompC as the promoter and EnvZ as the sensor and the stimulus is reduced osmolarity.

In another example of this embodiment, the lysis regulation system comprises the ArcA as the regulator, fad as the promoter and Arc B as the sensor and the stimulus is anaerobic conditions.

In another example of this embodiment, the lysis regulation system comprises PhoP as the regulator, phoPQ as the promoter and PhoQ as the sensor and the stimulus is reduced magnesium concentration. In another example of this embodiment, the lysis regulation system comprises PhoP as the regulator, mgtA as the promoter and PhoQ as the sensor and the stimulus is reduced magnesium concentration. In another example of this embodiment, the lysis regulation system comprises PhoP as the regulator, mgrB as the promoter and PhoQ as the sensor and the stimulus is reduced magnesium concentration.

In another example of this embodiment, the lysis regulation system comprises PhoB as the regulator, psiB as the promoter and PhoR as the sensor and the stimulus is reduced phosphate concentration. In another example of this embodiment, the lysis regulation system comprises PhoB as the regulator, phnD as the promoter and PhoR as the sensor and the stimulus is reduced phosphate concentration. In another example of this embodiment, the lysis regulation system comprises RstA as the regulator, asr as the promoter and RstB as the sensor. In another example of this embodiment, the lysis regulation system comprises RstA ast the regulator, csgD as the promoter and RstB as the sensor.

In another example of this embodiment, the lysis regulation system comprises EvgA as the regulator, emrKY as the promoter and EvgS as the sensor. In another example of this embodiment, the lysis regulation system comprises EvgA as the regulator, yhiUV as the promoter and EvgS as the sensor. In another example of this embodiment, the lysis regulation system comprises EvgA as the regulator, acrAB as the promoter and EvgS as the sensor. In another example of this embodiment, the lysis regulation system comprises EvgA as the regulator, mdfA as the promoter and EvgS as the sensor. In another example of this embodiment, the lysis regulation system comprises EvgA as the regulator, tolC as the promoter and EvgS as the sensor.

In another example of this embodiment, the lysis regulation system comprises Fur as the regulator in combination with a promoter selected from the group comprising sodA, sodB, sltA or sltB.

The antimicrobial protein may be selected from the group that includes but is not limited to α- and β-defensins, protegrins, cathelicidins (e.g., indolicidin and bactenecins), granulysin, lysozyme, lactoferrin, azurocidin, elastase, bactericidal permeability inducing peptide (BPI), adrenomedullin, brevinin, histatins and hepcidin. Additional antimicrobial proteins are disclosed in the following, each of which is incorporated herein by reference in its entirety: Devine, D. A. et al., Current Pharmaceutical Design, 8, 703-714 (2002); Jack R. W., et al., Microbiological Reviews, 59 (2), 171-200 (June 1995).

Optionally, the antimicrobial protein is an α-defensin, β-defensin, or protegrin. Preferable, antimicrobial protein sequences are recited in Table 9.

TABLE 9

| Antimicrobial Protein | | |
|---|---|---|
| Sequence | Sequence | SEQ ID NO: |
| α-defensin-1 protein | CTATAGAAGACCTGGGACAGAGGACTGCTGTCTG | 42 |
| | CCCTCTCTGGTCACCCTGCCTAGCTAGAGGATCT | |
| | GTGACCCCAGCCATGAGGACCCTCGCCATCCTTG | |
| | CTGCCATTCTCCTGGTGGCCCTGCAGGCCCAGGC | |
| | TGAGCCACTCCAGGCAAGAGCTGATGAGGTTGCT | |
| | GCAGCCCCGGAGCAGATTGCAGCGGACATCCCAG | |
| | AAGTGGTTGTTTCCCTTGCATGGGACGAAAGCTT | |
| | GGCTCCAAAGCATCCAGGCTCAAGGAAAAACATG | |
| | GCCTGCTATTGCAGAATACCAGCGTGCATTGCAG | |
| | GAGAACGTCGCTATGGAACCTGCATCTACCAGGG | |
| | AAGACTCTGGGCATTCTGCTGCTGAGCTTGCAGA | |
| | AAAAGAAAAATGAGCTCAAAATTTGCTTTGAGAG | |
| | CTACAGGGAATTGCTATTACTCCTGTACCTTCTG | |
| | CTCAATTTCCTTTCCTCATCCCAAATAAATGCCT | |
| | TGGTACAAGAAAAG | |
| α-defensin-3 protein | CCTTGCTATAGAAGACCTGGGACAGAGGACTGCT | 43 |
| | GTCTGCCCTCTCTGGTCACCCTGCCTAGCTAGAG | |
| | GATCTGTGACCCCAGCCATGAGGACCCTCGCCAT | |
| | CCTTGCTGCCATTCTCCTGGTGGCCCTGCAGGCC | |
| | CAGGCTGAGCCACTCCAGGCAAGAGCTGATGAGG | |
| | TTGCTGCAGCCCCGGAGCAGATTGCAGCGGACAT | |
| | CCCAGAAGTGGTTGTTTCCCTTGCATGGGACGAA | |
| | AGCTTGGCTCCAAAGCATCCAGGCTCAAGGAAAA | |
| | ACATGGACTGCTATTGCAGAATACCAGCGTGCAT | |
| | TGCAGGAGAACGTCGCTATGGAACCTGCATCTAC | |
| | CAGGGAAGACTCTGGGCATTCTGCTGCTGAGCTT | |
| | GCAGAAAAAGAAAAATGAGCTCAAAATTTGCTTT | |
| | GAGAGCTACAGGGAATTGCTATTACTCCTGTACC | |
| | TTCTGCTCAATTTCCTTTCCTCATCTCAAATAAA | |
| | TGCCTTGTTAC | |
| α-defensin-4 protein | GTCTGCCCTCTCTGCTCGCCCTGCCTAGCTTGAG | 44 |
| | GATCTGTCACCCCAGCCATGAGGATTATCGCCCT | |
| | CCTCGCTGCTATTCTCTTGGTAGCCCTCCAGGTC | |
| | CGGGCAGGCCCACTCCAGGCAAGAGGTGATGAGG | |
| | CTCCAGGCCAGGAGCAGCGTGGGCCAGAAGACCA | |
| | GGACATATCTATTTCCTTTGCATGGGATAAAAGC | |
| | TCTGCTCTTCAGGTTTCAGGCTCAACAAGGGGCA | |
| | TGGTCTGCTCTTGCAGATTAGTATTCTGCCGGCG | |
| | AACAGAACTTCGTGTTGGGAACTGCCTCATTGGT | |
| | GGTGTGAGTTTCACATACTGCTGCACGCGTGTCG | |
| | ATTAACGTTCTGCTGTCCAAGAGAATGTCATGCT | |
| | GGGAACGCCATCATCGGTGGTGTTAGCTTCACAT | |
| | GCTTCTGCAGCTGAGCTTGCAGAATAGAGAAAAA | |
| | TGAGCTCATAATTTGCTTTGAGAGCTACAGGAAA | |
| | TGGTTGTTTCTCCTATACTTTGTCCTTAACATCT | |
| | TTCTTGATCCTAAATATATATCTCGTAACAAG | |
| α-defensin-5 protein | ATATCCACTCCTGCTCTCCCTCCTGCAGGTGACC | 45 |
| | CCAGCCATGAGGACCATCGCCATCCTTGCTGCCA | |
| | TTCTCCTGGTGGCCCTGCAGGCCCAGGCTGAGTC | |
| | ACTCCAGGAAAGAGCTGATGAGGCTACAACCCAG | |
| | AAGCAGTCTGGGGAAGACAACCAGGACCTTGCTA | |
| | TCTCCTTTGCAGGAAATGGACTCTCTGCTCTTAG | |
| | AACCTCAGGTTCTCAGGCAAGAGCCACCTGCTAT | |
| | TGCCGAACCGGCCGTTGTGCTACCCGTGAGTCCC | |
| | TCTCCGGGGTGTGTGAAATCAGTGGCCGCCTCTA | |
| | CAGACTCTGCTGTCGCTGAGCTTCCTAGATAGAA | |
| | ACCAAAGCAGTGCAAGATTCAGTTCAAGGTCCTG | |
| | AAAAAAGAAAAACATTTTACTCTGTGTACCTTGT | |
| | GTCTTTCTAAATTTCTCTCTCCAAAATAAAGTTC | |
| | AAGCATT | |
| α-defensin-6 protein | ACACATCTGCTCCTGCTCTCTCTCCTCCAGCGAC | 46 |
| | CCTAGCCATGAGAACCCTCACCATCCTCACTGCT | |
| | GTTCTCCTCGTGGCCCTCCAGGCCAAGGCTGAGC | |
| | CACTCCAAGCTGAGGATGATCCACTGCAGGCAAA | |
| | AGCTTATGAGGCTGATGCCCAGGAGCAGCGTGGG | |
| | GCAAATGACCAGGACTTTGCCGTCTCCTTTGCAG | |
| | AGGATGCAAGCTCAAGTCTTAGAGCTTTGGGCTC | |
| | AACAAGGGCTTTCACTTGCCATTGCAGAAGGTCC | |
| | TGTTATTCAACAGAATATTCCTATGGGACCTGCA | |
| | CTGTCATGGGTATTAACCACAGATTCTGCTGCCT | |
| | CTGAGGGATGAGAACAGAGAGAAATATATTCATA | |
| | ATTTACTTTATGACCTAGAAGGAAACTGTCGTGT | |
| | GTCCCATACATTGCCATCAACTTTGTTTCCTCAT | |
| | CTCAAATAAAGTCCTTTCAGCAAAAAAAAAAAA | |
| β-defensin-1 protein | TCCCTTCAGTTCCGTCGACGAGGTTGTGCAATCC | 47 |
| | ACCAGTCTTATAAATACAGTGACGCTCCAGCCTC | |
| | TGGAAGCCTCTGTCAGCTCAGCCTCCAAAGGAGC | |
| | CAGCGTCTCCCCAGTTCCTGAAATCCTGGGTGTT | |
| | GCCTGCCAGTCGCCATGAGAACTTCCTACCTTCT | |
| | GCTGTTTACTCTCTGCTTACTTTTGTCTGAGATG | |
| | GCCTCAGGTGGTAACTTTCTCACAGGCCTTGGCC | |
| | ACAGATCTGATCATTACAATTGCGTCAGCAGTGG | |
| | AGGGCAATGTCTCTATTCTGCCTGCCCGATCTTT | |
| | ACCAAAATTCAAGGCACCTGTTACAGAGGGAAGG | |
| | CCAAGTGCTGCAAGTGAGCTGGGAGTGACCAGAA | |
| | GAAATGACGCAGAAGTGAAATGAACTTTTTATAA | |
| | GCATTCTTTTAATAAAGGAAAATTGCTTTTGAAG | |
| | TATACCTCCTTTGGGCCAAAAAAAAAAAAAAAAA | |
| | AAAAAAAA | |
| β-defensin-3 protein | TGAGTCTCAGCGTGGGGTGAAGCCTAGCAGCTAT | 48 |
| | GAGGATCCATTATCTTCTGTTTGCTTTGCTCTTC | |
| | CTGTTTTTGGTGCCTGTCCCAGGTCATGGAGGAA | |
| | TCATAAACACATTACAGAAATATTATTGCAGAGT | |
| | CAGAGGCGGCCGGTGTGCTGTGCTCAGCTGCCTT | |
| | CCAAAGGAGGAACAGATCGGCAAGTGCTCGACGC | |
| | GTGGCCGAAAATGCTGCCGAAGAAAGAAATAAAA | |
| | ACCCTGAAACATGACGAGAGTGTTGTAAAGTGTG | |
| | GAAATGCCTTCTTAAAGTTTATAAAAGTAAAATC | |
| | AAATTACATTTTTTTTTCAAAAAAAAAAAAA | |
| β-defensin-4 protein | AGACTCAGCTCCTGGTGAAGCTCCCAGCCATCAG | 49 |
| | CCATGAGGGTCTTGTATCTCCTCTTCTCGTTCCT | |
| | CTTCATATTCCTGATGCCTCTTCCAGGTGTTTTT | |
| | GGTGGTATAGGCGATCCTGTTACCTGCCTTAAGA | |
| | GTGGAGCCATATGTCATCCAGTCTTTTGCCCTAG | |
| | AAGGTATAAACAAATTGGCACCTGTGGTCTCCCT | |
| | GGAACAAAATGCTGCAAAAAGCCATGAGGAGGCC | |
| | AAGAAGCTGCTGTGGCTGATGCGGATTCAGAAAG | |
| | GGCTCCCTCATCAGAGACGTGCGACATGTAAACC | |
| | AAATTAAACTATGGTGTCCAAAGATACGCA | |
| protegrin-1 protein | ATGGAGACCCAGAGAGCCAGCCTGTGCCTGGGGC | 50 |
| | GCTGGTCACTGTGGCTTCTGCTGCTGGCACTCGT | |
| | GGTGCCCTCGGCCAGCGCCCAGGCCCTCAGCTAC | |
| | AGGGAGGCCGTGCTTCGTGCTGTGGATCGCCTCA | |
| | ACGAGCAGTCCTCGGAAGCTAATCTCTACCGCCT | |
| | CCTGGAGCTGGACCAGCCGCCCAAGGCCGACGAG | |
| | GACCCGGGCACCCCGAAACCTGTGAGCTTCACGG | |
| | TGAAGGAGACTGTGTGTCCCAGGCCGACCCGGCA | |
| | GCCCCCGGAGCTGTGTGACTTCAAGGAGAACGGG | |
| | CGGGTGAAACAGTGTGTGGGGACAGTCACCCTGG | |
| | ATCAGATCAAGGACCCGCTCGACATCACCTGCAA | |
| | TGAGGTTCAAGGTGTCAGGGGAGGTCGCCTGTGC | |
| | TATTGTAGGCGTAGGTTCTGCGTCTGTGTCGGAC | |
| | GAGGATGACGGTTGCGACGGCAGGCTTTCCCTCC | |
| | CCCAATTTTCCCGGGGCCAGGTTTCCGTCCCCCA | |
| | ATTTTTCCGCCTCCACCTTTCCGGCCCGCACCAT | |
| | TCGGTCCACCAAGGTTCCCTGGTAGACGGTGAAG | |
| | GATTTGCAGGCAACTCACCCAGAAGGCCTTTCGG | |
| | TACATTAAAATCCCAGCAAGGAGACCTAAGCATC | |
| | TGCTTTGCCCAGGCCCGCATCTGTCAAATAAATT | |
| | CTTGTGAAACC | |
| protegrin-3 protein | ATGGAGACCCAGAGAGCCAGCCTGTGCCTGGGGC | 51 |
| | GCTGGTCACTGTGGCTTCTGCTGCTGGCACTCGT | |
| | GGTGCCCTCGGCCAGCGCCCAGGCCCTCAGCTAC | |
| | AGGGAGGCCGTGCTTCGTGCTGTGGATCGCCTCA | |
| | ACGAGCAGTCCTCGGAAGCTAATCTCTACCGCCT | |
| | CCTGGAGCTGGACCAGCCGCCCAAGGCCGACGAG | |
| | GACCCGGGCACCCCGAAACCTGTGAGCTTCACGG | |
| | TGAAGGAGACTGTGTGTCCCAGGCCGACCCGGCA | |
| | GCCCCCGGAGCTGTGTGACTTCAAGGAGAACGGG | |
| | CGGGTGAAACAGTGTGTGGGGACAGTCACCCTGG | |
| | ATCAGATCAAGGACCCGCTCGACATCACCTGCAA | |
| | TGAGGTTCAAGGTGTCAGGGGAGGTGGCCTGTGC | |
| | TATTGTAGGCGTAGGTTCTGCGTCTGTGTCGGAC | |
| | GAGGATGACGGTTGCGACGGCAGGCTTTCCCTCC | |
| | CCCAATTTTCCCGGGGCCAGGTTTCCGTCCCCCA | |
| | ATTTTTCCGCCTCCACCTTTCCGGCCCGCACCAT | |
| | TCGGTCCACCAAGGTTCCCTGGTAGACGGTGAAG | |
| | GATTTGCAGGCAACTCACCCAGAAGGCCTTTCGG | |
| | TACATTAAAATCCCAGCAAGGAGACCTAAGCATC | |
| | TGCTTTGCCCAGGCCCGCATCTGTCAAATAAATT | |
| | CTTGTGAAACC | |
| protegrin-4 protein | ATGGAGACCCAGAGAGCCAGCCTGTGCCTGGGGC | 52 |
| | GCTGGTCACTGTGGCTTCTGCTGCTGGCACTCGT | |
| | GGTGCCCTCGGCCAGCGCCCAGGCCCTCAGCTAC | |
| | AGGGAGGCCGTGCTTCGTGCTGTGGATCGCCTCA | |
| | ACGAGCAGTCCTCGGAAGCTAATCTCTACCGCCT | |
| | CCTGGAGCTGGACCAGCCGCCCAAGGCCGACGAG | |
| | GACCCGGGCACCCCGAAACCTGTGAGCTTCACGG | |
| | TGAAGGAGACTGTGTGTCCCAGGCCGACCCGGCA | |
| | GCCCCCGGAGCTGTGTGACTTCAAGGAGAACGGG | |
| | CGGGTGAAACAGTGTGTGGGGACAGTCACCCTGG | |
| | ATCAGATCAAGGACCCGCTCGACATCACCTGCAA | |
| | TGAGGTTCAAGGTGTCAGGGGAGGTCGCCTGTGC | |
| | TATTGTAGGGGTTGGATCTGCTTCTGTGTCGGAC | |
| | GAGGATGACGGTTGCGACGGCAGGCTTTCCCTCC | |
| | CCCAATTTTCCCGGGGCCAGGTTTCCGTCCCCCA | |
| | ATTTTTCCGCCTCCACCTTTCCGGCCCGCACCAT | |
| | TCGGTCCACCAAGGTTCCCTGGTAGACGGTGAAG | |
| | GATTTGCAGGCAACTCACCCAGAAGGCCTTTCGG | |
| | CACATTAAAATCCCAGCAAGGAGACCTAAGCATC | |
| | TGCTTTGCCCAGGCCCGCATCTGTCAAATAAATT | |
| | CTTGTGAAACC | |

The bacteriophase lysin may be selected from the group that includes but is not limited to holins and endolysins or lysins (e.g., lysozyme, amidase and transglycoslate). Additional lysins are disclosed in the following, each of which is incorporated herein by reference in its entirety: Kloos D.-U., et al., Journal of Bacteriology, 176 (23), 7352-7361 (December 1994); Jain V., et al., Infection and Immunity, 68 (2), 986-989 (February 2000); Srividhya K. V., et al., J. Biosci., 32, 979-990 (2007); Young R. V., Microbiological Reviews, 56 (3), 430-481 (September 1992).

The autolysin may be selected from the group that includes but is not limited to peptidoglycan hydrolases, amidases (e.g., N-acetylmuramyl-L-alanine amidases), transglycosylases, endopeptidases and glucosaminidases. Additional autolysins are disclosed in the following, each of which is incorporated herein by reference in its entirety: Heidrich C., et al., Molecular Microbiology, 41 (1), 167-178 (2001); Kitano K., et al., Journal of Bacteriology, 167 (3), 759-765 (September 1986); Lommatzsch J., et al., Journal of Bacteriology, 179 (17), 5465-5470 (September 1997); Oshida T., et al., PNAS, 92, 285-289 (January 1995); Lenz L. L., et al., PNAS, 100 (21), 12432-12437 (Oct. 14, 2003); Ramadurai L., et al., Journal of Bacteriology, 179 (11), 3625-3631 (June 1997); Kraft A. R., et al., Journal of Bacteriology, 180 (12), 3441-3447 (July 1998); Dijkstra A. J., et al., FEBS Letters, 366, 115-118 (1995); Huard C., et al., Microbiology, 149, 695-705 (2003).

In one aspect of the invention, the control exerted by the lysis regulation system may further be enhanced by bacterial or BTP strain-specific regulation. In one aspect of this embodiment, the strain-specific regulation is attenuation caused by deletion of a nutritional gene. The nutritional gene may be selected from the group that includes but is not limited to dapA, aroA and guaBA. In one example of this embodiment, dapA attenuation results in deficiency in the biosynthesis of lysine and peptidoglycan. In this particular embodiment, transcription of genes including but not limited to lysC may be activated by mechanisms such as transcriptional induction, antitermination and riboswitch. In another example of this embodiment, aroA attenuation results in deficiency in aromatic amino acids and derepression of one or more genes including but not limited to aroF, aroG and aroH by regulators such as TrpR and TyrR. In another example of this embodiment, guaBA attenuation results in derepression of one or more genes that are repressed by PurR.

In addition to the lysis regulation system and strain-specific regulation, the bacteria or BTP may further contain an inducible system that includes but is not limited to a Tet-on expression system to facilitate bacterial or BTP lysis at a time desired by the clinician. Upon administration of tetracycline, which activates the Tet-on promoter, the bacteria or BTP express a protein that triggers lysis of the bacteria or BTP. In one example of this embodiment, the protein expressed under the Tet-on expression system is selected from the group that includes but is not limited to defensins and protegrins.

The present invention also provides a lysis regulation system in combination with strain-specific attenuation (e.g., nutritional attenuation). As shown in PCT Publication No. WO2008/156702 at FIG. 30, a global regulator can sense an extraceullar condition and regulate transcription, starvation for specific nutrient such as an amino acid in vivo, in contrast to laboratory growth in the presence of excess of the nutrient and a positive or negative regulator in response to starvation. In the schematic shown in PCT Publication No. WO2008/156702 at FIG. 31 there can be three cassettes, any of which may be place on either the bacterial chromosome or on a plasmid.

As described, the present invention provides a plasmid containing a lysis regulation system comprising OmpR as the regulator, ompF or ompC as the promoter and protegrin or β-defensin as the antimicrobial protein, in combination with a Tet-on expression system, which provides two levels of control of bacterial lysis. This embodiment is illustrated in PCT Publication No. WO2008/156702 at FIG. 32.

In another aspect of the invention, the DNA insert comprises one or more of the following constructs, each of which contains an HPV target sequence, a hairpin sequence and BamH1 and Sal1 restriction sites to facilitate incorporation into the hairpin RNA expression cassette of the TRIP plasmid as shown in Table 10.

TABLE 10
HPV Target Sequence Construct

| | | | | |
|---|---|---|---|---|
| BamHI | sense (19bp) | loop | antisense (21bp) | SaII |

| | | | | |
|---|---|---|---|---|
| 5′-GATCC | TAGGTATTTGAATTTGCAT | TTCAAGAGA | ATGCAAATTCAAATACCTTTT | G-3′ (SEQ ID NO: 53) |
| 3′-G | ATCCATAAACTTAAACGTA | AAGTTCTCT | TACGTTTAAGTTTATGGAAAA | CAGCT-5′ (SEQ ID NO: 54) |

### 4. Cell and Gene Targets

The present invention also provides methods of using the various bacterium, BTP and vectors provided in the invention. For example, the present invention provides methods of delivering one or more siRNAs to mammalian cells. The methods include introducing at least one invasive bacterium, or at least one bacterial therapeutic particle (BTP), containing one or more siRNAs or one or more DNA molecules encoding one or more siRNAs to the mammalian cells.

The present invention also provides methods of regulating gene expression in mammalian cells. The method includes introducing at least one invasive bacterium, or at least one bacterial therapeutic particle (BTP), containing one or more siRNAs or one or more DNA molecules encoding one or more siRNAs to the mammalian cells, where the expressed siRNAs interfere with at least one mRNA of a gene of interest thereby regulating gene expression.

The invention provides a method for delivering RNA to any type of target cell. As used herein, the term “target cell” refers to a cell that can be invaded by a bacterium, i.e., a cell that has the necessary surface receptor for recognition by the bacterium.

Preferred target cells are eukaryotic cells. Even more preferred target cells are animal cells. “Animal cells” are defined as nucleated, non-chloroplast containing cells derived from or present in multicellular organisms whose taxanomic position lies within the kingdom animalia. The cells may be present in the intact animal, a primary cell culture, explant culture or a transformed cell line. The particular tissue source of the cells is not critical to the present invention.

The recipient animal cells employed in the present invention are not critical thereto and include cells present in or derived from all organisms within the kingdom animalia, such as those of the families mammalia, pisces, avian, reptilia.

Preferred animal cells are mammalian cells, such as humans, bovine, ovine, porcine, feline, canine, goat, equine, and primate cells. The most preferred mammalian cells are human cells. The cells can be in vivo, in vitro or ex vivo.

In some embodiments of the invention, the cell is a cervical epithelial cell, a rectal epithelial cell or a pharyngeal epithelial cell, macrophage, gastrointestinal epithelial cell, skin cell, melanocyte, keratinocyte, hair follicle, colon cancer cell, an ovarian cancer cell, a bladder cancer cell, a pharyngeal cancer cell, a rectal cancer cell, a prostate cancer cell, a breast cancer cell, a lung cancer cell, a renal cancer cell, a pancreatic cancer cell, a hepatocyte, a hepatocellular carcinoma (HCC) cell, a neural cell, or a hematologic cancer cell such as a lymphoma or leukemia cell. In one aspect of this embodiment, the colon cancer cell is an SW 480 cell. In another aspect of this embodiment, the pancreatic cancer cell is a CAPAN-1 cell.

In a preferred embodiment, the target cell is in a mucosal surface. Certain enteric pathogens, e.g., E. coli, Shigella, Listeria, and Salmonella, are naturally adapted for this application, as these organisms possess the ability to attach to and invade host mucosal surfaces (Kreig et al. supra). Therefore, in the present invention, such bacteria can deliver RNA molecules or RNA-encoding DNA to cells in the host mucosal compartment.

Although certain types of bacteria may have a certain tropism, i.e., preferred target cells, delivery of RNA or RNA-encoding DNA to a certain type of cell can be achieved by choosing a bacterium which has a tropism for the desired cell type or which is modified such as to be able to invade the desired cell type. Thus, e.g., a bacterium could be genetically engineered to mimic mucosal tissue tropism and invasive properties, as discussed above, to thereby allow said bacteria to invade mucosal tissue, and deliver RNA or RNA-encoding DNA to cells in those sites.

Bacteria can also be targeted to other types of cells. For example, bacteria can be targeted to erythrocytes of humans and primates by modifying bacteria to express on their surface either, or both of, the Plasmodium vivax reticulocyte binding proteins-1 and -2, which bind specifically to erythrocytes in humans and primates (Galinski et al. Cell, 69:1213-1226 (1992)). In another embodiment, bacteria are modified to have on their surface asialoorosomucoid, which is a ligand for the asilogycoprotein receptor on hepatocytes (Wu et al. J. Biol. Chem., 263:14621-14624 (1988)). In yet another embodiment, bacteria are coated with insulin-poly-L-lysine, which has been shown to target plasmid uptake to cells with an insulin receptor (Rosenkranz et al. Expt. Cell Res., 199:323-329 (1992)). Also within the scope of the invention are bacteria modified to have on their surface p60 of Listeria monocytogenes, which allows for tropism for hepatocytes (Hess et al. Infect. Immun, 63:2047-2053 (1995)), or a 60 kD surface protein from Trypanosoma cruzi which causes specific binding to the mammalian extra-cellular matrix by binding to heparin, heparin sulfate and collagen (Ortega-Barria et al. Cell, 67:411-421 (1991)).

Yet in another embodiment, a cell can be modified to become a target cell of a bacterium for delivery of RNA. Accordingly, a cell can be modified to express a surface antigen that is recognized by a bacterium for its entry into the cell, i.e., a receptor of an invasion factor. The cell can be modified either by introducing into the cell a nucleic acid encoding a receptor of an invasion factor, such that the surface antigen is expressed in the desired conditions. Alternatively, the cell can be coated with a receptor of an invasion factor. Receptors of invasion factors include proteins belonging to the integrin receptor superfamily. A list of the type of integrin receptors recognized by various bacteria and other microorganisms can be found, e.g., in Isberg and Tran Van Nhieu (1994) Ann. Rev. Genet. 27:395. Nucleotide sequences for the integrin subunits can be found, e.g., in GenBank, publicly available on the interne.

As set forth above, yet other target cells include fish, avian, and reptilian cells. Examples of bacteria that are naturally invasive for fish, avian, and reptilian cells are set forth below.

Examples of bacteria that can naturally access the cytoplasm of fish cells include, but are not limited to, Aeromonas salminocida (ATCC No. 33658) and Aeromonas schuberii (ATCC No. 43700). Attenuated bacteria are preferably used in the invention, and include A. salmonicidia vapA (Gustafson et al. J. Mol. Biol., 237:452-463 (1994)) or A. salmonicidia aromatic-dependent mutant (Vaughan et al. Infect. Immun., 61:2172-2181 (1993)).

Examples of bacteria that can naturally access the cytoplasm of avian cells include, but are not restricted to, Salmonella galinarum (ATCC No. 9184), Salmonella enteriditis (ATCC No. 4931) and Salmonella typhimurium (ATCC No. 6994). Attenuated bacteria are preferred to the invention and include attenuated Salmonella strains such as S. galinarum cya crp mutant (Curtiss et al. (1987) supra) or S. enteritidis aroA aromatic-dependent mutant CVL30 (Cooper et al. Infect. Immun., 62:4739-4746 (1994)).

Examples of bacteria that can naturally access the cytoplasm of reptilian cells include, but are not restricted to, Salmonella typhimurium (ATCC No. 6994). Attenuated bacteria are preferable to the invention and include, attenuated strains such as S. typhimuirum aromatic-dependent mutant (Hormaeche et al. supra).

The invention also provides for delivery of RNA to other eukaryotic cells, e.g., plant cells, so long as there are microorganisms that are capable of invading such cells, either naturally or after having been modified to become invasive. Examples of microorganisms which can invade plant cells include Agrobacterium tumerfacium, which uses a pilus-like structure which binds to the plant cell via specific receptors, and then through a process that resembles bacterial conjugation, delivers at least some of its content to the plant cell.

Set forth below are examples of cell lines to which RNA can be delivered according to the method of this invention.

Examples of human cell lines include but are not limited to ATCC Nos. CCL 62, CCL 159, HTB 151, HTB 22, CCL 2, CRL 1634, CRL 8155, HTB 61, and HTB104.

Examples of bovine cell lines include ATCC Nos. CRL 6021, CRL 1733, CRL 6033, CRL 6023, CCL 44 and CRL 1390.

Examples of ovine cells lines include ATCC Nos. CRL 6540, CRL 6538, CRL 6548 and CRL 6546.

Examples of porcine cell lines include ATCC Nos. CL 184, CRL 6492, and CRL 1746.

Examples of feline cell lines include CRL 6077, CRL 6113, CRL 6140, CRL 6164, CCL 94, CCL 150, CRL 6075 and CRL 6123.

Examples of buffalo cell lines include CCL 40 and CRL 6072.

Examples of canine cells include ATCC Nos. CRL 6213, CCL 34, CRL 6202, CRL 6225, CRL 6215, CRL 6203 and CRL 6575.

Examples of goat derived cell lines include ATCC No. CCL 73 and ATCC No. CRL 6270.

Examples of horse derived cell lines include ATCC Nos. CCL 57 and CRL 6583.

Examples of deer cell lines include ATCC Nos. CRL 6193-6196.

Examples of primate derived cell lines include those from chimpanzee's such as ATCC Nos. CRL 6312, CRL 6304, and CRL 1868; monkey cell lines such as ATCC Nos. CRL 1576, CCL 26, and CCL 161; orangautan cell line ATCC No. CRL 1850; and gorilla cell line ATCC No. CRL 1854.

The invention also provides methods of regulating the expression of one or more genes. Preferably, regulating the expression of one or more genes means decreasing or lessening the expression of the gene and/or decreasing or lessening the activity of the gene and its corresponding gene product.

In one embodiment, the expressed siRNAs direct the multienzyme complex RISC(RNA-induced silencing complex) of the cell to interact with the mRNAs to be regulated. This complex degrades or sequesters the mRNA. This causes the expression of the gene to be decreased or inhibited.

In some embodiments, the gene is an animal gene. Preferred animal genes are mammalian genes, such as humans, bovine, ovine, porcine, feline, canine, goat, equine, and primate genes. The most preferred mammalian genes are human cells.

The gene to be regulated can be a viral gene, anti-inflammatory gene, obesity gene or automimmune disease or disorder gene. In some embodiments, more than one gene can be regulated from a single plasmid or vector.

In preferred embodiments, the gene can be, but is not limited to, ras, Acatenin, one or more HPV oncogenes, APC, eotaxin-1 (CCL11), HER-2, MCP-1 (CCL2), MDR-1, MRP-2, FATP4, SGLUT-1, GLUT-2, GLUT-5, apobec-1, MTP, IL-6, IL-6R, IL-7, IL-12, IL-13, IL-13 R^{a}-1, IL-18, IL-21R, IL-32α, the p19 subunit of IL-23, LY6C, p38/JNK MAP kinase, p65/NF-κB, CCL20 (MIP-3α), Claudin-2, Chitinase 3-like 1, apoA-IV, MHC class I and MHC class II. In one aspect of this embodiment, the ras is k-Ras. In another aspect of this embodiment, the HPV oncogene is E6 or E7.

Preferable β-catenin target gene sequences are recited in Table 11. The sequences in Table 11 are cross-species target sequences as they are capable of silencing the beta-catenin gene (CTNNB1) in human, mouse, rat, dog and monkey.

TABLE 11

| β-catenin target gene sequences | SEQ ID NO: |
|---|---|
| AGCCAATGGCTTGGAATGAGA | 55 |
| ATCAGCTGGCCTGGTTTGATA | 56 |
| CTGTGAACTTGCTCAGGACAA | 57 |
| AGCAATCAGCTGGCCTGGTTT | 58 |
| CCTCTGTGAACTTGCTCAGGA | 59 |
| TTCCGAATGTCTGAGGACAAG | 60 |
| CCAATGGCTTGGAATGAGACT | 61 |
| GGTGCTGACTATCCAGTTGAT | 62 |
| CAATCAGCTGGCCTGGTTTGA | 63 |
| CACCCTGGTGCTGACTATCCA | 64 |
| CACCACCCTGGTGCTGACTAT | 65 |
| TGCTTTATTCTCCCATTGAAA | 66 |
| CTGGTGCTGACTATCCAGTTG | 67 |
| TCTGTGCTCTTCGTCATCTGA | 68 |
| TGCCATCTGTGCTCTTCGTCA | 69 |
| TGGTGCTGACTATCCAGTTGA | 70 |
| CCTGGTGCTGACTATCCAGTT | 71 |
| ACCCTGGTGCTGACTATCCAG | 72 |
| GAGCCTGCCATCTGTGCTCTT | 73 |
| CTGGTTTGATACTGACCTGTA | 74 |
| TGGTTTGATACTGACCTGTAA | 75 |
| TCGAGGAGTAACAATACAAAT | 76 |
| ACCATGCAGAATACAAATGAT | 77 |
| AGGAGTAACAATACAAATGGA | 78 |
| GTCGAGGAGTAACAATACAAA | 79 |
| TTGTTGTAACCTGCTGTGATA | 80 |
| GAGTAATGGTGTAGAACACTA | 81 |
| AGTAATGGTGTAGAACACTAA | 82 |
| CACACTAACCAAGCTGAGTTT | 83 |
| TTTGGTCGAGGAGTAACAATA | 84 |
| TACCATTCCATTGTTTGTGCA | 85 |
| TAGGGTAAATCAGTAAGAGGT | 86 |
| CTAACCAAGCTGAGTTTCCTA | 87 |
| TGGTCGAGGAGTAACAATACA | 88 |
| CTGGCCTGGTTTGATACTGAC | 89 |
| TAACCTCACTTGCAATAATTA | 90 |
| ATCCCACTGGCCTCTGATAAA | 91 |
| GACCACAAGCAGAGTGCTGAA | 92 |
| CACAAGCAGAGTGCTGAAGGT | 93 |
| CTAACCTCACTTGCAATAATT | 94 |
| AGCTGATATTGATGGACAG | 95 |

Preferable HPV target gene sequences are recited in Table 12. The sequences in Table 12 are target sequences as they are capable of silencing the HPV E6 oncogene.

TABLE 12

| HPV target gene sequences | SEQ ID NO: |
|---|---|
| CGGTGCCAGAAACCGTTGAATCC | 96 |
| CACTGCAAGACATAGAAATAACC | 97 |
| AGGTGCCTGCGGTGCCAGAAACC | 98 |
| GCGGTGCCAGAAACCGTTGAATC | 99 |
| TCACTGCAAGACATAGAAATAAC | 100 |
| CCCATGCTGCATGCCATAAATGT | 101 |
| ATGCTGCATGCCATAAATGTATA | 102 |
| GTGGTGTATAGAGACAGTATACC | 103 |
| GCGCGCTTTGAGGATCCAACACG | 104 |
| CTGCGGTGCCAGAAACCGTTGAA | 105 |
| CCCCATGCTGCATGCCATAAATG | 106 |
| ACCCCATGCTGCATGCCATAAAT | 107 |
| AACACTGGGTTATACAATTTATT | 108 |
| ACGACGCAGAGAAACACAAGTAT | 109 |
| AAGGTGCCTGCGGTGCCAGAAAC | 110 |
| GGTGCCTGCGGTGCCAGAAACCG | 111 |
| CATGCTGCATGCCATAAATGTAT | 112 |
| GACGCAGAGAAACACAAGTATAA | 113 |
| TTCACTGCAAGACATAGAAATAA | 114 |
| GGTGCCAGAAACCGTTGAATCCA | 115 |
| TGGCGCGCTTTGAGGATCCAACA | 116 |
| TGTGGTGTATAGAGACAGTATAC | 117 |
| GTGCCTGCGGTGCCAGAAACCGT | 118 |
| CTGCATGCCATAAATGTATAGAT | 119 |
| GACTCCAACGACGCAGAGAAACA | 120 |
| CTGGGCACTATAGAGGCCAGTGC | 121 |
| TGCTGCATGCCATAAATGTATAG | 122 |
| GTGCCAGAAACCGTTGAATCCAG | 123 |
| TTACAGAGGTATTTGAATTTGCA | 124 |
| GAGGCCAGTGCCATTCGTGCTGC | 125 |

Additional preferable HPV target gene sequences are recited in Table 13. The sequences in Table 13 are target sequences as they are capable of silencing the HPV E7 oncogene.

TABLE 13

| HPV target gene sequences | SEQ ID NO: |
|---|---|
| ATTCCGGTTGACCTTCTATGTCA | 126 |
| GATGGAGTTAATCATCAACATTT | 127 |
| AAGCCAGAATTGAGCTAGTAGTA | 128 |
| CATGGACCTAAGGCAACATTGCA | 129 |
| AACCACAACGTCACACAATGTTG | 130 |
| ATGGACCTAAGGCAACATTGCAA | 131 |
| TAAGCGACTCAGAGGAAGAAAAC | 132 |
| GAAGCCAGAATTGAGCTAGTAGT | 133 |
| GAGCCGAACCACAACGTCACACA | 134 |
| ACGTCACACAATGTTGTGTATGT | 135 |
| GAACCACAACGTCACACAATGTT | 136 |
| AGGCAACATTGCAAGACATTGTA | 137 |
| AAGACATTGTATTGCATTTAGAG | 138 |
| TAAGGCAACATTGCAAGACATTG | 139 |
| CCAGCCCGACGAGCCGAACCACA | 140 |
| AAGCTCAGCAGACGACCTTCGAG | 141 |
| GCCCGACGAGCCGAACCACAACG | 142 |
| TTCCGGTTGACCTTCTATGTCAC | 143 |
| TGCATGGACCTAAGGCAACATTG | 144 |
| TTCCAGCAGCTGTTTCTGAACAC | 145 |
| AACACCCTGTCCTTTGTGTGTCC | 146 |
| CTTCTATGTCACGAGCAATTAAG | 147 |
| ACGAGCCGAACCACAACGTCACA | 148 |
| TTGAGCTAGTAGTAGAAAGCTCA | 149 |
| CAGCAGACGACCTTCGAGCATTC | 150 |
| AGCCAGAATTGAGCTAGTAGTAG | 151 |
| GTCACACAATGTTGTGTATGTGT | 152 |
| CCGACGAGCCGAACCACAACGTC | 153 |
| AATTCCGGTTGACCTTCTATGTC | 154 |
| ATTCCAGCAGCTGTTTCTGAACA | 155 |

Additional preferable HPV target gene sequences are recited in Table 14. The sequences in Table 14 are target sequences shared by both HPV E6 and E6.

TABLE 14

| HPV target gene sequences | SEQ ID NO: |
|---|---|
| TAGGTATTTGAATTTGCAT | 156 |
| GAGGTATTTGAATTTGCAT | 157 |

A preferable MDR-1 target gene sequence is recited in Table 15. The sequence in Table 15 is capable of silencing the MDR-1 gene in human.

TABLE 15

| MDR-1 target gene sequences | SEQ ID NO: |
|---|---|
| ATGTTGTCTGGACAAGCACT | 158 |

A preferable k-Ras target gene sequence is recited in Table 16. The sequence in Table 16 is capable of silencing the k-Ras gene in human.

TABLE 16

| k-Ras target gene sequences | SEQ ID NO: |
|---|---|
| GTTGGAGCTGTTGGCGTAG | 159 |

Preferable IL-6R target gene sequences are recited in Table 17. The sequences in Table 17 are capable of silencing IL-6R in human.

TABLE 17

| IL-6R target gene sequences | SEQ ID NO: |
|---|---|
| CTCCTGGAACTCATCTTTCTA | 160 |
| GCTCTCCTGCTTCCGGAAGAG | 161 |
| CTCCACGACTCTGGAAACTAT | 162 |
| CAGAAGTTCTCCTGCCAGTTA | 163 |
| CCGGAAGACAATGCCACTGTT | 164 |
| CTGAACGGTCAAAGACATTCA | 165 |
| CACAACATGGATGGTCAAGGA | 166 |
| ATGCAGGCACTTACTACTAAT | 167 |
| ATCGGGCTGAACGGTCAAAGA | 168 |
| AGCTCTCCTGCTTCCGGAAGA | 169 |
| CAGCTCTCCTGCTTCCGGAAG | 170 |
| CAGGCACTTACTACTAATAAA | 171 |
| CACTTGCTGGTGGATGTTCCC | 172 |
| AACGGTCAAAGACATTCACAA | 173 |
| TGCACAAGCTGCACCCTCAGG | 174 |

Additional referable IL-6R target gene sequences are recited in Table 18. The sequences in Table 18 are capable of silencing the IL-6R gene in mouse.

TABLE 18

| IL-6R target gene sequences | SEQ ID NO: |
|---|---|
| ATCCTGGAGGGTGACAAAGTA | 175 |
| TGGGTCTGACAATACCGTAAA | 176 |
| AACGAAGCGTTTCACAGCTTA | 177 |
| CCGCTGTTTCCTATAACAGAA | 178 |
| ACGAAGCGTTTCACAGCTTAA | 179 |
| CTGCTGTGAAAGGGAAATTTA | 180 |
| AACCTTGTGGTATCAGCCATA | 181 |
| CACAGTGTGGTGCTTAGATTA | 182 |
| CAGCTTCGATACCGACCTGTA | 183 |
| CAGTGTGGTGCTTAGATTAAA | 184 |
| CCCGGCAGGAATCCTCTGGAA | 185 |
| CCCGCTGTTTCCTATAACAGA | 186 |
| AACCACGAGGATCAGTACGAA | 187 |
| ACCTGCCGTCTTACTGAACTA | 188 |
| ACCACGAGGATCAGTACGAAA | 189 |
| ACAGCTTGTGATGACTGAATA | 190 |
| AGGATCAGTACGAAAGTTCTA | 191 |
| AACCCGCTGTTTCCTATAACA | 192 |
| CAGTACGAAAGTTCTACAGAA | 193 |
| TACGCGAGTGACAATTTCTCA | 194 |
| ACGAAAGTTCTACAGAAGCAA | 195 |
| CAGGCACTTACTACTAATAAA | 196 |
| CACTTGCTGGTGGATGTTCCC | 197 |
| AACGGTCAAAGACATTCACAA | 198 |
| TGCACAAGCTGCACCCTCAGG | 199 |

Preferable IL-7 target gene sequences are recited in Table 19. The sequences in Table 19 are capable of silencing the IL-7 gene in human.

TABLE 19

| IL-7 target gene sequences | SEQ ID NO: |
|---|---|
| TAAGAGAGTCATAAACCTTAA | 200 |
| AACAAGGTCCAAGATACCTAA | 201 |
| AAGATTGAACCTGCAGACCAA | 202 |
| AAGAGATTTCAAGAGATTTAA | 203 |
| AAGCGCAAAGTAGAAACTGAA | 204 |
| TAGCATCATCTGATTGTGATA | 205 |
| TAAGATAATAATATATGTTTA | 206 |
| ATGGTCAGCATCGATCAATTA | 207 |
| TTGCCTGAATAATGAATTTAA | 208 |
| ATCTGTGATGCTAATAAGGAA | 209 |
| AACAAACTATTTCTTATATAT | 210 |
| AACATTTATCAATCAGTATAA | 211 |
| ATCAATCAGTATAATTCTGTA | 212 |
| AAGGTATCAGTTGCAATAATA | 213 |

Additional preferable IL-7 target gene sequences are recited in Table 20. The sequences in Table 20 are capable of silencing the IL-7 gene in mouse.

TABLE 20

| IL-7 target gene sequences | SEQ ID NO: |
|---|---|
| CGGATCCTACGGAAGTTATGG | 214 |
| GACCATGTTCCATGTTTCTTT | 215 |
| AACCTAAATGACCTTTATTAA | 216 |
| CAGGAGACTAGGACCCTATAA | 217 |
| TAGGGTCTTATTCGTATCTAA | 218 |
| ATGAGCCAATATGCTTAATTA | 219 |
| GCCAATATGCTTAATTAGAAA | 220 |
| CAGCATCGATGAATTGGACAA | 221 |
| TTGCCTGAATAATGAATTTAA | 222 |
| CTGATAGTAATTGCCCGAATA | 223 |
| AAGGGTTTGCTTGTACTGAAT | 224 |
| AACATGTATGTGATGATACAA | 225 |
| TTGCAACATGTAATAATTTAA | 226 |
| AAGAGACTACTGAGAGAAATA | 227 |
| AAGAATCTACTGGTTCATATA | 228 |
| TGCCGTCAGCATATACATATA | 229 |
| AGGGCTCACGGTGATGGATAA | 230 |

Additional preferable IL-7 target gene sequences are recited in Table 21. The sequences in Table 21 are cross species sequences as they are capable of silencing the IL-7 gene in human and mouse.

TABLE 21

| IL-7 target gene sequences | SEQ ID NO: |
|---|---|
| CGCCTCCCGCAGACCATGTTC | 231 |
| TCCGTGCTGCTCGCAAGTTGA | 232 |
| GCCTCCCGCAGACCATGTTCC | 233 |
| CCTCCCGCAGACCATGTTCCA | 234 |
| CTCCCGCAGACCATGTTCCAT | 235 |
| TCCCGCAGACCATGTTCCATG | 236 |
| CCCGCAGACCATGTTCCATGT | 237 |
| CCGCAGACCATGTTCCATGTT | 238 |
| CGCAGACCATGTTCCATGTTT | 239 |
| GCAGACCATGTTCCATGTTTC | 240 |
| CAGACCATGTTCCATGTTTCT | 241 |
| AGACCATGTTCCATGTTTCTT | 242 |

Preferable IL-13Ra-1 target gene sequences are recited in Table 22. The sequences in Table 22 are capable of silencing the IL-13Ra-1 gene in human.

TABLE 22

| IL-13Ra-1 target gene sequences | SEQ ID NO: |
|---|---|
| AACCTGATCCTCCACATATTA | 243 |
| CCTGATCCTCCACATATTAAA | 244 |
| AGAAATGTTTGGAGACCAGAA | 245 |
| CAAATAATGGTCAAGGATAAT | 246 |
| TTCCTGATCCTGGCAAGATTT | 247 |
| TAAAGAAATGTTTGGAGACCA | 248 |
| ATGTTTGGAGACCAGAATGAT | 249 |
| CTCCAATTCCTGATCCTGGCA | 250 |

Additional preferable IL-13Ra-1 target gene sequences are recited in Table 23. The sequences in Table 23 are capable of silencing the IL-13Ra-1 gene in mouse.

TABLE 23

| IL-13Ra-1 target gene sequences | SEQ ID NO: |
|---|---|
| CAAGAAGACTCTAATGATGTA | 251 |
| CACAGTCAGAGTAAGAGTCAA | 252 |
| ACCCAGGGTATCATAGTTCTA | 253 |
| CTGCTTTGAAATTTCCAGAAA | 254 |
| ATCATAGTTCTAAGAATGAAA | 255 |
| AAGGCTTAAGATCATTATATT | 256 |
| AACTACTTATAAGAAAGTAAA | 257 |
| CACAGAACATCTAGCAAACAA | 258 |
| CTCGTTCTTGTTCAATCCTAA | 259 |
| AACTTGTAGGTTCACATATTA | 260 |
| AACCATTTCTGCAAATTTAAA | 261 |
| CTCAGTGTAGTGCCAATGAAA | 262 |
| CAGGCCTTAGGGACTCATAAA | 263 |
| AAGTATGACATCTATGAGAAA | 264 |
| GTGGAGGTCAATAATACTCAA | 265 |
| CAGAGTATAGGTAAGGAGCAA | 266 |

A preferable IL-18 target gene sequence is recited in Table 24. The sequence in Table 24 is capable of silencing the IL-18 gene in human.

TABLE 24

| IL-18 target gene sequences | SEQ ID NO: |
|---|---|
| TTGAATGACCAAGTTCTCTTC | 267 |

Additional preferable IL-18 target gene sequences are recited in Table 25. The sequences in Table 25 are capable of silencing the IL-18 gene in mouse.

TABLE 25

| IL-18 target gene sequences | SEQ ID NO: |
|---|---|
| CTCTCTGTGAAGGATAGTAAA | 268 |
| CCGCAGTAATACGGAATATAA | 269 |
| CAAGGAAATGATGTTTATTGA | 270 |
| CAGACTGATAATATACATGTA | 271 |
| TTGGCCGACTTCACTGTACAA | 272 |
| CCAGACCAGACTGATAATATA | 273 |
| AAGATGGAGTTTGAATCTTCA | 274 |
| ACGCTTTACTTTATACCTGAA | 275 |
| TACAACCGCAGTAATACGGAA | 276 |
| CTGCATGATTTATAGAGTAAA | 277 |
| CCCGAGGCTGCATGATTTATA | 278 |
| CACGCTTTACTTTATACCTGA | 279 |
| CGCCTGTATTTCCATAACAGA | 280 |
| CGCAGTAATACGGAATATAAA | 281 |
| TACATGTACAAAGACAGTGAA | 282 |
| CAGGCCTGACATCTTCTGCAA | 283 |
| TTCGAGGATATGACTGATATT | 284 |
| CTGTATTTCCATAACAGAATA | 285 |
| GAGGATATGACTGATATTGAT | 286 |
| CAAGTTCTCTTCGTTGACAAA | 287 |
| CACTAACTTACATCAAAGTTA | 288 |
| ACCGCAGTAATACGGAATATA | 289 |
| CTCTCACTAACTTACATCAAA | 290 |

Preferable CCL20 target gene sequences are recited in Table 26. The sequences in Table 26 are capable of silencing the CCL20 gene in human.

TABLE 26

| CCL20 target gene sequences | SEQ ID NO: |
|---|---|
| ATCATCTTTCACACAAAGAAA | 291 |
| AACAGACTTGGGTGAAATATA | 292 |
| ATGGAATTGGACATAGCCCAA | 293 |
| GAGGGTTTAGTGCTTATCTAA | 294 |
| CTCACTGGACTTGTCCAATTA | 295 |
| ATCATAGTTTGCTTTGTTTAA | 296 |
| TTGTTTAAGCATCACATTAAA | 297 |
| AAGCATCACATTAAAGTTAAA | 298 |
| CCCAAAGAACTGGGTACTCAA | 299 |
| CACATTAAAGTTAAACTGTAT | 300 |
| CAGATCTGTTCTTTGAGCTAA | 301 |
| TTGGTTTAGTGCAAAGTATAA | 302 |
| CAGACCGTATTCTTCATCCTA | 303 |
| AACATTAATAAGACAAATATT | 304 |
| GACCGTATTCTTCATCCTAAA | 305 |

Additional referable CCL20 target gene sequences are recited in Table 27. The sequences in Table 27 are capable of silencing the CCL20 gene in mouse.

TABLE 27

| CCL20 target gene sequences | SEQ ID NO: |
|---|---|
| AAGCTTGTGACATTAATGCTA | 306 |
| CAATAAGCTATTGTAAAGATA | 307 |
| ATCATCTTTCACACGAAGAAA | 308 |
| AGCTATTGTAAAGATATTTAA | 309 |
| CAGCCTAAGAGTCAAGAAGAT | 310 |
| CCCAGTGGACTTGTCAATGGA | 311 |
| ATGAAGTTGATTCATATTGCA | 312 |
| AAGTTGATTCATATTGCATCA | 313 |
| TCACATTAGAGTTAAGTTGTA | 314 |
| CACATTAGAGTTAAGTTGTAT | 315 |
| TATGTTATTTATAGATCTGAA | 316 |
| ATGTTTAGCTATTTAATGTTA | 317 |
| TTAGTGGAAGGATTAATATTA | 318 |
| ACCCAGCACTGAGTACATCAA | 319 |
| TATGTTTAAGGGAATAGTTTA | 320 |

Additional preferable CCL20 target gene sequences are recited in Table 28. The sequences in Table 28 are cross-species target sequences as they are capable of silencing the CCL20 gene in human and mouse.

TABLE 28

| CCL20 target gene sequences | SEQ ID NO: |
|---|---|
| ATGAAGTTGATTCATATTGCA | 321 |
| TGAAGTTGATTCATATTGCAT | 322 |
| GAAGTTGATTCATATTGCATC | 323 |
| AAGTTGATTCATATTGCATCA | 324 |
| AGTTGATTCATATTGCATCAT | 325 |
| GTTGATTCATATTGCATCATA | 326 |
| TTGATTCATATTGCATCATAG | 327 |
| TGATTCATATTGCATCATAGT | 328 |
| TCAATGCTATCATCTTTCACA | 329 |
| CAATGCTATCATCTTTCACAC | 330 |
| TAATGAAGTTGATTCATATTG | 331 |
| AATGAAGTTGATTCATATTGC | 332 |

Preferable CCL20 target gene sequences are recited in Table 29. The sequences in Table 29 are capable of silencing the CCL20 gene in human.

TABLE 29

| Claudin-2 target gene sequences | SEQ ID NO: |
|---|---|
| AGCATGAAATTTGAGATTGGA | 333 |
| TACAGAGCCTCTGAAAGACCA | 334 |
| CACTACAGAGCCTCTGAAAGA | 335 |
| CTGACAGCATGAAATTTGAGA | 336 |
| ATCTCTGTGGTGGGCATGAGA | 337 |
| CATGAAATTTGAGATTGGAGA | 338 |
| TCTGGCTGAGGTTGGCTCTTA | 339 |
| GTGGGCTACATCCTAGGCCTT | 340 |

Additional preferable CCL20 target gene sequences are recited in Table 30. The sequences in Table 30 are capable of silencing the CCL20 gene in mouse.

TABLE 30

| Claudin-2 target gene sequences | SEQ ID NO: |
|---|---|
| CAGCTTCCTGCTAAACCACAA | 341 |
| CAAGAGTGAGTTCAACTCATA | 342 |
| CTGGTTCCTGACAGCATGAAA | 343 |
| TGGCTGGGACTATATATATAA | 344 |
| GAGGGCAATTGCTATATCTTA | 345 |
| CAGCAGCCAAACGACAAGCAA | 346 |
| CAAGGGTTTCCTTAAGGACAA | 347 |
| CAGATACTTGTAAGGAGGAAA | 348 |
| AAGAAATGGATTAGTCAGTAA | 349 |
| AAGGAAAGCACAAGAAGCCAA | 350 |
| CTGGCTGAGGTTGGCTCTTAA | 351 |
| AACCTGGGATCTAAAGAAACA | 352 |
| AAGGGCTTGGGTATCAAAGAA | 353 |
| CAGGCTCCGAAGATACTTCTA | 354 |
| CCCAATATATAAATTGCCTAA | 355 |
| CTGACCCAGCTTCCTGCTAAA | 356 |

Preferable Chitinase-3 target gene sequences are recited in Table 31. The sequences in Table 31 are capable of silencing the Chitinase-3 gene in human.

TABLE 31

| | SEQ ID |
|---|---|
| Chitinase-3 target gene sequences | NO: |
| ACCCACATCATCTACAGCTTT | 357 |
| CATCATCTACAGCTTTGCCAA | 358 |
| CAGCTGGTCCCAGTACCGGGA | 359 |
| CACCAAGGAGGCAGGGACCCT | 360 |
| CCGGTTCACCAAGGAGGCAGG | 361 |
| AGCTGGTCCCAGTACCGGGAA | 362 |
| CAGGCCGGTTCACCAAGGAGG | 363 |
| GGCCGGTTCACCAAGGAGGCA | 364 |

Additional preferable Chitinase-3 target gene sequences are recited in Table 32. The sequences in Table 32 are capable of silencing the Chitinase-3 gene in mouse.

TABLE 32

| | SEQ ID |
|---|---|
| Chitinase-3 target gene sequences | NO: |
| TAGGTTTGACAGATACAGCAA | 365 |
| AACCCTGTTAAGGAATGCAAA | 366 |
| ATCAAGTAGGCAAATATCTTA | 367 |
| CGCAGCTTTGTCAGCAGGAAA | 368 |
| TTGGATCAAGTAGGCAAATAT | 369 |
| TTGAGGGACCATACTAATTAT | 370 |
| GAGGACAAGGAGAGTGTCAAA | 371 |
| TGCGTACAAGCTGGTCTGCTA | 372 |
| CAGGAGTTTAATCTCTTGCAA | 373 |
| ATCAAGGAACTGAATGCGGAA | 374 |
| CACCCTGATCAAGGAACTGAA | 375 |
| CACTTGGATCAAGTAGGCAAA | 376 |
| CAGGATTGAGGGACCATACTA | 377 |
| AACTATGACAAGCTGAATAAA | 378 |
| ATGCAAATTCTCAGACTCTAA | 379 |
| ATCCTTCCCTTAGGAACTTAA | 380 |

### 5. Treatment of Diseases and Disorders

The present invention also provides methods of treating or preventing a disease or disorder in a mammal. The methods include regulating the expression of at least one gene in a cell known to cause a disease or disorder by introducing to the cells of the mammal at least one invasive bacterium, or at least one bacterial therapeutic particle (BTP), containing one or more siRNAs or one or more DNA molecules encoding one or more siRNAs, where the expressed siRNAs interfere with the mRNA of the gene known to cause the disease or disorder of interest.

The RNAi methods of the invention, including BMGS and tkRNAi are used to treat any disease or disorder for which gene expression regulation would be beneficial. This method is effected by silencing or knocking down (decreasing) genes involved with one or more diseases and disorders.

The gene to be regulated to treat or prevent a disease or disorder of interest, can be, but is not limited to, ras, Acatenin, one or more HPV oncogenes, APC, eotaxin-1 (CCL11), HER-2, MCP-1 (CCL2), MDR-1, MRP-2, FATP4, SGLUT-1, GLUT-2, GLUT-5, apobec-1, MTP, IL-6, IL-6R, IL-7, IL-12, IL-13, IL-13 Ra-1, IL-18, IL-21R, IL-32α, the p19 subunit of IL-23, LY6C, p38/JNK MAP kinase, p65/NF-κB, CCL20 (MIP-3α), Claudin-2, Chitinase 3-like 1, apoA-IV, MHC class I and MHC class II. In one aspect of this embodiment, the ras is k-Ras. In another aspect of this embodiment, the HPV oncogene is E6 or E7.

The present invention provides methods of treating or preventing a disease or disorder associated with the over-expression of a gene including, but not limited to, ras, β-catenin, one or more HPV oncogenes, APC, eotaxin-1 (CCL11), HER-2, MCP-1 (CCL2), MDR-1, MRP-2, FATP4, SGLUT-1, GLUT-2, GLUT-5, apobec-1, MTP, IL-6, IL-6R, IL-7, IL-12, IL-13, IL-13 Ra-1, IL-18, IL-21R, IL-32α, the p19 subunit of IL-23, LY6C, p38/JNK MAP kinase, p65/NF-κB, CCL20 (MIP-3α), Claudin-2, Chitinase 3-like 1, apoA-IV, MHC class I and MHC class II. Preferably, the gene is β-catenin and the disease disorder to be treated is one associated with the over-expression of β-catenin. The term “over-expression” as used herein refers to an increased expression (DNA, RNA or protein) when compared to normal or wild-type expression. Preferably, the disease or disorder to be treated is selected from the group consisting of colon cancer, rectal cancer, colorectal cancer, Crohn's disease, ulcerative colitis, familial adenomatous polyposis (FAP), Gardner's syndrome, hepatocellular carcinoma (HCC), basal cell carcinoma, pilomatricoma, medulloblastoma, and ovarian cancer.

Preferably, the present invention provides methods of treating or preventing cancer or a cell proliferation disorder, viral disease, an inflammatory disease or disorder, a metabolic disease or disorder, an autoimmune disease or disorder, or a disease, disorder or cosmetic concern in the skin or hair in a mammal by regulating the expression of a gene or several genes known to be associated with the onset, propagation or prolongation of the disease or disorder by introducing a bacterium or BTP to the cell. The bacterium or BTP contain one or more siRNAs or one or more DNA molecules encoding one or more siRNAs, where the expressed siRNAs interfere with the mRNA of the gene known to cause, propagate or prolong the disease or disorder of interest.

In some preferred embodiments, the viral disease can be, but is not limited to, hepatitis B, hepatitis C, Human Papilloma Virus (HPV) infection or epithelial dysplasia or cancer caused by HPV infection or HPV induced transformation, including cervical cancer, rectal cancer and pharyngeal cancer.

In some preferred embodiments, the inflammatory disease or disorder can be, but is not limited to, inflammatory bowel disease, Crohn's disease, ulcerative colitis, an allergy, rheumatoid arthritis or airway disease.

In some preferred embodiments, the automimmune disease or disorder can be, but is not limited to, celiac disease, rheumatoid arthritis, systemic lupus erythematosus or encephalomyelitis.

In some preferred embodiments, the disease, disorder or cosmetic concern can be, but is not limited to, psoriasis, eczema, albinism, balding or gray hair.

The mammal can be any mammal including, but not limited to, human, bovine, ovine, porcine, feline, canine, goat, equine, or primate. Preferably, the mammal is a human.

The terms “treating” and “treatment” as used herein refer to the administration of an agent or formulation (e.g., bacterium and/or BTP containing an siRNA or a DNA encoding for an siRNA) to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage.

The terms “preventing” and “prevention” refer to the administration of an agent or composition to a clinically asymptomatic individual who is susceptible to a particular adverse condition, disorder, or disease, and thus relates to the prevention of the occurrence of symptoms and/or their underlying cause.

### 6. Pharmaceutical Compositions and Modes of Administration

In a preferred embodiment of the invention, the invasive bacteria or BTPs containing the RNA molecules, and/or DNA encoding such, are introduced into an animal by intravenous, intramuscular, intradermal, intraperitoneally, peroral, intranasal, intraocular, intrarectal, intravaginal, intraosseous, oral, immersion and intraurethral inoculation routes.

The amount of the invasive bacteria or BTPs of the present invention to be administered to a subject will vary depending on the species of the subject, as well as the disease or condition that is being treated. Generally, the dosage employed will be about 10³ to 10¹¹ viable organisms, preferably about 10⁵ to 10⁹ viable organisms per subject.

The invasive bacteria or BTPs of the present invention are generally administered along with a pharmaceutically acceptable carrier and/or diluent. The particular pharmaceutically acceptable carrier an/or diluent employed is not critical to the present invention. Examples of diluents include a phosphate buffered saline, buffer for buffering against gastric acid in the stomach, such as citrate buffer (pH 7.0) containing sucrose, bicarbonate buffer (pH 7.0) alone (Levine et al. J. Clin. Invest., 79:888-902 (1987); and Black et al J. Infect. Dis., 155:1260-1265 (1987)), or bicarbonate buffer (pH 7.0) containing ascorbic acid, lactose, and optionally aspartame (Levine et al. Lancet, II-467-470 (1988)). Examples of carriers include proteins, e.g., as found in skim milk, sugars, e.g., sucrose, or polyvinylpyrrolidone. Typically these carriers would be used at a concentration of about 0.1-30% (w/v) but preferably at a range of 1-10% (w/v).

Set forth below are other pharmaceutically acceptable carriers or diluents which may be used for delivery specific routes. Any such carrier or diluent can be used for administration of the bacteria of the invention, so long as the bacteria or BTPs are still capable of invading a target cell. In vitro or in vivo tests for invasiveness can be performed to determine appropriate diluents and carriers. The compositions of the invention can be formulated for a variety of types of administration, including systemic and topical or localized administration. Lyophilized forms are also included, so long as the bacteria are invasive upon contact with a target cell or upon administration to the subject. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the composition, e.g., bacteria or BTPs, of the invention can be formulated in liquid solutions, preferably in physiologically compatible buffers such as Hank's solution or Ringer's solution.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner.

For administration by inhalation, the pharmaceutical compositions for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the composition, e.g., bacteria, and a suitable powder base such as lactose or starch.

The pharmaceutical compositions may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The pharmaceutical compositions may also be formulated in rectal, intravaginal or intraurethral compositions such as suppositories or retention enemas, e.g., containing conventional suppository bases such as cocoa butter or other glycerides.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For topical administration, the bacteria of the invention are formulated into ointments, salves, gels, or creams as generally known in the art, so long as the bacteria are still invasive upon contact with a target cell.

The compositions may, if desired, be presented in a pack or dispenser device and/or a kit that may contain one or more unit dosage forms containing the active ingredient. The pack may for example comprise metal or plastic foil, such as a blister pack. The pack or dispenser device may be accompanied by instructions for administration.

The invasive bacteria or BTPs containing the RNA or RNA-encoding DNA to be introduced can be used to infect animal cells that are cultured in vitro, such as cells obtained from a subject. These in vitro-infected cells can then be introduced into animals, e.g., the subject from which the cells were obtained initially, intravenously, intramuscularly, intradermally, or intraperitoneally, or by any inoculation route that allows the cells to enter the host tissue. When delivering RNA to individual cells, the dosage of viable organisms administered will be at a multiplicity of infection ranging from about 0.1 to 10⁶, preferably about 10² to 10⁴ bacteria per cell.

In yet another embodiment of the present invention, bacteria can also deliver RNA molecules encoding proteins to cells, e.g., animal cells, from which the proteins can later be harvested or purified. For example, a protein can be produced in a tissue culture cell.

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The present invention is further illustrated by the following examples that should not be construed as limiting in any way. The contents of all cited references including literature references, issued patents, published patent applications as cited throughout this application are hereby expressly incorporated by reference. The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

The following non-limiting examples are merely illustrative of the preferred embodiments of the present invention, and are not to be construed as limiting the invention.

### EXAMPLES

### Example 1

### Knockdown of β-Catenin and k-Ras

Previous studies have demonstrated the powerful nature of the siRNA knockdown technology disclosed herein. For example, in vitro and in vivo knockdown of beta catenin and k-ras utilizing bacterial delivery is described in PCT Publication No. WO 06/066048, which is incorporated herein by reference in its entirety.

### Example 2

### TRIP with Multiple shRNA Expression Cassettes

The TRIP described herein, and described in further detail in PCT Publication No. WO 06/066048, can be modified to produce a plasmid which allows targeting of multiple genes simultaneously or multiple sequences within one gene simultaneously. For example, TRIP with multiple hairpin expression cassettes to produce shRNA can target different sequences in a given gene, or target multiple genes through a simultaneous bacterial treatment.

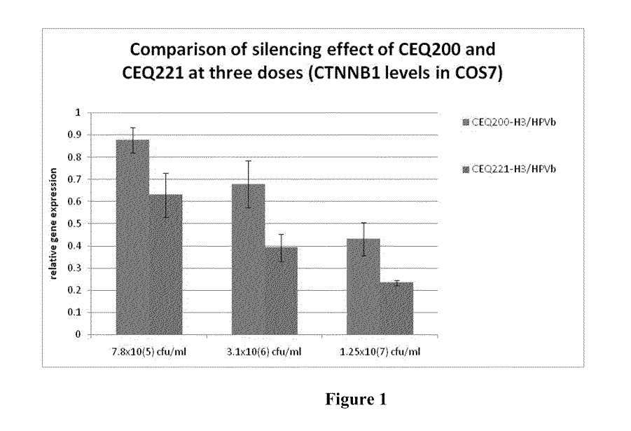

The TRIP plasmid can incorporate multiple (up to ten) cloning sites to express different shRNA constructs (as shown in PCT Publication No. WO2008/156702 at FIG. 1). The purpose of such a plasmid will be to allow silencing of various genes through a single therapeutic bacterium which will be empowered by the Multiple-expression cassette-TRIP (mec-TRIP) to synthesize short hairpin RNA against a variety of targets simultaneously.

These different hairpins can either be expressed competitively at high levels through the use of an identical high level promoter (such as T7 promoter or a different high level bacterial promoter), or they can be expressed at different levels through the use of promoters with different levels of activity, this will depend on the intended use of the plasmid and the desired relative silencing levels of the target gene.

This mec-TRIP could be useful to treat complex diseases as described herein (e.g. inflammatory diseases, or cancer), through the simultaneous silencing (targeting) of multiple targets as described herein (e.g. multiple oncogenes, such as k-ras and beta-catenin in the case of colon cancer, or HER-2 and MDR-1 in breast cancer, or other combinations).

### Example 3

### Operator Repressor Titration System

The TRIP system (bacteria and plasmid) have been modified to include the ORT (Operator Repressor Titration) system from Cobra Biomanufacturing (Keele, UK). This adaptation helps to maintain the plasmid in suitable strains in the absence of selective antibiotics. The bacterial carrier strain has been modified accordingly to allow for the ORT system to function (deletion of the DAP gene and replacement with an ORT-controlled DAP gene expression system). The plasmid has been modified to remove the antibiotic selection sequences to support the ORT system. Further changes have been introduced to the bacterial genome, including for example, (a) deletion of the aroA gene (in some CEQ strains) to make the bacteria more susceptible to nutrient shortage, particularly in the intracellular compartment where they will die due to lack of nutrients; (b) insertion of T7RNApolymerase gene into the chromosome and or (c) integration of a shRNA expression cassette under T7 promoter into the chromosome.

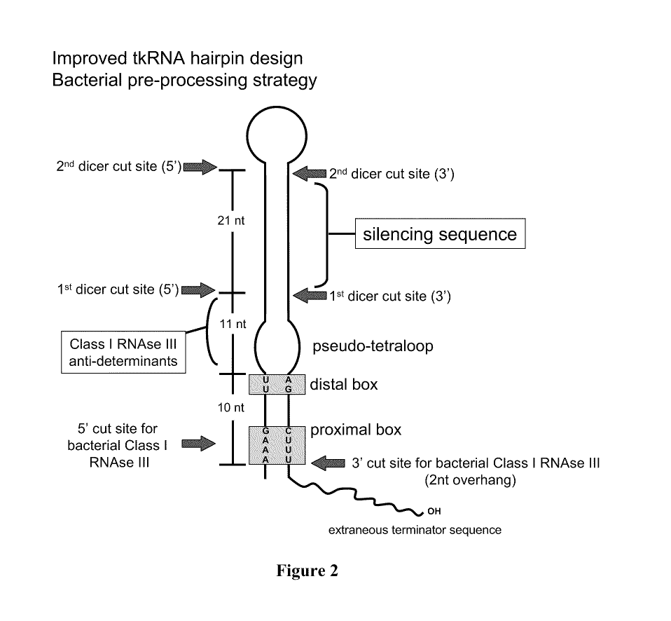

PCT Publication No. WO2008/156702 at FIG. 2 shows development examples of bacterial strains. Further strains developed include, but are not limited to, CEQ922 (CEQ919 without aroA deletion), CEQ923 (CEQ920 without aroA deletion), CEQ924 (CEQ921 without aroA deletion).

### Example 4

### Intestinal Tract Gene Delivery

S. typhimurium was investigated to determine if it could be used as a vector for RNAi delivery into the epithelial cells lining the intestinal tract. Mice were treated orally with a single dose of 10⁸ SL 7207 and sacrificed at various time points after administration. SL7207 were then stained using the Salmonella specific antibody. 2 h after treatment, numerous SL7207 could be seen invading the intestinal epithelial layer (Salmonella stained red), suggesting that oral administration of SL7207 may be a useful tool to deliver payloads to the intestinal and colonic mucosa. In a follow up experiment, mice were treated with SL7207 harboring a GFP expression plasmid (pEGFPC1, Invitrogen). At 24 h after a single treatment, a small percentage (approximately 1%) of cells was clearly found to express GFP.

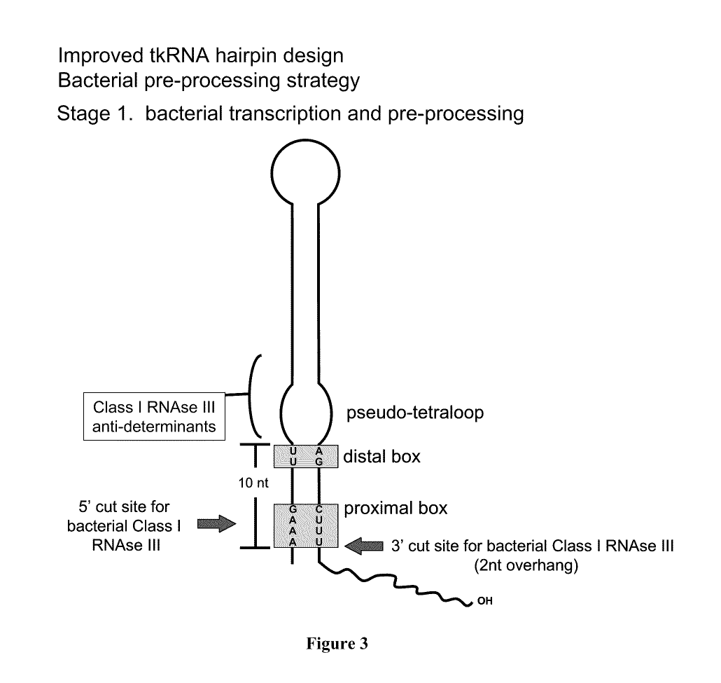

PCT Publication No. WO2008/156702 at FIG. 3 shows the efficient invasion and plasmid delivery into the intestinal mucosa by S. typhimurium. SL7207 were stained using red fluorescent antibody 6 h after oral administration. Intact SL7207 and fragments of SL7207 were seen in epithelial cells as well as underlying cells of the lamina propria (top left/right). SL7207 successfully deliver expressed DNA into the intestinal mucosa: intestinal mucosal cells expressing GFP after treatment with SL7207 carrying a eukaryotic expression plasmid for GFP (pEGFP-C1)(lower left). For fluorescence microscopy, SL7207 were stained with red fluorescent antibody and nuclei were counterstained with Hoechst 37111.

To test whether SL7207 could be used for the delivery of RNAi to target genes in the intestinal tract, GFP transgenic mice (4 per group) were treated with S. typhimurium harboring a shRNA expression plasmid directed against GFP (SL-siGFP) or a shRNA expression plasmid directed against k-RAS (SL-siRAS). 10⁸ c.f.u. was given three times weekly for two weeks by oral gavage. Colonic tissues were subsequently reviewed with fluorescent microscopy (data not shown) and stained analyzed after immunhistochemistry staining for GFP expression using a specific antibody (Living Colors®, Invitrogen). There was a significant reduction in the overall GFP expression level and significant reduction in the number of GFP expressing crypts in the SL-siGFP treated animals compared with the SL-siRAS treated animals (33.9% vs 50%, p<0.05), suggesting that this method could be useful to deliver therapeutic RNAi into the colonic epithelium.

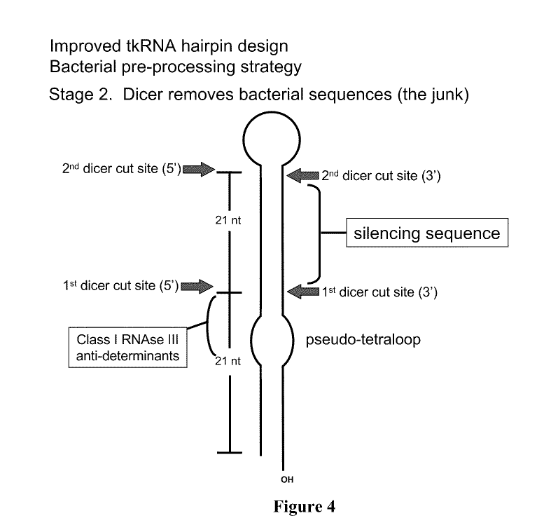

PCT Publication No. WO2008/156702 at FIG. 4 shows that bacteria-mediated RNA interference reduces target gene expression in the gastrointestinal epithelium. After treatment with SL7207 carrying expression plasmids targeting GFP (SL-siGFP, right bottom panel), colon tissues showed lower levels of GFP expression, and fewer colonic crypts were stained positive for GFP compared with animals treated with SL-siRAS (left bottom panel). Slides were stained with GFP-specific antibody.

### Example 5

### Construction of CEQ503 Bacterial Strain

Derivation and Description of CEQ 503 (Strain CEQ201 (pNJSZ))

CEQ503 consists of a combination of an attenuated E. coli strain (CEQ201) with a specially engineered TRIP plasmid (pNJSZ). The plasmid confers the abilities required to induce tkRNAi (in this case: invasiveness, escape from the entry vesicle, expression of short hairpin RNA). Strain Description of CEQ503 (pNJSZ):
1. Genotype: Escherichia coli CEQ201 [glnV44(AS), LAM⁻, rfbC1, endA1, spoT1, thi-1, hsdR17, (rₖ⁻mₖ⁺), creC510 ΔdapA, ΔrecA].
2. Derivation of CEQ201

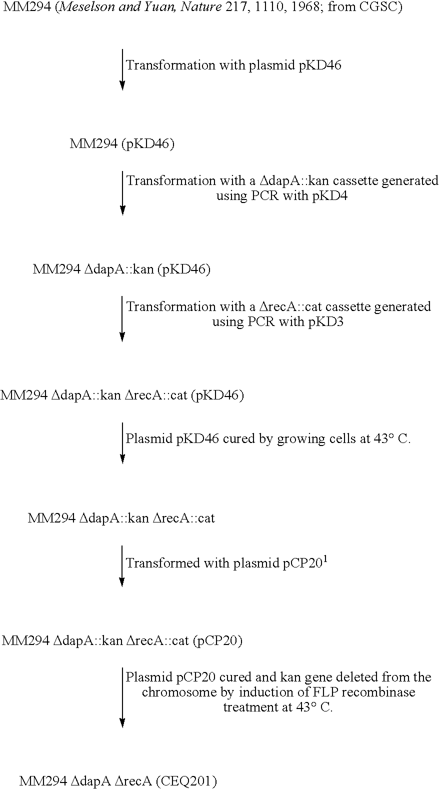

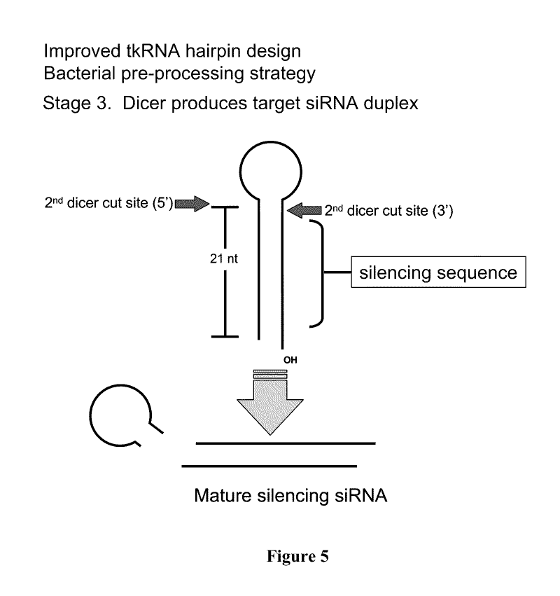

3. Plasmid: pNJSZ, shown schematically in PCT Publication No. WO2008/156702 at FIG. 5, is a 10.4 kb plasmid that confers kanamycin resistance to our bacterial strain (CEQ503). This plasmid contains two genes, hly and inv, and the H3 hairpin sequence: ggatccAGGAGTAACAATACAAATGGATTCAAGAGATCCATTTGTATTGTTACTCCTTTgt cgac (SEQ ID NO:383), which includes BamHI and SalI restriction sites. To verify the presence of this plasmid, PCRs are performed to verify chromosomal deletion of dapA, and minipreps and/or PCR are performed to confirm inv, hly and 341-H3 on the plasmid.
4. Nutritional Requirements: Althea Media Broth or LB, Miller (Luria-Bertani) broth (Amresco; cat. no.: J106-2KG) and 50 μg/ml of DL-Δ; ε-Diaminopemilic acid (DAP) (SIGMA; cat. no.: D1377-10G).
5. Growth Conditions: 37° C.

### Example 6

### BTP Production

BTPs or minicells containing a suitable plasmid such as TRIP have been engineered for delivery of tkRNAi. These cells will express invasin or Opa to enable entry into mammalian cells and listeriolysin will allow lysis of phagosome following minicell degradation/lysis. Additionally, a method for manufacturing minicells has been developed that utilizes a suicide construct for killing intact cells to aid in the purification of minicells. Such suicide plasmids have been described in the literature (Kloos et al., (1994) J. Bacteriol. 176, 7352-61; Jain and Mekalanos, (2000) Infect. Immun 68, 986-989). Summarily, the lambda S and R genes that code for holing and lysozyme are placed under regulation of an inducible promoter on the bacterial chromosome. When induced, they will lyse intact cells but not minicells since minicells lack chromosomes. A number of different types of regulators such as lad, araC, lambda c1857 and rhaS-rhaR can be used for development of an inducible suicide gene construct. Similarly, a number of different types of suicide genes, including E. coli autolysis genes and antimicrobial small peptides, can be used in a similar scheme. Purification is enhanced by treatments or mutations that induce filamentation (see, for example, Ward and Lutkenhaus, (1985) Cell 42, 941-949; Bi and Lutkenhaus, 1992). Initial purification involves low speed centrifugation to separate intact cells and retain minicells in the supernatant. This can be followed by density gradient purification or filtration (for example, Shull et al., (1971) J. Bacteriol. 106, 626-633).

Any cell death-triggering gene, also known as a suicide gene, including but not limited to genes encoding antimicrobial proteins, bacteriophage lysins or autolysins can be used in this method for obtaining BTPs from a mixture containing BTPs and bacteria. Suicide genes can kill live bacteria by mechanisms that include but are not limited to cell lysis, or by the destruction, degradation or poisoning of cellular components such as chromosomal DNA or filament components. Any inducible promoter may be used in conjunction with this system. In one embodiment of this invention, the suicide genes are integrated within the chromosome, thereby limiting their presence only in intact bacterial cells as BTPs or minicells will not incorporate these genes because they do not harbor chromosomal DNA.

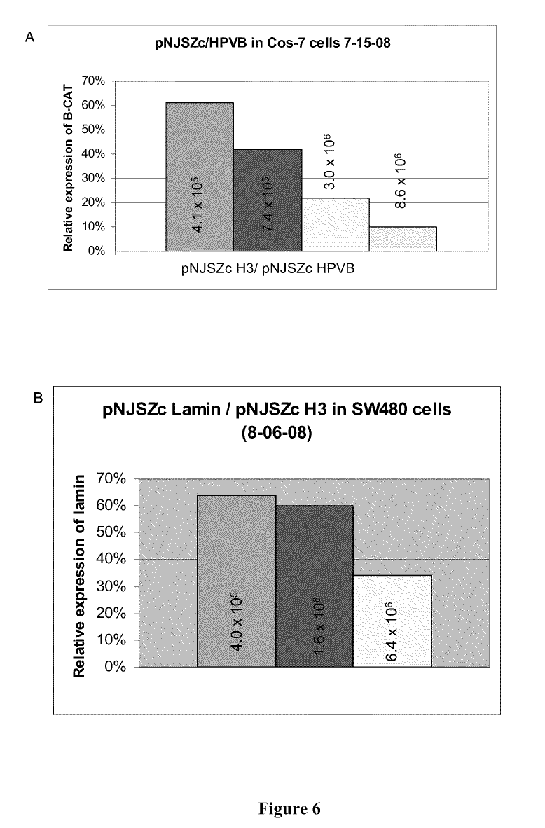

As shown in PCT Publication No. WO2008/156702 at FIG. 6, induction of suicide genes will lyse intact bacterial cells. The lambda S and R genes (suicide genes) are put under the control of P_{lacUV5} (inducible promoter). The leaky basal activity is repressed by a “super-repressor” coded by lacl^{q} gene on a P_{gapA} (strong promoter). This cassette is put at the minCD locus.

### Example 7

### siRNA Inhibition of Human Papillomavirus (HPV) Ongogenes

Cell Culture: Hela cells were cultured in Minimum Essential Medium (MEM, ATCC No. 30-2003) with 10% FBS supplemented with antibiotics: 100 U/ml penicillin G, 10 μg/ml streptomycin (Sigma).

Bacterial Culture: Plasmids were transformed into BL21(DE3) strain (Invitrogen). Bacteria were grown at 37° C. in LB Broth containing 100 μg/ml ampicillin. Bacterial cell density (in CFU/ml) was calculated using OD₆₀₀ measurement. For cell infection, overnight cultures were inoculated into fresh medium for another 2-3 h growth until the optical density at 600 nm [OD600] reached 0.6.

Invasion Assay For bacterial invasion, Hela cells were plated in 6-well dishes at 200,000 cells/well and allowed to incubate overnight in 2 ml complete growth medium. The bacterial cells were grown to mid-exponential phase with optical density at 600 nm [OD600] 0.6 in LB Broth with Ampicillin, and then centrifuged at 3,400 rpm for 10 minutes at 4° C. Bacterial pellets were resuspended in MEM without serum or the antibiotics and the bacteria were added to the cells at an MOI of 1:1000, 1:500, 1:250, 1:125, or 1:62.5 and allowed to invade the Hela cells for 2 hours at 37° C. in 5% CO2. The cells were washed 4 times with MEM containing 10% FBS and penicillin-streptomycin (100 IU of penicillin and 100 μg of streptomycin per ml). Cells were incubated in fresh complete medium for further 48 hours at 37° C. in 5% CO2 and total RNA was then isolated by the Qiagen RNeasy system with on-column DNAse digestion or by TRIZOL extraction method.

siRNA Transfection: One day before the transfection, cells were plated in complete growth medium without antibiotics so that the cells will be 30-50% confluent at the time of transfection. Diluted various concentrations of siRNA from a stock of 20 μM in 175 μl of Opti-MEM. Mixed 4 μl of Oligofectamine separately in 15 μl of Opti-MEM. Mixed gently and incubated for 5-10 min at room temperature. Combined the diluted siRNA with diluted oligofectamine and incubated for 15-20 min at room temperature. While the complexes were being formed, removed the growth medium from the cells and added 800 μl of medium without serum to each well containing cells. Added the 200 μl of siRNA/oligofectamine complexes to the cells and incubated at 37° C. for 4 h. Added 1 ml of growth medium containing 3× the normal concentration of serum without removing the transfection mixture. Gene silencing was assayed at 48 h.

RT-PCR: Quantitative real-time reverse transcription PCR (RT-PCR) was performed with the TaqMan RT-PCR master Mix Reagents Kit (Applied Biosystems) using the following primers and a probe set for detection of HPV18E6E7 transcripts:

(SEQ ID NO: 384)

Forward Primer: 5′-CTGATCTGTGCACGGAACTGA-3′
(148-168)

(SEQ ID NO: 385)

Reverse Primer: 5′-TGTCTAAGTTTTTCTGCTGGATTCA-3′
(439-463)

(SEQ ID NO: 386)

Probe: 5′-TTGGAACTTACAGAGGTGCCTGCGC-3′
(219-233 and 416-425)

The probe was labeled at the 5′ end with a reporter fluorescent dye, FAM and at the 3′ end with fluorescent dye quencher TAMRA. GAPDH was used to detect human GAPDH transcripts for the normalization.

HPV shRNA sequences:

H1 (working sequence)

(SEQ ID NO: 387)

5′-ggATCCTAGGTATTTGAATTTGCATTTCAAGAGAATGCAAATT
CAAATACCTTTTgTCgAC

(SEQ ID NO: 388)

5′-GTCGACAAAAGGTATTTGAATTTGCATTCTCTTGAAATGCAAA
TTCAAATACCTAGGATCC
H2 (ineffective sequence)

(SEQ ID NO: 389)

5′-ggATCCTCAGAAAAACTTAGACACCTTCAAGAGAGGTGTCTAA
GTTTTTCTGTTTgTCgAC

(SEQ ID NO: 390)

5′-GTCGACAAACAGAAAAACTTAGACACCTCTCTTGAAGGTGTCT
AAGTTTTTCTGAGGATCC

Western Blot: Hela cells were lysed using 1× Cell lysis Buffer (Cell Signaling Technology, Cat No. 9803). For electrophoresis, 50 μg of total protein in 2× loading buffer was loaded to each well of a 12% SDS-PAGE gel. After transferring the blot was blocked and probed with primary antibody at 2 h followed by incubation with HRP-conjugated secondary antibody before detection by ECL. All primary antibodies were used at 1/1000 dilution except HPV18E7 antibody at 1/250. Anti-Human pRb antibody: BD Pharmingen (Cat No. 554136), Sec Ab: HRP-anti Mouse HPV18E7: Santa Cruz (Cat No. sc-1590), Sec Ab:donkey anti-goat IgG-HRP Cat no. sc 2020 p53: Santa Cruz (Cat No. sc-126), Sec Ab: HRP-anti Mouse p21: Santa Cruz (Cat No. sc-397), Sec Ab: HRP-anti Rabbit c-Myc: Cell Signaling Technology (Cat No. 9402), Sec Ab: HRP-anti Rabbit

Colony Formation Assay: Hela cells were harvested after bacterial invasion for 2 h. The cells in either control treated or HPV shRNA treated cells were washed 3× times with complete MEM and one time with PBS. The cells were then trypsinized and counted. 500 cells from each treatment were added to a single well of a six well plate containing 2 ml of complete growth medium. The cells were allowed to grow for 10 days following which the colonies were fixed with GEIMSA stain.

MTT Assay: Hela cells were harvested after bacterial invasion for 2 h. The cells in either control treated or HPV shRNA treated cells were washed 3× times with complete MEM and one time with PBS. The cells were then trypsinized and counted. 5000 cells from each treatment were added to a single well of a 96 well plate in 100 μl of complete growth medium in triplicates. The cells were incubated at 37° C. for 48-72 h following which 10 μl of 0.5 mg/ml MTT was added to each well. The plate was further incubated at 37° C. for 3 h, the medium was aspirated off from the wells and after incubation, 100 μl of MTT solubilization solution [10% Triton X-100 in acidic isoproponal (0.1 N HCl)] was added to each well to stop the reaction. The absorbance was read at 570 nm on the plate reader.

In this example, the suppressive effect of a short hairpin RNA directed towards HPV 18 E6 and E7 oncogenes was investigated. The short hairpin RNA was delivered by infecting human cervical cancer cells (Hela) with bacterial strains that produce the short hairpin RNA. The shRNA expression cassette contained 19 nucleotide (nt) of the target sequence followed by the loop sequence (TTCAAGAGA) (SEQ ID NO:391) and the reverse complement to the 19 nt. For the 19 nt, two shRNA sequences published in Cancer Gene Therapy (2006) 13, 1023-1032, were used to measure siRNA delivery and gene silencing efficiency, oligofectamine reagent in a 6 well format was used. Briefly, Hela cells were plated at a cell density of about 40% confluence in antibiotic free medium. On the next day, siRNA was added to 6 well plates at varying concentrations of 50, 100, 200 nM. The control siRNA was added at a single concentration of 100 nM.

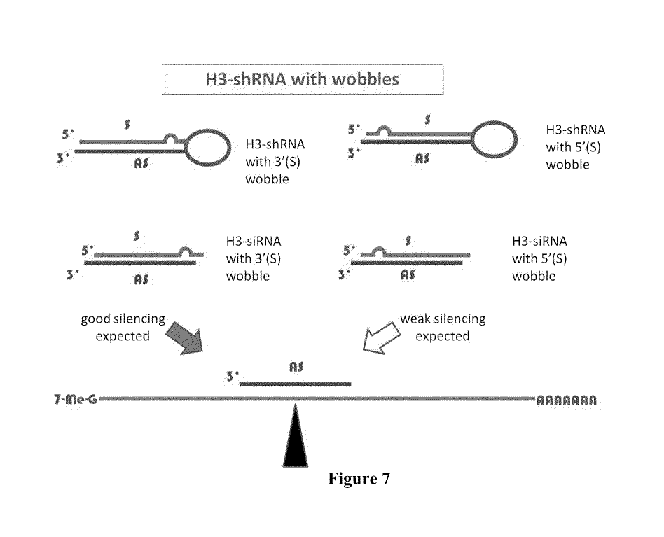

As shown in PCT Publication No. WO2008/156702 at FIG. 7, the oligofectamine transfection method resulted in a decrease in E6 mRNA in Hela cells with respect to the control siRNA. The siRNA (H1) showed up to about 40% of reduction in E6 mRNA. The knockdown response was not dose dependent.

Next, the hairpin of the siRNA (H1) was cloned into the TRIP vector. In order to determine if gene silencing could be achieved through the transkingdom system, the shRNA in human cervical cancer cells (Hela) was tested in an invasion assay. Briefly, Hela cells were plated in a six-well plate at 2×10⁵ cells/well, allowed to grow overnight and incubated the next day for 2 h at different MOIs with bacteria (E. coli) engineered to produce the hairpin RNA. The bacteria were washed off with medium containing 10% FBS and Pen Strep four times and the mammalian cells were further incubated for an additional 48 h in the complete medium. RNA or protein was isolated from the bacteria.

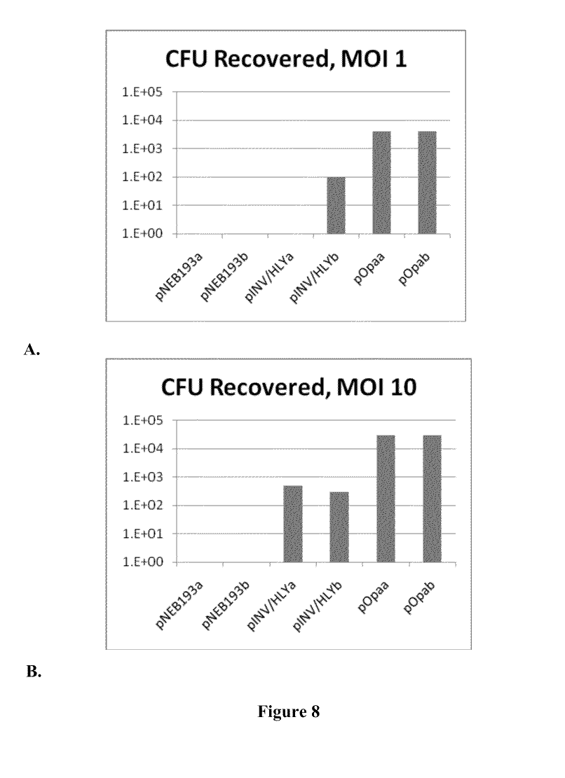

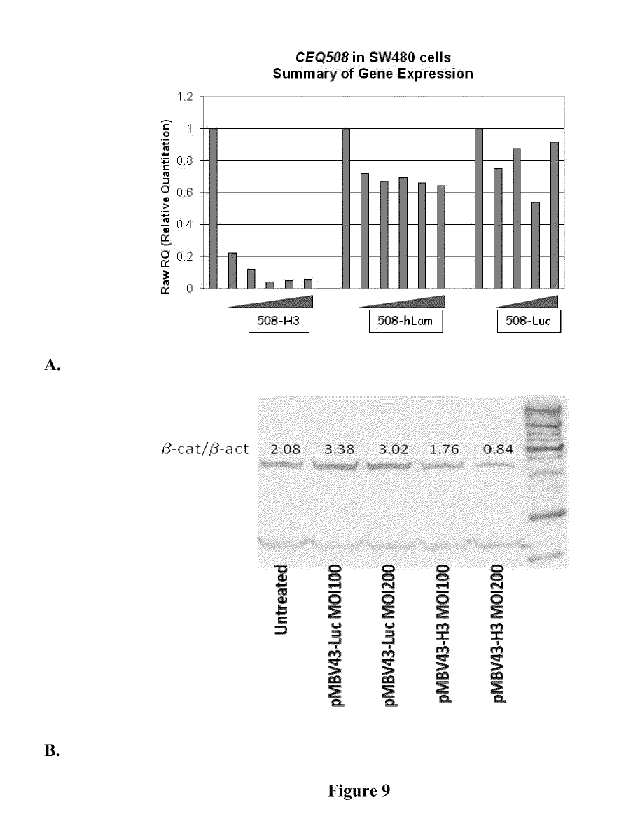

PCT Publication No. WO2008/156702 at FIGS. 8 and 9 demonstrate that siRNA downregulates HPV E6 expression in Hela cells. Cells were plated in six well plates and allowed to grow to a confluence of 40% (about 40,000 cells). Oligofectamine/siRNA transfection complexes were prepared in Opti-MEM serum-free medium by mixing 4 μl of oligofectamine with siRNAs (final concentration in 185 μl of medium is 50, 100, 200 nM). 48 hours post-transfection cells were harvested and analyzed by real-time RT-PCR for both target and GAPDH mRNA levels. Data were normalized against the GAPDH signal. Two different negative control siRNAs were used at a single concentration of 200 nM.

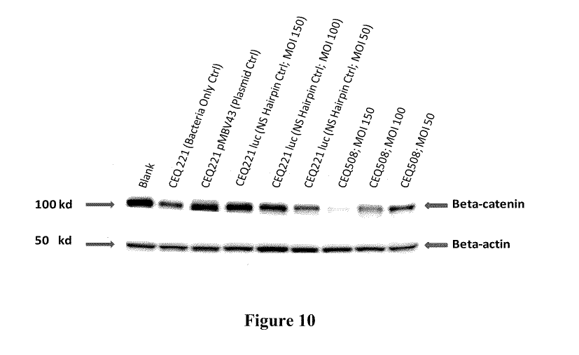

PCT Publication No. WO2008/156702 at FIG. 10, Panels A-C show real time PCR results following invasion assay of Hela cells. Hela cells were incubated for 2 h with shRNA-expressing BL21(DE3) at different multiplicities of infection (MOI). Forty-eight hours post-infection the cells were harvested and analyzed by real-time RT-PCR for both target and GAPDH mRNA levels. Data were normalized against the GAPDH signal. These data were then further normalized to untreated control cells.

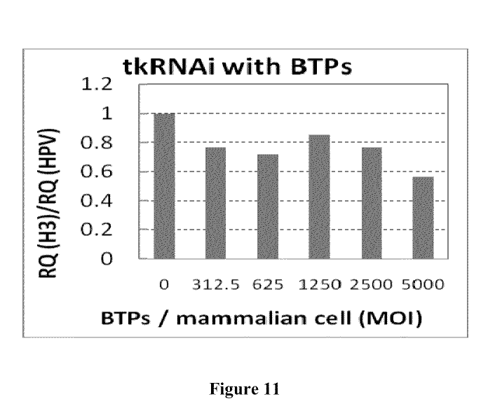

PCT Publication No. WO2008/156702 at FIG. 11 shows the effects of downregulation of HPV E6 and E7 genes on tumor suppressor pathways and other downstream targets. Hela cells were incubated for 2 h with shRNA-expressing BL21(DE3) at different multiplicities of infection (MOI). Forty-eight hours post-infection cells were harvested and analyzed by western blotting. 50 μg of protein was loaded in each lane and resolved by gel electrophoresis, transferred to a membrane and probed with antibodies specific for HPV 18 E7, p53, actin, p110Rb, p21 and c-myc as indicated.

PCT Publication No. WO2008/156702 at FIGS. 12 and 13 show a colony formation and MTT assay, respectively. Hela cells were incubated for 2 h with shRNA-expressing BL21(DE3) at different multiplicities of infection (MOI). 2 h post-infection cells were washed trypsinized and counted and an equal number of cells for each MOI was added to a well of a six well plate (For CFA: added 500 cells to each well of a 6 well plate, for MTT added 5000 cells in each well of a 96 well plate). For colony formation, the cells were allowed to grow for 10 days and stained with Geimsa, MTT assay was analyzed at 72 h post plating.

PCT Publication No. WO2008/156702 at FIGS. 14 and 15 show real time PCR results following invasion assay of Hela cells. Hela cells were incubated for 2 h with shRNA-expressing BL21(DE3) at different multiplicities of infection (MOI). Forty-eight hours post-infection cells were harvested and analyzed by real-time RT-PCR for both target and GAPDH mRNA levels. Data were normalized against the GAPDH signal. These data were then further normalized to untreated control cells.

PCT Publication No. WO2008/156702 at FIG. 16 shows the effects of downregulation of HPV E6 and E7 genes on tumor suppressor pathways and other downstream targets. Hela cells were incubated for 2 h with shRNA-expressing BL21(DE3) at different multiplicities of infection (MOI). Forty-eight hours post-infection cells were harvested and analyzed by western blotting. 50 μg of protein was loaded in each lane and resolved by gel electrophoresis, transferred to a membrane and probed with antibodies specific for HPV 18 E7, p53, actin, p110Rb as indicated.

PCT Publication No. WO2008/156702 at FIG. 17 shows real time PCR results following invasion assay of Hela cells with a frozen aliquot of negative sHRNA control and HPV sHRNA in BL21 (DE3). Hela cells were incubated for 2 h with shRNA-expressing BL21(DE3) at different multiplicities of infection (MOI). Forty-eight hours post-infection cells were harvested and analyzed by real-time RT-PCR for both target and GAPDH mRNA levels. Data were normalized against the GAPDH signal. These data were then further normalized to untreated control cells.

PCT Publication No. WO2008/156702 at FIG. 18 shows the plating efficiency of frozen aliquots of negative sHRNA control and HPV sHRNA in BL21 (DE3). The frozen bacteria were thawed and resuspended to a final concentration of 3.38×10⁸ cells/ml. Invasion assay was performed with this concentration taking 2 mls of 3.38×10⁸ cells/ml as an MOI of 1000. Some stock control bacteria or HPV bacteria were serially diluted (1:100) and plated on LB plates to assess for the number and viability of bacteria treated cells at 48 h. Gene silencing was analyzed either by quantitative real-time PCR using the ΔΔCt relative quantitation method or by western blot analysis. HPVE6 mRNA levels were normalized to an endogenous control, GAPDH. The final data were further normalized to the RNA from the untreated cells. For Protein analysis, cell lysates were prepared in Cell Lysis Buffer (Cell Signaling Technology) and the protein concentration was determined using a BCA kit from BioRad. For electrophoresis, the protein expression was normalized to Actin loading control.

### Example 8

### Knockdown of HPV E6 Gene Assessed by Western Blotting with HPV 18 E7 Antibody

Hela cells were incubated for 2 h with shRNA-expressing BL21(DE3) (HPVH1 construct below) at different multiplicities of infection (MOI). Forty-eight hours post-infection cells were harvested and analyzed by western blotting. The HPV E6 specific knockdown was compared with a negative shRNA control. Briefly, 50 μg of protein was loaded in each lane and resolved by gel electrophoresis, transferred to a membrane and probed with antibodies specific for HPV 18 E7, and actin as indicated.

HPVH1

(SEQ ID NO: 392)

5′-GATCC TAGGTATTTGAATTTGCAT TTCAAGAGA
ATGCAAATTCAAATACCTTTT G-3′

(SEQ ID NO: 393)

3′-G ATCCATAAACTTAAACGTA AAGTTCTCT
TACGTTTAAGTTTATGGAAAA CAGCT-5′

PCT Publication No. WO2008/156702 at FIG. 19 shows the knockdown of HPV E6 gene assessed by western blotting with HPV 18 E7 antibody. Hela cells were incubated for 2 h with shRNA-expressing BL21(DE3) at different multiplicities of infection (MOI). Forty-eight hours post-infection cells were harvested and analyzed by western blotting. The HPV E6 specific knockdown was compared with a negative sHRNA control. Briefly, 50 μg of protein was loaded in each lane and resolved by gel electrophoresis, transferred to a membrane and probed with antibodies specific for HPV 18 E7 and actin as indicated.

### Example 9

### Inhibition of CCL20 Expression in CMT93 Cells

One confluent T-175 flask of CMT93 cells was trypsinized in 10 mls until the cells detached. Trypsin was inactivated by addition of 30 mls of DMEM (10% FCS, pen/strep) and the cells thoroughly mixed by pipetting. From this solution, 8 mls was transferred into a sterile 50 ml tube and 32 mLs of DMEM 10% added. Cells were well mixed and 250 uLs added to each well of a 48 well plate and incubated overnight at 37 C resulting in adherent cells that were approximately 70% confluent the following morning. The next day, siRNA transfection complexes were created by the following method.

Sequences were ordered from Qiagen as pre-annealed siRNA duplexes. Each well was resuspended in 250 ul of siRNA buffer (from Qiagen) to give a stock concentration of 20 uM. The plate was then placed in a water bath at 95 C for 5 minutes and then allowed to slowly cool to resuspend the duplexes and break apart aggregates. The suspended duplexes were then used in transfection experiments described in standard protocols. The formulation is per well of a 48 well plate containing 250 uL of media; each screen was performed in biological triplicate so the solution was made for 4 wells; 3 for transfection and 1 extra.

0.3 uL of the appropriate siRNA (from a 20 uM stock solution) were diluted to 47 uL with serum/antibiotic free media and mixed. To this solution was added 3 uL of HiPerfect transfection reagent (Qiagen) followed by brief vortexing and incubation at room temperature for 20 minutes. 50 uLs of the complex containing mixture was added to each of 3 wells in a 48 well plate containing CMT93 cells. Transfection was for 24 hours at 37 C at which time the media was removed and replaced with 400 uLs of DMEM/10% FCS containing 100 ng/mL of LPS for 2 hours. Following stimulation, the cells were washed and RNA isolated for qRT-PCR according the Qiagen Quantitech method (see manufacturer's protocol) for 50 cycles.

PCT Publication No. WO2008/156702 at FIG. 20 shows the knockdown of CCL20 expression with the various siRNA sequences in CMT93 cells. The siRNA sequences tested are listed in Table 33.

TABLE 33

| | | SEQ ID | | SEQ ID |
|---|---|---|---|---|
| Well | siRNA sense 5′→3′ | NO: | siRNA antisense 5′→3′ | NO: |
| G1 | GCUUGUGACAUUAAUGCUAtt | 394 | UAGCAUUAAUGUCACAAGCtt | 395 |
| H1 | AUAAGCUAUUGUAAAGAUAtt | 396 | UAUCUUUACAAUAGCUUAUtg | 397 |
| G2 | CAUCUUUCACACGAAGAAAtt | 398 | UUUCUUCGUGUGAAAGAUGat | 399 |
| H2 | CUAUUGUAAAGAUAUUUAAtt | 400 | UUAAAUAUCUUUACAAUAGct | 401 |
| G3 | GCCUAAGAGUCAAGAAGAUtt | 402 | AUCUUCUUGACUCUUAGGCtg | 403 |
| G4 | CAGUGGACUUGUCAAUGGAtt | 404 | UCCAUUGACAAGUCCACUGgg | 405 |
| G5 | GAAGUUGAUUCAUAUUGCAtt | 406 | UGCAAUAUGAAUCAACUUCat | 407 |
| G6 | GUUGAUUCAUAUUGCAUCAtt | 408 | UGAUGCAAUAUGAAUCAACtt | 409 |
| G7 | ACAUUAGAGUUAAGUUGUAtt | 410 | UACAACUUAACUCUAAUGUga | 411 |
| G8 | CAUUAGAGUUAAGUUGUAUtt | 412 | AUACAACUUAACUCUAAUGtg | 413 |
| G9 | UGUUAUUUAUAGAUCUGAAtt | 414 | UUCAGAUCUAUAAAUAACAta | 415 |
| G10 | GUUUAGCUAUUUAAUGUUAtt | 416 | UAACAUUAAAUAGCUAAACat | 417 |
| G11 | AGUGGAAGGAUUAAUAUUAtt | 418 | UAAUAUUAAUCCUUCCACUaa | 419 |
| F12 | CCAGCACUGAGUACAUCAAtt | 420 | UUGAUGUACUCAGUGCUGGgt | 421 |
| G12 | UGUUUAAGGGAAUAGUUUAtt | 422 | UAAACUAUUCCCUUAAACAta | 423 |

### Example 10

### Inhibition of Expression of Claudin-2 in CMT93 Cells

One confluent T-175 flask of CMT93 cells was trypsinized in 10 mls until the cells detached. Trypsin was inactivated by addition of 30 mls of DMEM (10% FCS, pen/strep) and the cells thoroughly mixed by pipetting. From this solution, 8 mls was transferred into a sterile 50 ml tube and 32 mLs of DMEM 10% added. Cells were well mixed and 250 uL added to each well of a 48 well plate and incubated overnight at 37 C resulting in adherent cells that were approximately 70% confluent the following morning. The next day, siRNA transfection complexes were created by the following method:

Sequences were ordered from Qiagen as pre-annealed siRNA duplexes. Each well was resuspended in 250 ul of siRNA buffer (from Qiagen) to give a stock concentration of 20 uM. The plate was then placed in a water bath at 95 C for 5 minutes and then allowed to slowly cool to resuspend the duplexes and break apart aggregates. The suspended duplexes were then used in transfection experiments described in standard protocols. The formulation is per well of a 48 well plate containing 250 uL of media; each screen was performed in biological triplicate so the solution was made for 4 wells; 3 for transfection and 1 extra.

0.3 uL of the appropriate siRNA (from a 20 uM stock solution) to 47 uL of serum/antibiotic free media and mixed. To this solution was added 3 uL of HiPerfect transfection reagent (Qiagen) followed by brief vortexing and incubation at room temperature for 20 minutes. 50 uLs of the complex containing mixture was added to each of 3 wells in a 48 well plate containing CMT93 cells. Transfection was for 24 or 48 hours at 37 C at which time the cells were washed and RNA isolated for qRT-PCR according the Qiagen Quantitech method (see manufacturer's protocol) for 50 cycles.

PCT Publication No. WO2008/156702 at FIG. 21 shows the knockdown of Claudin-2 expression with the various siRNA sequences in CMT93 cells post 24 hr transfection. The siRNA sequences tested are listed in Table 34.

TABLE 34

| | | SEQ ID | | SEQ ID |
|---|---|---|---|---|
| Well | siRNA sense 5′→3′ | NO: | siRNA antisense 5′→3′ | NO: |
| D4 | GCUGGGACUAUAUAUAUAAtt | 424 | UUAUAUAUAUAGUCCCAGCca | 425 |
| D5 | GGGCAAUUGCUAUAUCUUAtt | 426 | UAAGAUAUAGCAAUUGCCCtc | 427 |
| D6 | GCAGCCAAACGACAAGCAAtt | 428 | UUGCUUGUCGUUUGGCUGCtg | 429 |
| D7 | AGGGUUUCCUUAAGGACAAtt | 430 | UUGUCCUUAAGGAAACCCUtg | 431 |
| C9 | GAAAUGGAUUAGUCAGUAAtt | 432 | UUACUGACUAAUCCAUUUCtt | 433 |
| D11 | GGCUCCGAAGAUACUUCUAtt | 434 | UAGAAGUAUCUUCGGAGCCtg | 435 |

### Example 11

### Inhibition of Expression of IL6-Ra in CMT93 Cells

One confluent T-175 flask of CMT93 cells was trypsinized in 10 mls until the cells detached. Trypsin was inactivated by addition of 30 mls of DMEM (10% FCS, pen/strep) and the cells thoroughly mixed by pipetting. From this solution, 8 mls was transferred into a sterile 50 ml tube and 32 mLs of DMEM 10% added. Cells were well mixed and 250 uLs added to each well of a 48 well plate and incubated overnight at 37 C resulting in adherent cells that were approximately 70% confluent the following morning. The next day, siRNA transfection complexes were created by the following method:

Sequences were ordered from Qiagen as pre-annealed siRNA duplexes. Each well was resuspended in 250 ul of siRNA buffer (from Qiagen) to give a stock concentration of 20 uM. The plate was then placed in a water bath at 95 C for 5 minutes and then allowed to slowly cool to resuspend the duplexes and break apart aggregates. The suspended duplexes were then used in transfection experiments described in standard protocols. The formulation is per well of a 48 well plate containing 250 uL of media; each screen was performed in biological triplicate so the solution was made for 4 wells; 3 for transfection and 1 extra.

0.3 uL of the appropriate siRNA (from a 20 uM stock solution) to 47 uL of serum/antibiotic free media and mixed. To this solution was added 3 uL of HiPerfect transfection reagent (Qiagen) followed by brief vortexing and incubation at room temperature for 20 minutes. 50 uLs of the complex containing mixture was added to each of 3 wells in a 48 well plate containing CMT93 cells. Transfection was for 24, 48 or 72 hours at 37 C at which time the cells were washed and RNA isolated for qRT-PCR according the Qiagen Quantitech method (see manufacturer's protocol) for 40 cycles.

PCT Publication No. WO2008/156702 at FIG. 22 shows the knockdown of IL6-RA expression with the various siRNA sequences in CMT93 cells post 24 h transfection. The siRNA sequences tested are listed in Table 35.

TABLE 35

| | | SEQ ID | | SEQ ID |
|---|---|---|---|---|
| Well | siRNA sense 5′→3′ | NO: | siRNA antisense 5′→3′ | NO: |
| E1 | CCUGGAGGGUGACAAAGUAtt | 436 | UACUUUGUCACCCUCCAGGat | 437 |
| F1 | GGUCUGACAAUACCGUAAAtt | 438 | UUUACGGUAUUGUCAGACCca | 439 |
| F2 | GCUGUUUCCUAUAACAGAAtt | 440 | UUCUGUUAUAGGAAACAGCgg | 441 |
| F3 | GCUGUGAAAGGGAAAUUUAtt | 442 | UAAAUUUCCCUUUCACAGCag | 443 |
| E4 | CCUUGUGGUAUCAGCCAUAtt | 444 | UAUGGCUGAUACCACAAGGtt | 445 |
| E5 | GCUUCGAUACCGACCUGUAtt | 446 | UACAGGUCGGUAUCGAAGCtg | 447 |
| E6 | CGGCAGGAAUCCUCUGGAAtt | 448 | UUCCAGAGGAUUCCUGCCGgg | 449 |
| E7 | CCACGAGGAUCAGUACGAAtt | 450 | UUCGUACUGAUCCUCGUGGtt | 451 |
| E8 | CACGAGGAUCAGUACGAAAtt | 452 | UUUCGUACUGAUCCUCGUGgt | 453 |
| E9 | GAUCAGUACGAAAGUUCUAtt | 454 | UAGAACUUUCGUACUGAUCct | 455 |
| E10 | GUACGAAAGUUCUACAGAAtt | 456 | UUCUGUAGAACUUUCGUACtg | 457 |
| E11 | GAAAGUUCUACAGAAGCAAtt | 458 | UUGCUUCUGUAGAACUUUCgt | 459 |
| E12 | GGGUCUGACAAUACCGUAAtt | 460 | UUACGGUAUUGUCAGACCCag | 461 |

### Example 12

### Inhibition of Expression of IL13-Ra1 in CMT93 Cells

One confluent T-175 flask of CMT93 cells was trypsinized in 10 mls until the cells detached. Trypsin was inactivated by addition of 30 mls of DMEM (10% FCS, pen/strep) and the cells thoroughly mixed by pipetting. From this solution, 8 mls was transferred into a sterile 50 ml tube and 32 mLs of DMEM 10% added. Cells were well mixed and 250 uLs added to each well of a 48 well plate and incubated overnight at 37 C resulting in adherent cells that were approximately 70% confluent the following morning. The next day, siRNA transfection complexes were created by the following method:

Sequences were ordered from Qiagen as pre-annealed siRNA duplexes. Each well was resuspended in 250 ul of siRNA buffer (from Qiagen) to give a stock concentration of 20 uM. The plate was then placed in a water bath at 95 C for 5 minutes and then allowed to slowly cool to resuspend the duplexes and break apart aggregates. The suspended duplexes were then used in transfection experiments described in standard protocols. The formulation is per well of a 48 well plate containing 250 uL of media; each screen was performed in biological triplicate so the solution was made for 4 wells; 3 for transfection and 1 extra.

0.3 uL of the appropriate siRNA (from a 20 uM stock solution) to 47 uL of serum/antibiotic free media and mixed. To this solution was added 3 uL of HiPerfect transfection reagent (Qiagen) followed by brief vortexing and incubation at room temperature for 20 minutes. 50 uLs of the complex containing mixture was added to each of 3 wells in a 48 well plate containing CMT93 cells. Transfection was for 24 or 72 hours at 37 C at which time the cells were washed and RNA isolated for qRT-PCR according the Qiagen Quantitech method (see manufacturer's protocol) for 40 cycles.

PCT Publication No. WO2008/156702 at FIG. 23 shows the knockdown of IL13-RA1 expression with the various siRNA sequences in CMT93 cells post 24 hr transfection. The siRNA sequences tested are listed in Table 36.

TABLE 36

| | | SEQ ID | | SEQ ID |
|---|---|---|---|---|
| Well | siRNA sense 5′→3′ | NO: | siRNA antisense 5′→3′ | NO: |
| F1 | AGAAGACUCUAAUGAUGUAtt | 462 | UACAUCAUUAGAGUCUUCUtg | 463 |
| F2 | CAGUCAGAGUAAGAGUCAAtt | 464 | UUGACUCUUACUCUGACUGtg | 465 |
| F8 | CAGAACAUCUAGCAAACAAtt | 466 | UUGUUUGCUAGAUGUUCUGtg | 467 |
| F9 | CUUGUAGGUUCACAUAUUAtt | 468 | UAAUAUGUGAACCUACAAGtt | 469 |
| F10 | CAGUGUAGUGCCAAUGAAAtt | 470 | UUUCAUUGGCACUACACUGag | 471 |
| F11 | GUAUGACAUCUAUGAGAAAtt | 472 | UUUCUCAUAGAUGUCAUACtt | 473 |

### Example 13

### Inhibition of Expression of IL-18 in CMT93 Cells

One confluent T-175 flask of CMT93 cells was trypsinized in 10 mls until the cells detached. Trypsin was inactivated by addition of 30 mls of DMEM (10% FCS, pen/strep) and the cells thoroughly mixed by pipetting. From this solution, 8 mls was transferred into a sterile 50 ml tube and 32 mLs of DMEM 10% added. Cells were well mixed and 250 uLs added to each well of a 48 well plate and incubated overnight at 37 C resulting in adherent cells that were approximately 70% confluent the following morning. The next day, siRNA transfection complexes were created by the following method:

Sequences were ordered from Qiagen as pre-annealed siRNA duplexes. Each well was resuspended in 250 ul of siRNA buffer (from Qiagen) to give a stock concentration of 20 uM. The plate was then placed in a water bath at 95 C for 5 minutes and then allowed to slowly cool to resuspend the duplexes and break apart aggregates. The suspended duplexes were then used in transfection experiments described in standard protocols. The formulation is per well of a 48 well plate containing 250 uL of media; each screen was performed in biological triplicate so the solution was made for 4 wells; 3 for transfection and 1 extra.

0.3 uL of the appropriate siRNA (from a 20 uM stock solution) to 47 uL of serum/antibiotic free media and mixed. To this solution was added 3 uL of Lipofectamine RNAiMAX transfection reagent (Invitrogen) followed by brief vortexing and incubation at room temperature for 20 minutes. 50 uLs of the complex containing mixture was added to each of 3 wells in a 48 well plate containing CMT93 cells. Transfection was for 24 hours at 37 C at which time the cells were washed and RNA isolated for qRT-PCR according the Qiagen Quantitech method (see manufacturer's protocol) for 40 cycles.

PCT Publication No. WO2008/156702 at FIG. 24 shows the knockdown of IL18 expression with the various siRNA sequences in CMT93 cells post 24 hr transfection. The siRNA sequences tested are listed in Table 37.

TABLE 37

| | | SEQ ID | | SEQ ID |
|---|---|---|---|---|
| Well | siRNA sense 5′→3′ | NO: | siRNA antisense 5′→3′ | NO: |
| B2 | AGGAAAUGAUGUUUAUUGAtt | 474 | UCAAUAAACAUCAUUUCCUtg | 475 |
| B3 | GGCCGACUUCACUGUACAAtt | 476 | UUGUACAGUGAAGUCGGCCaa | 477 |
| B4 | GAUGGAGUUUGAAUCUUCAtt | 478 | UGAAGAUUCAAACUCCAUCtt | 479 |
| B5 | CAACCGCAGUAAUACGGAAtt | 480 | UUCCGUAUUACUGCGGUUGta | 481 |
| B6 | CGAGGCUGCAUGAUUUAUAtt | 482 | UAUAAAUCAUGCAGCCUCGgg | 483 |
| B7 | CCUGUAUUUCCAUAACAGAtt | 484 | UCUGUUAUGGAAAUACAGGcg | 485 |
| B8 | CAUGUACAAAGACAGUGAAtt | 486 | UUCACUGUCUUUGUACAUGta | 487 |
| B9 | CGAGGAUAUGACUGAUAUUtt | 488 | AAUAUCAGUCAUAUCCUCGaa | 489 |
| B10 | GGAUAUGACUGAUAUUGAUtt | 490 | AUCAAUAUCAGUCAUAUCCtc | 491 |
| B11 | CUAACUUACAUCAAAGUUAtt | 492 | UAACUUUGAUGUAAGUUAGtg | 493 |
| B12 | CUCACUAACUUACAUCAAAtt | 494 | UUUGAUGUAAGUUAGUGAGag | 495 |

### Example 14

### Inhibition of Expression of IL-7 in CMT93 Cells

One confluent T-175 flask of CMT93 cells was trypsinized in 10 mls until the cells detached. Trypsin was inactivated by addition of 30 mls of DMEM (10% FCS, pen/strep) and the cells thoroughly mixed by pipetting. From this solution, 8 mls was transferred into a sterile 50 ml tube and 32 mLs of DMEM 10% added. Cells were well mixed and 250 uLs added to each well of a 48 well plate and incubated overnight at 37 C resulting in adherent cells that were approximately 70% confluent the following morning. The next day, siRNA transfection complexes were created by the following method:

Sequences were ordered from Qiagen as pre-annealed siRNA duplexes. Each well was resuspended in 250 ul of siRNA buffer (from Qiagen) to give a stock concentration of 20 uM. The plate was then placed in a water bath at 95 C for 5 minutes and then allowed to slowly cool to resuspend the duplexes and break apart aggregates. The suspended duplexes were then used in transfection experiments described in standard protocols. The formulation is per well of a 48 well plate containing 250 uL of media; each screen was performed in biological triplicate so the solution was made for 4 wells; 3 for transfection and 1 extra.

0.3 uL of the appropriate siRNA (from a 20 uM stock solution) to 47 uL of serum/antibiotic free media and mixed. To this solution was added 3 uL of Lipofectamine RNAiMAX transfection reagent (Invitrogen) followed by brief vortexing and incubation at room temperature for 20 minutes. 50 uLs of the complex containing mixture was added to each of 3 wells in a 48 well plate containing CMT93 cells. Transfection was for 24 hours at 37 C at which time the cells were washed and RNA isolated for qRT-PCR according the Qiagen Quantitech method (see manufacturer's protocol) for 40 cycles.

PCT Publication No. WO2008/156702 at FIG. 25 shows the knockdown of IL-7 expression with the various siRNA sequences in CMT93 cells post 24 hr transfection. The siRNA sequences tested are listed in Table 38.

TABLE 38

| | | SEQ ID | | SEQ ID |
|---|---|---|---|---|
| Well | siRNA sense 5′→3′ | NO: | siRNA antisense 5′→3′ | NO: |
| A1 | GAUCCUACGGAAGUUAUGGtt | 496 | CCAUAACUUCCGUAGGAUCcg | 497 |
| B1 | CCAUGUUCCAUGUUUCUUUtt | 498 | AAAGAAACAUGGAACAUGGtc | 499 |
| A2 | CCUCCCGCAGACCAUGUUCtt | 500 | GAACAUGGUCUGCGGGAGGcg | 501 |
| A3 | CUCCCGCAGACCAUGUUCCtt | 502 | GGAACAUGGUCUGCGGGAGgc | 503 |
| A4 | UCCCGCAGACCAUGUUCCAtt | 504 | UGGAACAUGGUCUGCGGGAgg | 505 |
| A5 | CCCGCAGACCAUGUUCCAUtt | 506 | AUGGAACAUGGUCUGCGGGag | 507 |
| A6 | CCGCAGACCAUGUUCCAUGtt | 508 | CAUGGAACAUGGUCUGCGGga | 509 |
| A7 | CGCAGACCAUGUUCCAUGUtt | 510 | ACAUGGAACAUGGUCUGCGgg | 511 |
| A10 | AGACCAUGUUCCAUGUUUCtt | 512 | GAAACAUGGAACAUGGUCUgc | 513 |
| A12 | ACCAUGUUCCAUGUUUCUUtt | 514 | AAGAAACAUGGAACAUGGUct | 515 |

### Example 15

### Inhibition of Expression of Chitinase3-like-1 (CH13L1) Expression in CMT93 Cells

In a 1.7 ml microcentrifuge tube, 2.4 μl of 20 μM double-stranded RNA solution (from Qiagen) was diluted into 394 μl Opti-MEM serum-free medium (Invitrogen) containing 1 μl Lipofectamine RNAiMAX (Invitrogen), mixed, and incubated 10 min at room temperature to enable the formation of transfection complexes. 100 μl of this mixture was added to each of three wells of a 24-well tissue culture dish, on top of which CMT-93 cells were plated in a 500 μl volume, resulting in a final volume of 600 μl per well and a final RNA concentration of 20 nM. After 24 h transfection, 0.1 μg/ml lipopolysaccharide (LPS) (Sigma) was added to each well and cells were incubated for a further 24 h to stimulate CHI3L1 production, after which cells were washed in PBS and harvested for RNA extraction. CMT-93 cells were prepared for transfection as follows. 1 confluent T-175 flask of CMT93 cells was trypsinized in 10 mls until the cells detached. Trypsin was inactivated by addition of 30 mls of DMEM (10% FBS) and the cells thoroughly mixed by pipetting. From this solution, 10 mls was transferred into a sterile 50 ml tube and 40 mLs of DMEM 10% FBS added. Cells were well mixed and 500 μLs added to each well of a 24-well plate. This concentration of cells resulted in approximately 70% confluency after 24 h of growth.

FIG. 26 in WO2008/156702 shows the knockdown of CH13L1 expression with the various siRNA sequences in CMT93 cells post 24 hr transfection. The siRNA sequences tested are listed in Table 39.

TABLE 39

| | | SEQ ID | | SEQ ID |
|---|---|---|---|---|
| Well | siRNA sense 5′→3′ | NO: | siRNA antisense 5′→3′ | NO: |
| G1 | CCACAUCAUCUACAGCUUUtt | 516 | AAAGCUGUAGAUGAUGUGGgt | 517 |
| H1 | GGUUUGACAGAUACAGCAAtt | 518 | UUGCUGUAUCUGUCAAACCta | 519 |
| G2 | UCAUCUACAGCUUUGCCAAtt | 520 | UUGGCAAAGCUGUAGAUGAtg | 521 |
| H2 | CCCUGUUAAGGAAUGCAAAtt | 522 | UUUGCAUUCCUUAACAGGGtt | 523 |
| H3 | CAAGUAGGCAAAUAUCUUAtt | 524 | UAAGAUAUUUGCCUACUUGat | 525 |
| H4 | CAGCUUUGUCAGCAGGAAAtt | 526 | UUUCCUGCUGACAAAGCUGcg | 527 |
| G5 | GGUUCACCAAGGAGGCAGGtt | 528 | CCUGCCUCCUUGGUGAACCgg | 529 |
| H5 | GGAUCAAGUAGGCAAAUAUtt | 530 | AUAUUUGCCUACUUGAUCCaa | 531 |
| H6 | GAGGGACCAUACUAAUUAUtt | 532 | AUAAUUAGUAUGGUCCCUCaa | 533 |
| G7 | GGCCGGUUCACCAAGGAGGtt | 534 | CCUCCUUGGUGAACCGGCCtg | 535 |
| H7 | GGACAAGGAGAGUGUCAAAtt | 536 | UUUGACACUCUCCUUGUCCtc | 537 |
| G8 | CCGGUUCACCAAGGAGGCAtt | 538 | UGCCUCCUUGGUGAACCGGcc | 539 |
| H8 | CGUACAAGCUGGUCUGCUAtt | 540 | UAGCAGACCAGCUUGUACGca | 541 |
| G9 | GGAGUUUAAUCUCUUGCAAtt | 542 | UUGCAAGAGAUUAAACUCCtg | 543 |
| H9 | CAAGGAACUGAAUGCGGAAtt | 544 | UUCCGCAUUCAGUUCCUUGat | 545 |
| G10 | CCCUGAUCAAGGAACUGAAtt | 546 | UUCAGUUCCUUGAUCAGGGtg | 547 |
| H10 | CUUGGAUCAAGUAGGCAAAtt | 548 | UUUGCCUACUUGAUCCAAGtg | 549 |
| G11 | GGAUUGAGGGACCAUACUAtt | 550 | UAGUAUGGUCCCUCAAUCCtg | 551 |
| G12 | GCAAAUUCUCAGACUCUAAtt | 552 | UUAGAGUCUGAGAAUUUGCat | 553 |
| H12 | CCUUCCCUUAGGAACUUAAtt | 554 | UUAAGUUCCUAAGGGAAGGat | 555 |

### Example 16

### Construction of CEQ200

CEQ200 has the following genotype: glnV44(AS), LAM⁻, rfbC1, endA1, spoT1, thi-1, hsdR17, (rₖ⁻mₖ⁺), creC510 ΔdapA. The MM294 has the following genotype: glnV44(AS), LAM⁻, rfbC1, endA1, spoT1, thi-1, hsdR17, (rₖ⁻mₖ⁺), creC510. We purchased the plasmids from CGSC (see Datsenko et al., (2000) Proc. Natl. Acad. Sci. USA 97, 6640-6645).

### Derivation of CEQ200

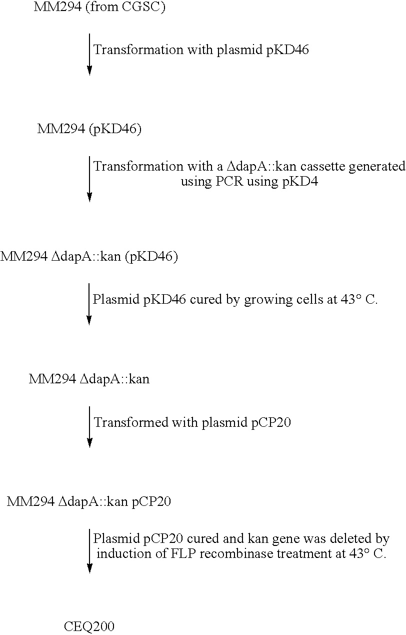

### Example 17

### Construction of CEQ201

CEQ201 has the following genotype: CEQ200 [glnV44(AS), LAM⁻,rfbC1, endA1, spoT1, thi-1, hsdR17, (rₖ⁻mₖ⁺),creC510 ΔdapA ΔrecA. The MM294 has the following genotype: glnV44(AS), LAM⁻, rfbC1, endA1, spoT1, thi-1, hsdR17, (rₖ⁻mₖ⁺),creC510. We purchased the plasmids from CGSC (see Datsenko et al., (2000) Proc. Natl. Acad. Sci. USA 97, 6640-6645).

### Derivation of CEQ200

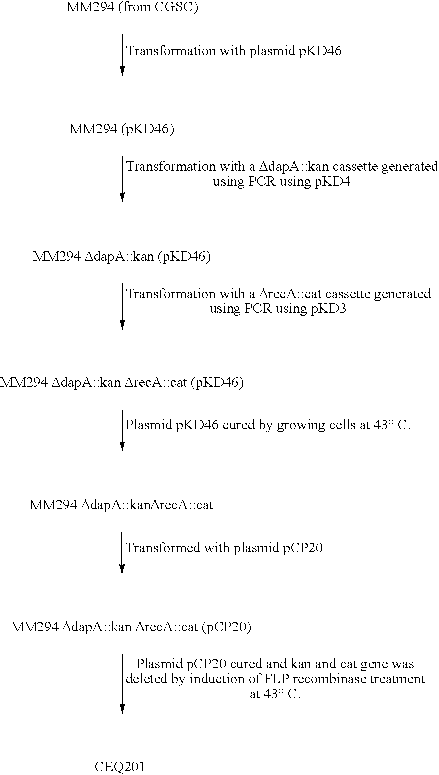

### Example 18

### Construction of BTPs (CEQ210) by Deletion of minC and/or minD Genes from MM294

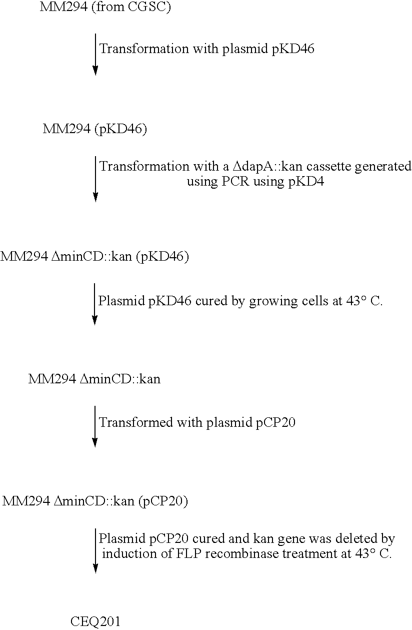

### Example 19

### Illustration of the pMBV40, pMBV43 and pMBV44 Plasmids

The pMBV40, pMBV43 and pMBV44 plasmids may be used as final or intermediary plasmid in the tkRNA system and may be constructed as follows: pUC19 digested with restriction enzyme PvuII. Resultant ˜2.4 kb fragment was ligated with a ˜200 by DNA fragment generated by annealing 5 oligonucleotides with each other. The oligonucleotides have the following names and sequences:

(SEQ ID NO: 556)

OHTOP1: GACTTCATATACCCAAGCTTGGAAAATTTTTTTTAAAAAAG
TCTTGACACTTTATGCTTCCGGCTCGTATAATGGATCCAGGAGTAACAA
TACAAATGGA

(SEQ ID NO: 557)

OHTOP2: TTCAAGAGATCCATTTGTATTGTTACTCCTTTTTTTTTTTG
TCGACGATCCTTAGCGAAAGCTAAGGATTTTTTTTTTACTCGAGCGGAT
TACTACATAC

(SEQ ID NO: 558)

OHBOT1: GTATGTAGTAATCCGCTCGAGTAAAAAAAAAATCCTTAGCT
TTCGCTAAGGATCGTCGACAAAAAAAAAA

(SEQ ID NO: 559)

OHBOT2: AGGAGTAACAATACAAATGGATCTCTTGAATCCATTTGTAT
TGTTACTCCTGGATCCATT

(SEQ ID NO: 560)

OHBOT3: ATACGAGCCGGAAGCATAAAGTGTCAAGACTTTTTTAAAAA
AAATTTTCCAAGCTTGGGTATATGAAGTC

↓
Ligation mix was transformed in E. coli and
Ampicillin resistant transformants were selected.
Plasmid DNA from a transformant that
had the expected DNA sequence of the insert and
restriction map was named pMBV38.
↓
pMBV38 was digested with NdeI and blunt end
ligated with a ~6
kb fragment generated by BamHI-SalI digestion of
the plasmid pKSII-inv-hly

The predicted sequence of is shown in Table 40.

TABLE 40
pKSII-inv-hly (SEQ ID NO: 561)

CTAAATTGTAAGCGTTAATATTTTGTTAAAATTCGCGTTAAATTTTTGTTAAATCAGCTCATTTTTTAACCAAT
AGGCCGAAATCGGCAAAATCCCTTATAAATCAAAAGAATAGACCGAGATAGGGTTGAGTGTTGTTCCAGTTTGG
AACAAGAGTCCACTATTAAAGAACGTGGACTCCAACGTCAAAGGGCGAAAAACCGTCTATCAGGGCGATGGCCC
ACTACGTGAACCATCACCCTAATCAAGTTTTTTGGGGTCGAGGTGCCGTAAAGCACTAAATCGGAACCCTAAAG
GGAGCCCCCGATTTAGAGCTTGACGGGGAAAGCCGGCGAACGTGGCGAGAAAGGAAGGGAAGAAAGCGAAAGGA
GCGGGCGCTAGGGCGCTGGCAAGTGTAGCGGTCACGCTGCGCGTAACCACCACACCCGCCGCGCTTAATGCGCC
GCTACAGGGCGCGTCCCATTCGCCATTCAGGCTGCGCAACTGTTGGGAAGGGCGATCGGTGCGGGCCTCTTCGC
TATTACGCCAGCTGGCGAAAGGGGGATGTGCTGCAAGGCGATTAAGTTGGGTAACGCCAGGGTTTTCCCAGTCA
CGACGTTGTAAAACGACGGCCAGTGAGCGCGCGTAATACGACTCACTATAGGGCGAATTGGAGCTCCACCGCGG
TGGCGGCCGCTCTAGAACTAGTGGATCCCCCGGGCTGCAGCTGGGCCGTAAGATCGGCATTTAATCGCGACAAT
CCTTTTAAAAAAACAGCGCCGCTCAATTAACCTGAGCGGCGTTGTTCTTCTGGACGTTTGCTACTTATGGGGCG
AGTCTAGGATTGCCGGACTCCCATTCGCGCCCCAAATAATCAGCTCATTAAACTGTTCTTATTGCTATCTGTTA
TCTGGTTATATTGACAGCGCACAGAGCGGGAACGCCAAGTATGCAGGCCCTGGTTGCAGTGCGCCTGTGTCCAT
ATTCATGGTTTCAAAATCCGTGCTGGTCTTTTTGACCCAATATTCACCAGATTGCCAATCAGAACTATACGCGG
TCAAGCTCCCCCACTCGCCCCACAATGTCCCGTCAGGCGCACGCGTTCCGTTGGTTGCACGTGAGGATTCAAGA
ACCGCAGACATATCTGAACCTTGGCATTGTCTGCTGGCCTCGAGACTGGATACCAGCGATCTGCCGCCATCGTA
TATCCACCGATTTGGGTAGAACCGATAACTCACCGAATAACTTGGGAATTTTTTACTTTTCGCCGTCACAGCCA
CTTCGCTATAGGTTTGGTAGGTAATCGTCACCTGACCCTGATCGTTAACCGATACATTGGGTGTGAATGACGAC
GACCACTCATACTGAGTATTATTAGCAACATCGTTATCCATCTGTAACTGGAATGTGGCGTTTTTAAAGATCGT
TTTCGGGAACCCTTTATCCGTAGCGAAATTTTGCCCGTTAACCAGAATACCGGTCAGCGTAGGTACCGGGAATA
GGGATATTTTTTTCTGCAATGTACTCAGTATCAGGGTATCAACCTGCGGCGTGATTGTGACATCACCGACACTA
TTCCCAACCACCGTCGCGGTATAGCTATCTGGCTGCTCGGTAATGGGGCTAATACTCACCGGCACACCGTTTTG
AGTAAAACTCAAGCCCTGCATCCCACTGATAAAATGGCCATTCTTATCGACAGGGACAAAGGATAATGTGGAAC
TCATCGTGCCATCAGCCAAGATATCCGGTGTGGAGACGGTGAAACTGGAGCGGCCAGCATCTGGAATAGGATCT
GCCGTGAAATTAACCGTCACACTCGGCACACTGAACGCAGCCCCATCCACTTTCACCGTTACTGTTGCTACCCC
CAACGTGGTACTGGTCAATGGTGCGCTATAAGTGCCGTCATTGTGATCCGTGATAACGCCCATATTGCCTAAGG
TTGTGTCAAAAGCCACATTCGCGCCAGCCTGCGGGTCCCCATAGGTATCCTTCAACTCCAACGTGATGGTTGAA
GCCATTAGACCATCAGCGATGATAGATGTCGGTACCGCAGCCAGAGTGGATTTATCCGCCGCGATAGTACCCTT
AACAAAGTGGGTATCAACACTTTGCCGTTGCCCCTCCACTTCTGCTGTGACTACCGTCACGCCATCTGTCGTAT
TGGTTAATGCAATGCGCGCGACGCCATTTGCATCTGTCTTTTCCGTGATTTTATTCGGTAGCGCACCATTATTG
GTGGTTATCACCACCTCCTGCCCGGCTAAGGGTTTCCCCTCAAAATCAGCAACGGTGAACTCAACGGTGATTGC
AGTTTTCCCATTAGCCGGTGCGCCATCACCAATGACGGCCGCCGTTAATGTCAACTGAGGCTGCTGAACGGTGA
CGCTCAATGTGAATGAGTTAGATCGGTTTCCTTGGTGATCAACCGCGAGCGCACTAAGCGAATAAAAGTTGGCT
GTCAGGTCGTCCGTTACCCGACTCACTTGTGCTGTGCGTTTATAAGGCGGTAAAACCAAGTTGAATTGTGTGGT
ACTCAGTGGTGTTAATGTGCCGCCAGCGGCAATCAGTTCGGCATCACTCCAGACAATTTCCCTTACAGCAGATG
CCCCTTGTACTTGTGCGTTCACCTGATAAACCTGACCCGGCAGGCCGGAGATAGTTGCTGGCGATAATGTCAGT
TTAACCACCTGCTGTTTCTGATACTCCAACACGATATTATTGTTACGATCGACAAGGTTATAGCGGCTCTCCGC
CAGTAGACGTGTTCCTGCCACCGCTGAAGGGCTAAGTTGCGACTGAAAACTCTCGCCCAGGCGATAGTTCATTT
GGAGGTTCCACTGTGTTTCATGCTTACTGCTTTTCCCCATACGCTGATCTACCCCGACAGTGAGTAGAGGCACG
GGGGTGTAATTGATCCCGGCAGTCACGGCATAAGGGTTGCGTTGCAGATTATCTTTACCAAATAAAGCAACACG
CTCACCGGTGTATTGCTCATACATCAACTTCCCCCCCAGTTGTGGGAGTGCAGGTAAATAAGCATTCGCGCGCA
AATCCCCCCCAGTGGCTGGGCGCTCTTTATAGTCGGAGAAATCACGCGACGAGTGCCATCCATTGAGGCGAAAA
TACCCATTGGCAGCCAACTGTAAATAATCGGTCCAGGCCTCGGCACCAAGACCGATACGGTGGTTGTGGCCGGT
CAAATCATTATCATAAAAAGTATTAAGTCCGTACAGCCAACCGTTCTCCAATGTACGTATCCCGACGCCAAGGT
TAAGTGTGTTGCGGCTGTCTTTATTGCGAATACCTAACTGACTAAAAAAGAGGAATGAAGCAGAGTCATACCAA
GGAGCCAGCCAATCAAGAGAGCTTTCTTTTAGCGAAAAATTTTTGTCAAAATTCAGATTAACTTGAGCCGTACC
GAATCGATTTAACCACTGTTTGATTTCTTGATTAACCGCATCGCCCACCATTGAGTGAGCAACATCAGATGCCC
TGCCTGATGCAGCTAACCTGGCCCCGGTGCTTATCATCTTATTCACCGCTTCAGTCTCCTGCTCCTTATTGGCG
CGATCTATTATTGCAGCATTTCTTTCTGTATCCGATGCGGAAAAGGGATTGATTGAACTCTCCATTTCATTATT
AGGATGGAGATTTTCAAATGCAGATGAAGAGACAGAATAAGGCTGGACCTGTTGCGGTGCGTTAGCATCATATT
TTTCTGAAGCCCCAGCCATGAACATTCCACATATCAAAAAGATACAAATAACTATTCGTGAAATAATATTAAAT
GAAATTATTTTATTAAAATACATAGACATTCCCGCATTCCTTATCAAGAGAAACTCACTGATTGGCTGGAAAAC
CATCATAATTTAAATGAAATAAAGCATACCTGTCATACGTCAAACTGCATGTGCGTTGGCTGTGCTCAACAACT
TGAGTTATTTGAGGTATAACTGGCCACAAACGAGCATTTGAAATCACCTTGACCATTAATTAAAGATGCAATAG
TTGAAAGTGAAACTTGTTTTCTAATTTAGTAAAGACATTAAGAGGATAGCACTTTTTTAAAAAACCAGACTGGG
CAGATTAAAAATATTCAAAATATATAATAAAACAGTCTATACCATACAGCGATAGAATTGATTTATTGTAACTA
AGCAGGTGAGAATATCAAAAAAAACAAAAATACAAAATGAACTATTATCATATAAATAATATCAATTAGAATAA
GCCCCCTTCATTTGATGTTGTCAGTTGTCTGCTGCGGTTTTTATTTCTACTTTCAGTCTGAAGTGTTACTCCGC
AATATCCGCATTAATCCTGATGGTTGCCTTGATGACTGCAGGAATTCGATCCCTCCTTTGATTAGTATATTCCT
ATCTTAAAGTGACTTTTATGTTGAGGCATTAACATTTGTTAACGACGATAAAGGGACAGCAGGACTAGAATAAA
GCTATAAAGCAAGCATATAATATTGCGTTTCATCTTTAGAAGCGAATTTCGCCAATATTATAATTATCAAAAGA
GAGGGGTGGCAAACGGTATTTGGCATTATTAGGTTAAAAAATGTAGAAGGAGAGTGAAACCCATGAAAAAAATA
ATGCTAGTTTTTATTACACTTATATTAGTTAGTCTACCAATTGCGCAACAAACTGAAGCAAAGGATGCATCTGC
ATTCAATAAAGAAAATTCAATTTCATCCATGGCACCACCAGCATCTCCGCCTGCAAGTCCTAAGACGCCAATCG
AAAAGAAACACGCGGATGAAATCGATAAGTATATACAAGGATTGGATTACAATAAAAACAATGTATTAGTATAC
CACGGAGATGCAGTGACAAATGTGCCGCCAAGAAAAGGTTACAAAGATGGAAATGAATATATTGTTGTGGAGAA
AAAGAAGAAATCCATCAATCAAAATAATGCAGACATTCAAGTTGTGAATGCAATTTCGAGCCTAACCTATCCAG
GTGCTCTCGTAAAAGCGAATTCGGAATTAGTAGAAAATCAACCAGATGTTCTCCCTGTAAAACGTGATTCATTA
ACACTCAGCATTGATTTGCCAGGTATGACTAATCAAGACAATAAAATCGTTGTAAAAAATGCCACTAAATCAAA
CGTTAACAACGCAGTAAATACATTAGTGGAAAGATGGAATGAAAAATATGCTCAAGCTTATCCAAATGTAAGTG
CAAAAATTGATTATGATGACGAAATGGCTTACAGTGAATCACAATTAATTGCGAAATTTGGTACAGCATTTAAA
GCTGTAAATAATAGCTTGAATGTAAACTTCGGCGCAATCAGTGAAGGGAAAATGCAAGAAGAAGTCATTAGTTT
TAAACAAATTTACTATAACGTGAATGTTAATGAACCTACAAGACCTTCCAGATTTTTCGGCAAAGCTGTTACTA
AAGAGCAGTTGCAAGCGCTTGGAGTGAATGCAGAAAATCCTCCTGCATATATCTCAAGTGTGGCGTATGGCCGT
CAAGTTTATTTGAAATTATCAACTAATTCCCATAGTACTAAAGTAAAAGCTGCTTTTGATGCTGCCGTAAGCGG
AAAATCTGTCTCAGGTGATGTAGAACTAACAAATATCATCAAAAATTCTTCCTTCAAAGCCGTAATTTACGGAG
GTTCCGCAAAAGATGAAGTTCAAATCATCGACGGCAACCTCGGAGACTTACGCGATATTTTGAAAAAAGGCGCT
ACTTTTAATCGAGAAACACCAGGAGTTCCCATTGCTTATACAACAAACTTCCTAAAAGACAATGAATTAGCTGT
TATTAAAAACAACTCAGAATATATTGAAACAACTTCAAAAGCTTATACAGATGGAAAAATTAACATCGATCACT
CTGGAGGATACGTTGCTCAATTCAACATTTCTTGGGATGAAGTAAATTATGATCCTGAAGGTAACGAAATTGTT
CAACATAAAAACTGGAGCGAAAACAATAAAAGCAAGCTAGCTCATTTCACATCGTCCATCTATTTGCCAGGTAA
CGCGAGAAATATTAATGTTTACGCTAAAGAATGCACTGGTTTAGCTTGGGAATGGTGGAGAACGGTAATTGATG
ACCGGAACTTACCACTTGTGAAAAATAGAAATATCTCCATCTGGGGCACCACGCTTTATCCGAAATATAGTAAT
AAAGTAGATAATCCAATCGAATAATTGTAAAAGTAATAAAAAATTAAGAATAAAACCGCTTAACACACACGAAA
AAATAAGCTTGTTTTGCACTCTTCGTAAATTATTTTGTGAAGAATGTAGAAACAGGCTTATTTTTTAATTTTTT
TAGAAGAATTAACAAATGTAAAAGAATATCTGACTGTTTATCCATATAATATAAGCATATCCCAAAGTTTAAGC
CACCTATAGTTTCTACTGCAAAACGTATAATTTAGTTCCCACATATACTAAAAAACGTGTCCTTAACTCTCTCT
GTCAGATTAGTTGTAGGTGGCTTAAACTTAGTTTTACGAATTAAAAAGGAGCGGTGAAATGAAAAGTAAACTTA
TTTGTATCATCATGGTAATAGCTTTTCAGGCTCATTTCACTATGACGGTAAAAGCAGATTCTGTCGGGGAAGAA
AAACTTCAAAATAATACACAAGCCAAAAAGACCCCTGCTGATTTAAAAGCTTATCAAGCTTATCGATACCGTCG
ACCTCGAGGGGGGGCCCGGTACCCAGCTTTTGTTCCCTTTAGTGAGGGTTAATTGCGCGCTTGGCGTAATCATG
GTCATAGCTGTTTCCTGTGTGAAATTGTTATCCGCTCACAATTCCACACAACATACGAGCCGGAAGCATAAAGT
GTAAAGCCTGGGGTGCCTAATGAGTGAGCTAACTCACATTAATTGCGTTGCGCTCACTGCCCGCTTTCCAGTCG
GGAAACCTGTCGTGCCAGCTGCATTAATGAATCGGCCAACGCGCGGGGAGAGGCGGTTTGCGTATTGGGCGCTC
TTCCGCTTCCTCGCTCACTGACTCGCTGCGCTCGGTCGTTCGGCTGCGGCGAGCGGTATCAGCTCACTCAAAGG
CGGTAATACGGTTATCCACAGAATCAGGGGATAACGCAGGAAAGAACATGTGAGCAAAAGGCCAGCAAAAGGCC
AGGAACCGTAAAAAGGCCGCGTTGCTGGCGTTTTTCCATAGGCTCCGCCCCCCTGACGAGCATCACAAAAATCG
ACGCTCAAGTCAGAGGTGGCGAAACCCGACAGGACTATAAAGATACCAGGCGTTTCCCCCTGGAAGCTCCCTCG
TGCGCTCTCCTGTTCCGACCCTGCCGCTTACCGGATACCTGTCCGCCTTTCTCCCTTCGGGAAGCGTGGCGCTT
TCTCATAGCTCACGCTGTAGGTATCTCAGTTCGGTGTAGGTCGTTCGCTCCAAGCTGGGCTGTGTGCACGAACC
CCCCGTTCAGCCCGACCGCTGCGCCTTATCCGGTAACTATCGTCTTGAGTCCAACCCGGTAAGACACGACTTAT
CGCCACTGGCAGCAGCCACTGGTAACAGGATTAGCAGAGCGAGGTATGTAGGCGGTGCTACAGAGTTCTTGAAG
TGGTGGCCTAACTACGGCTACACTAGAAGGACAGTATTTGGTATCTGCGCTCTGCTGAAGCCAGTTACCTTCGG
AAAAAGAGTTGGTAGCTCTTGATCCGGCAAACAAACCACCGCTGGTAGCGGTGGTTTTTTTGTTTGCAAGCAGC
AGATTACGCGCAGAAAAAAAGGATCTCAAGAAGATCCTTTGATCTTTTCTACGGGGTCTGACGCTCAGTGGAAC
GAAAACTCACGTTAAGGGATTTTGGTCATGAGATTATCAAAAAGGATCTTCACCTAGATCCTTTTAAATTAAAA
ATGAAGTTTTAAATCAATCTAAAGTATATATGAGTAAACTTGGTCTGACAGTTACCAATGCTTAATCAGTGAGG
CACCTATCTCAGCGATCTGTCTATTTCGTTCATCCATAGTTGCCTGACTCCCCGTCGTGTAGATAACTACGATA
CGGGAGGGCTTACCATCTGGCCCCAGTGCTGCAATGATACCGCGAGACCCACGCTCACCGGCTCCAGATTTATC
AGCAATAAACCAGCCAGCCGGAAGGGCCGAGCGCAGAAGTGGTCCTGCAACTTTATCCGCCTCCATCCAGTCTA
TTAATTGTTGCCGGGAAGCTAGAGTAAGTAGTTCGCCAGTTAATAGTTTGCGCAACGTTGTTGCCATTGCTACA
GGCATCGTGGTGTCACGCTCGTCGTTTGGTATGGCTTCATTCAGCTCCGGTTCCCAACGATCAAGGCGAGTTAC
ATGATCCCCCATGTTGTGCAAAAAAGCGGTTAGCTCCTTCGGTCCTCCGATCGTTGTCAGAAGTAAGTTGGCCG
CAGTGTTATCACTCATGGTTATGGCAGCACTGCATAATTCTCTTACTGTCATGCCATCCGTAAGATGCTTTTCT
GTGACTGGTGAGTACTCAACCAAGTCATTCTGAGAATAGTGTATGCGGCGACCGAGTTGCTCTTGCCCGGCGTC
AATACGGGATAATACCGCGCCACATAGCAGAACTTTAAAAGTGCTCATCATTGGAAAACGTTCTTCGGGGCGAA
AACTCTCAAGGATCTTACCGCTGTTGAGATCCAGTTCGATGTAACCCACTCGTGCACCCAACTGATCTTCAGCA
TCTTTTACTTTCACCAGCGTTTCTGGGTGAGCAAAAACAGGAAGGCAAAATGCCGCAAAAAAGGGAATAAGGGC
GACACGGAAATGTTGAATACTCATACTCTTCCTTTTTCAATATTATTGAAGCATTTATCAGGGTTATTGTCTCA
TGAGCGGATACATATTTGAATGTATTTAGAAAAATAAACAAATAGGGGTTCCGCGCACATTTCCCCGAAAAGTG
CCAC

↓
Ligation mix was transformed in E. coli and Ampicillin resistant
transformants were selected. Plasmid DNA from a transformant that
had insertion of inv and hly genes was named pMBV40.
↓
pMBV40 was digested with BspHI and the resultant 7.4 kb DNA
fragment was ligated with a PCR fragment containing kan gene
generated using plasmid pKD4 (purchased from CGSC (see
Datsenko et al., (2000) Proc. Natl. Acad. Sci. USA 97,6640-6645)
as the template.
↓
Ligation mix was transformed in E. coli and Kanamycin resistant
transformants were selected. They were screened restriction
mapping. They two different orientation of kan gene. The plasmids
having clockwise and anticlockwise orientation of open reading
frame of kan gene were called pMBV43 and pMBV44, respectively

As shown in PCT Publication No. WO2008/156702 at FIG. 27, the pMBV40 (amp selected having H3 hairpin) or pMBV43 and pMBV44 (kan selected having H3 hairpin) plasmids, are followed by the respective sequences.

TABLE 41

pMBV40 (SEQ ID NO: 562)
TCGCGCGTTTCGGTGATGACGGTGAAAACCTCTGACACATGCAGCTCCCGGAGACGGTCACAGCTTGTCTGTAAG
CGGATGCCGGGAGCAGACAAGCCCGTCAGGGCGCGTCAGCGGGTGTTGGCGGGTGTCGGGGCTGGCTTAACTATG
CGGCATCAGAGCAGATTGTACTGAGAGTGCACCATATCGACGGTATCGATAAGCTTGATAAGCTTTTAAATCAGC
AGGGGTCTTTTTGGCTTGTGTATTATTTTGAAGTTTTTCTTCCCCGACAGAATCTGCTTTTACCGTCATAGTGAA
ATGAGCCTGAAAAGCTATTACCATGATGATACAAATAAGTTTACTTTTCATTTCACCGCTCCTTTTTAATTCGTA
AAACTAAGTTTAAGCCACCTACAACTAATCTGACAGAGAGAGTTAAGGACACGTTTTTTAGTATATGTGGGAACT
AAATTATACGTTTTGCAGTAGAAACTATAGGTGGCTTAAACTTTGGGATATGCTTATATTATATGGATAAACAGT
CAGATATTCTTTTACATTTGTTAATTCTTCTAAAAAAATTAAAAAATAAGCCTGTTTCTACATTCTTCACAAAAT
AATTTACGAAGAGTGCAAAACAAGCTTATTTTTTCGTGTGTGTTAAGCGGTTTTATTCTTAATTTTTTATTACTT
TTACAATTATTCGATTGGATTATCTACTTTATTACTATATTTCGGATAAAGCGTGGTGCCCCAGATGGAGATATT
TCTATTTTTCACAAGTGGTAAGTTCCGGTCATCAATTACCGTTCTCCACCATTCCCAAGCTAAACCAGTGCATTC
TTTAGCGTAAACATTAATATTTCTCGCGTTACCTGGCAAATAGATGGACGATGTGAAATGAGCTAGCTTGCTTTT
ATTGTTTTCGCTCCAGTTTTTATGTTGAACAATTTCGTTACCTTCAGGATCATAATTTACTTCATCCCAAGAAAT
GTTGAATTGAGCAACGTATCCTCCAGAGTGATCGATGTTAATTTTTCCATCTGTATAAGCTTTTGAAGTTGTTTC
AATATATTCTGAGTTGTTTTTAATAACAGCTAATTCATTGTCTTTTAGGAAGTTTGTTGTATAAGCAATGGGAAC
TCCTGGTGTTTCTCGATTAAAAGTAGCGCCTTTTTTCAAAATATCGCGTAAGTCTCCGAGGTTGCCGTCGATGAT
TTGAACTTCATCTTTTGCGGAACCTCCGTAAATTACGGCTTTGAAGGAAGAATTTTTGATGATATTTGTTAGTTC
TACATCACCTGAGACAGATTTTCCGCTTACGGCAGCATCAAAAGCAGCTTTTACTTTAGTACTATGGGAATTAGT
TGATAATTTCAAATAAACTTGACGGCCATACGCCACACTTGAGATATATGCAGGAGGATTTTCTGCATTCACTCC
AAGCGCTTGCAACTGCTCTTTAGTAACAGCTTTGCCGAAAAATCTGGAAGGTCTTGTAGGTTCATTAACATTCAC
GTTATAGTAAATTTGTTTAAAACTAATGACTTCTTCTTGCATTTTCCCTTCACTGATTGCGCCGAAGTTTACATT
CAAGCTATTATTTACAGCTTTAAATGCTGTACCAAATTTCGCAATTAATTGTGATTCACTGTAAGCCATTTCGTC
ATCATAATCAATTTTTGCACTTACATTTGGATAAGCTTGAGCATATTTTTCATTCCATCTTTCCACTAATGTATT
TACTGCGTTGTTAACGTTTGATTTAGTGGCATTTTTTACAACGATTTTATTGTCTTGATTAGTCATACCTGGCAA
ATCAATGCTGAGTGTTAATGAATCACGTTTTACAGGGAGAACATCTGGTTGATTTTCTACTAATTCCGAATTCGC
TTTTACGAGAGCACCTGGATAGGTTAGGCTCGAAATTGCATTCACAACTTGAATGTCTGCATTATTTTGATTGAT
GGATTTCTTCTTTTTCTCCACAACAATATATTCATTTCCATCTTTGTAACCTTTTCTTGGCGGCACATTTGTCAC
TGCATCTCCGTGGTATACTAATACATTGTTTTTATTGTAATCCAATCCTTGTATATACTTATCGATTTCATCCGC
GTGTTTCTTTTCGATTGGCGTCTTAGGACTTGCAGGCGGAGATGCTGGTGGTGCCATGGATGAAATTGAATTTTC
TTTATTGAATGCAGATGCATCCTTTGCTTCAGTTTGTTGCGCAATTGGTAGACTAACTAATATAAGTGTAATAAA
AACTAGCATTATTTTTTTCATGGGTTTCACTCTCCTTCTACATTTTTTAACCTAATAATGCCAAATACCGTTTGC
CACCCCTCTCTTTTGATAATTATAATATTGGCGAAATTCGCTTCTAAAGATGAAACGCAATATTATATGCTTGCT
TTATAGCTTTATTCTAGTCCTGCTGTCCCTTTATCGTCGTTAACAAATGTTAATGCCTCAACATAAAAGTCACTT
TAAGATAGGAATATACTAATCAAAGGAGGGATCGAATTCCTGCAGTCATCAAGGCAACCATCAGGATTAATGCGG
ATATTGCGGAGTAACACTTCAGACTGAAAGTAGAAATAAAAACCGCAGCAGACAACTGACAACATCAAATGAAGG
GGGCTTATTCTAATTGATATTATTTATATGATAATAGTTCATTTTGTATTTTTGTTTTTTTTGATATTCTCACCT
GCTTAGTTACAATAAATCAATTCTATCGCTGTATGGTATAGACTGTTTTATTATATATTTTGAATATTTTTAATC
TGCCCAGTCTGGTTTTTTAAAAAAGTGCTATCCTCTTAATGTCTTTACTAAATTAGAAAACAAGTTTCACTTTCA
ACTATTGCATCTTTAATTAATGGTCAAGGTGATTTCAAATGCTCGTTTGTGGCCAGTTATACCTCAAATAACTCA
AGTTGTTGAGCACAGCCAACGCACATGCAGTTTGACGTATGACAGGTATGCTTTATTTCATTTAAATTATGATGG
TTTTCCAGCCAATCAGTGAGTTTCTCTTGATAAGGAATGCGGGAATGTCTATGTATTTTAATAAAATAATTTCAT
TTAATATTATTTCACGAATAGTTATTTGTATCTTTTTGATATGTGGAATGTTCATGGCTGGGGCTTCAGAAAAAT
ATGATGCTAACGCACCGCAACAGGTCCAGCCTTATTCTGTCTCTTCATCTGCATTTGAAAATCTCCATCCTAATA
ATGAAATGGAGAGTTCAATCAATCCCTTTTCCGCATCGGATACAGAAAGAAATGCTGCAATAATAGATCGCGCCA
ATAAGGAGCAGGAGACTGAAGCGGTGAATAAGATGATAAGCACCGGGGCCAGGTTAGCTGCATCAGGCAGGGCAT
CTGATGTTGCTCACTCAATGGTGGGCGATGCGGTTAATCAAGAAATCAAACAGTGGTTAAATCGATTCGGTACGG
CTCAAGTTAATCTGAATTTTGACAAAAATTTTTCGCTAAAAGAAAGCTCTCTTGATTGGCTGGCTCCTTGGTATG
ACTCTGCTTCATTCCTCTTTTTTAGTCAGTTAGGTATTCGCAATAAAGACAGCCGCAACACACTTAACCTTGGCG
TCGGGATACGTACATTGGAGAACGGTTGGCTGTACGGACTTAATACTTTTTATGATAATGATTTGACCGGCCACA
ACCACCGTATCGGTCTTGGTGCCGAGGCCTGGACCGATTATTTACAGTTGGCTGCCAATGGGTATTTTCGCCTCA
ATGGATGGCACTCGTCGCGTGATTTCTCCGACTATAAAGAGCGCCCAGCCACTGGGGGGGATTTGCGCGCGAATG
CTTATTTACCTGCACTCCCACAACTGGGGGGGAAGTTGATGTATGAGCAATACACCGGTGAGCGTGTTGCTTTAT
TTGGTAAAGATAATCTGCAACGCAACCCTTATGCCGTGACTGCCGGGATCAATTACACCCCCGTGCCTCTACTCA
CTGTCGGGGTAGATCAGCGTATGGGGAAAAGCAGTAAGCATGAAACACAGTGGAACCTCCAAATGAACTATCGCC
TGGGCGAGAGTTTTCAGTCGCAACTTAGCCCTTCAGCGGTGGCAGGAACACGTCTACTGGCGGAGAGCCGCTATA
ACCTTGTCGATCGTAACAATAATATCGTGTTGGAGTATCAGAAACAGCAGGTGGTTAAACTGACATTATCGCCAG
CAACTATCTCCGGCCTGCCGGGTCAGGTTTATCAGGTGAACGCACAAGTACAAGGGGCATCTGCTGTAAGGGAAA
TTGTCTGGAGTGATGCCGAACTGATTGCCGCTGGCGGCACATTAACACCACTGAGTACCACACAATTCAACTTGG
TTTTACCGCCTTATAAACGCACAGCACAAGTGAGTCGGGTAACGGACGACCTGACAGCCAACTTTTATTCGCTTA
GTGCGCTCGCGGTTGATCACCAAGGAAACCGATCTAACTCATTCACATTGAGCGTCACCGTTCAGCAGCCTCAGT
TGACATTAACGGCGGCCGTCATTGGTGATGGCGCACCGGCTAATGGGAAAACTGCAATCACCGTTGAGTTCACCG
TTGCTGATTTTGAGGGGAAACCCTTAGCCGGGCAGGAGGTGGTGATAACCACCAATAATGGTGCGCTACCGAATA
AAATCACGGAAAAGACAGATGCAAATGGCGTCGCGCGCATTGCATTAACCAATACGACAGATGGCGTGACGGTAG
TCACAGCAGAAGTGGAGGGGCAACGGCAAAGTGTTGATACCCACTTTGTTAAGGGTACTATCGCGGCGGATAAAT
CCACTCTGGCTGCGGTACCGACATCTATCATCGCTGATGGTCTAATGGCTTCAACCATCACGTTGGAGTTGAAGG
ATACCTATGGGGACCCGCAGGCTGGCGCGAATGTGGCTTTTGACACAACCTTAGGCAATATGGGCGTTATCACGG
ATCACAATGACGGCACTTATAGCGCACCATTGACCAGTACCACGTTGGGGGTAGCAACAGTAACGGTGAAAGTGG
ATGGGGCTGCGTTCAGTGTGCCGAGTGTGACGGTTAATTTCACGGCAGATCCTATTCCAGATGCTGGCCGCTCCA
GTTTCACCGTCTCCACACCGGATATCTTGGCTGATGGCACGATGAGTTCCACATTATCCTTTGTCCCTGTCGATA
AGAATGGCCATTTTATCAGTGGGATGCAGGGCTTGAGTTTTACTCAAAACGGTGTGCCGGTGAGTATTAGCCCCA
TTACCGAGCAGCCAGATAGCTATACCGCGACGGTGGTTGGGAATAGTGTCGGTGATGTCACAATCACGCCGCAGG
TTGATACCCTGATACTGAGTACATTGCAGAAAAAAATATCCCTATTCCCGGTACCTACGCTGACCGGTATTCTGG
TTAACGGGCAAAATTTCGCTACGGATAAAGGGTTCCCGAAAACGATCTTTAAAAACGCCACATTCCAGTTACAGA
TGGATAACGATGTTGCTAATAATACTCAGTATGAGTGGTCGTCGTCATTCACACCCAATGTATCGGTTAACGATC
AGGGTCAGGTGACGATTACCTACCAAACCTATAGCGAAGTGGCTGTGACGGCGAAAAGTAAAAAATTCCCAAGTT
ATTCGGTGAGTTATCGGTTCTACCCAAATCGGTGGATATACGATGGCGGCAGATCGCTGGTATCCAGTCTCGAGG
CCAGCAGACAATGCCAAGGTTCAGATATGTCTGCGGTTCTTGAATCCTCACGTGCAACCAACGGAACGCGTGCGC
CTGACGGGACATTGTGGGGCGAGTGGGGGAGCTTGACCGCGTATAGTTCTGATTGGCAATCTGGTGAATATTGGG
TCAAAAAGACCAGCACGGATTTTGAAACCATGAATATGGACACAGGCGCACTGCAACCAGGGCCTGCATACTTGG
CGTTCCCGCTCTGTGCGCTGTCAATATAACCAGATAACAGATAGCAATAAGAACAGTTTAATGAGCTGATTATTT
GGGGCGCGAATGGGAGTCCGGCAATCCTAGACTCGCCCCATAAGTAGCAAACGTCCAGAAGAACAACGCCGCTCA
GGTTAATTGAGCGGCGCTGTTTTTTTAAAAGGATTGTCGCGATTAAATGCCGATCTTACGGCCCAGCTGCAGCCC
GGGGGATCTATGCGGTGTGAAATACCGCACAGATGCGTAAGGAGAAAATACCGCATCAGGCGCCATTCGCCATTC
AGGCTGCGCAACTGTTGGGAAGGGCGATCGGTGCGGGCCTCTTCGCTATTACGCCAGGACTTCATATACCCAAGC
TTGGAAAATTTTTTTTAAAAAAGTCTTGACACTTTATGCTTCCGGCTCGTATAATGGATCCAGGAGTAACAATAC
AAATGGATTCAAGAGATCCATTTGTATTGTTACTCCTTTTTTTTTTTGTCGACGATCCTTAGCGAAAGCTAAGGA
TTTTTTTTTTACTCGAGCGGATTACTACATACCTGCATTAATGAATCGGCCAACGCGCGGGGAGAGGCGGTTTGC
GTATTGGGCGCTCTTCCGCTTCCTCGCTCACTGACTCGCTGCGCTCGGTCGTTCGGCTGCGGCGAGCGGTATCAG
CTCACTCAAAGGCGGTAATACGGTTATCCACAGAATCAGGGGATAACGCAGGAAAGAACATGTGAGCAAAAGGCC
AGCAAAAGGCCAGGAACCGTAAAAAGGCCGCGTTGCTGGCGTTTTTCCATAGGCTCCGCCCCCCTGACGAGCATC
ACAAAAATCGACGCTCAAGTCAGAGGTGGCGAAACCCGACAGGACTATAAAGATACCAGGCGTTTCCCCCTGGAA
GCTCCCTCGTGCGCTCTCCTGTTCCGACCCTGCCGCTTACCGGATACCTGTCCGCCTTTCTCCCTTCGGGAAGCG
TGGCGCTTTCTCATAGCTCACGCTGTAGGTATCTCAGTTCGGTGTAGGTCGTTCGCTCCAAGCTGGGCTGTGTGC
ACGAACCCCCCGTTCAGCCCGACCGCTGCGCCTTATCCGGTAACTATCGTCTTGAGTCCAACCCGGTAAGACACG
ACTTATCGCCACTGGCAGCAGCCACTGGTAACAGGATTAGCAGAGCGAGGTATGTAGGCGGTGCTACAGAGTTCT
TGAAGTGGTGGCCTAACTACGGCTACACTAGAAGGACAGTATTTGGTATCTGCGCTCTGCTGAAGCCAGTTACCT
TCGGAAAAAGAGTTGGTAGCTCTTGATCCGGCAAACAAACCACCGCTGGTAGCGGTGGTTTTTTTGTTTGCAAGC
AGCAGATTACGCGCAGAAAAAAAGGATCTCAAGAAGATCCTTTGATCTTTTCTACGGGGTCTGACGCTCAGTGGA
ACGAAAACTCACGTTAAGGGATTTTGGTCATGAGATTATCAAAAAGGATCTTCACCTAGATCCTTTTAAATTAAA
AATGAAGTTTTAAATCAATCTAAAGTATATATGAGTAAACTTGGTCTGACAGTTACCAATGCTTAATCAGTGAGG
CACCTATCTCAGCGATCTGTCTATTTCGTTCATCCATAGTTGCCTGACTCCCCGTCGTGTAGATAACTACGATAC
GGGAGGGCTTACCATCTGGCCCCAGTGCTGCAATGATACCGCGAGACCCACGCTCACCGGCTCCAGATTTATCAG
CAATAAACCAGCCAGCCGGAAGGGCCGAGCGCAGAAGTGGTCCTGCAACTTTATCCGCCTCCATCCAGTCTATTA
ATTGTTGCCGGGAAGCTAGAGTAAGTAGTTCGCCAGTTAATAGTTTGCGCAACGTTGTTGCCATTGCTACAGGCA
TCGTGGTGTCACGCTCGTCGTTTGGTATGGCTTCATTCAGCTCCGGTTCCCAACGATCAAGGCGAGTTACATGAT
CCCCCATGTTGTGCAAAAAAGCGGTTAGCTCCTTCGGTCCTCCGATCGTTGTCAGAAGTAAGTTGGCCGCAGTGT
TATCACTCATGGTTATGGCAGCACTGCATAATTCTCTTACTGTCATGCCATCCGTAAGATGCTTTTCTGTGACTG
GTGAGTACTCAACCAAGTCATTCTGAGAATAGTGTATGCGGCGACCGAGTTGCTCTTGCCCGGCGTCAATACGGG
ATAATACCGCGCCACATAGCAGAACTTTAAAAGTGCTCATCATTGGAAAACGTTCTTCGGGGCGAAAACTCTCAA
GGATCTTACCGCTGTTGAGATCCAGTTCGATGTAACCCACTCGTGCACCCAACTGATCTTCAGCATCTTTTACTT
TCACCAGCGTTTCTGGGTGAGCAAAAACAGGAAGGCAAAATGCCGCAAAAAAGGGAATAAGGGCGACACGGAAAT
GTTGAATACTCATACTCTTCCTTTTTCAATATTATTGAAGCATTTATCAGGGTTATTGTCTCATGAGCGGATACA
TATTTGAATGTATTTAGAAAAATAAACAAATAGGGGTTCCGCGCACATTTCCCCGAAAAGTGCCACCTGACGTCT
AAGAAACCATTATTATCATGACATTAACCTATAAAAATAGGCGTATCACGAGGCCCTTTCGTC

Table 42 contains the 8427 base pair sequence of a predicted pMBV43 plasmid. The sequence contains the following regions: hly orf (682-2271 bp); inv orf (2994-5954 bp) (6212-6282 bp) (6483-6534 bp); shRNA promoter (6303-6361 bp); Sense strand (6362-6383 bp); Loop (6384-6390 bp); Antisense strand (6391-6412 bp); Terminator I (6413-6422 bp); Terminator II (6423-6460 bp); Origin of replication (6720-7307 bp); and kan orf (7498-8292 bp).

TABLE 42

Predicted pMBV43 (SEQ ID NO: 563)
TCGCGCGTTTCGGTGATGACGGTGAAAACCTCTGACACATGCAGCTCCCGGAGACGGTCACAGCTTGTCTGTAAG
CGGATGCCGGGAGCAGACAAGCCCGTCAGGGCGCGTCAGCGGGTGTTGGCGGGTGTCGGGGCTGGCTTAACTATG
CGGCATCAGAGCAGATTGTACTGAGAGTGCACCATATCGACGGTATCGATAAGCTTGATAAGCTTTTAAATCAGC
AGGGGTCTTTTTGGCTTGTGTATTATTTTGAAGTTTTTCTTCCCCGACAGAATCTGCTTTTACCGTCATAGTGAA
ATGAGCCTGAAAAGCTATTACCATGATGATACAAATAAGTTTACTTTTCATTTCACCGCTCCTTTTTAATTCGTA
AAACTAAGTTTAAGCCACCTACAACTAATCTGACAGAGAGAGTTAAGGACACGTTTTTTAGTATATGTGGGAACT
AAATTATACGTTTTGCAGTAGAAACTATAGGTGGCTTAAACTTTGGGATATGCTTATATTATATGGATAAACAGT
CAGATATTCTTTTACATTTGTTAATTCTTCTAAAAAAATTAAAAAATAAGCCTGTTTCTACATTCTTCACAAAAT
AATTTACGAAGAGTGCAAAACAAGCTTATTTTTTCGTGTGTGTTAAGCGGTTTTATTCTTAATTTTTTATTACTT
TTACAATTATTCGATTGGATTATCTACTTTATTACTATATTTCGGATAAAGCGTGGTGCCCCAGATGGAGATATT
TCTATTTTTCACAAGTGGTAAGTTCCGGTCATCAATTACCGTTCTCCACCATTCCCAAGCTAAACCAGTGCATTC
TTTAGCGTAAACATTAATATTTCTCGCGTTACCTGGCAAATAGATGGACGATGTGAAATGAGCTAGCTTGCTTTT
ATTGTTTTCGCTCCAGTTTTTATGTTGAACAATTTCGTTACCTTCAGGATCATAATTTACTTCATCCCAAGAAAT
GTTGAATTGAGCAACGTATCCTCCAGAGTGATCGATGTTAATTTTTCCATCTGTATAAGCTTTTGAAGTTGTTTC
AATATATTCTGAGTTGTTTTTAATAACAGCTAATTCATTGTCTTTTAGGAAGTTTGTTGTATAAGCAATGGGAAC
TCCTGGTGTTTCTCGATTAAAAGTAGCGCCTTTTTTCAAAATATCGCGTAAGTCTCCGAGGTTGCCGTCGATGAT
TTGAACTTCATCTTTTGCGGAACCTCCGTAAATTACGGCTTTGAAGGAAGAATTTTTGATGATATTTGTTAGTTC
TACATCACCTGAGACAGATTTTCCGCTTACGGCAGCATCAAAAGCAGCTTTTACTTTAGTACTATGGGAATTAGT
TGATAATTTCAAATAAACTTGACGGCCATACGCCACACTTGAGATATATGCAGGAGGATTTTCTGCATTCACTCC
AAGCGCTTGCAACTGCTCTTTAGTAACAGCTTTGCCGAAAAATCTGGAAGGTCTTGTAGGTTCATTAACATTCAC
GTTATAGTAAATTTGTTTAAAACTAATGACTTCTTCTTGCATTTTCCCTTCACTGATTGCGCCGAAGTTTACATT
CAAGCTATTATTTACAGCTTTAAATGCTGTACCAAATTTCGCAATTAATTGTGATTCACTGTAAGCCATTTCGTC
ATCATAATCAATTTTTGCACTTACATTTGGATAAGCTTGAGCATATTTTTCATTCCATCTTTCCACTAATGTATT
TACTGCGTTGTTAACGTTTGATTTAGTGGCATTTTTTACAACGATTTTATTGTCTTGATTAGTCATACCTGGCAA
ATCAATGCTGAGTGTTAATGAATCACGTTTTACAGGGAGAACATCTGGTTGATTTTCTACTAATTCCGAATTCGC
TTTTACGAGAGCACCTGGATAGGTTAGGCTCGAAATTGCATTCACAACTTGAATGTCTGCATTATTTTGATTGAT
GGATTTCTTCTTTTTCTCCACAACAATATATTCATTTCCATCTTTGTAACCTTTTCTTGGCGGCACATTTGTCAC
TGCATCTCCGTGGTATACTAATACATTGTTTTTATTGTAATCCAATCCTTGTATATACTTATCGATTTCATCCGC
GTGTTTCTTTTCGATTGGCGTCTTAGGACTTGCAGGCGGAGATGCTGGTGGTGCCATGGATGAAATTGAATTTTC
TTTATTGAATGCAGATGCATCCTTTGCTTCAGTTTGTTGCGCAATTGGTAGACTAACTAATATAAGTGTAATAAA
AACTAGCATTATTTTTTTCATGGGTTTCACTCTCCTTCTACATTTTTTAACCTAATAATGCCAAATACCGTTTGC
CACCCCTCTCTTTTGATAATTATAATATTGGCGAAATTCGCTTCTAAAGATGAAACGCAATATTATATGCTTGCT
TTATAGCTTTATTCTAGTCCTGCTGTCCCTTTATCGTCGTTAACAAATGTTAATGCCTCAACATAAAAGTCACTT
TAAGATAGGAATATACTAATCAAAGGAGGGATCGAATTCCTGCAGTCATCAAGGCAACCATCAGGATTAATGCGG
ATATTGCGGAGTAACACTTCAGACTGAAAGTAGAAATAAAAACCGCAGCAGACAACTGACAACATCAAATGAAGG
GGGCTTATTCTAATTGATATTATTTATATGATAATAGTTCATTTTGTATTTTTGTTTTTTTTGATATTCTCACCT
GCTTAGTTACAATAAATCAATTCTATCGCTGTATGGTATAGACTGTTTTATTATATATTTTGAATATTTTTAATC
TGCCCAGTCTGGTTTTTTAAAAAAGTGCTATCCTCTTAATGTCTTTACTAAATTAGAAAACAAGTTTCACTTTCA
ACTATTGCATCTTTAATTAATGGTCAAGGTGATTTCAAATGCTCGTTTGTGGCCAGTTATACCTCAAATAACTCA
AGTTGTTGAGCACAGCCAACGCACATGCAGTTTGACGTATGACAGGTATGCTTTATTTCATTTAAATTATGATGG
TTTTCCAGCCAATCAGTGAGTTTCTCTTGATAAGGAATGCGGGAATGTCTATGTATTTTAATAAAATAATTTCAT
TTAATATTATTTCACGAATAGTTATTTGTATCTTTTTGATATGTGGAATGTTCATGGCTGGGGCTTCAGAAAAAT
ATGATGCTAACGCACCGCAACAGGTCCAGCCTTATTCTGTCTCTTCATCTGCATTTGAAAATCTCCATCCTAATA
ATGAAATGGAGAGTTCAATCAATCCCTTTTCCGCATCGGATACAGAAAGAAATGCTGCAATAATAGATCGCGCCA
ATAAGGAGCAGGAGACTGAAGCGGTGAATAAGATGATAAGCACCGGGGCCAGGTTAGCTGCATCAGGCAGGGCAT
CTGATGTTGCTCACTCAATGGTGGGCGATGCGGTTAATCAAGAAATCAAACAGTGGTTAAATCGATTCGGTACGG
CTCAAGTTAATCTGAATTTTGACAAAAATTTTTCGCTAAAAGAAAGCTCTCTTGATTGGCTGGCTCCTTGGTATG
ACTCTGCTTCATTCCTCTTTTTTAGTCAGTTAGGTATTCGCAATAAAGACAGCCGCAACACACTTAACCTTGGCG
TCGGGATACGTACATTGGAGAACGGTTGGCTGTACGGACTTAATACTTTTTATGATAATGATTTGACCGGCCACA
ACCACCGTATCGGTCTTGGTGCCGAGGCCTGGACCGATTATTTACAGTTGGCTGCCAATGGGTATTTTCGCCTCA
ATGGATGGCACTCGTCGCGTGATTTCTCCGACTATAAAGAGCGCCCAGCCACTGGGGGGGATTTGCGCGCGAATG
CTTATTTACCTGCACTCCCACAACTGGGGGGGAAGTTGATGTATGAGCAATACACCGGTGAGCGTGTTGCTTTAT
TTGGTAAAGATAATCTGCAACGCAACCCTTATGCCGTGACTGCCGGGATCAATTACACCCCCGTGCCTCTACTCA
CTGTCGGGGTAGATCAGCGTATGGGGAAAAGCAGTAAGCATGAAACACAGTGGAACCTCCAAATGAACTATCGCC
TGGGCGAGAGTTTTCAGTCGCAACTTAGCCCTTCAGCGGTGGCAGGAACACGTCTACTGGCGGAGAGCCGCTATA
ACCTTGTCGATCGTAACAATAATATCGTGTTGGAGTATCAGAAACAGCAGGTGGTTAAACTGACATTATCGCCAG
CAACTATCTCCGGCCTGCCGGGTCAGGTTTATCAGGTGAACGCACAAGTACAAGGGGCATCTGCTGTAAGGGAAA
TTGTCTGGAGTGATGCCGAACTGATTGCCGCTGGCGGCACATTAACACCACTGAGTACCACACAATTCAACTTGG
TTTTACCGCCTTATAAACGCACAGCACAAGTGAGTCGGGTAACGGACGACCTGACAGCCAACTTTTATTCGCTTA
GTGCGCTCGCGGTTGATCACCAAGGAAACCGATCTAACTCATTCACATTGAGCGTCACCGTTCAGCAGCCTCAGT
TGACATTAACGGCGGCCGTCATTGGTGATGGCGCACCGGCTAATGGGAAAACTGCAATCACCGTTGAGTTCACCG
TTGCTGATTTTGAGGGGAAACCCTTAGCCGGGCAGGAGGTGGTGATAACCACCAATAATGGTGCGCTACCGAATA
AAATCACGGAAAAGACAGATGCAAATGGCGTCGCGCGCATTGCATTAACCAATACGACAGATGGCGTGACGGTAG
TCACAGCAGAAGTGGAGGGGCAACGGCAAAGTGTTGATACCCACTTTGTTAAGGGTACTATCGCGGCGGATAAAT
CCACTCTGGCTGCGGTACCGACATCTATCATCGCTGATGGTCTAATGGCTTCAACCATCACGTTGGAGTTGAAGG
ATACCTATGGGGACCCGCAGGCTGGCGCGAATGTGGCTTTTGACACAACCTTAGGCAATATGGGCGTTATCACGG
ATCACAATGACGGCACTTATAGCGCACCATTGACCAGTACCACGTTGGGGGTAGCAACAGTAACGGTGAAAGTGG
ATGGGGCTGCGTTCAGTGTGCCGAGTGTGACGGTTAATTTCACGGCAGATCCTATTCCAGATGCTGGCCGCTCCA
GTTTCACCGTCTCCACACCGGATATCTTGGCTGATGGCACGATGAGTTCCACATTATCCTTTGTCCCTGTCGATA
AGAATGGCCATTTTATCAGTGGGATGCAGGGCTTGAGTTTTACTCAAAACGGTGTGCCGGTGAGTATTAGCCCCA
TTACCGAGCAGCCAGATAGCTATACCGCGACGGTGGTTGGGAATAGTGTCGGTGATGTCACAATCACGCCGCAGG
TTGATACCCTGATACTGAGTACATTGCAGAAAAAAATATCCCTATTCCCGGTACCTACGCTGACCGGTATTCTGG
TTAACGGGCAAAATTTCGCTACGGATAAAGGGTTCCCGAAAACGATCTTTAAAAACGCCACATTCCAGTTACAGA
TGGATAACGATGTTGCTAATAATACTCAGTATGAGTGGTCGTCGTCATTCACACCCAATGTATCGGTTAACGATC
AGGGTCAGGTGACGATTACCTACCAAACCTATAGCGAAGTGGCTGTGACGGCGAAAAGTAAAAAATTCCCAAGTT
ATTCGGTGAGTTATCGGTTCTACCCAAATCGGTGGATATACGATGGCGGCAGATCGCTGGTATCCAGTCTCGAGG
CCAGCAGACAATGCCAAGGTTCAGATATGTCTGCGGTTCTTGAATCCTCACGTGCAACCAACGGAACGCGTGCGC
CTGACGGGACATTGTGGGGCGAGTGGGGGAGCTTGACCGCGTATAGTTCTGATTGGCAATCTGGTGAATATTGGG
TCAAAAAGACCAGCACGGATTTTGAAACCATGAATATGGACACAGGCGCACTGCAACCAGGGCCTGCATACTTGG
CGTTCCCGCTCTGTGCGCTGTCAATATAACCAGATAACAGATAGCAATAAGAACAGTTTAATGAGCTGATTATTT
GGGGCGCGAATGGGAGTCCGGCAATCCTAGACTCGCCCCATAAGTAGCAAACGTCCAGAAGAACAACGCCGCTCA
GGTTAATTGAGCGGCGCTGTTTTTTTAAAAGGATTGTCGCGATTAAATGCCGATCTTACGGCCCAGCTGCAGCCC
GGGGGATCTATGCGGTGTGAAATACCGCACAGATGCGTAAGGAGAAAATACCGCATCAGGCGCCATTCGCCATTC
AGGCTGCGCAACTGTTGGGAAGGGCGATCGGTGCGGGCCTCTTCGCTATTACGCCAGGACTTCATATACCCAAGC
TTGGAAAATTTTTTTTAAAAAAGTCTTGACACTTTATGCTTCCGGCTCGTATAATGGATCCAGGAGTAACAATAC
AAATGGATTCAAGAGATCCATTTGTATTGTTACTCCTTTTTTTTTTTGTCGACGATCCTTAGCGAAAGCTAAGGA
TTTTTTTTTTACTCGAGCGGATTACTACATACCTGCATTAATGAATCGGCCAACGCGCGGGGAGAGGCGGTTTGC
GTATTGGGCGCTCTTCCGCTTCCTCGCTCACTGACTCGCTGCGCTCGGTCGTTCGGCTGCGGCGAGCGGTATCAG
CTCACTCAAAGGCGGTAATACGGTTATCCACAGAATCAGGGGATAACGCAGGAAAGAACATGTGAGCAAAAGGCC
AGCAAAAGGCCAGGAACCGTAAAAAGGCCGCGTTGCTGGCGTTTTTCCATAGGCTCCGCCCCCCTGACGAGCATC
ACAAAAATCGACGCTCAAGTCAGAGGTGGCGAAACCCGACAGGACTATAAAGATACCAGGCGTTTCCCCCTGGAA
GCTCCCTCGTGCGCTCTCCTGTTCCGACCCTGCCGCTTACCGGATACCTGTCCGCCTTTCTCCCTTCGGGAAGCG
TGGCGCTTTCTCATAGCTCACGCTGTAGGTATCTCAGTTCGGTGTAGGTCGTTCGCTCCAAGCTGGGCTGTGTGC
ACGAACCCCCCGTTCAGCCCGACCGCTGCGCCTTATCCGGTAACTATCGTCTTGAGTCCAACCCGGTAAGACACG
ACTTATCGCCACTGGCAGCAGCCACTGGTAACAGGATTAGCAGAGCGAGGTATGTAGGCGGTGCTACAGAGTTCT
TGAAGTGGTGGCCTAACTACGGCTACACTAGAAGGACAGTATTTGGTATCTGCGCTCTGCTGAAGCCAGTTACCT
TCGGAAAAAGAGTTGGTAGCTCTTGATCCGGCAAACAAACCACCGCTGGTAGCGGTGGTTTTTTTGTTTGCAAGC
AGCAGATTACGCGCAGAAAAAAAGGATCTCAAGAAGATCCTTTGATCTTTTCTACGGGGTCTGACGCTCAGTGGA
ACGAAAACTCACGTTAAGGGATTTTGGTCATGATCTGGTAAGGTTGGGAAGCCCTGCAAAGTAAACTGGATGGCT
TTCTTGCCGCCAAGGATCTGATGGCGCAGGGGATCAAGATCTGATCAAGAGACAGGATGAGGATCGTTTCGCATG
ATTGAACAAGATGGATTGCACGCAGGTTCTCCGGCCGCTTGGGTGGAGAGGCTATTCGGCTATGACTGGGCACAA
CAGACAATCGGCTGCTCTGATGCCGCCGTGTTCCGGCTGTCAGCGCAGGGGCGCCCGGTTCTTTTTGTCAAGACC
GACCTGTCCGGTGCCCTGAATGAACTGCAGGACGAGGCAGCGCGGCTATCGTGGCTGGCCACGACGGGCGTTCCT
TGCGCAGCTGTGCTCGACGTTGTCACTGAAGCGGGAAGGGACTGGCTGCTATTGGGCGAAGTGCCGGGGCAGGAT
CTCCTGTCATCTCACCTTGCTCCTGCCGAGAAAGTATCCATCATGGCTGATGCAATGCGGCGGCTGCATACGCTT
GATCCGGCTACCTGCCCATTCGACCACCAAGCGAAACATCGCATCGAGCGAGCACGTACTCGGATGGAAGCCGGT
CTTGTCGATCAGGATGATCTGGACGAAGAGCATCAGGGGCTCGCGCCAGCCGAACTGTTCGCCAGGCTCAAGGCG
CGCATGCCCGACGGCGAGGATCTCGTCGTGACCCATGGCGATGCCTGCTTGCCGAATATCATGGTGGAAAATGGC
CGCTTTTCTGGATTCATCGACTGTGGCCGGCTGGGTGTGGCGGACCGCTATCAGGACATAGCGTTGGCTACCCGT
GATATTGCTGAAGAGCTTGGCGGCGAATGGGCTGACCGCTTCCTCGTGCTTTACGGTATCGCCGCTCCCGATTCG
CAGCGCATCGCCTTCTATCGCCTTCTTGACGAGTTCTTCTGAGCGGGACTCTGGGGTTCGAAATGACCGACCAAG
CGACGCCCAACCTGCCATCACGAGATTTCGATTCCACCGCCGCCTTCTATGAAATCATGACATTAACCTATAAAA
ATAGGCGTATCACGAGGCCCTTTCGTC

Table 43 contains the 8443 base pair sequence of a verified pMBV43 plasmid. The sequence contains the following regions: hly orf (682-2271 bp); inv orf (2992-5952 bp); shRNA promoter (6317-6375 bp); Sense strand (6376-6397 bp); Loop (6398-6404 bp); Antisense strand (6405-6426 bp); Terminator I (6427-6437 bp); Terminator II (6438-6475 bp); Origin of replication (6735-7322 bp); and kan orf (7513-8307 bp).

TABLE 43

Verified pMBV43 (SEQ ID NO: 564)
TCGCGCGTTTCGGTGATGACGGTGAAAACCTCTGACACATGCAGCTCCCGGAGACGGTCACAGCTTGTCTGTAAG
CGGATGCCGGGAGCAGACAAGCCCGTCAGGGCGCGTCAGCGGGTGTTGGCGGGTGTCGGGGCTGGCTTAACTATG
CGGCATCAGAGCAGATTGTACTGAGAGTGCACCATATCGACGGTATCGATAAGCTTGATAAGCTTTTAAATCAGC
AGGGGTCTTTTTGGCTTGTGTATTATTTTGAAGTTTTTCTTCCCCGACAGAATCTGCTTTTACCGTCATAGTGAA
ATGAGCCTGAAAAGCTATTACCATGATGATACAAATAAGTTTACTTTTCATTTCACCGCTCCTTTTTAATTCGTA
AAACTAAGTTTAAGCCACCTACAACTAATCTGACAGAGAGAGTTAAGGACACGTTTTTTAGTATATGTGGGAACT
AAATTATACGTTTTGCAGTAGAAACTATAGGTGGCTTAAACTTTGGGATATGCTTATATTATATGGATAAACAGT
CAGATATTCTTTTACATTTGTTAATTCTTCTAAAAAAATTAAAAAATAAGCCTGTTTCTACATTCTTCACAAAAT
AATTTACGAAGAGTGCAAAACAAGCTTATTTTTTCGTGTGTGTTAAGCGGTTTTATTCTTAATTTTTTATTACTT
TTACAATTATTCGATTGGATTATCTACTTTATTACTATATTTCGGATAAAGCGTGGTGCCCCAGATGGAGATATT
TCTATTTTTCACAAGTGGTAAGTTCCGGTCATCAATTACCGTTCTCCACCATTCCCAAGCTAAACCAGTGCATTC
TTTAGCGTAAACATTAATATTTCTCGCGTTACCTGGCAAATAGATGGACGATGTGAAATGAGCTAGCTTGCTTTT
ATTGTTTTCGCTCCAGTTTTTATGTTGAACAATTTCGTTACCTTCAGGATCATAATTTACTTCATCCCAAGAAAT
GTTGAATTGAGCAACGTATCCTCCAGAGTGATCGATGTTAATTTTTCCATCTGTATAAGCTTTTGAAGTTGTTTC
AATATATTCTGAGTTGTTTTTAATAACAGCTAATTCATTGTCTTTTAGGAAGTTTGTTGTATAAGCAATGGGAAC
TCCTGGTGTTTCTCGATTAAAAGTAGCGCCTTTTTTCAAAATATCGCGTAAGTCTCCGAGGTTGCCGTCGATGAT
TTGAACTTCATCTTTTGCGGAACCTCCGTAAATTACGGCTTTGAAGGAAGAATTTTTGATGATATTTGTTAGTTC
TACATCACCTGAGACAGATTTTCCGCTTACGGCAGCATCAAAAGCAGCTTTTACTTTAGTACTATGGGAATTAGT
TGATAATTTCAAATAAACTTGACGGCCATACGCCACACTTGAGATATATGCAGGAGGATTTTCTGCATTCACTCC
AAGCGCTTGCAACTGCTCTTTAGTAACAGCTTTGCCGAAAAATCTGGAAGGTCTTGTAGGTTCATTAACATTCAC
GTTATAGTAAATTTGTTTAAAACTAATGACTTCTTCTTGCATTTTCCCTTCACTGATTGCGCCGAAGTTTACATT
CAAGCTATTATTTACAGCTTTAAATGCTGTACCAAATTTCGCAATTAATTGTGATTCACTGTAAGCCATTTCGTC
ATCATAATCAATTTTTGCACTTACATTTGGATAAGCTTGAGCATATTTTTCATTCCATCTTTCCACTAATGTATT
TACTGCGTTGTTAACGTTTGATTTAGTGGCATTTTTTACAACGATTTTATTGTCTTGATTAGTCATACCTGGCAA
ATCAATGCTGAGTGTTAATGAATCACGTTTTACAGGGAGAACATCTGGTTGATTTTCTACTAATTCCGAATTCGC
TTTTACGAGAGCACCTGGATAGGTTAGGCTCGAAATTGCATTCACAACTTGAATGTCTGCATTATTTTGATTGAT
GGATTTCTTCTTTTTCTCCACAACAATATATTCATTTCCATCTTTGTAACCTTTTCTTGGCGGCACATTTGTCAC
TGCATCTCCGTGGTATACTAATACATTGTTTTTATTGTAATCCAATCCTTGTATATACTTATCGATTTCATCCGC
GTGTTTCTTTTCGATTGGCGTCTTAGGACTTGCAGGCGGAGATGCTGGTGGTGCCATGGATGAAATTGAATTTTC
TTTATTGAATGCAGATGCATCCTTTGCTTCAGTTTGTTGCGCAATTGGTAGACTAACTAATATAAGTGTAATAAA
AACTAGCATTATTTTTTTCATGGGTTTCACTCTCCTTCTACATTTTTTAACCTAATAATGCCAAATACCGTTTGC
CACCCCTCTCTTTTGATAATTATAATATTGGCGAAATTCGCTTCTAAAGATGAAACGCAATATTATATGCTTGCT
TTATAGCTTTATTCTAGTCCTGCTGTCCCTTTATCGTCGTTAACAAATGTTAATGCCTCAACATAAAAGTCACTT
TAAGATAGGAATATACTAATCAAAGGAGGGATCGAATTCCTGCAGTCATCAAGGCAACCATCAGGATTAATGCGG
ATATTGCGGAGTAACACTTCAGACTGAAAGTAGAAATAAAAACCGCAGCAGACAACTGACAACATCAAATGAAGG
GGGCTTATTCTAATTGATATTATTTATATGATAATAGTTCATTTTGTATTTTGTTTTTTTGATATTCTCACCTGC
TTAGTTACAATAAATCAATTCTATCGCTGTATGGTATAGACTGTTTTATTATATATTTTGAATATTTTTAATCTG
CCCAGTCTGGTTTTTTAAAAAAGTGCTATCCTCTTAATGTCTTTACTAAATTAGAAAACAAGTTTCACTTTCAAC
TATTGCATCTTTAATTAATGGTCAAGGTGATTTCAAATGCTCGTTTGTGGCCAGTTATACCTCAAATAACTCAAG
TTGTTGAGCACAGCCAACGCACATGCAGTTTGACGTATGACAGGTATGCTTTATTTCATTTAAATTATGATGGTT
TTCCAGCCAATCAGTGAGTTTCTCTTGATAAGGAATGCGGGAATGTCTATGTATTTTAATAAAATAATTTCATTT
AATATTATTTCACGAATAGTTATTTGTATCTTTTTGATATGTGGAATGTTCATGGCTGGGGCTTCAGAAAAATAT
GATGCTAACGCACCGCAACAGGTCCAGCCTTATTCTGTCTCTTCATCTGCATTTGAAAATCTCCATCCTAATAAT
GAAATGGAGAGTTCAATCAATCCCTTTTCCGCATCGGATACAGAAAGAAATGCTGCAATAATAGATCGCGCCAAT
AAGGAGCAGGAGACTGAAGCGGTGAATAAGATGATAAGCACCGGGGCCAGGTTAGCTGCATCAGGCAGGGCATCT
GATGTTGCTCACTCAATGGTGGGCGATGCGGTTAATCAAGAAATCAAACAGTGGTTAAATCGATTCGGTACGGCT
CAAGTTAATCTGAATTTTGACAAAAATTTTTCGCTAAAAGAAAGCTCTCTTGATTGGCTGGCTCCTTGGTATGAC
TCTGCTTCATTCCTCTTTTTTAGTCAGTTAGGTATTCGCAATAAAGACAGCCGCAACACACTTAACCTTGGCGTC
GGGATACGTACATTGGAGAACGGTTGGCTGTACGGACTTAATACTTTTTATGATAATGATTTGACCGGCCACAAC
CACCGTATCGGTCTTGGTGCCGAGGCCTGGACCGATTATTTACAGTTGGCTGCCAATGGGTATTTTCGCCTCAAT
GGATGGCACTCGTCGCGTGATTTCTCCGACTATAAAGAGCGCCCAGCCACTGGGGGGGATTTGCGCGCGAATGCT
TATTTACCTGCACTCCCACAACTGGGGGGGAAGTTGATGTATGAGCAATACACCGGTGAGCGTGTTGCTTTATTT
GGTAAAGATAATCTGCAACGCAACCCTTATGCCGTGACTGCCGGGATCAATTACACCCCCGTGCCTCTACTCACT
GTCGGGGTAGATCAGCGTATGGGGAAAAGCAGTAAGCATGAAACACAGTGGAACCTCCAAATGAACTATCGCCTG
GGCGAGAGTTTTCAGTCGCAACTTAGCCCTTCAGCGGTGGCAGGAACACGTCTACTGGCGGAGAGCCGCTATAAC
CTTGTCGATCGTAACAATAATATCGTGTTGGAGTATCAGAAACAGCAGGTGGTTAAACTGACATTATCGCCAGCA
ACTATCTCCGGCCTGCCGGGTCAGGTTTATCAGGTGAACGCACAAGTACAAGGGGCATCTGCTGTAAGGGAAATT
GTCTGGAGTGATGCCGAACTGATTGCCGCTGGCGGCACATTAACACCACTGAGTACCACACAATTCAACTTGGTT
TTACCGCCTTATAAACGCACAGCACAAGTGAGTCGGGTAACGGACGACCTGACAGCCAACTTTTATTCGCTTAGT
GCGCTCGCGGTTGATCACCAAGGAAACCGATCTAACTCATTCACATTGAGCGTCACCGTTCAGCAGCCTCAGTTG
ACATTAACGGCGGCCGTCATTGGTGATGGCGCACCGGCTAATGGGAAAACTGCAATCACCGTTGAGTTCACCGTT
GCTGATTTTGAGGGGAAACCCTTAGCCGGGCAGGAGGTGGTGATAACCACCAATAATGGTGCGCTACCGAATAAA
ATCACGGAAAAGACAGATGCAAATGGCGTCGCGCGCATTGCATTAACCAATACGACAGATGGCGTGACGGTAGTC
ACAGCAGAAGTGGAGGGGCAACGGCAAAGTGTTGATACCCACTTTGTTAAGGGTACTATCGCGGCGGATAAATCC
ACTCTGGCTGCGGTACCGACATCTATCATCGCTGATGGTCTAATGGCTTCAACCATCACGTTGGAGTTGAAGGAT
ACCTATGGGGACCCGCAGGCTGGCGCGAATGTGGCTTTTGACACAACCTTAGGCAATATGGGCGTTATCACGGAT
CACAATGACGGCACTTATAGCGCACCATTGACCAGTACCACGTTGGGGGTAGCAACAGTAACGGTGAAAGTGGAT
GGGGCTGCGTTCAGTGTGCCGAGTGTGACGGTTAATTTCACGGCAGATCCTATTCCAGATGCTGGCCGCTCCAGT
TTCACCGTCTCCACACCGGATATCTTGGCTGATGGCACGATGAGTTCCACATTATCCTTTGTCCCTGTCGATAAG
AATGGCCATTTTATCAGTGGGATGCAGGGCTTGAGTTTTACTCAAAACGGTGTGCCGGTGAGTATTAGCCCCATT
ACCGAGCAGCCAGATAGCTATACCGCGACGGTGGTTGGGAATAGTGTCGGTGATGTCACAATCACGCCGCAGGTT
GATACCCTGATACTGAGTACATTGCAGAAAAAAATATCCCTATTCCCGGTACCTACGCTGACCGGTATTCTGGTT
AACGGGCAAAATTTCGCTACGGATAAAGGGTTCCCGAAAACGATCTTTAAAAACGCCACATTCCAGTTACAGATG
GATAACGATGTTGCTAATAATACTCAGTATGAGTGGTCGTCGTCATTCACACCCAATGTATCGGTTAACGATCAG
GGTCAGGTGACGATTACCTACCAAACCTATAGCGAAGTGGCTGTGACGGCGAAAAGTAAAAAATTCCCAAGTTAT
TCGGTGAGTTATCGGTTCTACCCAAATCGGTGGATATACGATGGCGGCAGATCGCTGGTATCCAGTCTCGAGGCC
AGCAGACAATGCCAAGGTTCAGATATGTCTGCGGTTCTTGAATCCTCACGTGCAACCAACGGAACGCGTGCGCCT
GACGGGACATTGTGGGGCGAGTGGGGGAGCTTGACCGCGTATAGTTCTGATTGGCAATCTGGTGAATATTGGGTC
AAAAAGACCAGCACGGATTTTGAAACCATGAATATGGACACAGGCGCACTGCAACCAGGGCCTGCATACTTGGCG
TTCCCGCTCTGTGCGCTGTCAATATAACCAGATAACAGATAGCAATAAGAACAGTTTAATGAGCTGATTATTTGG
GGCGCGAATGGGAGTCCGGCAATCCTAGACTCGCCCCATAAGTAGCAAACGTCCAGAGAACAACGCCGCTCAGGT
TAATTGAGCGGCGTTGTTTTTTTAAAAGGATTTGTCGCGATAAGCGTGAGCTGGCGTTAAATGCCGATCTTACGG
CCCAGCTGCAGCCCGGGGGATCTATGCGGTGTGAAATACCGCACAGATGCGTAAGGAGAAAATACCGCATCAGGC
GCCATTCGCCATTCAGGCTGCGCAACTGTTGGGAAGGGCGATCGGTGCGGGCCTCTTCGCTATTACGCCAGACTT
CATTATACCCAAGCTTGGAAAATTTTTTTTAAAAAAGTCTTGACACTTTATGCTTCCGGCTCGTATAATGGATCC
AGGAGTAACAATACAAATGGATTCAAGAGATCCATTTGTATTGTTACTCCTTTTTTTTTTTTGTCGACGATCCTT
AGCGAAAGCTAAGGATTTTTTTTTTACTCGAGCGGATTACTACATACCTGCATTAATGAATCGGCCAACGCGCGG
GGAGAGGCGGTTTGCGTATTGGGCGCTCTTCCGCTTCCTCGCTCACTGACTCGCTGCGCTCGGTCGTTCGGCTGC
GGCGAGCGGTATCAGCTCACTCAAAGGCGGTAATACGGTTATCCACAGAATCAGGGGATAACGCAGGAAAGAACA
TGTGAGCAAAAGGCCAGCAAAAGGCCAGGAACCGTAAAAAGGCCGCGTTGCTGGCGTTTTTCCATAGGCTCCGCC
CCCCTGACGAGCATCACAAAAATCGACGCTCAAGTCAGAGGTGGCGAAACCCGACAGGACTATAAAGATACCAGG
CGTTTCCCCCTGGAAGCTCCCTCGTGCGCTCTCCTGTTCCGACCCTGCCGCTTACCGGATACCTGTCCGCCTTTC
TCCCTTCGGGAAGCGTGGCGCTTTCTCATAGCTCACGCTGTAGGTATCTCAGTTCGGTGTAGGTCGTTCGCTCCA
AGCTGGGCTGTGTGCACGAACCCCCCGTTCAGCCCGACCGCTGCGCCTTATCCGGTAACTATCGTCTTGAGTCCA
ACCCGGTAAGACACGACTTATCGCCACTGGCAGCAGCCACTGGTAACAGGATTAGCAGAGCGAGGTATGTAGGCG
GTGCTACAGAGTTCTTGAAGTGGTGGCCTAACTACGGCTACACTAGAAGAACAGTATTTGGTATCTGCGCTCTGC
TGAAGCCAGTTACCTTCGGAAAAAGAGTTGGTAGCTCTTGATCCGGCAAACAAACCACCGCTGGTAGCGGTGGTT
TTTTTGTTTGCAAGCAGCAGATTACGCGCAGAAAAAAAGGATCTCAAGAAGATCCTTTGATCTTTTCTACGGGGT
CTGACGCTCAGTGGAACGAAAACTCACGTTAAGGGATTTTGGTCATGATCTGGTAAGGTTGGGAAGCCCTGCAAA
GTAAACTGGATGGCTTtCTTGCCGCCAAGGATCTGATGGCGCAGGGGATCAAGATCTGATCAAGAGACAGGATGA
GGATCGTTTCGCATGATTGAACAAGATGGATTGCACGCAGGTTCTCCGGCCGCTTGGGTGGAGAGGCTATTCGGC
TATGACTGGGCACAACAGACAATCGGCTGCTCTGATGCCGCCGTGTTCCGGCTGTCAGCGCAGGGGCGCCCGGTT
CTTTTTGTCAAGACCGACCTGTCCGGTGCCCTGAATGAACTGCAGGACGAGGCAGCGCGGCTATCGTGGCTGGCC
ACGACGGGCGTTCCTTGCGCAGCTGTGCTCGACGTTGTCACTGAAGCGGGAAGGGACTGGCTGCTATTGGGCGAA
GTGCCGGGGCAGGATCTCCTGTCATCTCACCTTGCTCCTGCCGAGAAAGTATCCATCATGGCTGATGCAATGCGG
CGGCTGCATACGCTTGATCCGGCTACCTGCCCATTCGACCACCAAGCGAAACATCGCATCGAGCGAGCACGTACT
CGGATGGAAGCCGGTCTTGTCGATCAGGATGATCTGGACGAAGAGCATCAGGGGCTCGCGCCAGCCGAACTGTTC
GCCAGGCTCAAGGCGCGCATGCCCGACGGCGAGGATCTCGTCGTGACCCATGGCGATGCCTGCTTGCCGAATATC
ATGGTGGAAAATGGCCGCTTTTCTGGATTCATCGACTGTGGCCGGCTGGGTGTGGCGGACCGCTATCAGGACATA
GCGTTGGCTACCCGTGATATTGCTGAAGAGCTTGGCGGCGAATGGGCTGACCGCTTCCTCGTGCTTTACGGTATC
GCCGCTCCCGATTCGCAGCGCATCGCCTTCTATCGCCTTCTTGACGAGTTCTTCTGAGCGGGACTCTGGGGTTCG
AAATGACCGACCAAGCGACGCCCAACCTGCCATCACGAGATTTCGATTCCACCGCCGCCTTCTATGAAATCATGA
CATTAACCTATAAAAATAGGCGTATCACGAGGCCCTTTCGTCT

TABLE 44

pMBV44 (SEQ ID NO: 565)
TCGCGCGTTTCGGTGATGACGGTGAAAACCTCTGACACATGCAGCTCCCGGAGACGGTCACAGCTTGTCTGTAAG
CGGATGCCGGGAGCAGACAAGCCCGTCAGGGCGCGTCAGCGGGTGTTGGCGGGTGTCGGGGCTGGCTTAACTATG
CGGCATCAGAGCAGATTGTACTGAGAGTGCACCATATCGACGGTATCGATAAGCTTGATAAGCTTTTAAATCAGC
AGGGGTCTTTTTGGCTTGTGTATTATTTTGAAGTTTTTCTTCCCCGACAGAATCTGCTTTTACCGTCATAGTGAA
ATGAGCCTGAAAAGCTATTACCATGATGATACAAATAAGTTTACTTTTCATTTCACCGCTCCTTTTTAATTCGTA
AAACTAAGTTTAAGCCACCTACAACTAATCTGACAGAGAGAGTTAAGGACACGTTTTTTAGTATATGTGGGAACT
AAATTATACGTTTTGCAGTAGAAACTATAGGTGGCTTAAACTTTGGGATATGCTTATATTATATGGATAAACAGT
CAGATATTCTTTTACATTTGTTAATTCTTCTAAAAAAATTAAAAAATAAGCCTGTTTCTACATTCTTCACAAAAT
AATTTACGAAGAGTGCAAAACAAGCTTATTTTTTCGTGTGTGTTAAGCGGTTTTATTCTTAATTTTTTATTACTT
TTACAATTATTCGATTGGATTATCTACTTTATTACTATATTTCGGATAAAGCGTGGTGCCCCAGATGGAGATATT
TCTATTTTTCACAAGTGGTAAGTTCCGGTCATCAATTACCGTTCTCCACCATTCCCAAGCTAAACCAGTGCATTC
TTTAGCGTAAACATTAATATTTCTCGCGTTACCTGGCAAATAGATGGACGATGTGAAATGAGCTAGCTTGCTTTT
ATTGTTTTCGCTCCAGTTTTTATGTTGAACAATTTCGTTACCTTCAGGATCATAATTTACTTCATCCCAAGAAAT
GTTGAATTGAGCAACGTATCCTCCAGAGTGATCGATGTTAATTTTTCCATCTGTATAAGCTTTTGAAGTTGTTTC
AATATATTCTGAGTTGTTTTTAATAACAGCTAATTCATTGTCTTTTAGGAAGTTTGTTGTATAAGCAATGGGAAC
TCCTGGTGTTTCTCGATTAAAAGTAGCGCCTTTTTTCAAAATATCGCGTAAGTCTCCGAGGTTGCCGTCGATGAT
TTGAACTTCATCTTTTGCGGAACCTCCGTAAATTACGGCTTTGAAGGAAGAATTTTTGATGATATTTGTTAGTTC
TACATCACCTGAGACAGATTTTCCGCTTACGGCAGCATCAAAAGCAGCTTTTACTTTAGTACTATGGGAATTAGT
TGATAATTTCAAATAAACTTGACGGCCATACGCCACACTTGAGATATATGCAGGAGGATTTTCTGCATTCACTCC
AAGCGCTTGCAACTGCTCTTTAGTAACAGCTTTGCCGAAAAATCTGGAAGGTCTTGTAGGTTCATTAACATTCAC
GTTATAGTAAATTTGTTTAAAACTAATGACTTCTTCTTGCATTTTCCCTTCACTGATTGCGCCGAAGTTTACATT
CAAGCTATTATTTACAGCTTTAAATGCTGTACCAAATTTCGCAATTAATTGTGATTCACTGTAAGCCATTTCGTC
ATCATAATCAATTTTTGCACTTACATTTGGATAAGCTTGAGCATATTTTTCATTCCATCTTTCCACTAATGTATT
TACTGCGTTGTTAACGTTTGATTTAGTGGCATTTTTTACAACGATTTTATTGTCTTGATTAGTCATACCTGGCAA
ATCAATGCTGAGTGTTAATGAATCACGTTTTACAGGGAGAACATCTGGTTGATTTTCTACTAATTCCGAATTCGC
TTTTACGAGAGCACCTGGATAGGTTAGGCTCGAAATTGCATTCACAACTTGAATGTCTGCATTATTTTGATTGAT
GGATTTCTTCTTTTTCTCCACAACAATATATTCATTTCCATCTTTGTAACCTTTTCTTGGCGGCACATTTGTCAC
TGCATCTCCGTGGTATACTAATACATTGTTTTTATTGTAATCCAATCCTTGTATATACTTATCGATTTCATCCGC
GTGTTTCTTTTCGATTGGCGTCTTAGGACTTGCAGGCGGAGATGCTGGTGGTGCCATGGATGAAATTGAATTTTC
TTTATTGAATGCAGATGCATCCTTTGCTTCAGTTTGTTGCGCAATTGGTAGACTAACTAATATAAGTGTAATAAA
AACTAGCATTATTTTTTTCATGGGTTTCACTCTCCTTCTACATTTTTTAACCTAATAATGCCAAATACCGTTTGC
CACCCCTCTCTTTTGATAATTATAATATTGGCGAAATTCGCTTCTAAAGATGAAACGCAATATTATATGCTTGCT
TTATAGCTTTATTCTAGTCCTGCTGTCCCTTTATCGTCGTTAACAAATGTTAATGCCTCAACATAAAAGTCACTT
TAAGATAGGAATATACTAATCAAAGGAGGGATCGAATTCCTGCAGTCATCAAGGCAACCATCAGGATTAATGCGG
ATATTGCGGAGTAACACTTCAGACTGAAAGTAGAAATAAAAACCGCAGCAGACAACTGACAACATCAAATGAAGG
GGGCTTATTCTAATTGATATTATTTATATGATAATAGTTCATTTTGTATTTTTGTTTTTTTTGATATTCTCACCT
GCTTAGTTACAATAAATCAATTCTATCGCTGTATGGTATAGACTGTTTTATTATATATTTTGAATATTTTTAATC
TGCCCAGTCTGGTTTTTTAAAAAAGTGCTATCCTCTTAATGTCTTTACTAAATTAGAAAACAAGTTTCACTTTCA
ACTATTGCATCTTTAATTAATGGTCAAGGTGATTTCAAATGCTCGTTTGTGGCCAGTTATACCTCAAATAACTCA
AGTTGTTGAGCACAGCCAACGCACATGCAGTTTGACGTATGACAGGTATGCTTTATTTCATTTAAATTATGATGG
TTTTCCAGCCAATCAGTGAGTTTCTCTTGATAAGGAATGCGGGAATGTCTATGTATTTTAATAAAATAATTTCAT
TTAATATTATTTCACGAATAGTTATTTGTATCTTTTTGATATGTGGAATGTTCATGGCTGGGGCTTCAGAAAAAT
ATGATGCTAACGCACCGCAACAGGTCCAGCCTTATTCTGTCTCTTCATCTGCATTTGAAAATCTCCATCCTAATA
ATGAAATGGAGAGTTCAATCAATCCCTTTTCCGCATCGGATACAGAAAGAAATGCTGCAATAATAGATCGCGCCA
ATAAGGAGCAGGAGACTGAAGCGGTGAATAAGATGATAAGCACCGGGGCCAGGTTAGCTGCATCAGGCAGGGCAT
CTGATGTTGCTCACTCAATGGTGGGCGATGCGGTTAATCAAGAAATCAAACAGTGGTTAAATCGATTCGGTACGG
CTCAAGTTAATCTGAATTTTGACAAAAATTTTTCGCTAAAAGAAAGCTCTCTTGATTGGCTGGCTCCTTGGTATG
ACTCTGCTTCATTCCTCTTTTTTAGTCAGTTAGGTATTCGCAATAAAGACAGCCGCAACACACTTAACCTTGGCG
TCGGGATACGTACATTGGAGAACGGTTGGCTGTACGGACTTAATACTTTTTATGATAATGATTTGACCGGCCACA
ACCACCGTATCGGTCTTGGTGCCGAGGCCTGGACCGATTATTTACAGTTGGCTGCCAATGGGTATTTTCGCCTCA
ATGGATGGCACTCGTCGCGTGATTTCTCCGACTATAAAGAGCGCCCAGCCACTGGGGGGGATTTGCGCGCGAATG
CTTATTTACCTGCACTCCCACAACTGGGGGGGAAGTTGATGTATGAGCAATACACCGGTGAGCGTGTTGCTTTAT
TTGGTAAAGATAATCTGCAACGCAACCCTTATGCCGTGACTGCCGGGATCAATTACACCCCCGTGCCTCTACTCA
CTGTCGGGGTAGATCAGCGTATGGGGAAAAGCAGTAAGCATGAAACACAGTGGAACCTCCAAATGAACTATCGCC
TGGGCGAGAGTTTTCAGTCGCAACTTAGCCCTTCAGCGGTGGCAGGAACACGTCTACTGGCGGAGAGCCGCTATA
ACCTTGTCGATCGTAACAATAATATCGTGTTGGAGTATCAGAAACAGCAGGTGGTTAAACTGACATTATCGCCAG
CAACTATCTCCGGCCTGCCGGGTCAGGTTTATCAGGTGAACGCACAAGTACAAGGGGCATCTGCTGTAAGGGAAA
TTGTCTGGAGTGATGCCGAACTGATTGCCGCTGGCGGCACATTAACACCACTGAGTACCACACAATTCAACTTGG
TTTTACCGCCTTATAAACGCACAGCACAAGTGAGTCGGGTAACGGACGACCTGACAGCCAACTTTTATTCGCTTA
GTGCGCTCGCGGTTGATCACCAAGGAAACCGATCTAACTCATTCACATTGAGCGTCACCGTTCAGCAGCCTCAGT
TGACATTAACGGCGGCCGTCATTGGTGATGGCGCACCGGCTAATGGGAAAACTGCAATCACCGTTGAGTTCACCG
TTGCTGATTTTGAGGGGAAACCCTTAGCCGGGCAGGAGGTGGTGATAACCACCAATAATGGTGCGCTACCGAATA
AAATCACGGAAAAGACAGATGCAAATGGCGTCGCGCGCATTGCATTAACCAATACGACAGATGGCGTGACGGTAG
TCACAGCAGAAGTGGAGGGGCAACGGCAAAGTGTTGATACCCACTTTGTTAAGGGTACTATCGCGGCGGATAAAT
CCACTCTGGCTGCGGTACCGACATCTATCATCGCTGATGGTCTAATGGCTTCAACCATCACGTTGGAGTTGAAGG
ATACCTATGGGGACCCGCAGGCTGGCGCGAATGTGGCTTTTGACACAACCTTAGGCAATATGGGCGTTATCACGG
ATCACAATGACGGCACTTATAGCGCACCATTGACCAGTACCACGTTGGGGGTAGCAACAGTAACGGTGAAAGTGG
ATGGGGCTGCGTTCAGTGTGCCGAGTGTGACGGTTAATTTCACGGCAGATCCTATTCCAGATGCTGGCCGCTCCA
GTTTCACCGTCTCCACACCGGATATCTTGGCTGATGGCACGATGAGTTCCACATTATCCTTTGTCCCTGTCGATA
AGAATGGCCATTTTATCAGTGGGATGCAGGGCTTGAGTTTTACTCAAAACGGTGTGCCGGTGAGTATTAGCCCCA
TTACCGAGCAGCCAGATAGCTATACCGCGACGGTGGTTGGGAATAGTGTCGGTGATGTCACAATCACGCCGCAGG
TTGATACCCTGATACTGAGTACATTGCAGAAAAAAATATCCCTATTCCCGGTACCTACGCTGACCGGTATTCTGG
TTAACGGGCAAAATTTCGCTACGGATAAAGGGTTCCCGAAAACGATCTTTAAAAACGCCACATTCCAGTTACAGA
TGGATAACGATGTTGCTAATAATACTCAGTATGAGTGGTCGTCGTCATTCACACCCAATGTATCGGTTAACGATC
AGGGTCAGGTGACGATTACCTACCAAACCTATAGCGAAGTGGCTGTGACGGCGAAAAGTAAAAAATTCCCAAGTT
ATTCGGTGAGTTATCGGTTCTACCCAAATCGGTGGATATACGATGGCGGCAGATCGCTGGTATCCAGTCTCGAGG
CCAGCAGACAATGCCAAGGTTCAGATATGTCTGCGGTTCTTGAATCCTCACGTGCAACCAACGGAACGCGTGCGC
CTGACGGGACATTGTGGGGCGAGTGGGGGAGCTTGACCGCGTATAGTTCTGATTGGCAATCTGGTGAATATTGGG
TCAAAAAGACCAGCACGGATTTTGAAACCATGAATATGGACACAGGCGCACTGCAACCAGGGCCTGCATACTTGG
CGTTCCCGCTCTGTGCGCTGTCAATATAACCAGATAACAGATAGCAATAAGAACAGTTTAATGAGCTGATTATTT
GGGGCGCGAATGGGAGTCCGGCAATCCTAGACTCGCCCCATAAGTAGCAAACGTCCAGAAGAACAACGCCGCTCA
GGTTAATTGAGCGGCGCTGTTTTTTTAAAAGGATTGTCGCGATTAAATGCCGATCTTACGGCCCAGCTGCAGCCC
GGGGGATCTATGCGGTGTGAAATACCGCACAGATGCGTAAGGAGAAAATACCGCATCAGGCGCCATTCGCCATTC
AGGCTGCGCAACTGTTGGGAAGGGCGATCGGTGCGGGCCTCTTCGCTATTACGCCAGGACTTCATATACCCAAGC
TTGGAAAATTTTTTTTAAAAAAGTCTTGACACTTTATGCTTCCGGCTCGTATAATGGATCCAGGAGTAACAATAC
AAATGGATTCAAGAGATCCATTTGTATTGTTACTCCTTTTTTTTTTTGTCGACGATCCTTAGCGAAAGCTAAGGA
TTTTTTTTTTACTCGAGCGGATTACTACATACCTGCATTAATGAATCGGCCAACGCGCGGGGAGAGGCGGTTTGC
GTATTGGGCGCTCTTCCGCTTCCTCGCTCACTGACTCGCTGCGCTCGGTCGTTCGGCTGCGGCGAGCGGTATCAG
CTCACTCAAAGGCGGTAATACGGTTATCCACAGAATCAGGGGATAACGCAGGAAAGAACATGTGAGCAAAAGGCC
AGCAAAAGGCCAGGAACCGTAAAAAGGCCGCGTTGCTGGCGTTTTTCCATAGGCTCCGCCCCCCTGACGAGCATC
ACAAAAATCGACGCTCAAGTCAGAGGTGGCGAAACCCGACAGGACTATAAAGATACCAGGCGTTTCCCCCTGGAA
GCTCCCTCGTGCGCTCTCCTGTTCCGACCCTGCCGCTTACCGGATACCTGTCCGCCTTTCTCCCTTCGGGAAGCG
TGGCGCTTTCTCATAGCTCACGCTGTAGGTATCTCAGTTCGGTGTAGGTCGTTCGCTCCAAGCTGGGCTGTGTGC
ACGAACCCCCCGTTCAGCCCGACCGCTGCGCCTTATCCGGTAACTATCGTCTTGAGTCCAACCCGGTAAGACACG
ACTTATCGCCACTGGCAGCAGCCACTGGTAACAGGATTAGCAGAGCGAGGTATGTAGGCGGTGCTACAGAGTTCT
TGAAGTGGTGGCCTAACTACGGCTACACTAGAAGGACAGTATTTGGTATCTGCGCTCTGCTGAAGCCAGTTACCT
TCGGAAAAAGAGTTGGTAGCTCTTGATCCGGCAAACAAACCACCGCTGGTAGCGGTGGTTTTTTTGTTTGCAAGC
AGCAGATTACGCGCAGAAAAAAAGGATCTCAAGAAGATCCTTTGATCTTTTCTACGGGGTCTGACGCTCAGTGGA
ACGAAAACTCACGTTAAGGGATTTTGGTCATGATTTCATAGAAGGCGGCGGTGGAATCGAAATCTCGTGATGGCA
GGTTGGGCGTCGCTTGGTCGGTCATTTCGAACCCCAGAGTCCCGCTCAGAAGAACTCGTCAAGAAGGCGATAGAA
GGCGATGCGCTGCGAATCGGGAGCGGCGATACCGTAAAGCACGAGGAAGCGGTCAGCCCATTCGCCGCCAAGCTC
TTCAGCAATATCACGGGTAGCCAACGCTATGTCCTGATAGCGGTCCGCCACACCCAGCCGGCCACAGTCGATGAA
TCCAGAAAAGCGGCCATTTTCCACCATGATATTCGGCAAGCAGGCATCGCCATGGGTCACGACGAGATCCTCGCC
GTCGGGCATGCGCGCCTTGAGCCTGGCGAACAGTTCGGCTGGCGCGAGCCCCTGATGCTCTTCGTCCAGATCATC
CTGATCGACAAGACCGGCTTCCATCCGAGTACGTGCTCGCTCGATGCGATGTTTCGCTTGGTGGTCGAATGGGCA
GGTAGCCGGATCAAGCGTATGCAGCCGCCGCATTGCATCAGCCATGATGGATACTTTCTCGGCAGGAGCAAGGTG
AGATGACAGGAGATCCTGCCCCGGCACTTCGCCCAATAGCAGCCAGTCCCTTCCCGCTTCAGTGACAACGTCGAG
CACAGCTGCGCAAGGAACGCCCGTCGTGGCCAGCCACGATAGCCGCGCTGCCTCGTCCTGCAGTTCATTCAGGGC
ACCGGACAGGTCGGTCTTGACAAAAAGAACCGGGCGCCCCTGCGCTGACAGCCGGAACACGGCGGCATCAGAGCA
GCCGATTGTCTGTTGTGCCCAGTCATAGCCGAATAGCCTCTCCACCCAAGCGGCCGGAGAACCTGCGTGCAATCC
ATCTTGTTCAATCATGCGAAACGATCCTCATCCTGTCTCTTGATCAGATCTTGATCCCCTGCGCCATCAGATCCT
TGGCGGCAAGAAAGCCATCCAGTTTACTTTGCAGGGCTTCCCAACCTTACCAGATCATGACATTAACCTATAAAA
ATAGGCGTATCACGAGGCCCTTTCGTC

### Example 23

### Construction of pNJSZc Plasmid

pNJSZ is a 10.4 kb plasmid that confers the abilities required to induce tkRNAi. It contains two genes, inv and hly, that allow bacteria to invade mammalian cells and to escape from the entry vacuole. Expression of the short hairpin RNA is different between the original Trip plasmid and pNJSZ. In pNJSZ, expression of shRNA is under the control of a constitutive bacterial promoter, which allows for continuous expression. This is different from the original Trip plasmid, which has an ITPG inducible promoter, which controls the expression of the shRNA. Moreover, pNJSZ and the original Trip plasmid contain different antibiotic resistant genes. pNJSZ has the kanamycin resistance gene, whereas the original Trip plasmid has the ampicillin resistance gene. pNJSZc was constructed from pNJSZ by removing any regions of pNJSZ that were not required for its maintenance or abilities to induce tkRNAi.

Step 1 as shown in PCT Publication No. WO2008/156702 at FIG. 28: Removed an extra BamH1 site at 9778 by digesting pNJSZ with both SpeI (9784) and XmaI (9772), T4 DNA polymerase filled-in these two sites and then allowed the plasmid to self ligate, creating pNJSZ ΔBamH1.

Step 2 as shown in PCT Publication No. WO2008/156702 at FIG. 29: Removed both an extra SalI site at 972 and the f1 origin of replication by digesting pNJSZ ΔBamH1 with BglI (208) and PmeI (982), T4 DNA polymerase filled-in these two sites and allowed the plasmid to self ligate, creating pNJSZc.

The pNJSZc DNA sequence is shown in Table 45.

TABLE 45

pNJSZc (SEQ ID NO: 566)
GGCCGCTCGAGCATGCATCTAGAGGGCCCAATTCGCCCTATAGTGAGTCGTATTACAATTCACTGGCCGTCGTTT
TACAACGTCGTGACTGGGAAAACCCTGGCGTTACCCAACTTAATCGCCTTGCAGCACATCCCCCTTTCGCCAGCT
GGCGTAATAGCGAAGAGGCCCGCACCGATCGCCCTTCCCAACAGTTGCGCAGCCTGAAAAACCGCGCCATGGTGT
GTAGGCTGGAGCTGCTTCGAAGTTCCTATACTTTCTAGAGAATAGGAACTTCGGAATAGGAACTTCAAGATCCCC
CACGCTGCCGCAAGCACTCAGGGCGCAAGGGCTGCTAAAGGAAACGGAACACGTAGAAAGCCAGTCCGCAGAAAC
GGTGCTGACCCCGGATGAATGTCAGCTACTGGGCTATCTGGACAAGGGAAAACGCAAGCGCAAAGAGAAAGCAGG
TAGCTTGCAGTGGGCTTACATGGCGATAGCTAGACTGGGCGGTTTTATGGACAGCAAGCGAACCGGAATTGCCAG
CTGGGGCGCCCTCTGGTAAGGTTGGGAAGCCCTGCAAAGTAAACTGGATGGCTTTCTTGCCGCCAAGGATCTGAT
GGCGCAGGGGATCAAGATCTGATCAAGAGACAGGATGAGGATCGTTTCGCATGATTGAACAAGATGGATTGCACG
CAGGTTCTCCGGCCGCTTGGGTGGAGAGGCTATTCGGCTATGACTGGGCACAACAGACAATCGGCTGCTCTGATG
CCGCCGTGTTCCGGCTGTCAGCGCAGGGGCGCCCGGTTCTTTTTGTCAAGACCGACCTGTCCGGTGCCCTGAATG
AACTGCAGGACGAGGCAGCGCGGCTATCGTGGCTGGCCACGACGGGCGTTCCTTGCGCAGCTGTGCTCGACGTTG
TCACTGAAGCGGGAAGGGACTGGCTGCTATTGGGCGAAGTGCCGGGGCAGGATCTCCTGTCATCTCACCTTGCTC
CTGCCGAGAAAGTATCCATCATGGCTGATGCAATGCGGCGGCTGCATACGCTTGATCCGGCTACCTGCCCATTCG
ACCACCAAGCGAAACATCGCATCGAGCGAGCACGTACTCGGATGGAAGCCGGTCTTGTCGATCAGGATGATCTGG
ACGAAGAGCATCAGGGGCTCGCGCCAGCCGAACTGTTCGCCAGGCTCAAGGCGCGCATGCCCGACGGCGAGGATC
TCGTCGTGACCCATGGCGATGCCTGCTTGCCGAATATCATGGTGGAAAATGGCCGCTTTTCTGGATTCATCGACT
GTGGCCGGCTGGGTGTGGCGGACCGCTATCAGGACATAGCGTTGGCTACCCGTGATATTGCTGAAGAGCTTGGCG
GCGAGTGGGCTGACCGCTTCCTCGTGCTTTACGGTATCGCCGCTCCCGATTCGCAGCGCATCGCCTTCTATCGCC
TTCTTGACGAGTTCTTCTGAGCGGGACTCTGGGGTTCGAAATGACCGACCAAGCGACGCCCAACCTGCCATCACG
AGATTTCGATTCCACCGCCGCCTTCTATGAAAGGTTGGGCTTCGGAATCGTTTTCCGGGACGCCGGCTGGATGAT
CCTCCAGCGCGGGGATCTCATGCTGGAGTTCTTCGCCCACCCCAGCTTCAAAAGCGCTCTGAAGTTCCTATACTT
TCTAGAGAATAGGAACTTCGGAATAGGAACTAAGGAGGATATTCATATGGACCATGGCGCGGCATGCAAGCTCGG
TATCATTGCAGCACTGGGGCCAGATGGTAAGCCCTCCCGTATCGTAGTTATCTACACGACGGGGAGTCAGGCAAC
TATGGATGAACGAAATAGACAGATCGCTGAGATAGGTGCCTCACTGATTAAGCATTGGTAACTGTCAGACCAAGT
TTACTCATATATACTTTAGATTGATTTAAAACTTCATTTTTAATTTAAAAGGATCTAGGTGAAGATCCTTTTTGA
TAATCTCATGACCAAAATCCCTTAACGTGAGTTTTCGTTCCACTGAGCGTCAGACCCCGTAGAAAAGATCAAAGG
ATCTTCTTGAGATCCTTTTTTTCTGCGCGTAATCTGCTGCTTGCAAACAAAAAAACCACCGCTACCAGCGGTGGT
TTGTTTGCCGGATCAAGAGCTACCAACTCTTTTTCCGAAGGTAACTGGCTTCAGCAGAGCGCAGATACCAAATAC
TGTTCTTCTAGTGTAGCCGTAGTTAGGCCACCACTTCAAGAACTCTGTAGCACCGCCTACATACCTCGCTCTGCT
AATCCTGTTACCAGTGGCTGCTGCCAGTGGCGATAAGTCGTGTCTTACCGGGTTGGACTCAAGACGATAGTTACC
GGATAAGGCGCAGCGGTCGGGCTGAACGGGGGGTTCGTGCACACAGCCCAGCTTGGAGCGAACGACCTACACCGA
ACTGAGATACCTACAGCGTGAGCTATGAGAAAGCGCCACGCTTCCCGAAGGGAGAAAGGCGGACAGGTATCCGGT
AAGCGGCAGGGTCGGAACAGGAGAGCGCACGAGGGAGCTTCCAGGGGGAAACGCCTGGTATCTTTATAGTCCTGT
CGGGTTTCGCCACCTCTGACTTGAGCGTCGATTTTTGTGATGCTCGTCAGGGGGGCGGAGCCTATGGAAAAACGC
CAGCAACGCGGCCTTTTTACGGTTCCTGGCCTTTTGCTGGCCTTTTGCTCACATGTTCTTTCCTGCGTTATCCCC
TGATTCTGTGGATAACCGTATTACCGCCTTTGAGTGAGCTGATACCGCTCGCCGCAGCCGAACGACCGAGCGCAG
CGAGTCAGTGAGCGAGGAAGCGGAAGAGCGCCCAATACGCAAACCGCCTCTCCCCGCGCGTTGGCCGATTCATTA
ATGCAGCTGGCACGACAGTATCGATAAGCTTGATAAGCTTTTAAATCAGCAGGGGTCTTTTTGGCTTGTGTATTA
TTTTGAAGTTTTTCTTCCCCGACAGAATCTGCTTTTACCGTCATAGTGAAATGAGCCTGAAAAGCTATTACCATG
ATGATACAAATAAGTTTACTTTTCATTTCACCGCTCCTTTTTAATTCGTAAAACTAAGTTTAAGCCACCTACAAC
TAATCTGACAGAGAGAGTTAAGGACACGTTTTTTAGTATATGTGGGAACTAAATTATACGTTTTGCAGTAGAAAC
TATAGGTGGCTTAAACTTTGGGATATGCTTATATTATATGGATAAACAGTCAGATATTCTTTTACATTTGTTAAT
TCTTCTAAAAAAATTAAAAAATAAGCCTGTTTCTACATTCTTCACAAAATAATTTACGAAGAGTGCAAAACAAGC
TTATTTTTTCGTGTGTGTTAAGCGGTTTTATTCTTAATTTTTTATTACTTTTACAATTATTCGATTGGATTATCT
ACTTTATTACTATATTTCGGATAAAGCGTGGTGCCCCAGATGGAGATATTTCTATTTTTCACAAGTGGTAAGTTC
CGGTCATCAATTACCGTTCTCCACCATTCCCAAGCTAAACCAGTGCATTCTTTAGCGTAAACATTAATATTTCTC
GCGTTACCTGGCAAATAGATGGACGATGTGAAATGAGCTAGCTTGCTTTTATTGTTTTCGCTCCAGTTTTTATGT
TGAACAATTTCGTTACCTTCAGGATCATAATTTACTTCATCCCAAGAAATGTTGAATTGAGCAACGTATCCTCCA
GAGTGATCGATGTTAATTTTTCCATCTGTATAAGCTTTTGAAGTTGTTTCAATATATTCTGAGTTGTTTTTAATA
ACAGCTAATTCATTGTCTTTTAGGAAGTTTGTTGTATAAGCAATGGGAACTCCTGGTGTTTCTCGATTAAAAGTA
GCGCCTTTTTTCAAAATATCGCGTAAGTCTCCGAGGTTGCCGTCGATGATTTGAACTTCATCTTTTGCGGAACCT
CCGTAAATTACGGCTTTGAAGGAAGAATTTTTGATGATATTTGTTAGTTCTACATCACCTGAGACAGATTTTCCG
CTTACGGCAGCATCAAAAGCAGCTTTTACTTTAGTACTATGGGAATTAGTTGATAATTTCAAATAAACTTGACGG
CCATACGCCACACTTGAGATATATGCAGGAGGATTTTCTGCATTCACTCCAAGCGCTTGCAACTGCTCTTTAGTA
ACAGCTTTGCCGAAAAATCTGGAAGGTCTTGTAGGTTCATTAACATTCACGTTATAGTAAATTTGTTTAAAACTA
ATGACTTCTTCTTGCATTTTCCCTTCACTGATTGCGCCGAAGTTTACATTCAAGCTATTATTTACAGCTTTAAAT
GCTGTACCAAATTTCGCAATTAATTGTGATTCACTGTAAGCCATTTCGTCATCATAATCAATTTTTGCACTTACA
TTTGGATAAGCTTGAGCATATTTTTCATTCCATCTTTCCACTAATGTATTTACTGCGTTGTTAACGTTTGATTTA
GTGGCATTTTTTACAACGATTTTATTGTCTTGATTAGTCATACCTGGCAAATCAATGCTGAGTGTTAATGAATCA
CGTTTTACAGGGAGAACATCTGGTTGATTTTCTACTAATTCCGAATTCGCTTTTACGAGAGCACCTGGATAGGTT
AGGCTCGAAATTGCATTCACAACTTGAATGTCTGCATTATTTTGATTGATGGATTTCTTCTTTTTCTCCACAACA
ATATATTCATTTCCATCTTTGTAACCTTTTCTTGGCGGCACATTTGTCACTGCATCTCCGTGGTATACTAATACA
TTGTTTTTATTGTAATCCAATCCTTGTATATACTTATCGATTTCATCCGCGTGTTTCTTTTCGATTGGCGTCTTA
GGACTTGCAGGCGGAGATGCTGGTGGTGCCATGGATGAAATTGAATTTTCTTTATTGAATGCAGATGCATCCTTT
GCTTCAGTTTGTTGCGCAATTGGTAGACTAACTAATATAAGTGTAATAAAAACTAGCATTATTTTTTTCATGGGT
TTCACTCTCCTTCTACATTTTTTAACCTAATAATGCCAAATACCGTTTGCCACCCCTCTCTTTTGATAATTATAA
TATTGGCGAAATTCGCTTCTAAAGATGAAACGCAATATTATATGCTTGCTTTATAGCTTTATTCTAGTCCTGCTG
TCCCTTTATCGTCGTTAACAAATGTTAATGCCTCAACATAAAAGTCACTTTAAGATAGGAATATACTAATCAAAG
GAGGGATCGAATTCCTGCAGTCATCAAGGCAACCATCAGGATTAATGCGGATATTGCGGAGTAACACTTCAGACT
GAAAGTAGAAATAAAAACCGCAGCAGACAACTGACAACATCAAATGAAGGGGGCTTATTCTAATTGATATTATTT
ATATGATAATAGTTCATTTTGTATTTTGTTTTTTTGATATTCTCACCTGCTTAGTTACAATAAATCAATTCTATC
GCTGTATGGTATAGACTGTTTTATTATATATTTTGAATATTTTTAATCTGCCCAGTCTGGTTTTTTAAAAAAGTG
CTATCCTCTTAATGTCTTTACTAAATTAGAAAACAAGTTTCACTTTCAACTATTGCATCTTTAATTAATGGTCAA
GGTGATTTCAAATGCTCGTTTGTGGCCAGTTATACCTCAAATAACTCAAGTTGTTGAGCACAGCCAACGCACATG
CAGTTTGACGTATGACAGGTATGCTTTATTTCATTTAAATTATGATGGTTTTCCAGCCAATCAGTGAGTTTCTCT
TGATAAGGAATGCGGGAATGTCTATGTATTTTAATAAAATAATTTCATTTAATATTATTTCACGAATAGTTATTT
GTATCTTTTTGATATGTGGAATGTTCATGGCTGGGGCTTCAGAAAAATATGATGCTAACGCACCGCAACAGGTCC
AGCCTTATTCTGTCTCTTCATCTGCATTTGAAAATCTCCATCCTAATAATGAAATGGAGAGTTCAATCAATCCCT
TTTCCGCATCGGATACAGAAAGAAATGCTGCAATAATAGATCGCGCCAATAAGGAGCAGGAGACTGAAGCGGTGA
ATAAGATGATAAGCACCGGGGCCAGGTTAGCTGCATCAGGCAGGGCATCTGATGTTGCTCACTCAATGGTGGGCG
ATGCGGTTAATCAAGAAATCAAACAGTGGTTAAATCGATTCGGTACGGCTCAAGTTAATCTGAATTTTGACAAAA
ATTTTTCGCTAAAAGAAAGCTCTCTTGATTGGCTGGCTCCTTGGTATGACTCTGCTTCATTCCTCTTTTTTAGTC
AGTTAGGTATTCGCAATAAAGACAGCCGCAACACACTTAACCTTGGCGTCGGGATACGTACATTGGAGAACGGTT
GGCTGTACGGACTTAATACTTTTTATGATAATGATTTGACCGGCCACAACCACCGTATCGGTCTTGGTGCCGAGG
CCTGGACCGATTATTTACAGTTGGCTGCCAATGGGTATTTTCGCCTCAATGGATGGCACTCGTCGCGTGATTTCT
CCGACTATAAAGAGCGCCCAGCCACTGGGGGGGATTTGCGCGCGAATGCTTATTTACCTGCACTCCCACAACTGG
GGGGGAAGTTGATGTATGAGCAATACACCGGTGAGCGTGTTGCTTTATTTGGTAAAGATAATCTGCAACGCAACC
CTTATGCCGTGACTGCCGGGATCAATTACACCCCCGTGCCTCTACTCACTGTCGGGGTAGATCAGCGTATGGGGA
AAAGCAGTAAGCATGAAACACAGTGGAACCTCCAAATGAACTATCGCCTGGGCGAGAGTTTTCAGTCGCAACTTA
GCCCTTCAGCGGTGGCAGGAACACGTCTACTGGCGGAGAGCCGCTATAACCTTGTCGATCGTAACAATAATATCG
TGTTGGAGTATCAGAAACAGCAGGTGGTTAAACTGACATTATCGCCAGCAACTATCTCCGGCCTGCCGGGTCAGG
TTTATCAGGTGAACGCACAAGTACAAGGGGCATCTGCTGTAAGGGAAATTGTCTGGAGTGATGCCGAACTGATTG
CCGCTGGCGGCACATTAACACCACTGAGTACCACACAATTCAACTTGGTTTTACCGCCTTATAAACGCACAGCAC
AAGTGAGTCGGGTAACGGACGACCTGACAGCCAACTTTTATTCGCTTAGTGCGCTCGCGGTTGATCACCAAGGAA
ACCGATCTAACTCATTCACATTGAGCGTCACCGTTCAGCAGCCTCAGTTGACATTAACGGCGGCCGTCATTGGTG
ATGGCGCACCGGCTAATGGGAAAACTGCAATCACCGTTGAGTTCACCGTTGCTGATTTTGAGGGGAAACCCTTAG
CCGGGCAGGAGGTGGTGATAACCACCAATAATGGTGCGCTACCGAATAAAATCACGGAAAAGACAGATGCAAATG
GCGTCGCGCGCATTGCATTAACCAATACGACAGATGGCGTGACGGTAGTCACAGCAGAAGTGGAGGGGCAACGGC
AAAGTGTTGATACCCACTTTGTTAAGGGTACTATCGCGGCGGATAAATCCACTCTGGCTGCGGTACCGACATCTA
TCATCGCTGATGGTCTAATGGCTTCAACCATCACGTTGGAGTTGAAGGATACCTATGGGGACCCGCAGGCTGGCG
CGAATGTGGCTTTTGACACAACCTTAGGCAATATGGGCGTTATCACGGATCACAATGACGGCACTTATAGCGCAC
CATTGACCAGTACCACGTTGGGGGTAGCAACAGTAACGGTGAAAGTGGATGGGGCTGCGTTCAGTGTGCCGAGTG
TGACGGTTAATTTCACGGCAGATCCTATTCCAGATGCTGGCCGCTCCAGTTTCACCGTCTCCACACCGGATATCT
TGGCTGATGGCACGATGAGTTCCACATTATCCTTTGTCCCTGTCGATAAGAATGGCCATTTTATCAGTGGGATGC
AGGGCTTGAGTTTTACTCAAAACGGTGTGCCGGTGAGTATTAGCCCCATTACCGAGCAGCCAGATAGCTATACCG
CGACGGTGGTTGGGAATAGTGTCGGTGATGTCACAATCACGCCGCAGGTTGATACCCTGATACTGAGTACATTGC
AGAAAAAAATATCCCTATTCCCGGTACCTACGCTGACCGGTATTCTGGTTAACGGGCAAAATTTCGCTACGGATA
AAGGGTTCCCGAAAACGATCTTTAAAAACGCCACATTCCAGTTACAGATGGATAACGATGTTGCTAATAATACTC
AGTATGAGTGGTCGTCGTCATTCACACCCAATGTATCGGTTAACGATCAGGGTCAGGTGACGATTACCTACCAAA
CCTATAGCGAAGTGGCTGTGACGGCGAAAAGTAAAAAATTCCCAAGTTATTCGGTGAGTTATCGGTTCTACCCAA
ATCGGTGGATATACGATGGCGGCAGATCGCTGGTATCCAGTCTCGAGGCCAGCAGACAATGCCAAGGTTCAGATA
TGTCTGCGGTTCTTGAATCCTCACGTGCAACCAACGGAACGCGTGCGCCTGACGGGACATTGTGGGGCGAGTGGG
GGAGCTTGACCGCGTATAGTTCTGATTGGCAATCTGGTGAATATTGGGTCAAAAAGACCAGCACGGATTTTGAAA
CCATGAATATGGACACAGGCGCACTGCAACCAGGGCCTGCATACTTGGCGTTCCCGCTCTGTGCGCTGTCAATAT
AACCAGATAACAGATAGCAATAAGAACAGTTTAATGAGCTGATTATTTGGGGCGCGAATGGGAGTCCGGCAATCC
TAGACTCGCCCCATAAGTAGCAAACGTCCAGAGAACAACGCCGCTCAGGTTAATTGAGCGGCGTTGTTTTTTTAA
AAGGATTTGTCGCGATAAGCGTGAGCTGGCGTTAAATGCCGATCTTACGGCCCAGCTGCAGCCCGGCTAGTAACG
GCCGCCAGTGTGCTGGAATTCGCCCTTAATCGGCATCATTCACCAAGCTTGCCAGGCGACTGTCTTCAATATTAC
AGCCGCAACTACTGACATGGCGGGTGATGGTGTTCACTATTCCAGGGCGATCGGCACCCAACGCAGTGATCACCA
GATAATGTTGCGATGACAGTGTCAAACTGGTTATTCCTTCAAGGGGTGAGTTGTTCTTAAGCATGCCGGTTTGCT
GTAAAGTTTAGGGAGATTTGATGGCTTACTCTGTTCAAAAGTCGCGCCTGGCAAAGGTTGCGGGTGTTTCGCTTG
TTTTATTACTCGCTGCCTGTAGTTCTGACTCACGCTATAAGCGTCAGGTCAGTGGTGATGAAGCCTACCTGGAAG
CGCCATGGCATGCAAGGGCGAATTCTGCAGATATCCATCACACTGGCGGCCCTAGACCAGGCTTTACACTTTATG
CTTCCGGCTCGTATAATGTGTGGAAGGATCCAGGAGTAACAATACAAATGGATTCAAGAGATCCATTTGTATTGT
TACTCCTTTGTCGACTGGACAGTTCAAGAGACTGTCCATCAATATCAGCTTTGTCACAAACCCCGCCACCGGCGG
GGTTTTTTTCTGCTCTAGGGCCGCTCGAGCATGCATCTAGAGGGCCCAATTCGCCCTATAGTGAGTCGTATTACA
ATTCACTGGCCGTCGTTTTACAACGTCGTGACTGGGAAAACCCTGGCGTTACCCAACTTAATCGCCTTGCAGCAC
ATCCCCCTTTCGCCAGCTGGCGTAATAGCGAAGAGGCCCGCACCGATCGCCCTTCCCAACAGTTGCGCAGCCTGA
AAAACCGCGCCATGGTGTGTAGGCTGGAGCTGCTTCGAAGTTCCTATACTTTCTAGAGAATAGGAACTTCGGAAT
AGGAACTTCAAGATCCCCCACGCTGCCGCAAGCACTCAGGGCGCAAGGGCTGCTAAAGGAAACGGAACACGTAGA
AAGCCAGTCCGCAGAAACGGTGCTGACCCCGGATGAATGTCAGCTACTGGGCTATCTGGACAAGGGAAAACGCAA
GCGCAAAGAGAAAGCAGGTAGCTTGCAGTGGGCTTACATGGCGATAGCTAGACTGGGCGGTTTTATGGACAGCAA
GCGAACCGGAATTGCCAGCTGGGGCGCCCTCTGGTAAGGTTGGGAAGCCCTGCAAAGTAAACTGGATGGCTTTCT
TGCCGCCAAGGATCTGATGGCGCAGGGGATCAAGATCTGATCAAGAGACAGGATGAGGATCGTTTCGCATGATTG
AACAAGATGGATTGCACGCAGGTTCTCCGGCCGCTTGGGTGGAGAGGCTATTCGGCTATGACTGGGCACAACAGA
CAATCGGCTGCTCTGATGCCGCCGTGTTCCGGCTGTCAGCGCAGGGGCGCCCGGTTCTTTTTGTCAAGACCGACC
TGTCCGGTGCCCTGAATGAACTGCAGGACGAGGCAGCGCGGCTATCGTGGCTGGCCACGACGGGCGTTCCTTGCG
CAGCTGTGCTCGACGTTGTCACTGAAGCGGGAAGGGACTGGCTGCTATTGGGCGAAGTGCCGGGGCAGGATCTCC
TGTCATCTCACCTTGCTCCTGCCGAGAAAGTATCCATCATGGCTGATGCAATGCGGCGGCTGCATACGCTTGATC
CGGCTACCTGCCCATTCGACCACCAAGCGAAACATCGCATCGAGCGAGCACGTACTCGGATGGAAGCCGGTCTTG
TCGATCAGGATGATCTGGACGAAGAGCATCAGGGGCTCGCGCCAGCCGAACTGTTCGCCAGGCTCAAGGCGCGCA
TGCCCGACGGCGAGGATCTCGTCGTGACCCATGGCGATGCCTGCTTGCCGAATATCATGGTGGAAAATGGCCGCT
TTTCTGGATTCATCGACTGTGGCCGGCTGGGTGTGGCGGACCGCTATCAGGACATAGCGTTGGCTACCCGTGATA
TTGCTGAAGAGCTTGGCGGCGAGTGGGCTGACCGCTTCCTCGTGCTTTACGGTATCGCCGCTCCCGATTCGCAGC
GCATCGCCTTCTATCGCCTTCTTGACGAGTTCTTCTGAGCGGGACTCTGGGGTTCGAAATGACCGACCAAGCGAC
GCCCAACCTGCCATCACGAGATTTCGATTCCACCGCCGCCTTCTATGAAAGGTTGGGCTTCGGAATCGTTTTCCG
GGACGCCGGCTGGATGATCCTCCAGCGCGGGGATCTCATGCTGGAGTTCTTCGCCCACCCCAGCTTCAAAAGCGC
TCTGAAGTTCCTATACTTTCTAGAGAATAGGAACTTCGGAATAGGAACTAAGGAGGATATTCATATGGACCATGG
CGCGGCATGCAAGCTCGGTATCATTGCAGCACTGGGGCCAGATGGTAAGCCCTCCCGTATCGTAGTTATCTACAC
GACGGGGAGTCAGGCAACTATGGATGAACGAAATAGACAGATCGCTGAGATAGGTGCCTCACTGATTAAGCATTG
GTAACTGTCAGACCAAGTTTACTCATATATACTTTAGATTGATTTAAAACTTCATTTTTAATTTAAAAGGATCTA
GGTGAAGATCCTTTTTGATAATCTCATGACCAAAATCCCTTAACGTGAGTTTTCGTTCCACTGAGCGTCAGACCC
CGTAGAAAAGATCAAAGGATCTTCTTGAGATCCTTTTTTTCTGCGCGTAATCTGCTGCTTGCAAACAAAAAAACC
ACCGCTACCAGCGGTGGTTTGTTTGCCGGATCAAGAGCTACCAACTCTTTTTCCGAAGGTAACTGGCTTCAGCAG
AGCGCAGATACCAAATACTGTTCTTCTAGTGTAGCCGTAGTTAGGCCACCACTTCAAGAACTCTGTAGCACCGCC
TACATACCTCGCTCTGCTAATCCTGTTACCAGTGGCTGCTGCCAGTGGCGATAAGTCGTGTCTTACCGGGTTGGA
CTCAAGACGATAGTTACCGGATAAGGCGCAGCGGTCGGGCTGAACGGGGGGTTCGTGCACACAGCCCAGCTTGGA
GCGAACGACCTACACCGAACTGAGATACCTACAGCGTGAGCTATGAGAAAGCGCCACGCTTCCCGAAGGGAGAAA
GGCGGACAGGTATCCGGTAAGCGGCAGGGTCGGAACAGGAGAGCGCACGAGGGAGCTTCCAGGGGGAAACGCCTG
GTATCTTTATAGTCCTGTCGGGTTTCGCCACCTCTGACTTGAGCGTCGATTTTTGTGATGCTCGTCAGGGGGGCG
GAGCCTATGGAAAAACGCCAGCAACGCGGCCTTTTTACGGTTCCTGGCCTTTTGCTGGCCTTTTGCTCACATGTT
CTTTCCTGCGTTATCCCCTGATTCTGTGGATAACCGTATTACCGCCTTTGAGTGAGCTGATACCGCTCGCCGCAG
CCGAACGACCGAGCGCAGCGAGTCAGTGAGCGAGGAAGCGGAAGAGCGCCCAATACGCAAACCGCCTCTCCCCGC
GCGTTGGCCGATTCATTAATGCAGCTGGCACGACAGTATCGATAAGCTTGATAAGCTTTTAAATCAGCAGGGGTC
TTTTTGGCTTGTGTATTATTTTGAAGTTTTTCTTCCCCGACAGAATCTGCTTTTACCGTCATAGTGAAATGAGCC
TGAAAAGCTATTACCATGATGATACAAATAAGTTTACTTTTCATTTCACCGCTCCTTTTTAATTCGTAAAACTAA
GTTTAAGCCACCTACAACTAATCTGACAGAGAGAGTTAAGGACACGTTTTTTAGTATATGTGGGAACTAAATTAT
ACGTTTTGCAGTAGAAACTATAGGTGGCTTAAACTTTGGGATATGCTTATATTATATGGATAAACAGTCAGATAT
TCTTTTACATTTGTTAATTCTTCTAAAAAAATTAAAAAATAAGCCTGTTTCTACATTCTTCACAAAATAATTTAC
GAAGAGTGCAAAACAAGCTTATTTTTTCGTGTGTGTTAAGCGGTTTTATTCTTAATTTTTTATTACTTTTACAAT
TATTCGATTGGATTATCTACTTTATTACTATATTTCGGATAAAGCGTGGTGCCCCAGATGGAGATATTTCTATTT
TTCACAAGTGGTAAGTTCCGGTCATCAATTACCGTTCTCCACCATTCCCAAGCTAAACCAGTGCATTCTTTAGCG
TAAACATTAATATTTCTCGCGTTACCTGGCAAATAGATGGACGATGTGAAATGAGCTAGCTTGCTTTTATTGTTT
TCGCTCCAGTTTTTATGTTGAACAATTTCGTTACCTTCAGGATCATAATTTACTTCATCCCAAGAAATGTTGAAT
TGAGCAACGTATCCTCCAGAGTGATCGATGTTAATTTTTCCATCTGTATAAGCTTTTGAAGTTGTTTCAATATAT
TCTGAGTTGTTTTTAATAACAGCTAATTCATTGTCTTTTAGGAAGTTTGTTGTATAAGCAATGGGAACTCCTGGT
GTTTCTCGATTAAAAGTAGCGCCTTTTTTCAAAATATCGCGTAAGTCTCCGAGGTTGCCGTCGATGATTTGAACT
TCATCTTTTGCGGAACCTCCGTAAATTACGGCTTTGAAGGAAGAATTTTTGATGATATTTGTTAGTTCTACATCA
CCTGAGACAGATTTTCCGCTTACGGCAGCATCAAAAGCAGCTTTTACTTTAGTACTATGGGAATTAGTTGATAAT
TTCAAATAAACTTGACGGCCATACGCCACACTTGAGATATATGCAGGAGGATTTTCTGCATTCACTCCAAGCGCT
TGCAACTGCTCTTTAGTAACAGCTTTGCCGAAAAATCTGGAAGGTCTTGTAGGTTCATTAACATTCACGTTATAG
TAAATTTGTTTAAAACTAATGACTTCTTCTTGCATTTTCCCTTCACTGATTGCGCCGAAGTTTACATTCAAGCTA
TTATTTACAGCTTTAAATGCTGTACCAAATTTCGCAATTAATTGTGATTCACTGTAAGCCATTTCGTCATCATAA
TCAATTTTTGCACTTACATTTGGATAAGCTTGAGCATATTTTTCATTCCATCTTTCCACTAATGTATTTACTGCG
TTGTTAACGTTTGATTTAGTGGCATTTTTTACAACGATTTTATTGTCTTGATTAGTCATACCTGGCAAATCAATG
CTGAGTGTTAATGAATCACGTTTTACAGGGAGAACATCTGGTTGATTTTCTACTAATTCCGAATTCGCTTTTACG
AGAGCACCTGGATAGGTTAGGCTCGAAATTGCATTCACAACTTGAATGTCTGCATTATTTTGATTGATGGATTTC
TTCTTTTTCTCCACAACAATATATTCATTTCCATCTTTGTAACCTTTTCTTGGCGGCACATTTGTCACTGCATCT
CCGTGGTATACTAATACATTGTTTTTATTGTAATCCAATCCTTGTATATACTTATCGATTTCATCCGCGTGTTTC
TTTTCGATTGGCGTCTTAGGACTTGCAGGCGGAGATGCTGGTGGTGCCATGGATGAAATTGAATTTTCTTTATTG
AATGCAGATGCATCCTTTGCTTCAGTTTGTTGCGCAATTGGTAGACTAACTAATATAAGTGTAATAAAAACTAGC
ATTATTTTTTTCATGGGTTTCACTCTCCTTCTACATTTTTTAACCTAATAATGCCAAATACCGTTTGCCACCCCT
CTCTTTTGATAATTATAATATTGGCGAAATTCGCTTCTAAAGATGAAACGCAATATTATATGCTTGCTTTATAGC
TTTATTCTAGTCCTGCTGTCCCTTTATCGTCGTTAACAAATGTTAATGCCTCAACATAAAAGTCACTTTAAGATA
GGAATATACTAATCAAAGGAGGGATCGAATTCCTGCAGTCATCAAGGCAACCATCAGGATTAATGCGGATATTGC
GGAGTAACACTTCAGACTGAAAGTAGAAATAAAAACCGCAGCAGACAACTGACAACATCAAATGAAGGGGGCTTA
TTCTAATTGATATTATTTATATGATAATAGTTCATTTTGTATTTTGTTTTTTTGATATTCTCACCTGCTTAGTTA
CAATAAATCAATTCTATCGCTGTATGGTATAGACTGTTTTATTATATATTTTGAATATTTTTAATCTGCCCAGTC
TGGTTTTTTAAAAAAGTGCTATCCTCTTAATGTCTTTACTAAATTAGAAAACAAGTTTCACTTTCAACTATTGCA
TCTTTAATTAATGGTCAAGGTGATTTCAAATGCTCGTTTGTGGCCAGTTATACCTCAAATAACTCAAGTTGTTGA
GCACAGCCAACGCACATGCAGTTTGACGTATGACAGGTATGCTTTATTTCATTTAAATTATGATGGTTTTCCAGC
CAATCAGTGAGTTTCTCTTGATAAGGAATGCGGGAATGTCTATGTATTTTAATAAAATAATTTCATTTAATATTA
TTTCACGAATAGTTATTTGTATCTTTTTGATATGTGGAATGTTCATGGCTGGGGCTTCAGAAAAATATGATGCTA
ACGCACCGCAACAGGTCCAGCCTTATTCTGTCTCTTCATCTGCATTTGAAAATCTCCATCCTAATAATGAAATGG
AGAGTTCAATCAATCCCTTTTCCGCATCGGATACAGAAAGAAATGCTGCAATAATAGATCGCGCCAATAAGGAGC
AGGAGACTGAAGCGGTGAATAAGATGATAAGCACCGGGGCCAGGTTAGCTGCATCAGGCAGGGCATCTGATGTTG
CTCACTCAATGGTGGGCGATGCGGTTAATCAAGAAATCAAACAGTGGTTAAATCGATTCGGTACGGCTCAAGTTA
ATCTGAATTTTGACAAAAATTTTTCGCTAAAAGAAAGCTCTCTTGATTGGCTGGCTCCTTGGTATGACTCTGCTT
CATTCCTCTTTTTTAGTCAGTTAGGTATTCGCAATAAAGACAGCCGCAACACACTTAACCTTGGCGTCGGGATAC
GTACATTGGAGAACGGTTGGCTGTACGGACTTAATACTTTTTATGATAATGATTTGACCGGCCACAACCACCGTA
TCGGTCTTGGTGCCGAGGCCTGGACCGATTATTTACAGTTGGCTGCCAATGGGTATTTTCGCCTCAATGGATGGC
ACTCGTCGCGTGATTTCTCCGACTATAAAGAGCGCCCAGCCACTGGGGGGGATTTGCGCGCGAATGCTTATTTAC
CTGCACTCCCACAACTGGGGGGGAAGTTGATGTATGAGCAATACACCGGTGAGCGTGTTGCTTTATTTGGTAAAG
ATAATCTGCAACGCAACCCTTATGCCGTGACTGCCGGGATCAATTACACCCCCGTGCCTCTACTCACTGTCGGGG
TAGATCAGCGTATGGGGAAAAGCAGTAAGCATGAAACACAGTGGAACCTCCAAATGAACTATCGCCTGGGCGAGA
GTTTTCAGTCGCAACTTAGCCCTTCAGCGGTGGCAGGAACACGTCTACTGGCGGAGAGCCGCTATAACCTTGTCG
ATCGTAACAATAATATCGTGTTGGAGTATCAGAAACAGCAGGTGGTTAAACTGACATTATCGCCAGCAACTATCT
CCGGCCTGCCGGGTCAGGTTTATCAGGTGAACGCACAAGTACAAGGGGCATCTGCTGTAAGGGAAATTGTCTGGA
GTGATGCCGAACTGATTGCCGCTGGCGGCACATTAACACCACTGAGTACCACACAATTCAACTTGGTTTTACCGC
CTTATAAACGCACAGCACAAGTGAGTCGGGTAACGGACGACCTGACAGCCAACTTTTATTCGCTTAGTGCGCTCG
CGGTTGATCACCAAGGAAACCGATCTAACTCATTCACATTGAGCGTCACCGTTCAGCAGCCTCAGTTGACATTAA
CGGCGGCCGTCATTGGTGATGGCGCACCGGCTAATGGGAAAACTGCAATCACCGTTGAGTTCACCGTTGCTGATT
TTGAGGGGAAACCCTTAGCCGGGCAGGAGGTGGTGATAACCACCAATAATGGTGCGCTACCGAATAAAATCACGG
AAAAGACAGATGCAAATGGCGTCGCGCGCATTGCATTAACCAATACGACAGATGGCGTGACGGTAGTCACAGCAG
AAGTGGAGGGGCAACGGCAAAGTGTTGATACCCACTTTGTTAAGGGTACTATCGCGGCGGATAAATCCACTCTGG
CTGCGGTACCGACATCTATCATCGCTGATGGTCTAATGGCTTCAACCATCACGTTGGAGTTGAAGGATACCTATG
GGGACCCGCAGGCTGGCGCGAATGTGGCTTTTGACACAACCTTAGGCAATATGGGCGTTATCACGGATCACAATG
ACGGCACTTATAGCGCACCATTGACCAGTACCACGTTGGGGGTAGCAACAGTAACGGTGAAAGTGGATGGGGCTG
CGTTCAGTGTGCCGAGTGTGACGGTTAATTTCACGGCAGATCCTATTCCAGATGCTGGCCGCTCCAGTTTCACCG
TCTCCACACCGGATATCTTGGCTGATGGCACGATGAGTTCCACATTATCCTTTGTCCCTGTCGATAAGAATGGCC
ATTTTATCAGTGGGATGCAGGGCTTGAGTTTTACTCAAAACGGTGTGCCGGTGAGTATTAGCCCCATTACCGAGC
AGCCAGATAGCTATACCGCGACGGTGGTTGGGAATAGTGTCGGTGATGTCACAATCACGCCGCAGGTTGATACCC
TGATACTGAGTACATTGCAGAAAAAAATATCCCTATTCCCGGTACCTACGCTGACCGGTATTCTGGTTAACGGGC
AAAATTTCGCTACGGATAAAGGGTTCCCGAAAACGATCTTTAAAAACGCCACATTCCAGTTACAGATGGATAACG
ATGTTGCTAATAATACTCAGTATGAGTGGTCGTCGTCATTCACACCCAATGTATCGGTTAACGATCAGGGTCAGG
TGACGATTACCTACCAAACCTATAGCGAAGTGGCTGTGACGGCGAAAAGTAAAAAATTCCCAAGTTATTCGGTGA
GTTATCGGTTCTACCCAAATCGGTGGATATACGATGGCGGCAGATCGCTGGTATCCAGTCTCGAGGCCAGCAGAC
AATGCCAAGGTTCAGATATGTCTGCGGTTCTTGAATCCTCACGTGCAACCAACGGAACGCGTGCGCCTGACGGGA
CATTGTGGGGCGAGTGGGGGAGCTTGACCGCGTATAGTTCTGATTGGCAATCTGGTGAATATTGGGTCAAAAAGA
CCAGCACGGATTTTGAAACCATGAATATGGACACAGGCGCACTGCAACCAGGGCCTGCATACTTGGCGTTCCCGC
TCTGTGCGCTGTCAATATAACCAGATAACAGATAGCAATAAGAACAGTTTAATGAGCTGATTATTTGGGGCGCGA
ATGGGAGTCCGGCAATCCTAGACTCGCCCCATAAGTAGCAAACGTCCAGAGAACAACGCCGCTCAGGTTAATTGA
GCGGCGTTGTTTTTTTAAAAGGATTTGTCGCGATAAGCGTGAGCTGGCGTTAAATGCCGATCTTACGGCCCAGCT
GCAGCCCGGCTAGTAACGGCCGCCAGTGTGCTGGAATTCGCCCTTAATCGGCATCATTCACCAAGCTTGCCAGGC
GACTGTCTTCAATATTACAGCCGCAACTACTGACATGGCGGGTGATGGTGTTCACTATTCCAGGGCGATCGGCAC
CCAACGCAGTGATCACCAGATAATGTTGCGATGACAGTGTCAAACTGGTTATTCCTTCAAGGGGTGAGTTGTTCT
TAAGCATGCCGGTTTGCTGTAAAGTTTAGGGAGATTTGATGGCTTACTCTGTTCAAAAGTCGCGCCTGGCAAAGG
TTGCGGGTGTTTCGCTTGTTTTATTACTCGCTGCCTGTAGTTCTGACTCACGCTATAAGCGTCAGGTCAGTGGTG
ATGAAGCCTACCTGGAAGCGCCATGGCATGCAAGGGCGAATTCTGCAGATATCCATCACACTGGCGGCCCTAGAC
CAGGCTTTACACTTTATGCTTCCGGCTCGTATAATGTGTGGAAGGATCCAGGAGTAACAATACAAATGGATTCAA
GAGATCCATTTGTATTGTTACTCCTTTGTCGACTGGACAGTTCAAGAGACTGTCCATCAATATCAGCTTTGTCAC
AAACCCCGCCACCGGCGGGGTTTTTTTCTGCTCTAG

### Example 24

### Deletion of rnc Encoding RNAse III in CEQ200 (CEQ221 Δrnc)

Most bacteria contain a large number of RNA degrading enzymes, RNases, which may degrade the siRNA causing a reduction in the activity of tkRNAi. In such cases where the RNases of a specific bacterium will exhibit such degradation of siRNA, a targeted deletion of the gene encoding the RNase of interest (e.g., the rnc gene encoding RNase III) is performed to yield higher levels of siRNA per tkRNAi bacterium, resulting in more siRNA being delivered to the target cells, as well as more efficient gene silencing of the gene of interest within the target cell.

### Construction of Δrnc

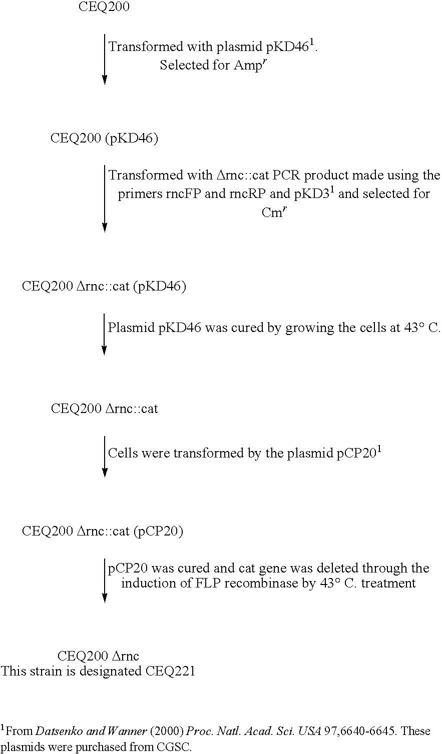

A gel analysis showed confirmed the construction of CEQ221 and Δrnc Genotype verification. Gel analysis confirmed the expression of Proteus 23S rRNA in Δrnc strain and the inability to process 23S dsDNA. Total cellular RNA from CEQ200 and CEQ221 bearing pPM2 was extracted and run on the 1.5% agarose gel. 23S rRNA transcribed from the pPM2 has an intervening sequence in helix 25, which is processed by RNase III during maturation, resulting in a 23 S rRNA fragmentation: 23S′ and 23S″. RNase III deficient strain CEQ221 cannot process such a helix, therefore, a Proteus 23S rRNA appears to be intact. This is a sign that the Δrnc strain CEQ221 has lost RNAse III activity.

Δrnc strain CEQ221 demonstrated increased production of shRNA and increased delivery of higher amounts of shRNA into target cells. When transformed with the same plasmid, pNJSZc-H3, Δrnc bacteria (CEQ221) contain significantly more shRNA compared with wt-rnc bacteria (CEQ200) transformed with the same plasmid. Δrnc bacteria deliver larger amounts of shRNA into cells during an in vitro invasion assay experiment.

Cells treated with CEQ221 (H3-Δrnc) contain higher levels of shRNA compared with cells treated with equal amounts of CEQ200 (H3). SW480 cells were treated with E. coli-Δrnc carrying a tkRNAi plasmid against a gene target (beta-catenin H3) or with E. coli with a wild-type rnc gene carrying the same tkRNAi plasmid (H3) against beta-catenin. Cells were harvested at the indicated time points and cell extracts were analyzed to measure the amount of shRNA that had been deposited into the cells by the carrier bacteria. The Δrnc strain (CEQ221-red columns) was able to deposit a significantly larger amount of shRNA into target cells compared with its wild-type rnc counterpart (blue columns).

FIG. 1 shows increased gene silencing potency (Maximum effect) and efficacy (Ic50). Treatment with CEQ221(Δrnc) achieves significantly higher levels of gene suppression compared with treatment with CEQ200 (wt rnc). As shown in FIG. 1, Cos-7 cells were treated with rising doses of bacteria carrying the plasmids pNJSZc-H3 (or control plasmid pNJSZc-HPVb) and analyzed after 48 h for expression of the target gene beta-catenin. Beta-catenin gene expression (mRNA) levels are shown in relation to cells that had been treated with the control bacteria (containing plasmid pNJSZc-HPVb and producing shRNA against the virus HPV) at the same bacterial dose. Results show that there is a dose-dependent decrease (“knock-down”) of beta-catenin gene expression observed. The potency of the −Δrnc strain (CEQ221) is significantly greater than the one of the wt-rnc strain (CEQ200) with maximum levels of gene silencing of 76% compared to 57%. These results show that the efficacy of CEQ221 is approximately 10-fold greater compared with CEQ200: IC₅₀ for CEQ221 is 10⁶ cfu/ml as compared to IC₅₀ for CEQ200 is 10⁷ cfu/ml.

### Example 25

### Design of RNAse III Substrates as Precursors of Functional shRNA for Use in tkRNAi

In the preceeding example, it was demonstrated that limiting RNase activity within the delivery bacteria is beneficially to protect unwanted processing and degredation of shRNA. In an alternative embodiment, it is beneficial to design a hairpin RNA molecule that permits improved, more accurate and efficient Dicer processing that is essential for effective RNA interference. Dicer is an RNase enzyme having activity specific for dsRNAs, whereby the RNase III cleavage product contains 5′ phosphate and 3′ hydroxyl termini and a 2-nt overhand at the 3′ end. The dicer products are further characterized by a discrete size of approximately 21 nt. Thus, the present example provides a hairpin RNA molecule that provides a substrate for processing by RNase III in the bacterial (tkRNAi) carrier, resulting in a substrate for Dicer processing within the host target cell.

RNase III enzymes can be divided into three classes. Class I enzymes, found in bacteria, bacteriophage and fungi contain a single RNase III domain and a dsRNA binding domain (dsRBD). Class II and III enzymes are characterized by Drosha and Dicer, respectively. Dicer is the most complicated RNase III enzyme that typically contains a DExD/H-box helicase domain, a small domain of unknown function (DUF283), a PAZ (Piwi Argonaute Zwille) domain, two tandem RNase III domains (RNase Ma and Mb), and a dsRBD. Some Dicer or Dicer-like proteins from lower eukaryotes have a simpler domain structure; for example, the Dicer protein from Giardia intestinalis contains only a PAZ and two RNase III domains. Previous mutational and enzymatic studies on Escherichia coli RNase III and human Dicer had led to the “single processing center model” for RNase III cleavage. This model centers on two RNase III domains forming a catalytic dimer: intermolecular homodimer for class I enzymes and intramolecular pseudodimer between RNase IIIa and IIIb domains for Dicer and Drosha. This dimerization creates a single processing center for dsRNA cleavage, with each RNase III domain cleaving one strand of the dsRNA. The distance between the two cleavage sites dictates the generation of the characteristic 2-nt 3′ overhang. For Dicer, the distance between the terminus-binding PAZ domain and the RNase III domains determines the length of the cleavage product (Du, Lee, Tjhen et al in PNAS 105(7) 2008).

Bacteria contain a class I RNase III enzyme that cuts dsRNA. There is evidence that this class I RNase III recognizes specific motifs that determine where the dsRNA will be cleaved. The enzyme performs said cleavage in such a way that leaves a 2 nt 3′ overhang (see Pertzev and Nicholson Nucleic Acid Research vol. 34(13) 2006 and reviewed by Nicholson in FEMS Micro Reviews 23 1999). In addition, sequences have been described that exclude binding and cleavage by RNAse III; so called anti-determinants.

The following example makes use of bacterial Class I RNAse III processing of the hairpin RNA within the tkRNAi bacteria prior to release into the mammalian cytoplasm. The defined proximal and distal box sequences required by bacterial RNAse III were placed “below” a pseudo-tetraloop structure, which is optional as variants of this design may be constructed with and without the loop, and a “spacer” sequence “above” the pseudo-tetraloop to extend the hairpin sequence by ˜21 nucleotides. The proximal/distal box motif will encompass only ˜10 nt, therefore the remaining lint stretch adjacent to the silencing sequence should be composed of all anti-determinant base pairings. Bacterial RNAse III will recognize the distal and proximal box sequences and cut the dsRNA at or 2 nt below the proximal box (FIG. 2) leaving a longer (i.e. more stable) hairpin structure. Furthermore, the presence of anti-determinant base pairings “above” the proximal/distal box motif protects the hairpin from further processing/degradation and maintains the appropriate length of the hairpin such that when Dicer processes the hairpin inside the target cell, a 21 nt silencing siRNA will be produced.

FIG. 2 shows a schematic illustration of the RNase III substrate hairpin RNA structure with functional annotation.

FIG. 3 shows a schematic illustration of the bacterial Class I RNase III cutting action of the hairpin precursor. The cleavage is positively directed to occur at approximately 10 nt distal of the pseudo tetraloop structure to result in an ideal Dicer-substrate precursor. This step will occur within the bacteria before delivery to the target cell. Cleavage by Class I RNase III will result in a hairpin of approximately 100 nucleotides containing a 2-nucleotide overhang at the 3′ end, which directs the next enzymatic processing step (see FIG. 4).

FIG. 4 shows a functional annotation of the second step of maturation (first Dicer-cleavage step). This step occurs after release of the RNA hairpin molecule into the cytoplasm of the target cell. The 2-nucleotide overhang at the 3′ end of the hairpin RNA structure left by the Class I RNAse III processing helps direct and trigger the cleavage of the RNA structure by Dicer 21 nucleotides upstream (cleavage site is indicated by arrows designating “1^{st} Dicer cut site”).

FIG. 5 shows the second Dicer cleavage step and maturation into active siRNA. This second Dicer cleavage occurs in the cytoplasm of the host cell and removes the hairpin loop, leaving a functional siRNA for loading into the RISC complex. Again, the 2-nt overhang left by the first Dicer cleavage at the 3′ end of the RNA helps direct Dicer.

A timecourse experiment bacterial Class I RNase III cleavage of hairpin RNA resulting in a decrease from a 150 nt RNA to a 100 nt RNA. Single-stranded RNA containing a hairpin sequence was synthesized from a plasmid template using the MEGAshortscript Kit (Ambion). RNA was then exposed to purified bacterial RNase III for the indicated amounts of time, run on a 10% TBE-Urea gel, and visualized by ethidium bromide staining. The appearance of an approximately 100 nt RNA species appeared after 4 minutes of digestion.

### Example 26

### Construction of CEQ505

Drug candidate CEQ505 consists of an E. coli strain derived from MM294 through deletion of the dapA gene and the rnc gene. The internal designation of this E. Coli strain is CEQ221 transformed with the plasmid pNJSZc-H3, which is an expression plasmid encoding for the expression of invasin through the inv gene, listeriolysin 0 through the hly gene and short hairpin RNA to target the beta-catenin mRNA through the shRNA expression cassette including the hairpin sequence H3.

FACS analysis showed surface expression of Yersinia invasin is required by CEQ 200 Δrnc pNJSZc H3 for mammalian cell entry. Both Yersinia and CEQ 200 Δrnc pNJSZc H3 have surface expression of invasin.

LLO activity is required by CEQ200 Δrnc pNJSZc H3 for shRNA to escape mammalian cell endosome. LLO activity was detected by hemolysin assay, which demonstrated that CEQ505 has hemolysin activity whereas CEQ 221 without plasmid does not.

shRNA H3 is required to silence β-catenin in mammalian cells. Relative H3 hairpin expression was determined by PK, which showed that CEQ505 expressed H3 shRNA, while the untransformed strain CEQ221 does not.

FIG. 6 shows silencing of genes using CEQ 505. Panel A shows that CEQ 505 was able to silence mammalian β-catenin up to 90% in a dose-dependent manner in Cos-7 cells. Panel B shows that CEQ 221pNJSZc lamin (the equivalent strain targeting the lamin gene) was able to silence mammalian lamin up to 65% in a dose-dependent manner in SW480 cells.

### Example 26

### Modification of pMBV40, 43 and 44 to Produce Hairpins without 5′ or 3′ Tails

The original TRIP plasmid expressed shRNA under the control of the T7 RNA polymerase promoter, enhancer and terminator. In this format, transcription begins inside the T7 RNA polymerase promoter sequence. As a consequence, the T7 enhancer, BamHI site, SalI site and most of the terminator are transcribed. Whereas the shRNA hairpin is about 55 nt in length, the resultant transcript is predicted to be about 115 bases in length. The enhancer and restriction site BamHI used for cloning form a 5′ tail and the T7 RNA polymerase terminator form the 3′ tail.

Thus, new promoter-terminator constructs were designed for use in pMBV40, 43 and 44 (see Example 19) to make hairpins without 5′ or 3′ tails. The BamHI site used for cloning the hairpin was included in the promoter element shortly after the −10 consensus sequence (Lisser and Margalit, 1993, Nucleic Acids Res., 21, 1507-1516). The promoter was made stronger by including an UP element (Estrem et al, 1998, Proc. Natl. Acad. Sci. USA 95, 9761-9766; Meng et al., 2001, Nucleic Acids Res. 29, 4166-4178). For efficient termination, a run of Ts was added at the end of the hairpin prior to the SalI site used for cloning the hairpin (terminator I). Rho-independent terminators include an A-rich sequence followed by a stem loop of 4 to 18 by followed by a run of Ts (for example, Lesnik et al., 2001, Nucleic Acids Res. 29, 3583-3594). Since there is no A-rich sequence, the shRNA stem loop is 19-21 by long and since the gene is unusually small, efficiency of this terminator was hard to predict. Therefore, another rho-dependent terminator from the flagellin genes was also included (terminator II). Since there are two terminators, two transcripts were predicted. Transcripts I and II are terminated by terminator I and terminator II, respectively.

### Example 27

### Cloning of an Arabinose-Inducible Invasin Gene for Use in tkRNAi

In tkRNAi, intracellular delivery of therapeutic shRNA is achieved by equipping the carrier bacteria with invasive proteins that allow the bacteria to enter the host target cell through interaction with host cell surface receptors. The invasin protein encoded by the inv gene of Yersinia is one example of an invasive protein that triggers uptake of the bacteria into the host cell after interaction with host cell surface proteins called beta-1-integrins. However, high levels of invasin protein expression can be toxic to the bacterial carrier strains. Therefore, bacterial strains were constructed capable of inducible invasin expression through the addition of arabinose in the bacterial growth medium for the purpose of increasing efficacy and potency of tkRNAi-mediated gene silencing.

A plasmid was constructed having an arabinose-inducible invasin cassette containing the araC gene encoding the AraC protein, which is the Arabinose operon repressor-activator; the P_{araBAD}, arabinose promoter, which is under the regulation of the catabolite repressor protein (CRP) and the AraC protein; and the inv gene, which is cloned under the P_{araBAD} promoter.

There are different states of expression of invasin: (1) in the presence of glucose and absence of arabinose, the promoter is repressed by both catabolite repression and AraC-mediated repression; (2) an uninduced state occurs in the absence of any sugar (no glucose and no arabinose) as there is no catabolite repression and no AraC-mediated induction; and (3) an induced state occurs in the absence of glucose but in the presence of arabinose as there is AraC-mediated induction but no catabolite repression.

These states were tested for invasin according to the following protocol. The cells were grown overnight in the presence of glucose and then diluted and grown for 4 hours in the presence of glucose (repressed) or absence of any sugar (two cultures). One of the cultures grown in the absence of arabinose was induced with arbinose (10 mM) for 2 hr. The cells were harvested by centrifugation and the expression of invasin was measured by FACS. E. coli cells lacking any plasmid were used as a negative control and Yersinia grown at 26° C. was used as a positive control.

In the presence of arabinose, high level expression of invasin was found, as determined by FACS, to be comparable with the levels of invasin expression observed in the positive control (Yersinia). During the two-hour induction of invasin with arabinose, bacterial growth appeared to stop as measured by OD. There was also a 100-fold loss in viability within two hours consistent with our initial hypothesis. We then optimized the assay conditions to give good invasin expression as well as good viability. We found that 0.3 to 1 mM arabinose causes no detectable loss in viability although there is a measurable decrease in growth rate. These conditions also showed induction of invasin indistinguishable from that of 10 mM arabinose by FACS. The results demonstrate that bacterial growth (as determined by OD) is a function of arabinose-induction of invasion.

### Example 28

### Alternative shRNA Structures for tkRNAi

Transkingdom RNA interference (tkRNAi) uses vector bacteria to synthesize and deliver short hairpin RNA (shRNA), which are deposited into the cytoplasm of the target cell. To achieve this, bacteria are equipped with expression plasmids or chromosomal integrations that allow them to express at least three novel properties, a surface-expressed invasion marker (e.g. Yersinia invasin protein encoded by the inv gene), an endosomal release function (e.g. Listeriolysin 0 protein—LLO, encoded by the hly gene) and the therapeutic payload-a shRNA that triggers RNA interference once it it delivered into the host cell cytoplasm. Experiments have shown that the expression of large amounts of hairpin RNA presents a burden on the bacteria and leads slower growth and/or plasmid or hairpin modification by the bacteria. shRNA designs having higher structural energy make it harder for the bacterial transcription machinery to separate the two strands, however, such shRNAs are more difficult to clone than those with lower structural energy. In this example, we disclose a method of expressing alternative hairpin RNA with lower energy coefficients to allow for easier maintenance of the shRNA expressing plasmid inside the bacteria while maintaining the ability to induce gene silencing through RNA interference.

The advantage of the design shown in FIG. 7 over the existing shRNA structure primarily lies in significant lower structural energy, allowing for ease of cloning the tkRNAi plasmids, more stable maintenance of the plasmid within the bacteria, and facilitated sequencing of the plasmids. Introduction of the wobbles into the 3′ end of the sense strand [3′(S) wobbles] are tolerated and do not change the silencing ability of the construct, whereas introduction of 5′(S) wobbles might reduce silencing ability. RNAi can be triggered through shRNA that does not have the traditional double strand structure. Additionally, half-overlapping structures can induce gene silencing as long as the antisense (AS) strand is of full length (19 nt) and is covered on the 5′ end with a sense strand.

### Example 29

### Derepression of inv Expression in TRIP and pNJSZc Plasmids

The Yersinia pseudotuberculosis surface-expressed invasin protein mediates entry into human cells by binding to members of the beta-1 integrin family. For this reason, we have cloned the invasin gene (inv), complete with its native promoter, into pTRIP and pNJSZc plasmids to mediate internalization of E. coli into human intestinal epithelial cells. Expression of invasin in Y. pseudotuberculosis is repressed when H-NS (a histon-like protein) binds to the inv promoter region in complex with YmoA. Together, the two proteins form a repressive complex that reduces the expression of inv to basal levels. Up regulation of inv expression occurs when temperature-regulated RovA (Regulatory of virulence A) binds within the same promoter region of inv, displacing H—NS/YmoA. Homologues of H-NS and YmoA are present in E. coli. However, RovA is not present in E. coli, which results in constant basal level expression of invasin. It was reported that removing the regulatory binding region within inv's promoter region results in the constant up regulation of inv. In this example, we describe a method to remove the regulatory binding region from the inv promoter, as cloned in pTRIP and pNJSZc, to allow for constant up regulation of inv.

The primers shown in Table 46 were designed to delete 153 nucleotides located within Y. pseudotuberculosis inv promoter region, believed to be associated with the repression of inv in E. coli. The primers were used in combination with pTRIP (template) and QuikChange Lightning Site-Directed Mutagenesis kit (Stratagen) to delete the regulatory binding region showin in Table 46 within the inv promoter region cloned in pTRIP containing either the H3 or lamin hairpin.

TABLE 46

| | | SEQ ID |
|---|---|---|
| Description | Sequence | NO: |
| Forward Primer | CTGAAAGTAGAAATAAAAACCGCAGCATA | 567 |
| | TTTTGAATATTTTTAATCTGCCCAG | |
| Forward Primer | CTGGGCAGATTAAAAATATTCAAAATATG | 568 |
| | CTGCGGTTTTTATTTCTACTTTCAG | |
| Regulatory | GACAACTGACAACATCAAATGAAGGGGGC | 569 |
| Binding Region | TTATTCTAATTGATATTATTTATATGATA | |
| | ATAGTTCATTTTGTATTTTGTTTTTTTGA | |
| | TATTCTCACCTGCTTAGTTACAATAAATC | |
| | AATTCTATCGCTGTATGGTATAGACTGTT | |
| | TTATTATA | |

The resulting inv-derepressed gene was sequenced verified and cloned into pNJSZc within the NruI and Scat sites.

Invasin expression was tested by FACS where CEQ221/pTRIP and CEQ221/pNJSZc were grown overnight at 37° C., washed with PBS and probed with the anti-inv monoclonal antibody 3A2. The positive control was Y. pseudotuberculosis grown at 26° C. for optimal expression of invasin.

FACS analysis showed that removing the inv regulatory region resulted in an increase of invasion expression in E. coli transformed with both pTRIP (de-repressed mutant is called pGB60) and pNJSZc (de-repressed mutant is called pGB69). The original and de-repressed plasmids were tested for their abilities to induce internalization of E. coli within Vero cells by the standard Gentamycin protection assay. The data showed that de-repression of inv resulted in an increase of internalization of E. coli within Vero cells.

### Example 30

### Opa52 Mediated Invasion of E. coli into T84 Human Intestinal Epithelial Cells

Opa52 was engineered for use in apical, broad range targeting of intestinal epithelial cells to deliver tkRNAi. Opa52 will bind to CEACAMs 1, 3, 5 and 6 of which CEACAM1, -5 and -6 are expressed by epithelial cells. This will allow delivery of tkRNAi into healthy and polarized epithelial cells. The following example describes the construction and use of an Opa52 bacterial expression vector for invasion of E. coli into highly polarized T84 human intestinal epithelial cells.

The opa52 gene sequence was obtained from GenBank (accession # Z18929) and modified to include a start codon, signal sequence and stop codon. The signal sequence is identical to the natural secretion signals of several other Opa proteins and was codon-optimized for expression in E. coli. This gene was then placed under control of a modified lacUV5 promoter that contains a second lacO site to enable enhanced transcriptional repression. The lambda t0 terminator sequence was included downstream of the opa52 stop codon. This entire DNA fragment was synthesized by Blue Heron Biotechnology (Bothell, Wash.), cloned on pUC19 and confirmed. In order to facilitate expression of Opa52 on a low copy, ColE1-compatible plasmid, we subcloned the entire synthetic cassette from Blue Heron into a modified version of pACYC177 called pJS15. This is a small (2 kb), low copy (10-12 copies per cell), Kanamycin resistant vector that is compatible with ColE1 plasmids. The resultant opa52 construct has been designated pJS34 is 1040 basepairs in length and is shown in Table 47. The sequence includes: restriction sites for KpnI (1-6), SpeI (101-106), NdeI (922-927), NotI (1023-1030), and PmeI (1033-1040); lacO sites (for Lad binding) (7-25 and 70-88); RBS (95-100); ptac −35 (32-37) and −10 (56-62); the leader sequence (109-177) and the lambda t0 terminator sequence (928-1022).

TABLE 47

Final sequence with lacUV5 promoter (SEQ ID NO: 570)
GGTACCTTGTGAGCGGATAACAATTCCAGGCTTTACACTTTATGCTTCCGGCTCGTATAATGTGTGGAATTGTGA
GCGGATAACAATTTCACACAGGAGGACTAGTCTatgaacccggcgccgaaaaaaccgtccctgctgttttcctcc
ctgctgttttcctccgcggcgcaggcggcaggtgaagaccatgggcgcggcccgtatgtgcaggcggatctggct
tacgcctacgagcacattacccgcgattatcccgatgcagccggtgcaaacaaaggcaaaataagcacggtaagc
gattatttcagaaacatccgtacgcattccatccaccccagggtgtcggtcggctacgacttcggcggctggcgc
atcgccgcggattatgcccgttacaggaaatggcacaacaataaatattccgtgaacataaaagagttggaaaga
aagaataataaaacttctggcggcgaccagcttaacataaaataccaaaagacggaacatcaggaaaacggcaca
ttccacgccgtttcttctctcggcttgtcaaccgtttacgatttcagagtcaacgataaattcaaaccctatatc
ggtgtgcgtgtcggctacggacacgtcagacacggtatcgattcgactaaaaaaacgaaaaatactcttaccgcc
taccatggtgctggcacaaaacctacgtattatgatgatatagattcgggaaaaaaccaaaaaaacacttatcgc
caaaaccgcagcagccgccgcttgggcttcggcgcgatggcgggcgtgggcatagacgtcgcgcccggcctgacc
ttggacgccggctaccgctaccactattggggacgcctggaaaacacccgcttcaaaacccacgaagcctcattg
ggcgtgcgctaccgcttcTGACATATGGACTCCTGTTGATAGATCCAGTAATGACCTCAGAACTCCATCTGGATT
TGTTCAGAACGCTCGGTTGCCGCCGGGCGTTTTTTATTGGTGAGAATGCGGCCGCTTGTTTAAAC

Table 48 shows the translated 270 amino acid sequence with a leader 23 amino acid peptide added. The leader peptide is amino acids 1-23.

TABLE 48
Translated Sequence (SEQ ID NO: 571)
MNPAPKKPSLLFSSLLFSSAAQAAGEDHGRGPYVQADLAYAYEHITRDYP
DAAGANKGKISTVSDYFRNIRTHSIHPRVSVGYDFGGWRIAADYARYRKW
HNNKYSVNIKELERKNNKTSGGDQLNIKYQKTEHQENGTFHAVSSLGLST
VYDFRVNDKFKPYIGVRVGYGHVRHGIDSTKKTKNTLTAYHGAGTKPTYY
DDIDSGKNQKNTYRQNRSSRRLGFGAMAGVGIDVAPGLTLDAGYRYHYWG
RLENTRFKTHEASLGVRYRF

Plating and culture of bacteria released from highly polarized T84 cells demonstrated significantly higher invasive ability of the opa-expressing E. Coli strain compared with the invasin-expessing E. Coli strain. FIG. 8 shows the data from a repeat experiment:

To allow targeting of healthy (non-dysplastic) epithelium through tkRNAi, we have developed 2 alternate invasion strategies both based on single proteins, from entero-invasive pathogens that target epithelial cell surface receptors.

The first is a member of the Opa family of proteins expressed by Neisseria species and, depending on the Opa variant used, differentially targets members of the carcinoembryonic antigen cell adhesion molecule (CEACAM) family expressed on the apical aspect of epithelial cells. Primary attachment is mediated by the pilus, followed by a more intimate interaction via the opacity (Opa) proteins present in the bacterial outer membrane. The Opa proteins recognize distinct receptors present on epithelial cells. Certain Opa proteins bind to the cell surface heparan sulfate proteoglycans (HSPG) syndecan-1 and -4, while other Opa proteins bind to members of the carcinoembryonic antigen (CEA) or CD66 family, recently renamed the CEACAM family. CEACAMs can be found on epithelial cells and neutrophils, two cell types that are targeted by neisserial strains during natural infection. The interaction between Opa proteins and the CEACAM family members is highly specific; i.e., each Opa variant demonstrates a particular tropism for only certain members of the CEACAM receptor family.

The second of these targeting proteins is derived from Listeria monocytogenes and is termed Internalin A or InlA and targets the E-cadherin protein component of the adherens junction. InlA, in conjunction with InlB, enable L. monocytogenes to invade a wide range of nonphagocytic cells in the susceptible host. InlA promotes entry of L. monocytogenes into intestinal epithelial cells by targeting the N-terminal domain of the E-cadherin, the dominant molecule in the adherens junctional complex. Recent work has shown that native InlA utilizes a temporal window during intestinal epithelial maturation and shedding at the villus tip to gain entry into the host. In other words, intestinal epithelial cells are produced by self-renewing stem-cell like cells located at the base of the villus crypts and progressively mature as they move from the crypts toward the villus tips. Once intestinal epithelial cells reach the villus tip they are shed in a normal turnover process or through injury-induced apoptosis. In either case, there is then a transient exposure of the adherens junction proteins (i.e., E-cadherin) that allows InlA binding and entry of Listeria into the cells. Furthermore, in pathologic conditions such as IBD, the integrity of the epithelial barrier is compromised not only at the site of a lesion but also in surrounding uninvolved areas. In this case, the compromised barrier will expose the adherens junctions and, thus, E-cadherin such that an InlA-expressing delivery strain could gain access to cells in the inflammatory foci as well as the surrounding epithelium. This is advantageous in that during active flares of inflammation the delivery strain would preferentially invade and silence the target of interest at the site of inflammation/compromised barrier while leaving areas of normal epithelium untouched. As such, any tkRNAi delivery platform relying on InlA for targeting will useful as a therapeutic agent for treatment during active inflammation.

Results have shown significantly increased invasive ability for E. Coli carrying the Opa expressing plasmids compared with the invasin expressing plasmids.

### Example 31

### CEQ508 for the Treatment of Disorders Mediated by Upregulation of Beta Catenin

The drug candidate CEQ508 consists of E. coli strain CEQ221 containing the pMBV43-H3 plasmid. Strain CEQ221 is derived from E. coli strain MM294 through sequential deletion of dapA and rnc genes using the bacteriophage lambda Red recombination system with the help of 5-strain gene-disruption set of Datsenko and Wanner, 2000 (Proc. Natl. Acad. Sci. USA 97, 6640) purchased from CGSC. The plasmid pMBV43 in this example encodes shRNA hairpin to target the beta-catenin mRNA through the shRNA expression cassette including the hairpin sequence “H3” (disclosed previously), Yersinia pseudotuberculosis invasin (coded by the inv gene) and Listeria monocytogenes Listeriolysin O (LLO) (coded by the hly gene). The pMBV43 plasmid is derived from pUC19 with the following alterations:
1. The hairpin cassette has a modified P_{lacUV5} promoter linked to an UP element and a set of two terminators.
2. Cloning of a fragment containing inv and hly genes from another pre-existing plasmid pKSII-inv-hly.
3. Replacement of amp as antibiotic resistance marker with kanamycin (kan).

The modified P_{lacUV5} promoter used in the pMBV43 plasmid in this example contains at least three important distinctions (as shown in Table 49) from P_{lacUV5} promoter used in the pNJSZ plasmid used previously:
1. −35 consensus element (shaded is mutated from TTTACA to TTGACA;
2. An UP element is added upstream of −35 element;
3. The lacO (lac operon operator) element from P_{lacUV5} is deleted and replaced with a BamHI site.

TABLE 49

| | | SEQ |
|---|---|---|
| Promoter | Sequence | ID NO: |
| P_{lacUV5} | CCAGGCTTTACACTTTATGCTTCCGGCTCGTA | 572 |
| | TAATGTGTGGAATTGTGAGCGGATAACAATTT | |
| | CACACAGGAAACAGAATTCTATG | |
| P_{pMBV43 or} | AAGCTTGGAAAATTTTTTTTAAAAAAGTCTTG | 573 |
| Modified | ACACTTTATGCTTCCGGCTCGTATAATGGAT | |
| P_{lacUV5} | CC | |

The P_{lacUV5} promoter is 87 basepairs in length and is shown in Table 49. The −35 and −10 consensus elements of the P_{lacUV5} promoter are basepairs 7-12 and 31-37, respectively. The lacO (lac operon operator) is shown as basepairs 43-64.

The modified P_{lacUV5} promoter is 65 basepairs in length and is shown in Table 49. The −35 and −10 consensus elements of the P_{lacUV5} promoter are basepairs 30-35 and 54-60, respectively. The UP element is shown as basepairs 7-26.

An additional distinction of the pMBV43 plasmid is that it has two sets of terminators:
1. Terminator 1: is a run of Ts that functions as a terminator when it immediately follows the shRNA hairpin.
2. Terminator 2 is the same as the E. coli rrnC terminator and has the following sequence:

| | |
|---|---|
| GATCCTTAGCGAAAGCTAAGGATTTTTTTT. | (SEQ ID NO: 574) |

Moreover, the pNJSZ plasmid produces a shRNA comprising a total length of about 131 to 135 bases, consisting of a 5′ overhang of about 8 bases, 51 base pairs of shRNA, and a 3′ overhang consisting of about 72 to 76 bases. In contrast, the pMBV43 plasmid does not produce a 5′ overhang, produces a significantly smaller 3′ overhang consisting of 2-5 bases, and produces 51 base pairs of shRNA with the total length of shRNA in the range of 53 to 70 bases. For proper functioning, the 3′ overhang requires at least 2 bases. Accordingly, the total length of the shRNA ranges from 53 to 70 nucleotides in length, preferably 53 to 65 nucleotides in length, more preferably, 53 to 58 nucleotides in length, and most preferably 53 to 55 nucleotides in length.

FACS analysis showed surface expression of Yersinia invasin is required by CEQ 221 pMBV43-H3 for mammalian cell entry. Both Yersinia and CEQ 221 pMBV43-H3 (CEQ508) have surface expression of invasion. CEQ221 without the pMBV43 plasmid shows no invasin expression. Negative control: Yersinia with no antibody.

Listeriolysin (LLO) activity is required by CEQ508 to allow escape of the therapeutic payload (shRNA) from the mammalian cell endosome after invasion. LLO activity is detected by the hemolytic assay depicted above. CEQ508 shows clear hemolytic activity whereas CEQ 221 without plasmid or PBS does not.

Quantitative real-time PCR of the relative H3 hairpin RNA expression demonstrated that CEQ508 contains approximately 20 times more H3 shRNA compared to its predecessor, CEQ505. As disclosed previously, CEQ505 consists of an E. coli strain derived from MM294 through deletion of the dapA gene and the rnc gene, resulting in the E. coli strain designated as CEQ221, subsequently transformed with the plasmid pNJSZc-H3 encoding for the expression of invasin through the inv gene, listeriolysin O through the hly gene and short hairpin RNA to target the beta-catenin mRNA through the shRNA expression cassette including the hairpin sequence H3. In contrast, as previously disclosed, CEQ508 consists of E. coli strain CEQ221 containing the pMBV43-H3 plasmid, which encodes Yersinia pseudotuberculosis invasin (coded by the inv gene), Listeria monocytogenes Listeriolysin O (LLO) (coded by the hly gene) and the shRNA hairpin sequence H3.

FIG. 9 shows silencing of genes using CEQ508 in human cells (SW480). Panel A shows that CEQ 508 was able to silence mammalian β-catenin mRNA by as much as 90% in a dose-dependent manner in SW480 cells. Controls were treated with CEQ221-pMBV43-lamin and CEQ221-pMBV43-luciferase. Panel B shows that CEQ508 was able to silence mammalian β-catenin protein by as much as 72% in a dose-dependent manner in SW480 cells. Controls were treated with CEQ221-pMBV43-luciferase. Similar experiments conducted in DLD-1 cells showed that CEQ508 was able to silence β-catenin mRNA by over 50%. Additional experiments showed β-catenin silencing in HeLa cells with CEQ508-H3. HeLa cells were cultured in standard DMEM (10% FBS, 1% Pen-Strep) at 37° C., and CEQ508-H3 was added to the culture when cells were 80% confluent. Controls were treated with E. coli bacterial strains of identical genetic background, but expressing hairpin RNA against human lamin (hlam) instead of β-catenin. Cells were treated with various multiplicities of infection (MOIs) ranging from 1:6.5 to 1:51 for 2 hours, followed by four washes. Fresh medium containing antibiotics tetracycline and ofloxacin was then added, and the cells were harvested at 48 hours after invasion. RNA was extracted using TRIZOL (Invitrogen, Carlsbad, Calif.), and gene expression analysis was performed using quantitative real-time PCR. Dose dependent silencing of β-catenin was observed in HeLa Cells treated with CEQ508-H3, but not in cells treated with control strain. At the highest MOI, β-catenin expression was reduced to 45% of baseline.

FIG. 10 shows reduction of β-catenin gene expression in SW480 cells, as seen at the protein level (via Western Blot), after a single treatment with CEQ508 that is not observed with any of the control strains. SW480 cells were cultured in standard DMEM (10% FBS, 1% Pen-Strep) at 37° C. CEQ508 was added when the cells were 70% confluent. Controls were treated with E. coli bacterial strains of identical genetic background, but expressing either: (a) hairpin RNA against luciferase (luc), (b) bacteria with invasive properties invasin and listeriolysin, but not expressing hairpin RNA, or (c) empty E. coli CEQ221 not carrying the pMBV43 plasmid (non-invasive). Cells were treated with various multiplicities of infection (MOIs) ranging from 1:50 to 1:150 for 2 hours, followed by four washes. Fresh medium containing antibiotics tetracycline and ofloxacin was added, and the cells were harvested at 48 h after invasion followed by whole protein extraction using standard protocols. Dose dependent silencing of β-catenin was observed in SW480 cells treated with CEQ508, but not in cells treated with control strains. At the highest MOI, β-catenin expression was reduced to nearly undetectable levels.

Moreover, a time course experiment was performed and showed as least 50%, preferably at least 60%, more preferably at least 70%, and most preferably at least 80% long-term silencing for at least 1 day, preferably at least 2 days, more preferably at least 3 days, and most preferably at least 4 day following administration, of β-catenin expression in SW480 cells following a single treatment with CEQ508. SW480 cells were cultured in standard DMEM (10% FBS, 1% Pen-Strep) at 37° C. CEQ508 was added when the cells were 70% confluent. Controls were treated with bacterial strains of identical genetic background (E. coli strain CEQ221) not carrying the pMBV43 plasmid (i.e., which do not carry the gene for invasion and are thus non-invasive). Cells were treated with MOI 1:200 for 2 hrs, followed by four washes. Fresh medium containing antibiotics tetracycline and ofloxacin was added, and the cells were allowed to grow and passaged when 90% confluence was reached. Cells from parallel wells were harvested at the indicated time points after invasion. RNA was extracted using TRIZOL, and gene expression analysis was performed using quantitative real-time PCR. The results show robust (>80%) gene silencing after a single treatment with CEQ508, which persists for at least four days before β-catenin levels slowly return to normal. An apparent “overshoot” around day 10 is interpreted to be an artifact caused by passaging of the cell lines.

Treatment with CEQ508 (CEQ221/pMBV43H3) at two different doses (low-10⁷/ml and high-10⁸/ml) did not lead to significant changes in the gene expression (as measured using quantitative real-time PCR) of lamin in human cells (SW480), confirming no deleterious effects on other genes. Control treatment was performed with CEQ221 bacteria without plasmid (no plasmid high) at 10⁸/ml, CEQ221-pMBV43-luciferase (pMBV43luc high and low) at 10⁷/ml and 10⁸/ml respectively, and with CEQ221-pMBV43 expressing no short hairpin RNA (pMBV43 no hairpin high/low) at 10⁸/ml and 10⁷/ml respectively. These data demonstrate that CEQ508 treatment does not cause problems for other genes.

Timecourse profiles were performed to evaluate the presence or absence of inflammatory cytokines from 0 h to 12 h after a single oral feeding with CEQ508 in wildtype mice and polyp-bearing APCmin mice. Animals (wild type as well as APCmin mice, 3-7 per group per time point) were treated with a single dose of CEQ508 and serum was taken at the indicated time points Animals used for positive control were i.v. injected with 400 μg LPS. The inflammatory cytokines TNFα, IL6, IL12, IFNγ, MCP-1, and IL-10 were analyzed as indicators of an inflammatory response to orally administered CEQ508. Overall, none of the tested inflammatory cytokines showed an increase after oral treatment with CEQ508, demonstrating that there is no systemic inflammatory cytokine response after CEQ508 treatment in either wild type or polyp-bearing APCmin mice.

Table 50 shows the pharmacokinetics of shRNA in gastrointestinal mucosa after oral feeding with CEQ508 or CEQ501.

TABLE 50

| | | | Time of | Amount of H3 |
|---|---|---|---|---|
| | Daily | No. of | sacrifice | shRNA (pg |
| Treatment | Dose | Treat- | after the last | per μg |
| Groups | (cfu) | ments | treatment | total RNA) |
| PBS + 15% | | 1 | 5 | n.d. |
| glycerol | | | | |
| CEQ508 | 5 × 10⁹ | 1 | 5 | 6.8 ± 8.9 |
| PBS | | 1 | 24 | n.d. |
| CEQ508 | 5 × 10⁹ | 1 | 24 | n.d. |
| CEQ508 | 5 × 10⁹ | 3 | 5 | 3.6 ± 2.6 |
| CEQ508 | 5 × 10⁹ | 3 | 24 | 1.2 ± 0.4 |
| CEQ501 | 5 × 10⁹ | 1 | 5 | 1.5 ± 0.2 |
| CEQ501 | 5 × 10⁹ | 1 | 24 | 1.5 ± 0.3 |
| CEQ501 | 5 × 10⁹ | 3 | 5 | 1.4 ± 0.3 |
| CEQ501 | 5 × 10⁹ | 3 | 24 | 1.1 ± 0.3 |

Mice were dosed with one or repeated treatment of CEQ508 or CEQ501 and gastrointestinal mucosal tissue was analyzed to quantify the amount of shRNA deposited within the GI mucosa at various time points after treatment. H3 shRNA was detectable in the intestinal mucosa of mice dosed with either CEQ501 or CEQ508 whereas PBS/glycerol treated mice were devoid of H3 shRNA. Furthermore, the amount of H3 shRNA detected in the mucosa was significantly higher after treatment with CEQ508 compared to CEQ501, which is consistent with the Δrnc mutation conferring higher yields and greater stability of the H3 shRNA. When assayed at 24 hours post last dose (multiple once daily regimen), both CEQ501 and CEQ508 show comparable levels of H3 shRNA in the intestinal mucosa suggesting that both delivery platforms achieve equivalent steady-state levels of delivered hairpin.

Table 51 shows the experimental groups for the evaluation of pharmacokinetics for live CEQ508 bacteria after oral treatment in mice.

TABLE 51

| | | | Analysis | | | |
|---|---|---|---|---|---|---|
| Daily | | | Point | | | Total No. of |
| Treatment | Route of | No. of | (hrs post last | | | animals |
| Dose (cfu) | Administration | Treatments | treatment) | Males | Females | Wt/APC^{min} |
| 5.0 × 10⁹ | PO | 1 | 24 hrs | 2 | 2 | 2/2 |
| 5.0 × 10⁹ | PO | 3 | 24 hrs | 2 | 2 | 2/2 |
| 5.0 × 10⁹ | PO | 7 | 24 hrs | 2 | 2 | 2/2 |
| 1.0 × 10⁷ | IV | 1 | 2 hr | 1 | 1 | 2 |
| 1.0 × 10⁷ | IV | 1 | 24 hrs | 1 | 1 | 2 |
| 1.0 × 10⁷ | IV | 1 | 48 hrs | 1 | 1 | 2 |
| 1.0 × 10⁷ | IV | 1 | 72 hrs | 1 | 1 | 2 |
| 1.0 × 10⁷ | IV | 1 | 96 hrs | 1 | 1 | 2 |

Mice were treated 1, 3 or 7 times by oral gavage with CEQ508. Each dose contained 5×10⁹ cfu of CEQ508. Tissues were analyzed 24 h after the last dosing. Tissues were extracted sterilely and examined for the presence of live therapeutic CEQ508 bacteira. Positive control animals were treated with intravenous injection of CEQ508.

Table 52 shows the pharmacokinetics demonstrating that after these single or multiple (once daily up to 7 days) oral treatments, viable CEQ508 was not recovered from any of the examined organs.

TABLE 52

| | | Analysis | | | | | | | | | | | |
|---|---|---|---|---|---|---|---|---|---|---|---|---|---|
| | | point (hrs | | | | | | | | | | | |
| | NO. of | post last | Dose | | | Blood | | Liver | | Spleen | | Kidney | |
| Route | Treatments | treatments) | (cfu) | Mouse # | Gender | DAP | Kan/DAP | DAP | Kan/DAP | DAP | Kan/DAP | DAP | Kan/DAP |
| IV | 1 | 2 | 1 × 10⁷ | 2357 | F | 58 | 69 | >1000 | >1000 | >1000 | >1000 | 0 | 0 |
| | | | | 2299 | M | 23 | 17 | >1000 | >1000 | >1000 | >1000 | 0 | 0 |
| | 1 | 24 | 1 × 10⁷ | 2481 | F | 0 | 0 | 14 | 15 | 41 | 40 | 0 | 0 |
| | | | | 2478 | M | 0 | 0 | 57 | 47 | 38 | 41 | 0 | 0 |
| | 1 | 48 | 1 × 10⁷ | 2442 | F | 0 | 0 | 2 | 5 | 4 | 6 | 2 | 0 |
| | | | | 2448 | M | 0 | 0 | 1 | 1 | 7 | 1 | 0 | 0 |
| | 1 | 72 | 1 × 10⁷ | 2443 | F | 0 | 0 | 2 | 0 | 2 | 0 | 2 | 0 |
| | | | | 2449 | M | 1 | 0 | 3 | 1 | 5 | 0 | 4 | 4 |
| | 1 | 96 | 1 × 10⁷ | 2368 | F | 0 | 0 | 1 | 0 | 0 | 2 | 0 | 0 |
| | | | | 2450 | M | 10 | 0 | 15 | 0 | 37 | 1 | 4 | 0 |
| PO | 1 | 24 | 5 × 10⁹ | 2192 | F wt | 0 | 0 | 0 | 0 | 0 | 0 | 0 | 0 |
| | | | | 2295 | F min | 0 | 0 | 0 | 0 | 0 | 0 | 2 | 0 |
| | | | | 2296 | M wt | 0 | 0 | 0 | 0 | 0 | 0 | 0 | 0 |
| | | | | 2139 | M min | 0 | 0 | 0 | 0 | 0 | 0 | 0 | 0 |
| | 3 | 24 | 5 × 10⁹ | 2302 | F wt | 0 | 0 | 0 | 0 | 0 | 0 | 0 | 0 |
| | | | | 2193 | F min | 0 | 0 | 0 | 0 | 0 | 0 | 0 | 0 |
| | | | | 2297 | M wt | 0 | 0 | 0 | 0 | 0 | 0 | 0 | 0 |
| | | | | 2138 | M min | 0 | 0 | 0 | 0 | 0 | 0 | 0 | 0 |
| | 7 | 24 | 5 × 10⁹ | 2311 | F wt | 0 | 0 | 0 | 0 | 0 | 0 | 0 | 0 |
| | | | | 2483 | F min | 57 | 2 | 0 | 0 | 0 | 0 | 0 | 0 |
| | | | | 2298 | M wt | 0 | 0 | 0 | 0 | 0 | 0 | 0 | 0 |
| | | | | 2141 | M min | 0 | 0 | 0 | 0 | 0 | 0 | 0 | 0 |

| | | Analysis | | | | | | | | | |
|---|---|---|---|---|---|---|---|---|---|---|---|
| | | point (hrs | | | | | | | | | |
| | NO. of | post last | Dose | | | Lung | | Heart | | Brain | |
| Route | Treatments | treatments) | (cfu) | Mouse # | Gender | DAP | Kan/DAP | DAP | Kan/DAP | DAP | Kan/DAP |
| IV | 1 | 2 | 1 × 10⁷ | 2357 | F | 280 | 260 | 40 | 39 | 0 | 2 |
| | | | | 2299 | M | 200 | 160 | 27 | 51 | 0 | 0 |
| | 1 | 24 | 1 × 10⁷ | 2481 | F | 75 | 108 | 6 | 1 | 0 | 0 |
| | | | | 2478 | M | 81 | 90 | 10 | 6 | 0 | 0 |
| | 1 | 48 | 1 × 10⁷ | 2442 | F | 47 | 31 | 6 | 7 | 0 | 0 |
| | | | | 2448 | M | 56 | 62 | 6 | 7 | 0 | 0 |
| | 1 | 72 | 1 × 10⁷ | 2443 | F | 6 | 6 | 5 | 4 | 0 | 0 |
| | | | | 2449 | M | 65 | 82 | 5 | 4 | 0 | 0 |
| | 1 | 96 | 1 × 10⁷ | 2368 | F | 10 | 14 | 2 | 1 | 0 | 0 |
| | | | | 2450 | M | 15 | 15 | 0 | 1 | 0 | 0 |
| PO | 1 | 24 | 5 × 10⁹ | 2192 | F wt | 0 | 0 | 0 | 0 | 0 | 0 |
| | | | | 2295 | F min | 0 | 0 | 0 | 0 | 0 | 0 |
| | | | | 2296 | M wt | 50* | 35* | 0 | 0 | 0 | 0 |
| | | | | 2139 | M min | 0 | 0 | 0 | 0 | 0 | 0 |
| | 3 | 24 | 5 × 10⁹ | 2302 | F wt | 0 | 0 | 0 | 0 | 0 | 0 |
| | | | | 2193 | F min | 0 | 0 | 0 | 0 | 2 | 0 |
| | | | | 2297 | M wt | 0 | 0 | 0 | 0 | 0 | 0 |
| | | | | 2138 | M min | 0 | 0 | 0 | 0 | 0 | 0 |
| | 7 | 24 | 5 × 10⁹ | 2311 | F wt | 0 | 0 | 0 | 0 | 0 | 0 |
| | | | | 2483 | F min | 0 | 0 | 0 | 0 | 0 | 0 |
| | | | | 2298 | M wt | 0 | 1 | 0 | 0 | 0 | 0 |
| | | | | 2141 | M min | 0 | 0 | 0 | 0 | 0 | 0 |

These results confirm that CEQ508 is incapable of escaping the gastrointestinal tract in mice (with the exception of two animals showing false positive bacteria after gavage injuries). This was observed for both wild type mice (normal, healthy mice with an intact gut barrier) as well as in APC^{min} mice that have compromised epithelial barrier integrity due to dysplasia. CEQ508 was, however, recovered from stool samples taken five hours post dose, confirming transit of viable bacteria throughout the length of the intestine. As expected, the number of viable CEQ508 recovered in the stool rapidly diminished by 24 hours post dose. Viable CEQ508 was recovered from mice given a single intravenous injection via the tail vein. In these animals, CEQ508 was recovered in the blood and organs examined at two hours post injection. The number of viable bacterial subsequently declined but was recoverable in the liver for up to 96 hours post dose (iv).

Stool samples were collected from animals after receiving a single oral dose of CEQ508; 5.0×109 cfu via gavage in the total volume of 0.2 mL. Stool samples were collected hourly for the first six hours and then every two hours up to 24 hrs, and then every 12 hrs up to 108 hrs. To be able to collect stool samples according to the schedule total number of 24, the mice were subdivided into two cohorts of 12 mice each and received a single oral dose of CEQ508 with 12 hrs shift. Stool samples were resuspended in 1 mL of sterile PBS, diluted up to 10 mL with sterile PBS and 50 μL of this suspension was subsequently plated on nonselective LB/DAP plates, where all bacteria from the stool sample is expected to grow, as well as on selective LB/Kan/DAP containing plates, where only therapeutic CEQ508 bacteria from the stool samples is expected to grow. Bacterial colonies were then counted. In accordance with the abundance of CEQ508 in stool samples, mice were subdivided into the following groups: 0 CFUs, <100 CFUs, <1000 CFUs, >1000 CFUs. Finally, the percentage of mice having a certain defined amount of bacteria was calculated, as illustrated in Table 53.

TABLE 53

| | % of mice with determined CFUs of CEQ508 | | | |
|---|---|---|---|---|
| Time | in stool samples | | | |
| points | 0 CFUs | <100 CFUs | <1000 CFUs | >1000 CFUs |
| 0 | 100.0% | 0.0% | 0.0% | 0.0% |
| 1 | 100.0% | 0.0% | 0.0% | 0.0% |
| 2 | 41.7% | 25.0% | 0.0% | 33.3% |
| 3 | 8.3% | 16.7% | 8.3% | 66.7% |
| 4 | 0.0% | 0.0% | 16.7% | 83.3% |
| 5 | 0.0% | 0.0% | 0.0% | 100.0% |
| 6 | 0.0% | 0.0% | 0.0% | 100.0% |
| 8 | 0.0% | 0.0% | 0.0% | 100.0% |
| 10 | 0.0% | 0.0% | 16.7% | 83.3% |
| 12 | 0.0% | 0.0% | 16.7% | 83.3% |
| 14 | 0.0% | 0.0% | 75.0% | 25.0% |
| 16 | 0.0% | 25.0% | 33.3% | 41.7% |
| 18 | 0.0% | 8.3% | 66.7% | 25.0% |
| 20 | 0.0% | 41.7% | 58.3% | 0.0% |
| 22 | 0.0% | 58.3% | 41.7% | 0.0% |
| 24 | 4.2% | 62.5% | 33.3% | 0.0% |
| 36 | 66.7% | 33.3% | 0.0% | 0.0% |
| 48 | 100.0% | 0.0% | 0.0% | 0.0% |
| 60 | 100.0% | 0.0% | 0.0% | 0.0% |
| 72 | 100.0% | 0.0% | 0.0% | 0.0% |
| 84 | 100.0% | 0.0% | 0.0% | 0.0% |
| 96 | 100.0% | 0.0% | 0.0% | 0.0% |
| 108 | 100.0% | 0.0% | 0.0% | 0.0% |

Table 53 shows that after a single oral treatment with CEQ508, mice begin to shed viable bacteria as early as two hours. The amount of CEQ508 in stool samples peaks by 5 hrs (i.e. all mice shed >1000 CFUs of viable CEQ508), remains elevated for up to 8 hours post dose and gradually declines thereafter. At 24 hours post dose, the majority of treated mice shed only low numbers of CEQ508 (i.e. 33%<1000 CFUs and 63%<100 CFUs). Only one third of the total number of treated mice (33.3%) continue to shed viable CEQ508 (<100 CFUs) in stool samples 36 hrs after the treatment while none of the animals shed viable CEQ508 at 48, 60, 72, 84, 96 or 108 hrs after the treatment. Taken together, these data demonstrate that CEQ508 not only transits the gastrointestinal tract intact but is also rapidly eliminated and is incapable of residing and proliferating in the gut. CEQ508 peaks by 5 h post dosing in stool samples, remains elevated for up to 8 hours post dose and gradually declines thereafter, while none of the animals shed viable CEQ508 at 48, 60, 72, 84, 96 or 108 hrs post dose.

### Example 32

### CEQ509 for the Treatment of Disorders Mediated by the Upregulation of Beta Catenin

The drug candidate CEQ509 consists of Bacterial Therapeutic Particles (BTPs), which are minicells derived from E. coli strain CEQ210 containing the pNJSZc plasmid. Strain CEQ210 is derived from E. coli strain MM294 through deletion of minC gene using the bacteriophage lambda Red recombination system with the help of 5-strain gene-disruption set of Datsenko and Wanner, 2000 (Proc. Natl. Acad. Sci. USA 97, 6640) purchased from CGSC. The pNJSZc plasmid encodes shRNA hairpin, Yersinia pseudotuberculosis invasin encoded by the inv gene, and Listeria monocytogenes Listeriolysin O (LLO), encoded by the hly gene. The BTPs were purified by low speed centrifugation yielding >99.9% purity based on their ability to form colonies.

A number of assays have been performed to assess the tkRNAi activity of CEQ509, including an assay for LLO protein encoded by the hly gene of the pNJSZc plasmid. CEQ509 BTPs demonstrate as much LLO activity as CEQ501 when an equal amount of BTPs (equal biomass) were used.

Listeria monocytogenes Listeriolysin O (LLO) activity is required by CEQ509 to escape the phagosome and release the hairpin in the cytoplasm. LLO activity was assayed by hemolysis at pH5.5. Hemolysis was observed visually and quantified by measuring absorbence at 540 nm. For the measurement of absorbance, the spectrophotometer was blanked using the PBS-treated samples. CEQ509-H3 and CEQ509-HPV are BTPs that contain the H3 shRNA against β-catenin and HPV E6 protein (used here as a control).

FACS analysis demonstred the presence of invasin on the surface of CEQ509 BTPs.

Surface expression of Yersinia pseudotuberculosis invasin by CEQ509 is required for mammalian cell entry by CEQ509 BTPs.

Quantitative real-time PCR (qPCR) analysis demonstrated that CEQ509 BTPs do not contain any H3 shRNA and generate only a background signal.

Finally, the BTPs were tested in a tkRNAi assay as illustrated in FIG. 11. FIG. 11 shows silencing of beta catenin using CEQ509. COS-7 cells were infected by CEQ509-H3 or CEQ509-HPV BTPs at the indicated multiplicity of infection (MOI). RNA from the cells was harvested after 48 hr and subjected to qPCR-based quantitation of β-catenin mRNA. The ratio of relative quantitation (RQ) of cells infected with CEQ509-H3 to that of CEQ509-HPV at the corresponding MOI is plotted above. This and other data show 30-50% silencing of β-catenin by CEQ509. The experiments demonstrate that the level of invasin expression was sufficient to induce tkRNAi mediated gene silencing.

What is claimed is:

1. An invasive E. coli bacterium comprising a prokaryotic vector, said vector comprising one or more DNA molecules encoding one or more siRNAs, a modified P_{lacUV5} promoter, at least one Inv locus and at least one HlyA gene, wherein said siRNAs interfere with the mRNA of β-catenin and wherein said invasive E. coli bacterium having reduced RNase III activity when compared to wild-type E. coli bacterium.

2. A prokaryotic vector comprising one or more DNA molecules encoding one or more siRNAs, a modified P_{lacUV5} promoter, at least one Inv locus and at least one HlyA gene, wherein said siRNAs interfere with the mRNA of β-catenin.

3. A method of delivering one or more siRNAs to mammalian cells, the method comprising introducing to said mammalian cells at least one invasive E. coli bacterium of claim 1.

4. A method of regulating gene expression in mammalian cells, the method comprising introducing to said mammalian cells at least one invasive E. coli bacterium of claim 1.

5. A method of treating or preventing a disease or disorder associated with the over expression of β-catenin in a mammal in need thereof, the method comprising regulating the expression of f3-catenin in said mammal comprising introducing to the cells of said mammal at least one invasive E. coli bacterium of claim 1.

6. The invasive E. coli bacterium of claim 1, wherein said bacterium comprises a deletion of an rnc gene encoding RNase III.

7. The invasive E. coli bacterium of claim 1, wherein said RNase III activity is reduced at least 90% when compared to wild-type E. coli bacterium.

8. The invasive E. coli bacterium of claim 1, wherein said RNase III activity is reduced at least 95% when compared to wild-type E. coli bacterium.

9. The invasive E. coli bacterium of claim 1, wherein said RNase III activity is reduced at least 99% when compared to wild-type E. coli bacterium.

10. The invasive E. coli bacterium of claim 1, wherein said one or more DNA molecules are transcribed into one or more shRNAs within the invasive bacterium.

11. The invasive E. coli bacterium of claim 10, wherein said one or more shRNAs comprise a 3′ overhang or a blunt end.

12. The invasive E. coli bacterium of claim 11, wherein said 3′ overhang is 2-5 base pairs.

13. The invasive E. coli bacterium of claim 11, wherein said 3′ overhang is no more than 2 base pairs.

14. The invasive E. coli bacterium of claim 10, wherein said one or more shRNAs are processed into one or more siRNAs.

15. The invasive E. coli bacterium of claim 1, wherein said prokaryotic vector further comprises at least one terminator sequence.

16. The invasive E. coli bacterium of claim 15, wherein said at least one terminator sequence comprises at least 5 consecutive thymidine base pairs.

17. The invasive E. coli bacterium of claim 15, wherein said bacterium further comprises a second terminator sequence.

18. The invasive E. coli bacterium of claim 17, wherein said second terminator sequence is an rrnC terminator sequence.

19. The invasive E. coli bacterium of claim 1, wherein said prokaryotic promoter further comprises at least one UP element.

20. The invasive bacterium of claim 1, wherein said invasive bacterium is an attenuated, non-pathogenic or non-virulent bacterium.

21. A composition comprising the invasive bacterium of claim 1 and a pharmaceutically acceptable carrier.

22. The method of claim 5, wherein said mammalian cells are infected with about 10³ to 10¹¹ invasive bacteria.

23. The method of claim 22, wherein said mammalian cells are infected with about 10⁵ to 10⁹ invasive bacteria.

24. The method of claim 5, wherein said mammalian cells are infected at a multiplicity of infection ranging from about 0.1 to 10⁶.

25. The method of claim 25, wherein said mammalian cells are infected at a multiplicity of infection ranging from about 10² to 10⁴.

26. The method of claim 5, wherein said expression of β-catenin is reduced as compared to wild-type β-catenin expression or as compared to β-catenin expression prior to introducing said invasive bacterium to said cell.

27. The method of claim 26, wherein said reduced expression of β-catenin is reduced expression of β-catenin mRNA.

28. The method of claim 26, wherein said reduced expression of β-catenin is reduced expression of β-catenin protein.

29. The method of claim 26, wherein said expression of β-catenin is reduced at least 50% as compared to wild-type β-catenin expression or as compared to β-catenin expression prior to introducing said invasive bacterium to said cell.

30. The method of claim 26, wherein said expression of β-catenin is reduced at least 75% as compared to wild-type β-catenin expression or as compared to β-catenin expression prior to introducing said invasive bacterium to said cell.

31. The method of claim 26, wherein said expression of β-catenin is reduced at least 90% as compared to wild-type β-catenin expression or as compared to β-catenin expression prior to introducing said invasive bacterium to said cell.

32. The method of claim 5, wherein the disease or disorder associated with the over expression of β-catenin in a mammal is selected from the group consisting of colon cancer, rectal cancer, colorectal cancer, Crohn's disease, ulcerative colitis, familial adenomatous polyposis (FAP), Gardner's syndrome, hepatocellular carcinoma (HCC), basal cell carcinoma, pilomatricoma, medulloblastoma, and ovarian cancer.

33. The method of claim 5, wherein the mammalian cells are selected from the group consisting of a colon epithelial cell, a rectal epithelial cell, an intestinal epithelial cell, a hepatocyte, a skin epithelial cell, a hair cell, a neural cell, and an ovarian cell.

34. The method of claim 5, wherein said mammalian is selected from the group consisting of human, bovine, ovine, porcine, feline, buffalo, canine, goat, equine, donkey, deer, and primate.

35. The method of claim 34, wherein said mammal is a human.
